(19) United States
(12) Patent Application Publication
(10) Pub. No.: US 2024/0238409 A1
(43) Pub. Date: Jul. 18, 2024

(54) SARS-COV-2 MULTI-EPITOPE VACCINES

(71) Applicants: THE UNIVERSITY OF BRITISH COLUMBIA, Vancouver (CA); THE JAMES HUTTON INSTITUTE, Dundee (GB)

(72) Inventors: Wilfred JEFFERIES, Surrey (CA); Paolo RIBECA, Edinburgh, Midlothian (GB)

(21) Appl. No.: 18/558,241

(22) PCT Filed: May 5, 2022
(86) PCT No.: PCT/CA2022/050705

§ 371 Date: Oct. 31, 2023

Related U.S. Application Data
(60) Provisional application No. 63/184,547, filed on May 5, 2021.

(51) Int. Cl. A61K 39/215; A61K 39/00; C12N 7/00; C12N 15/86

(52) U.S. Cl. CPC A61K 39/215; C12N 7/00; C12N 15/86; A61K 2039/53; A61K 2039/545; A61K 2039/55555; C12N 2710/10041; C12N 2710/24043; C12N 2760/20241; C12N 2770/20034

(57) ABSTRACT

The present invention provides multi-epitope vaccines comprising or capable of expressing one or more concatemers of epitopes from a viral pathogen, namely, SARS-COV-2. wherein at least a portion of the epitopes are from conserved viral proteins and wherein the vaccine comprises or expresses epitopes for all MHC I and MHC II alleles with a frequency >1% in the target population.

### FIELD OF THE INVENTION

This invention pertains generally to vaccines and, more particularly multi-epitope vaccines for viral pathogens including SARS-COV-2, the causative agent of COVID-19.

### BACKGROUND OF THE INVENTION

An outbreak of pneumonia like disease termed COVID-19 caused by a novel coronavirus, SARS-COV-2, has spread across the world and become a global pandemic. First generation vaccines targeting SARS-COV-2 have been developed by BioNTech/Pfizer, Moderna, Oxford/Astra Zeneca and others. These first generation vaccines all target spike protein: the Oxford/Astra Zeneca vaccine uses an adenoviral vector; the vaccines by Moderna and Pfizer are RNA based; the vaccine by Imperial College London relies upon self-amplifying RNA.

The spike protein is a poor candidate choice for a vaccine. Spike is hypervariable and prone to mutations. A number of SARS-COV-2 variants have been identified, and most of the sequence variant characterising them are usually located within Spike. Moreover, a low number of antibodies from survivors (10-30%) target spike protein. In addition, vaccines targeting spike protein will not immunise all individuals due to HLA variability.

To work, a vaccine must contain pathogen-derived molecules (typically proteins or glycans) processed first by the host cell into peptides and then presented (antigen presentation) at the host cell surface on its Human Leukocyte Antigens (HLA). HLAs are encoded within the highly polymorphic major histocompatibility complex (MHC) on Chromosome 6. This process leads to presentation of peptides originating from self and pathogen. HLA-peptide complexes are then specifically recognized by T-cells via the T-cell receptor (TCR); while self-peptide ligands do not typically elicit a response from the immune system, an immunogenic foreign peptide ligand-HLA complex will bind a TCR and trigger an immune response that leads to the development of cytotoxic and memory T- and B-cells. There are many components in the host immune system, but HLA molecules are the chief determinants of antigen presentation to the T-cells for subsequent activation of the immune response.

The classical HLA genes are the most polymorphic genes in the human genome, with some having more than a thousand known alleles. Alleles are distributed unevenly around the world but typically clustered according to ethnicity. These genes are divided into two subgroups, mainly based on the source of the peptides they tend to present: HLA I genes are expressed on all cells except red blood cells, and present peptides of intracellular origin (e.g. from self or viruses), whereas HLA-II genes are expressed only on professional antigen presenting cells and present peptides that originate extracellularly (e.g. from bacteria). Thus, the response to a viral pathogen such as SARS-COV-2 is mediated by HLA-I.

The HLA genes are co-dominantly expressed and encode HLA proteins that are referred to as HLA Class I (HLA-A, -B, -C) and HLA Class II (HLA-D). They are critical in priming adaptive immune responses. CD4+T helper/inducer cells recognize viral peptides bound to HLA-II encoded proteins and CD8+ Cytotoxic T cells recognize viral peptides bound to HLA-A, -B, -C, encoded proteins. The vast polymorphism in their extracellular peptide binding domains leads to the diversity of peptide antigens that can be bound and subsequently recognized by T-cell receptors. HLA class I molecules generally present short (8-12 amino acids) intracellularly-derived peptides, such as viral antigens. HLA class II molecules are capable of presenting longer (i.e., generally more than 13 amino acids) extracellularly derived peptides, such as antigenic fragments generated from viral proteins. The HLA allele polymorphism renders each variant protein a distinct product with the main difference focused on the peptide-binding groove and the conformation of adjacent regions directly engaged with peptide binding and interaction with the TCR. A T-cell will recognize bound antigen as a complex with a restricted allelic variant of HLA molecule. Depending on the combinations of HLA-I and II alleles expressed, an individual may be differently equipped to resist certain viruses, including coronaviruses. Thus, individual genetic variation across HLA genes aids in understanding how variation in HLA may affect the course of COVID-19, and could help identify individuals at higher risk of succumbing to the disease.

Previous research on related virus strains (SARS-COV-1 and MERS-Cov) demonstrated that viral antigen presentation of SARS-COV-1 mainly depends on HLA-I and HLA-II molecules. Numerous HLA-I polymorphisms correlate to susceptibility of SARS-COV-1, such as HLA-B*46:01 5, HLA-B*07:03, HLA-DR B1*12:02, and HLA-Cw*08:01, whereas the HLA-DR*03:01, HLA-Cw15:02 and HLA-A*02:01 alleles are related to the protection from SARS-COV-1 infection HLA-II molecules, such as HLA-DRB1*11:01 and HLA-DQB1*02, are associated with the susceptibility to MERS-COV infection. These data suggest that individual HLA genotypes may differentially control susceptibility or protection in T-cell mediated anti-SARS-COV-2 responses. Thus, HLA polymorphism could potentially alter disease outcomes and SARS-COV-2 transmission. The enormous diversity in HLA genes means that some individuals can present an antigen and mount a strong immune response against it, while others cannot present it at all. This is especially relevant for vaccination strategies involving subunit vaccines, since the number of available antigens can be very small. In fact, HLA polymorphism is a likely basis for the observed variations in vaccine efficacy.

Epitope-based or string of beads vaccines use concatemers of short immunogenic peptide sequences derived from antigens that are recognised by either CD4 or CD8 T-cells in the context of HLA-II or HLA-I respectively. They have several advantages over whole attenuated or subunit vaccines because they do not contain potentially infectious material. Furthermore, peptides can be chosen to take the genetic variation of pathogens and HLA-binding specificities into account. Development of such vaccines requires bioinformatics for prediction of HLA epitopes. Machine-learning methods, such as probabilistic models, neural networks, and support vectors machines, are routinely used with high accuracy for epitope prediction. Different algorithms have been used to create string of bead vaccines that generally concentrate on binding peptides for a small number of HLA-I epitopes.

A variety of platforms, including but not limited to nucleic acid, viral vectors, attenuated viruses and recombinant protein, are being examined for the development of the SARS-COV-2 vaccine. The use of nucleic acid-based vaccines allow for vaccines to be obtained in a short timeframe. Furthermore, nucleic acid-based vaccine manufacturing is safe and time-saving, and bypasses the need to grow highly pathogenic organisms at a large scale, resulting in a lower risk of contamination with live infectious reagents and release of dangerous pathogens.

The SAM vaccine platform is composed of one or more non-viral, typically virus-derived, engineered replicons that drive high levels of expression of encoding antigens. Very low doses are required (mgs) as tens of thousands of copies are made by transfected cells. They may be delivered via intramuscular (i.m.), in the same manner as earlier mRNA vaccines, and can be encapsulated within a lipid nanoparticle to further boost performance. This manufacturing process makes GMP grade SAM a promising vaccine approach for filling the gap between emerging infectious disease and the desperate need for effective COVID-19 vaccines. SAMs are an innovative platform for vaccine development. Within an alphavirus backbone, the mRNA replicates through a double stranded RNA intermediate, and the antigen of interest replaces structural proteins, so no infectious virus is made. Very low doses are required (ugs) as tens of thousands of copies are made by transfected cells. They are delivered via intramuscular (i.m.), in the same manner as earlier mRNA vaccines, and may be delivered as naked RNA or encapsulated within a lipid nanoparticle. Comparatively, mRNA vaccines confer several advantages over vaccines introduced by virus vectors and DNA vaccines: the production procedure to generate mRNA vaccines is cell-free, simple and rapid if compared to production of whole microbe, or live attenuated or subunit vaccines.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a vaccine for viral pathogens conferring universal protection irrespective of viral mutations.

In accordance with an aspect of the present invention, there is provided a vaccine comprising or capable of expressing one or more concatemers of epitopes from a viral pathogen. In certain embodiments, the vaccine comprises or expresses epitopes for all MHC I and MHC II alleles with a frequency >1% in the target population. In certain embodiments, the target population is geographically restricted. In certain embodiments, the epitopes that bind MHC I or MHC II alleles associated with autoimmune disease are excluded. In certain embodiments, at least a portion of said epitopes are from conserved proteins from said viral pathogen. In certain embodiments, the epitopes are universal (common) epitopes of said viral pathogen. In certain embodiments, the epitopes are variant specific epitopes.

In certain embodiments, the viral pathogen is a sarbecovirus, including but not limited to SARS-CoV-2. In specific embodiments, the vaccine comprises or is capable of expressing expressing one or more concatemers of epitopes from one or more strains of sarbecovirus (e.g. SARS-CoV-2). Each concatemer of epitopes may include epitopes from a single strain or from multiple strains. For vaccines capable of expressing expressing one or more concatemers of epitopes from one or more strains, the one or more concatemers may be expressed by one or more expression vectors. In certain embodiments, each concatemer is expressed by a separate expression vector. In certain embodiments, one or more concatemers are expressed by a single expression vector.

In certain embodiments, a linker separates each of said epitopes. Exemplary linkers include RY, KRY, RYP, PRRARSV, PRRARSVKRY, PRRARSVRYP, ATLQA, QEAGAG and LALAA. The linkers may be the same or different between epitopes in a concatemer.

In certain embodiments, at least one of the one or more epitopes is as set forth in any one of the tables set forth below and/or as set forth in SEQ ID NOs 1-789.

In accordance with an aspect of the present invention, there is provided a vaccine comprising or capable of expressing one or more concatemers of epitopes set forth in any one of the tables set forth below and/or as set forth in SEQ ID NOs 1-789.

In accordance with an aspect of the present invention, there is provided a vaccine comprising or capable of expressing one or more concatemers set forth in SEQ ID NOs 798-851.

In certain embodiments, the vaccine is a viral vector-based vaccine, including but not limited to an adenoviral vector, a vesicular stomatitis virus vector or a vaccinia vector.

In certain embodiments, the vaccine is a nucleic acid-based vaccine.

In certain embodiments, the vaccine is a SAM RNA-based vaccine. In more specific embodiments, the SAM RNA-based vaccine is encapsulated in a lipid nanoparticle (LNP). In more specific embodiments, the LNP comprises a cationic lipid. In more specific embodiments, the LNP comprises phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA). In certain embodiments, the LNP comprises GEN-7036 (Lipid composition (ratio)=DOTAP:Chol:DOPE (1:0.75:0.5)).

In certain embodiments, the vaccine further comprises an adjuvant.

In certain embodiments, the vaccine further comprises a buffer.

In specific embodiments, the vaccine is a SAM RNA-based vaccine and comprises one or more SAM RNA vectors of the invention, cholesterol, 1,2-distearoyl-sn-glycero-3-phosphocholine (DSPC), ((4-hydroxybutyl)azanediyl)bis(hexane-6,1-diyl)bis(2-hexyldecanoate) (ALC-3015), 2-[(polyethylene glycol)-2000]-N,N-ditetradecylacetamide (ALC-0159), potassium chloride, monobasic potassium phosphate, sodium chloride, dibasic sodium phosphate dihydrate, water and sucrose.

In specific embodiments, the vaccine is a SAM RNA-based vaccine and comprises one or more SAM RNA vectors of the invention, cholesterol, 1,2-distearoyl-sn-glycero-3-phosphocholine (DSPC), Lipid SM-102, 1,2-dimyristoyl-rac-glycero-3-methoxypolyethylene glycol-2000 (PEG2000-DMG), tromethamine tris(hydroxymethyl)aminomethane), tromethamine hydrochloride, acetic acid, sodium acetate, water, and sucrose.

In accordance with an aspect of the present invention, there is provided a method of treating, protecting against, and/or preventing infection by a target viral pathogen, including but not limited to a sarbecovirus such as SARS-COV-2, in a subject in need thereof, the method comprising administering one or more of the vaccines of the invention to the subject.

In accordance with an aspect of the present invention, there is provided a method of generating an immune response against a target viral pathogen, including but not limited to sarbecovirus such as SARS-COV-2, the method comprising administering one or more of the vaccines of the invention to the subject.

In certain embodiments, the one or more vaccines is administered more than once. In certain embodiments, a prime and boost strategy of vaccination is used. In more specific embodiments, a heterologous prime and boost strategy is utilized. Exemplary prime and boost strategies are known in the art (see for example Sapkota et al. J Travel Med. 2021 Dec. 16; taab191. Doi: 10.1093/jtm/taab191; He et al, Emerg Microbes Infect. 2021; 10(1): 629-637; Kardani et al. Vaccine 2016, 34(4): 413-423).

In certain embodiments, wherein the subject is a mammal including but not limited to human, cat, dog, horse, sheep, goat, camel or cow.

In certain embodiments, the vaccines are formulated for parenteral administration, e.g., subcutaneous, intraperitoneal, intravenous, intradermal, and intramuscular. In certain embodiments, the vaccines are formulated for mucosal administration, e.g. oral administration, intranasal, and intravaginal routes.

### BRIEF DESCRIPTION OF THE DRAWINGS

These and other features of the invention will become more apparent in the following detailed description in which reference is made to the appended drawings.

FIG. 1 provides a map of a SAM based SARS-COV-2 vaccine of an embodiment of the present invention.

FIG. 2 provides the nucleic acid sequence of the vaccine from FIG. 1. Sequence represented as DNA for corresponding RNA sequence T is replaced with U.

FIG. 3 provides a map of a SAM based SARS-COV-2 vaccine of an embodiment of the present invention.

FIG. 4 provides the nucleic acid sequence of the vaccine from FIG. 3. Sequence represented as DNA for corresponding RNA sequence T is replaced with U.

FIG. 5 provides a map of a SAM based SARS-COV-2 vaccine of an embodiment of the present invention.

FIG. 6 provides the nucleic acid sequence of the vaccine from FIG. 5. Sequence represented as DNA for corresponding RNA sequence T is replaced with U.

FIG. 7 provides a map of a SAM based SARS-COV-2 vaccine of an embodiment of the present invention.

FIG. 8 provides the nucleic acid sequence of vaccine from FIG. 7. Sequence represented as DNA for corresponding RNA sequence T is replaced with U.

FIG. 9 provides a map of a SAM based SARS-COV-2 vaccine of an embodiment of the present invention.

FIG. 10 provides the nucleic acid sequence of vaccine from FIG. 9. Sequence represented as DNA for corresponding RNA sequence T is replaced with U.

FIG. 11 provides a map of a SAM based SARS-COV-2 vaccine of an embodiment of the present invention.

FIG. 12 provides the nucleic acid sequence of the vaccine from FIG. 11. Sequence represented as DNA for corresponding RNA sequence T is replaced with U.

FIG. 13 provides the sequence of concatemers 1 to 4 of SARS-COV-2 epitopes separated by linking sequences expressed by an embodiment of the SARS-COV-2 vaccine of the present invention.

FIG. 14 provides the sequence of concatemers 1 to 6 of SARS-COV-2 epitopes separated by linking sequences expressed by an embodiment of the SARS-COV-2 vaccine of the present invention.

FIG. 15 provides the sequence of concatemers 1 to 6 of SARS-COV-2 epitopes separated by linking sequences expressed by an embodiment of the SARS-COV-2 vaccine of the present invention.

FIG. 16 provides the sequence of concatemers 1 to 6 of SARS-COV-2 epitopes separated by linking sequences expressed by an embodiment of the SARS-COV-2 vaccine of the present invention.

FIG. 17 provides the sequence of concatemers 1 to 6 of SARS-COV-2 epitopes separated by linking sequences expressed by an embodiment of the SARS-COV-2 vaccine of the present invention.

FIG. 18 provides the sequence of concatemers 1 to 6 of SARS-COV-2 epitopes separated by linking sequences expressed by an embodiment of the SARS-COV-2 vaccine of the present invention.

FIG. 19 provides the sequence of concatemers 1 to 4 of SARS-COV-2 epitopes separated by linking sequences expressed by an embodiment of the SARS-COV-2 vaccine of the present invention.

FIG. 20 provides the sequence of concatemers 1 to 4 of SARS-COV-2 epitopes separated by linking sequences expressed by an embodiment of the SARS-COV-2 vaccine of the present invention.

FIG. 21 provides the sequence of concatemers 1 to 4 of SARS-COV-2 epitopes separated by linking sequences expressed by an embodiment of the SARS-COV-2 vaccine of the present invention.

FIG. 22 provides the sequence of concatemers 1 to 4 of SARS-COV-2 epitopes separated by linking sequences expressed by an embodiment of the SARS-COV-2 vaccine of the present invention.

FIG. 23 provides the sequence of concatemers 1 to 4 of SARS-COV-2 epitopes separated by linking sequences expressed by an embodiment of the SARS-COV-2 vaccine of the present invention.

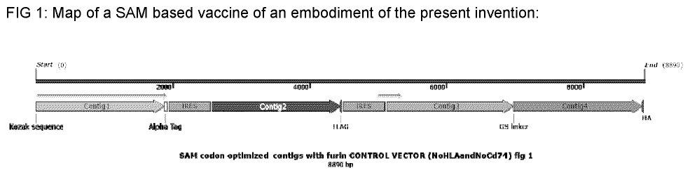

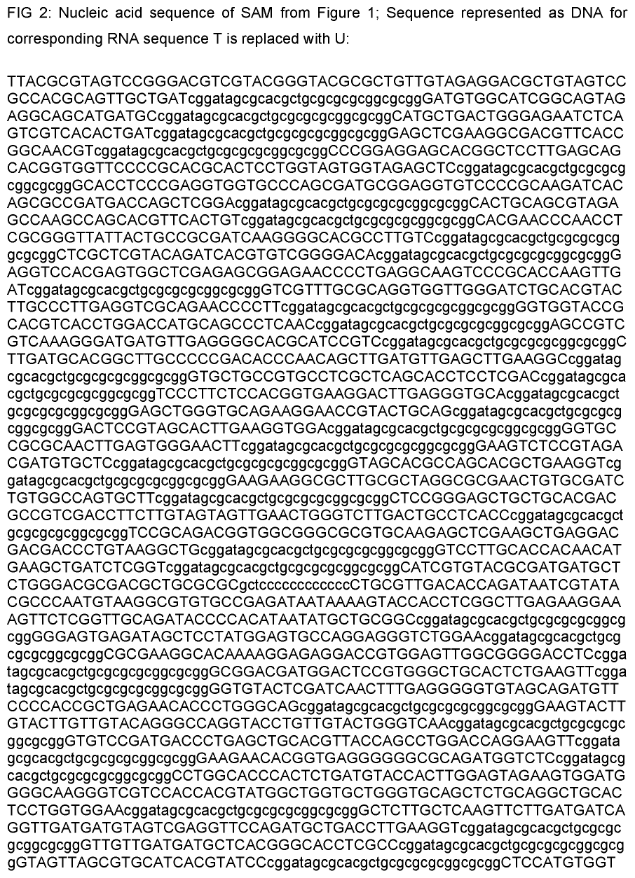

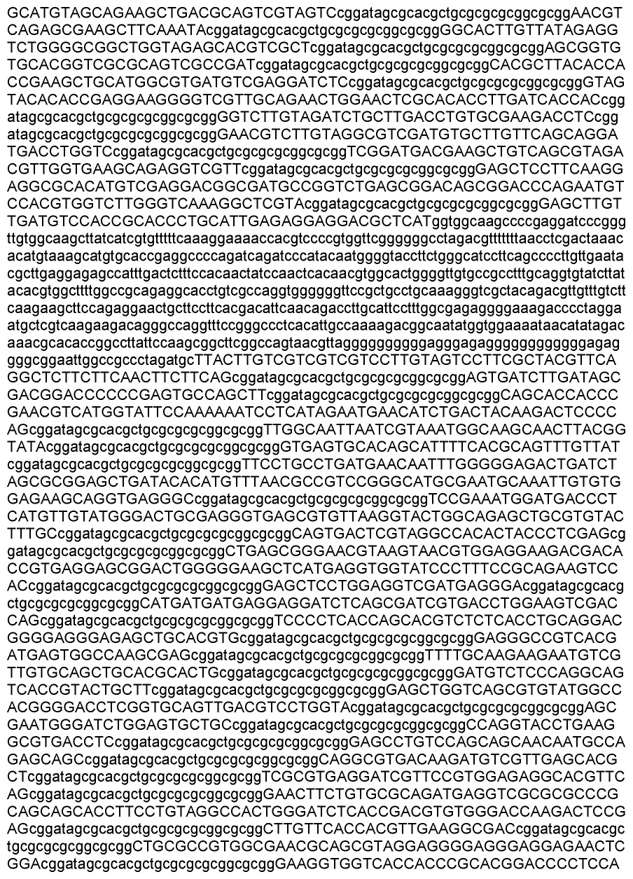

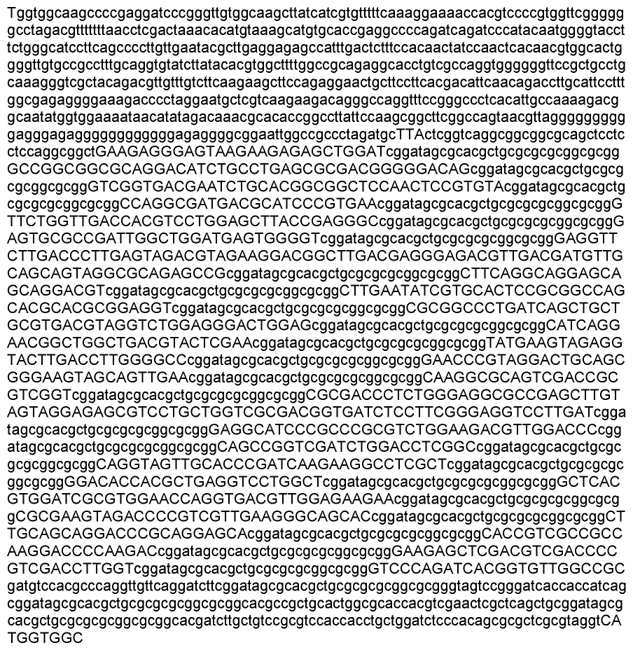

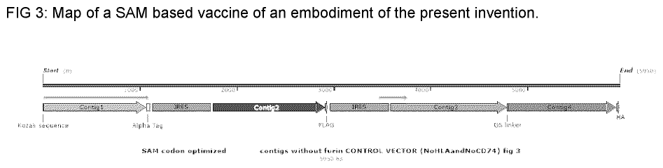

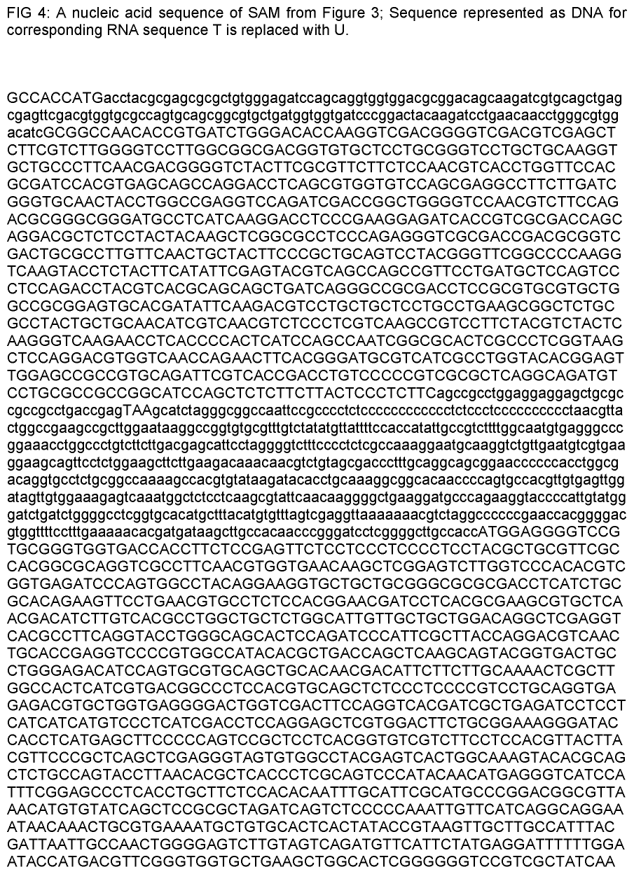

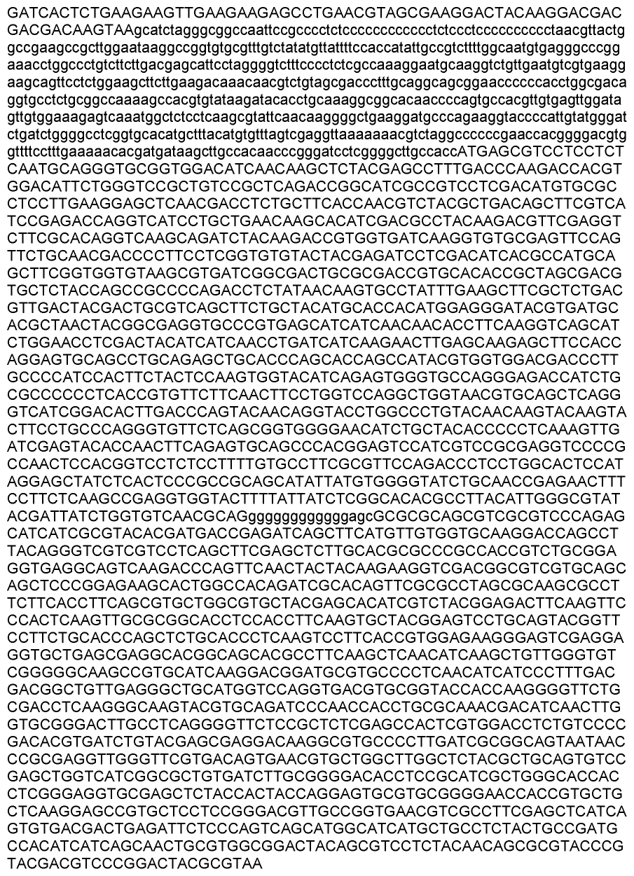

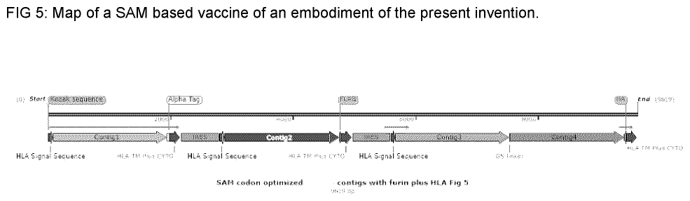

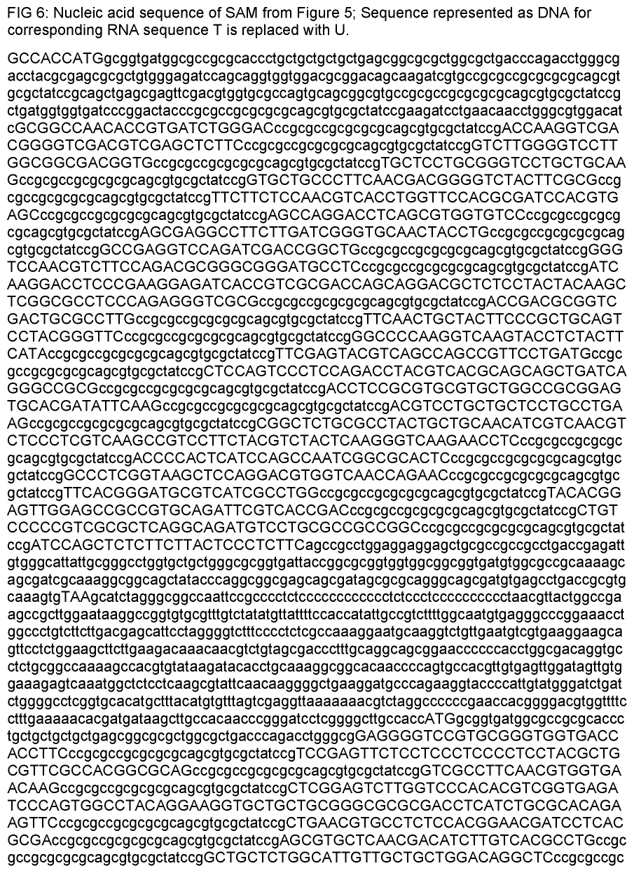

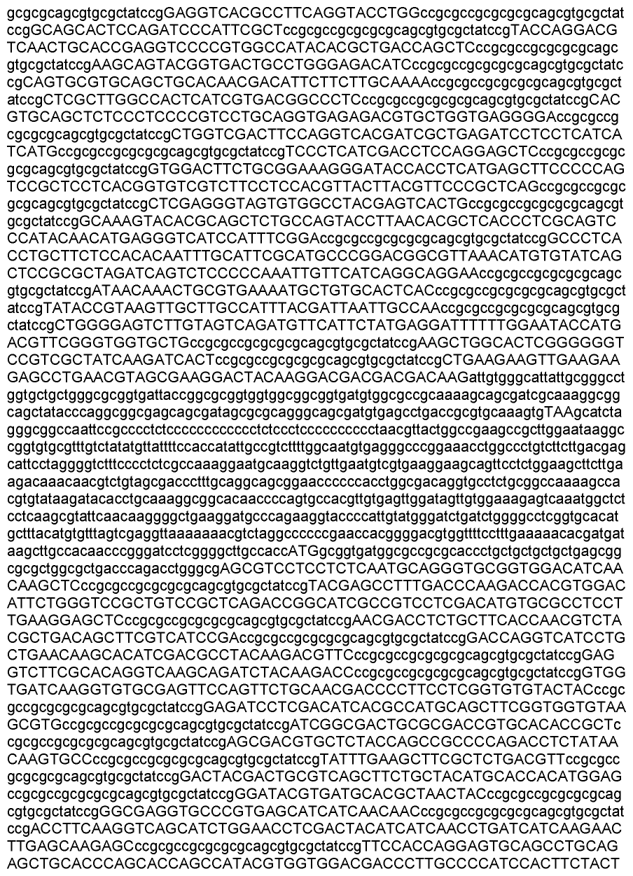

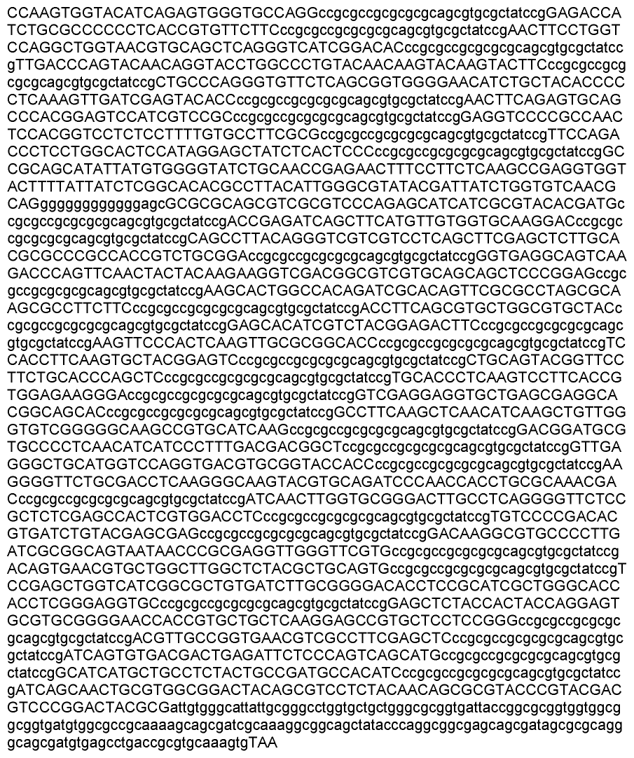

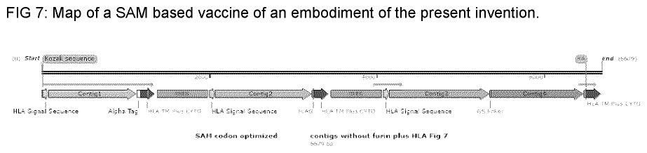

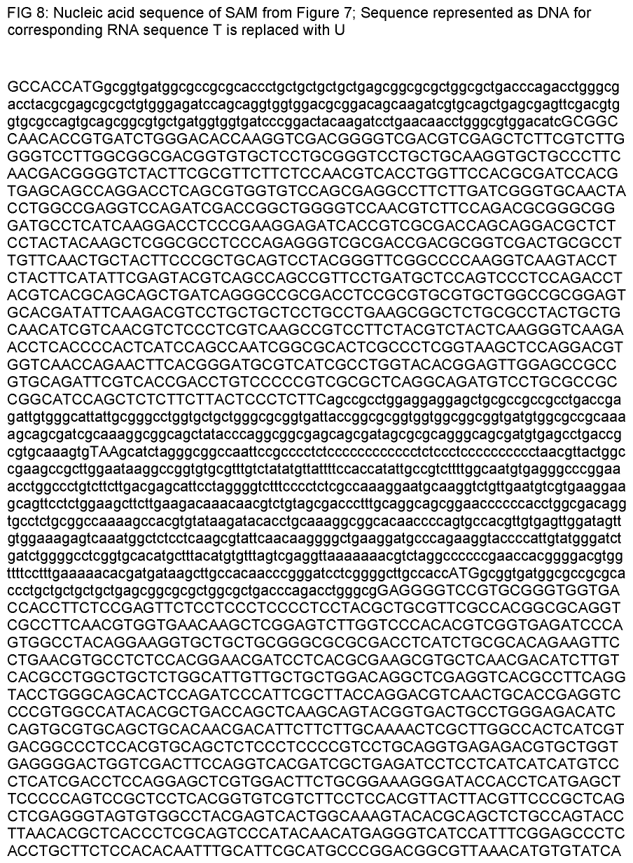

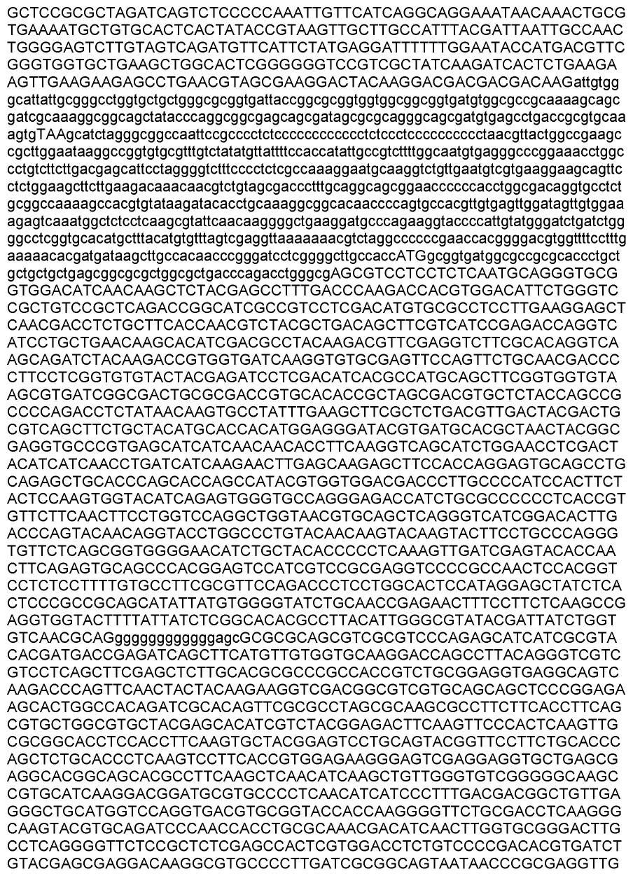

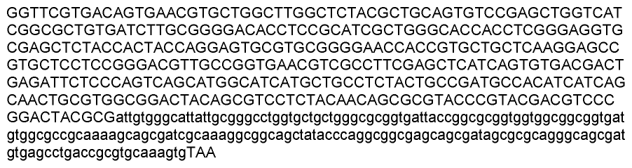

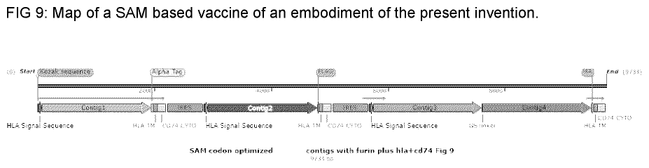

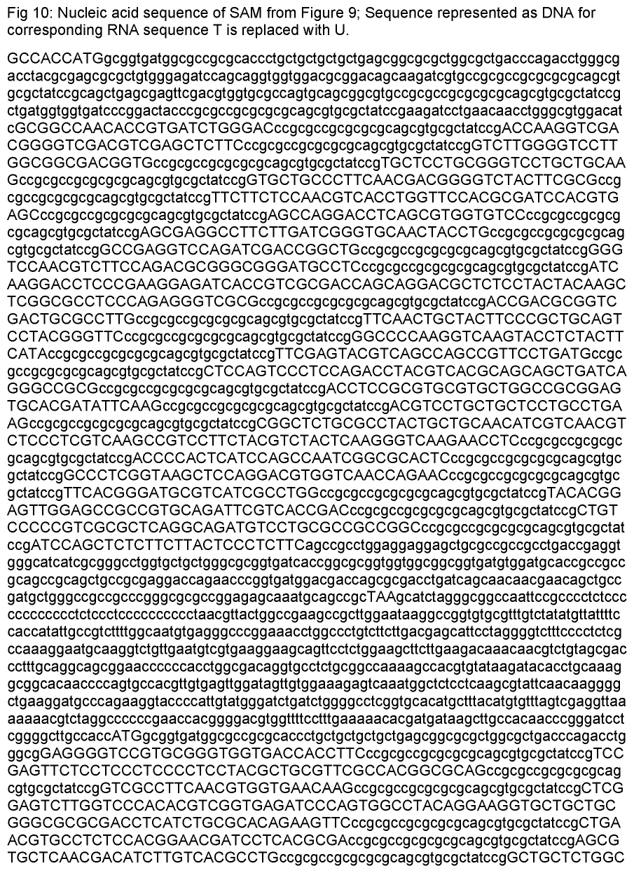

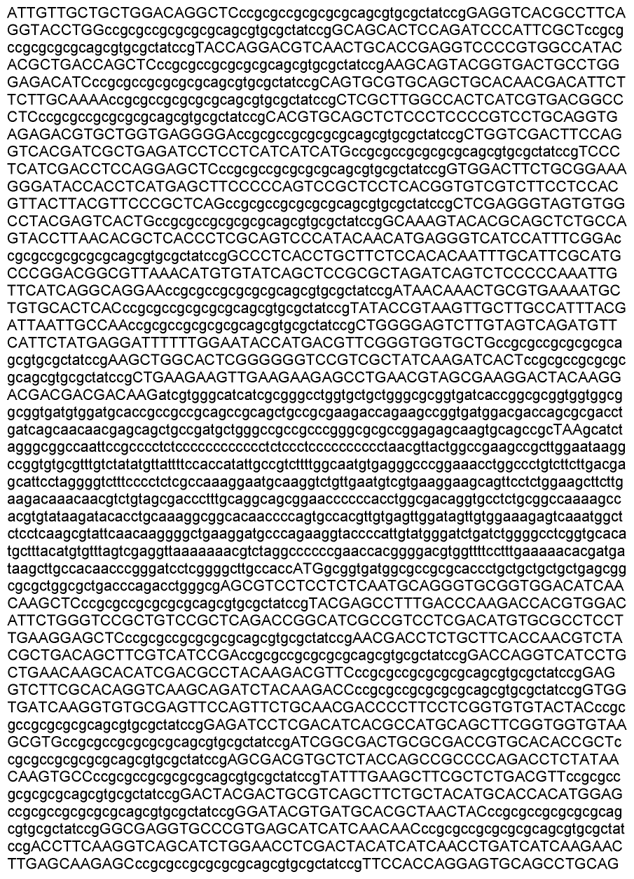

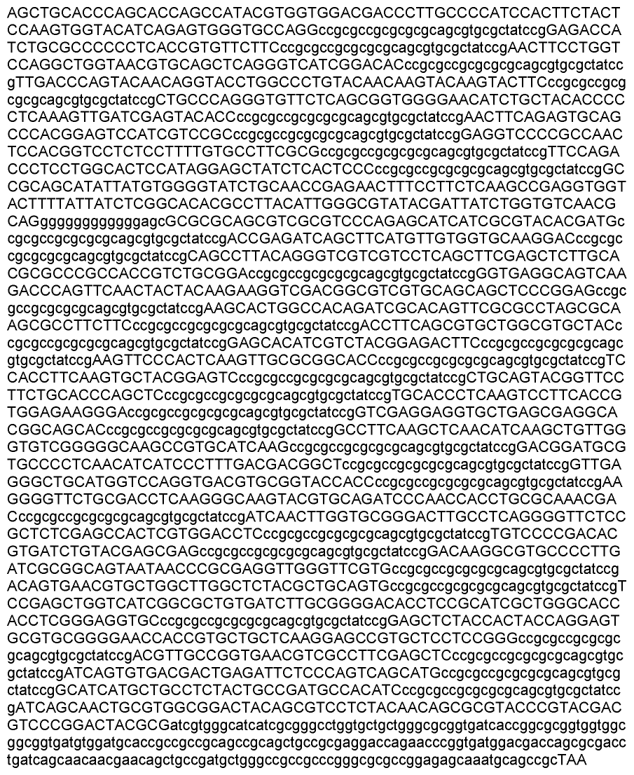

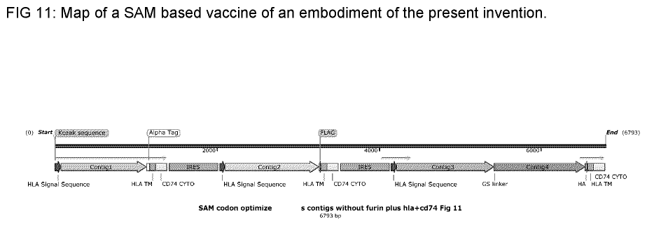

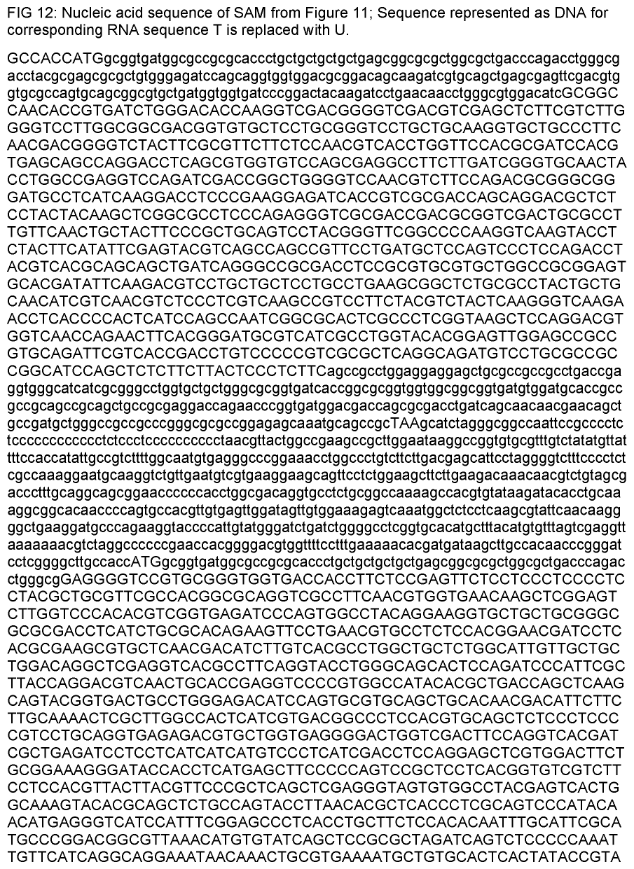

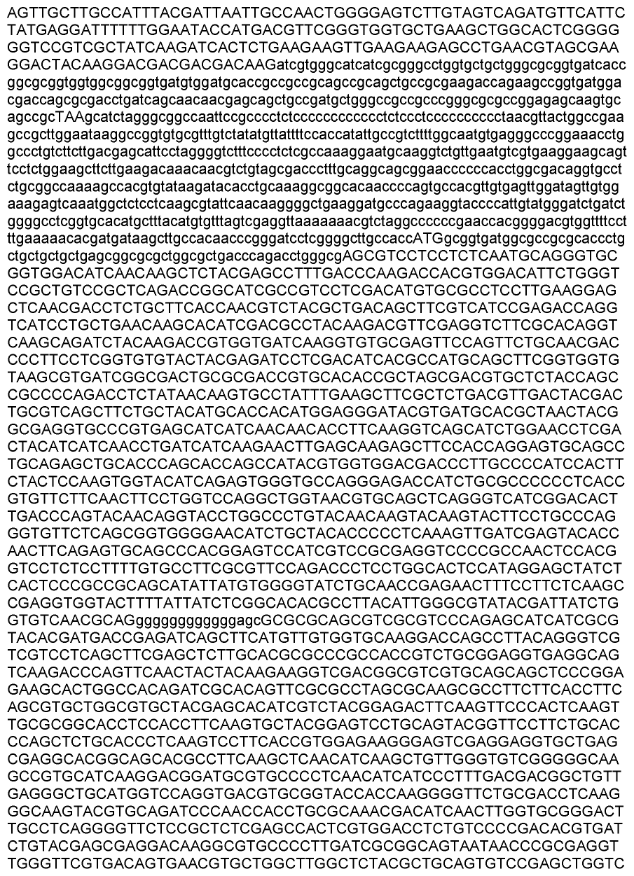

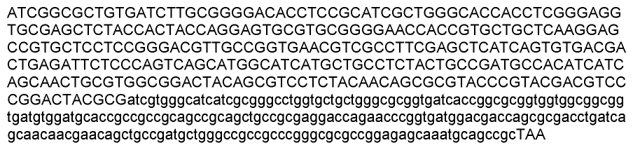

### DETAILED DESCRIPTION OF THE INVENTION

The present invention provides vaccines for viral pathogens which confer protection against a wide spectrum of strains of the target viral pathogen, including but not limited to a sarbecovirus. For example, in certain embodiments, by comprising or expressing multiple epitopes from a number of highly conserved genes of SARS-COV-2, the vaccines confer protection against a wide spectrum of strains of SARS-COV-2, therein including mutated strains arising in the future in the population and escape mutants generated in the future by other vaccines that only target a subset of the viral proteins (for instance, the spike protein alone). In addition, in certain embodiments, the vaccines are tailored to a majority of immunotypes present in the target population, the vaccines confer protection to a wide spectrum of individuals, irrespective of the make-up of their immune system in terms of MHC genes.

In certain embodiments, the invention provides vaccines against coronaviruses, including but not limited to a Sarbecovirus. In certain embodiments, the invention provides vaccines against SARS-associated coronaviruses (SARS-COV). In specific embodiments, the invention provides vaccines against SARS-COV-2. Also provided are pharmaceutical compositions comprising the vaccines and methods of generating a protective immune response against RNA viruses. In specific embodiments, there is provided pharmaceutical compositions comprising the vaccines and methods of generating a protective immune response against the SARS-COV viral pathogens.

The vaccines may trigger a humoral (B-cell) and/or cellular (T-cell response). In certain embodiments, the vaccine comprises or expresses T-cell MHC-I (e.g. HLA-I) and/or MHC-II (e.g. HLA-II) epitopes of one or more RNA viral pathogens. In certain embodiments, the vaccine comprises or expresses T-cell MHC-I (e.g. HLA-I) and/or MHC-II (e.g. HLA-II) epitopes of one or more specific SARS-COV pathogens. In certain embodiments, the epitopes are T-cell MHC-I (e.g. HLA-I) and/or MHC-II (e.g. HLA-II) epitopes of one or more SARS-COV-2 proteins.

In principle, a vaccine needs to comprise or express one epitope that binds an individual's MHC alleles for the vaccine to have activity in the individual. Accordingly, in certain embodiments, the vaccine comprises or expresses epitopes for all MHC I and MHC II alleles with a frequency >1% (or another suitably selected frequency) in the target population, so as to achieve a vaccine capable of conferring protection to a wide spectrum of individuals irrespective of their MHC type. HLA types are distributed geographically, accordingly, in certain embodiments the target population is geographically limited.

In addition, it is known in the certain HLA types are associated with autoimmunity such as ankylosing spondylitis (HLA B27) or arthritis (HLA DR4). Accordingly toxicity related to autoimmunity may be reduced by omitting peptides that bind to these HLA types.

### Vaccine Platforms

A variety of platforms may be used to generate the vaccines of the present invention. Exemplary vaccine platforms which may be used include but are not limited to protein-based platforms, virus-like particle-based vaccines, viral vector-based platforms and nucleic acid-based vaccine platforms.

In certain embodiments, the vaccine platform is a viral vector-based platform. The viral vectors may be attenuated viruses, may be replicating or non-replicating. Exemplary viral vectors include not are not limited to adenovirus, vaccinia or adeno associated virus, lentivirus or vesicular stomatitis virus (VSV). Accordingly, in certain embodiments the vaccine platform is an adenovirus, vaccinia or adeno associated virus, lentivirus or Vesicular stomatitis virus based vaccine.

In specific embodiments, the viral vector platform is an adenovirus vector platform. Various serotypes of adenoviruses have been used in vaccine development including Ad5, Ad26 and Ad35. In certain embodiments, the adenovirus vector is based on a simian adenovirus. Use of simian adenovirus vaccine vectors circumvent pre-existing human adenovirus immunity.

Exemplary, simian adenovirus serotypes used in vaccine development include simian adenovirus type 23.

In certain embodiments, the vaccine platform is a nucleic acid-based platform. Nucleic acid-based vaccine platforms may be DNA or RNA-based. Optionally, the nucleic acids include one or more modified nucleosides.

In certain embodiments, the nucleic acid-based vaccine platform is a DNA-based vaccine platform. Appropriate DNA expression vectors for use as a DNA-based vaccine platform are known in the art. A worker skilled in the art would readily appreciate that such expression vectors include the necessary elements to allow for expression of the one or more immunogens. Such elements include a promoter, such as the CMV promoter which directs transcription of the mRNA encoded by the transgene, a polyadenylation signal which mediates mRNA cleavage and polyadenylation, and Kozak sequence which directs efficient transgene translation. In specific embodiments, the DNA-based vaccine is a plasmid-based vaccine. In certain embodiments the vaccine has been optimized for expression of the assembled epitopes. In certain embodiments, the vaccines comprise nucleic acid sequence expressing said concatemer of epitopes which is less than 20 kb in length.

In certain embodiments, the vaccine comprises a sequence which has been codon optimized or deoptimized.

In certain embodiments, the nucleic acid-based vaccine is a RNA-based vaccine platform. In specific embodiments, a mRNA platform. The mRNA-based vaccine platform may be non-replicating or self-amplifying. In certain embodiments, the nucleic acid-based vaccine platform is a self-amplifying (SAM) RNA platform. A variety of RNA based expression systems are known in the art, including but not limited to expression systems based on either positive-sense and negative-sense RNA viruses. Positive-strand RNA viruses used in the development expression system include but are not limited to alphaviruses and flaviviruses. Exemplary alphaviruses used for expression systems include but are not limited to Semliki Forest virus, Venezuelan equine encephalitis virus and Sindbis virus and poliovirus. Alphavirus replicon particle-based vaccine vectors derived from Sindbis virus (SIN), Semliki Forest virus (SFV), and Venezuelan equine encephalitis virus (VEE) have been shown to induce robust antigen-specific cellular, humoral, and mucosal immune responses in many animal models of infectious disease and cancer (Perri et al.; Journal of Virology September 2003, 77 (19) 10394-10403; DOI: 10.1128/JVI.77.19.10394-10403.2003; Karl Ljungberg & Peter Liljeström (2015) Self-replicating alphavirus RNA vaccines, Expert Review of Vaccines, 14:2, 177-194, DOI: 10.1586/14760584.2015.965690). Exemplary flavivirus used for expression systems include Kunjin flavivirus. Negative sense RNA virus systems include measles and rhabdoviruses.

In specific embodiments, the SAM RNA vaccine platform is derived from an alphavirus. In such embodiments, the mRNA replicates through a double stranded RNA intermediate, and the antigen of interest replaces structural proteins, so no infectious virus is made.

### Epitopes

The multi-epitope vaccines of the present invention comprise or expresses epitopes of one or more viral proteins.

In certain embodiments, the vaccine comprises or expresses one or more epitopes from conserved (such as structural proteins) viral proteins and one or more variable (hypervariable) viral proteins (such as spike protein from SARS-COV2). By comprising or expressing both types of epitopes, the vaccine is likely to be much less prone to generate escape mutants and is more likely to confer sterilizing immunity while targeting a number of circulating variants. In addition, in certain embodiments, by comprising or expressing epitopes for all MHC I and MHC II alleles with a frequency >1% in the general human population are chosen, so as to be effective in a large portion of the population.

The vaccine may comprise or express MHC I epitopes and/or MHC II epitopes of one or more viral proteins. In certain embodiments, the epitopes are T-cell MHC-I (e.g. HLA-I) and/or MHC-II (e.g. HLA-II) epitopes of one or more RNA viral proteins. In certain embodiments, the epitopes are T-cell MHC-I (e.g. HLA-I) and/or MHC-II (e.g. HLA-II) epitopes of one or more coronavirus proteins. In certain embodiments, the epitopes are T-cell MHC-I (e.g. HLA-I) and/or MHC-II (e.g. HLA-II) epitopes of one or more sarbecovirus proteins. In certain embodiments, the epitopes are T-cell MHC-I (e.g. HLA-I) and/or MHC-II (e.g. HLA-II) epitopes of one or more SARS-COV proteins. In certain embodiments, the epitopes are T-cell MHC-I (e.g. HLA-I) and/or MHC-II (e.g. HLA-II) epitopes of one or more SARS-COV-2 proteins. In certain embodiments, the epitopes are T-cell MHC-I (e.g. HLA-I) and/or MHC-II (e.g. HLA-II) epitopes of one or more SARS-COV-2 proteins. In specific embodiments, the vaccine comprises or expresses MHC-I and MHC-II epitopes.

In certain embodiments, the vaccine comprises or expresses multiple epitopes from conserved (such as structural proteins) and variable (hypervariable) proteins (such as spike protein from SARS-COV2).

In certain embodiments, each epitope comprises an amino acid sequence between 5 and 60 amino acids. In certain embodiments, the epitopes comprise a sequence comprising 8 to 44 amino acids. In certain embodiments, the epitopes comprise a sequence comprising 8 to 30 amino acids. In certain embodiments, the epitopes comprise a sequence comprising 8 to 22 amino acids. In certain embodiments, the vaccine comprises or expresses MHC I epitopes comprising a sequence comprising 8 to 15 amino acids. In certain embodiments, the vaccine comprises or expresses MHC II epitopes comprising a sequence comprising 9 to 22 amino acids. In certain embodiments, the vaccine comprises or expresses MHC II epitopes comprising a sequence comprising more than 13 amino acids.

In certain embodiments, the sequence of the epitope(s) the vaccine comprises or expresses is 100% identical to the sequence of the corresponding epitope(s) in the wild-type viral protein. In certain embodiments, the sequence of the epitope(s) the vaccine comprises or expresses comprises one or more substitutions, insertions and/or deletions of one or more amino acid residues as compared to the sequence of the epitope in the wild-type viral protein. In certain embodiments, the epitope(s) the vaccine comprises at least 50%, at least 60%, at least 70%, at least 75%, at least 80%, at least 85%, at least 90%, at least 95%, at least 98%, at least 99% sequence identity as compared to a reference epitope sequence. The reference sequence may be any of the viral epitope sequences disclosed herein or known in the art.

In certain embodiments, the sequence of the epitope(s) of the vaccine is derived from the consensus sequence of more than one viral sequence. In certain embodiments, such sequence is obtained by considering the consensus of the most frequent variants appearing in different viral strains or in any number of sequenced viruses that have been detected while circulating in the population. In certain embodiments, such a sequence is obtained by removing from the original sequence the positions at which variants occur with a frequency larger than a specified frequency threshold (i.e., hypervariable positions in the sequence are removed, and only the positions in the sequence that are sufficiently constant are kept). In certain embodiments, additional sequences, encompassing the positions at which variants appear more frequently than some specified threshold, are added to the original sequence of the virus (i.e., one adds to the pool of potential peptides the ones generated by non-synonymous variants that occur with sufficient frequency). In certain embodiments, the variants which appear more frequently than some specific threshold are computed based on the strains circulating at some given time in some specific geographical region. In certain embodiments. In certain embodiments, the variants which appear more frequently than some specific threshold are computed based on viral lineages as defined by WHO, other public bodies, or research groups.

In certain embodiments, one or more epitopes are derived from one or more sarbecovirus proteins, including but not limited to SARS-COV proteins including but not limited to SARS-COV-2 proteins.

The complete genome of SARs-COV-2 is known in the art and is published under GenBank Accession NC_045512.2 (Nature 579 (7798), 265-269 (2020)). Variants of SARS-COV-2 are known in the art. The targets of T cell responses to SARS-COV-2 have been examined in exposed individuals. The targets were found to include but are not limited to M, Spike, N, nsp3, nsp4, ORF3a and ORF8 (Grifoni et al., Cell 181:1489-1501, 2020). Accordingly, in certain embodiments, the selection of epitopes is based on the clinical profile of convalescent COVD-19 patients. In specific embodiments, the selection of epitopes is based on the frequency of antigen specific T cell responses that convalescent patients make for both T helper (MHC II) and CTL (MHC I). In certain embodiments, eptiopes were selected based on antigenic score of the epitopes. In specific embodiments, the vaccine comprises MHC I and MHC II binding epitopes for different HLA Alleles. In specific embodiments, the vaccine comprises MHC I and MHC II binding epitopes for all MHC I and MHC II alleles with a frequency >1% in the general human population are chosen.

In certain embodiments, the vaccine comprises or expresses one or more epitopes from one or more of the following SARS-COV-2 proteins: spike, NSP1, NSP2, Proteinase 3CL-Pro, NSP7, NSP8, NSP9, NSP10, helicase, exonuclease, endonuclease, methyltransferase, ORF6, N protein, and ORF10.

In certain embodiments, the vaccine comprises or expresses one or more epitopes from one or more of the following SARS-COV-2 proteins: spike, papain-like protease, NSP4, RNA dependent RNA polymerase, M protein, ORF7a, and ORF8.

In certain embodiments, the vaccine comprises or expresses one or more epitopes from one or more of the following SARS-COV-2 proteins: 2′-O-ribose methyltransferase, 3C-like proteinase, 3′-to-5′ exonuclease, endoRNAse, envelope protein, helicase, leader protein, membrane glycoprotein, nsp10, nsp2, nsp3, nsp4, nsp6, nsp7, nsp8, nsp9, nucleocapsid phosphoprotein, ORF10 protein, ORF3a protein, ORF6 protein, ORF7a protein, ORF8 protein, RNA-dependent RNA polymerase, and surface glycoprotein.

In certain embodiments, the vaccine comprises or expresses one or more epitopes as set forth in any one of Tables below. In certain embodiments, the vaccine comprises or expresses one or more of the concatemer of epitopes (optionally the epitopes are separated by a linker sequence) as set forth in any one of Tables below.

In certain embodiments, the vaccine comprises or expresses one or more epitopes as set forth in any one of SEQ ID NOs 1-789. In certain embodiments, the vaccine comprises or expresses one or more of the concatemer of epitopes (optionally the epitopes are separated by a linker sequence) as set forth in any one of SEQ ID NOs 1-789. In specific embodiments, the linkers comprise the sequence as set forth in 790 or 791.

In certain embodiments, the vaccine comprises one or more nucleic acids capable of expressing one or more of the epitopes or concatemers of epitopes optionally separated by linker sequences described above. The one or more nucleic acids may be DNA or RNA. The vaccine comprising or capable of expressing the one or more epitopes may be virus-like particle-based vaccines, viral vector-based vaccines or nucleic acid-based vaccines. In nucleic acid-based vaccines, the nucleic acids may optionally include modifications including for example one or more modified nucleosides. In certain embodiments, the nucleic acid sequences are codon optimized. In certain embodiments, the nucleic acid sequences are codon optimized for expression in mammalian cells, optionally human cells. In certain embodiments, the nucleic acid sequences are deoptimized.

### Linkers Between Epitopes

In certain embodiments, the vaccines comprise or express one or more concatemer(s) of epitopes with intervening linker peptides. In certain embodiments, the linker peptides comprise a protease cleavage site(s). In certain embodiments, the insertion of protease cleavage sites enhances antigen processing. Exemplary cleavage sites include chymotryptic, tryptic and furin cleavage sties. In specific embodiments, the linker consists of other viral and cellular protease sites. In specific embodiments, the linker peptides comprise the following sequence: RY, KRY, RYP, PRRARSV, PRRARSVKRY or PRRARSVRYP. In certain embodiments, the linker can be any peptide consistent with the motif [AVTP][TKRV]LQ[AS]. In specific embodiments, the linker consists of the following sequence: ATLQA. In specific embodiments, the linker consists of the following sequence: QEAGAG. In specific embodiments, the linker consists of the following sequence: LALAA.

### Exemplary Concatemers of Epitopes

In certain embodiments, the vaccine comprises or expresses at least one of concatemers of epitopes from SARS-COV-2 with intervening linker sequences set forth in Table 1 below. In certain embodiments, the vaccine comprises or expresses at least two of the concatemers of epitopes from SARS-COV-2 with intervening linker sequences set forth in Table 1 below. In certain embodiments, the vaccine comprises or expresses three of the concatemers set forth below. In certain embodiments, the vaccine comprises or expresses all the concatemers set forth below in Table 1.

TABLE 1

| | pos_in_ | | | pos_ |
|---|---|---|---|---|
| concatemer | concatemer | epitope | gene | in_gene |
| 1 | 1-12 | LNVPLHGTILTR | membrane_glycoprotein(26523-27191 | 120-131 |
| 1 | 13-22 | PRRARSVRYP linker 1-10 | | |
| 1 | 23-35 | TVLKKCKSAFYIL | nsp3(2720-8554 | 526-538 |
| 1 | 36-45 | PRRARSVRYP linker 1-10 | | |
| 1 | 46-53 | IETISLAG | nsp3(2720-8554) | 684-691 |
| 1 | 54-63 | PRRARSVRYP linker 1-10 | | |
| 1 | 64-73 | NWNCVNCDTF | nsp3*2720-8554) | 1631-1640 |
| 1 | 74-83 | PRRARSVRYP linker 1-10 | | |
| 1 | 84-97 | DYDCVSFCYMHHME | 3C-like proteinase (10055-10972) | 153-166 |
| 1 | 98-107 | PRRARSVRYP linker 1-10 | | |
| 1 | 108-117 | TPLIQPIGAL | nsp4(8555-10054) | 269-278 |
| 1 | 118-127 | PRRARSVRYP linker 1-10 | | |
| 1 | 128-154 | VTGLFKDCSKVITGLHPTQAPTHLSVD | 3′-to-5′_exonuclease(18040-19620) | 4-30 |
| 1 | 155-164 | PRRARSVRYP linker 1-10 | | |
| 1 | 165-179 | SDVLYQPPQTSITSA | nsp4(8555-10054) | 483-497 |
| 1 | 180-189 | PRRARSVRYP linker 1-10 | | |
| 1 | 190-200 | TKVDGVDVELF | endoRNAse(19621-20658) | 33-43 |
| 1 | 201-210 | PRRARSVRYP linker 1-10 | | |
| 1 | 211-221 | ETICAPLTVFF | endoRNAse(19621-20658) | 113-123 |
| 1 | 222-231 | PRRARSVRYP linker 1-10 | | |
| 1 | 232-246 | KETLYCIDGALLTKS | nsp3(2720-8554) | 1024-1038 |
| 1 | 247-256 | PRRARSVRYP linker 1-10 | | |
| 1 | 257-270 | QIELKFNPPALQDA | nsp3(2720-8554) | 867-880 |
| 1 | 271-280 | PRRARSVRYP linker 1-10 | | |
| 1 | 281-298 | HVQLSLPVLQVRDVLVRG | leader_protein(266-805) | 13-30 |
| 1 | 299-308 | PRRARSVRYP linker 1-10 | | |
| 1 | 309-316 | DHVISTSH | helicase(16237-18039) | 32-39 |
| 1 | 317-326 | PRRARSVRYP linker 1-10 | | |
| 1 | 327-339 | ANFCALILAYCNK | nsp3(2720-8554) | 890-902 |
| 1 | 340-349 | PRRARSVRYP linker 1-10 | | |
| 1 | 350-357 | LPINVIVF | nsp3(2720-8554) | 1718-1725 |
| 1 | 358-367 | PRRARSVRYP linker 1-10 | | |
| 1 | 368-381 | CATTRQVVNVVTTK | nsp3(2720-8554) | 1926-1939 |
| 1 | 382-391 | PRRARSVRYP linker 1-10 | | |
| 1 | 392-399 | YDKLVSSF | nsp3(2720-8554) | 365-372 |
| 1 | 400-409 | PRRARSVRYP linker 1-10 | | |
| 1 | 410-418 | KFPLKLRGT | 2′-O-ribose_methyltransferase | 249-257 |
| | | | (20659-21552) | |
| 1 | 419-428 | PRRARSVRYP linker 1-10 | | |
| 1 | 429-438 | KSASVYYSQL | nsp3(2720-8554) | 1737-1746 |
| 1 | 439-448 | PRRARSVRYP linker 1-10 | | |
| 1 | 449-463 | TYACWHHSIGFDYVY | 3′-to-5′_exonuclease(10840-19620) | 223-237 |
| 1 | 464-473 | PRRARSVRYP linker 1-10 | | |
| 1 | 474-486 | AIKCVPQADVEWK | 3′-to-5′_exonuclease(10840-19620) | 337-349 |
| 1 | 487-496 | PRRARSVRYP linker 1-10 | | |
| 1 | 497-510 | SDRVVFVLWAHGFE | 3′-to-5′_exonuclease(10840-19620) | 178-191 |
| 1 | 511-520 | PRRARSVRYP linker 1-10 | | |
| 1 | 521-544 | TYASALWEIQQVVDADSKIVQLSE | nsp8(12092-12685) | 148-171 |
| 1 | 545-554 | PRRARSVRYP linker 1-10 | | |
| 1 | 555-562 | YLKLRSDV | nsp4(8555-10054) | 427-434 |
| 1 | 563-572 | PRRARSVRYP linker 1-10 | | |
| 1 | 573-586 | DGCVPLNIIPLTTA | nsp8(12092-12685) | 112-125 |
| 1 | 587-596 | PRRARSVRYP linker 1-10 | | |
| 1 | 597-609 | KEILVTYNCCDDD | RNA-dependent_RNA_polymerase | 143-155 |
| | | | (13442-13468 + 13468-16236) | |
| 1 | 610-619 | PRRARSVRYP linker 1-10 | | |
| 1 | 620-627 | SDFVRATA | ORF3a protein(25393-26220) | 26-33 |
| 1 | 628-637 | PRRARSVRYP linker 1-10 | | |
| 1 | 638-650 | GVVDYGARFYFYT | nsp3(2720-8554) | 596-608 |
| 1 | 651-660 | PRRARSVRYP linker 1-10 | | |
| 1 | 661-674 | QEHYVRITGLYPTL | helicase(16237-18039) | 243-256 |
| 1 | 675-684 | PRRARSVRYP linker 1-10 | | |
| 1 | 685-707 | TFKVSIWLNLDYIINLIIKNLSKS | ORF6_protein(27202-27387) | 21-43 |
| 1 | 708-717 | PRRARSVRYP linker 1-10 | | |
| 1 | 718-730 | FVVEVVDKYFDCY | RNA-dependent_RNA_polymerase | 471-483 |
| | | | (13442-13468 + 13468-16236) | |
| 1 | 731-740 | PRRARSVRYP linker 1-10 | | |
| 1 | 741-753 | YTVSCLPFTINCQ | ORF8_protein(27894-28259) | 79-91 |
| 1 | 754-763 | PRRARSVRYP linker 1-10 | | |
| 1 | 764-778 | EILDITPCSFGGVSV | surface_glycoprotein(21563-25384) | 583-597 |
| 1 | 779-788 | PRRARSVRYP linker 1-10 | | |
| 1 | 789-798 | MPLGYVTHGL | nsp3(2720-8554) | 630-639 |
| 1 | 799-808 | PRRARSVRYP linker 1-10 | | |
| 1 | 809-816 | MADLVYAL | RNA-dependent_RNA_polymerase | 124-131 |
| | | | (13442-13468 + 13468-16236) | |
| 1 | 817-826 | PRRARSVRYP linker 1-10 | | |
| 1 | 827-840 | TSACVLAAECTIFK | nsp4(8555-10054) | 147-160 |
| 1 | 841-850 | PRRARSVRYP linker 1-10 | | |
| 1 | 851-866 | SAEVAVKMFDAYVNTF | nsp3(2720-8554) | 1765-1780 |
| 1 | 867-876 | PRRARSVRYP linker 1-10 | | |
| 1 | 877-886 | NEFYAYLRKH | RNA-dependent_RNA_polymerase | 743-752 |
| | | | (13442-13468 + 13468-16236) | |
| 1 | 887-896 | PRRARSVRYP linker 1-10 | | |
| 1 | 897-906 | LQYGSFCTQL | surface_glycoprotein(21563-25384) | 754-763 |
| 1 | 907-916 | PRRARSVRYP linker 1-10 | | |
| 1 | 917-925 | IQLSSYSLF | 2′-O-ribose_methyltransferase | 237-245 |
| | | | (20659-21552) | |
| 1 | 926-935 | PRRARSVRYP linker 1-10 | | |
| 1 | 936-946 | CTLKSFTVEKG | surface_glycoprotein(21563-25384) | 301-311 |
| 1 | 947-956 | PRRARSVRYP linker 1-10 | | |
| 1 | 957-964 | PSFLGRYM | nsp3(2720-8554) | 822-829 |
| 1 | 964-974 | PRRARSVRYP linker 1-10 | | |
| 1 | 975-989 | ISNCVADYSVLYNSA | surface_glycoprotein(21563-25384) | 358-372 |
| 1 | 990-999 | PRRARSVRYP linker 1-10 | | |
| 1 | 1000-1011 | ESVTTEILPVSM | surface_glycoprotein(21563-25384) | 720-731 |
| 1 | 1012-1021 | PRRARSVRYP linker 1-10 | | |
| 1 | 1022-1041 | SEAVEAPLVGTPVCINGLML | nsp2(806-2719) | 591-610 |
| 1 | 1042-1051 | PRRARSVRYP linker 1-10 | | |
| 1 | 1052-1061 | FDVEGCHATR | 3′-to-5′_exonuclease(10840-19620) | 89-98 |
| 1 | 1062-1071 | PRRARSVRYP linker 1-10 | | |
| 1 | 1072-1086 | ERFVSLAIDAYPLTK | RNA-dependent_RNA_polymerase | 857-871 |
| | | | (13442-13468 + 13468-16236) | |
| 1 | 1087-1096 | | | |
| 1 | 1097-1113 | PRRARSVRYP linker 1-10 | RNA-dependent_RNA_polymerase | 717-733 |
| | | | (13442-13468 + 13468-16236) | |
| 1 | 1114-1123 | PRRARSVRYP linker 1-10 | | |
| 1 | 1124-1160 | FHQECSLQSCTQHQPYVVDDPCPIHFY | ORF8_protein(27894-28259) | 16-52 |
| | | SKWYIRVGAR | | |
| 1 | 1161-1170 | PRRARSVRYP linker 1-10 | | |
| 1 | 1171-1189 | KLNVGDYFVLTSHTVMPLS | helicase(16237-18039) | 218-236 |
| 1 | 1190-1199 | PRRARSVRYP linker 1-10 | | |
| 1 | 1200-1207 | TSCCSCLK | surface_glycoprotein | 1238-1245 |
| | | | (21563-25384) | |
| 1 | 1208-1217 | PRRARSVRYP linker 1-10 | | |
| 1 | 1218-1225 | AALQIPFA | surface_glycoprotein | 892-899 |
| | | | (21563-25384) | |
| 1 | 1226-1235 | PRRARSVRYP linker 1-10 | | |
| 1 | 1236-1243 | NQMCLSTL | nsp2(806-2719) | 133-140 |
| 1 | 1244-1253 | PRRARSVRYP linker 1-10 | | |
| 1 | 1254-1265 | GSNVFQTRAGCL | surface_glycoprotein | 639-650 |
| | | | (21563-25384) | |
| 1 | 1266-1275 | PRRARSVRYP linker 1-10 | | |
| 1 | 1276-1285 | KASCTLSEQL | NSP2(806-2719) | 30-39 |
| 1 | 1286-1295 | PRRARSVRYP linker 1-10 | | |
| 1 | 1296-1323 | IKDLPKEITVATSRTLSYYKLGASQRV | membrane_glycoprotein | 161-188 |
| | | A | (26523-27191) | |
| 1 | 1324-1333 | PRRARSVRYP linker 1-10 | | |
| 1 | 1334-1343 | ARDLICAQKF | surface_glycoprotein | 846-855 |
| | | | (21563-25384) | |
| 1 | 1344-1353 | PRRARSVRYP linker 1-10 | | |
| 1 | 1354-1361 | SQDLSVVS | endoRNAse(19621-20658) | 308-315 |
| 1 | 1362-1371 | PRRARSVRYP linker 1-10 | | |
| 1 | 1372-1379 | DVVECLKL | nsp3(2720-8554) | 1832-1839 |
| 1 | 1380-1389 | PRRARSVRYP linker 1-10 | | |
| 1 | 1390-1407 | INVFAFPFTIYSLLLCRM | ORF10_protein(29558-29674) | 4-21 |
| 2 | 1-10 | DKLQFTSLEI | helicase(16237-18039) | 583-592 |
| 2 | 11-20 | PRRARSVRYP linker 1-10 | | |
| 2 | 21-33 | SYELQTPFEIKLA | nsp2(806-2719) | 68-80 |
| 2 | 34-43 | PRRARSVRYP linker 1-10 | | |
| 2 | 44-51 | GYVMHANY | 2′-O-ribose_methyltransferase | 221-228 |
| | | | (20659-21552) | |
| 2 | 52-61 | PRRARSVRYP linker 1-10 | | |
| 2 | 62-94 | YEPLTQDHVDILGPLSAQTGAIVLDM | 3C-like_proteinase | 239-271 |
| | | CASLKEL | | |
| 2 | 95-104 | PRRARSVRYP linker 1-10 | | |
| 2 | 105-112 | RVEAFEYY | nsp3(2720-8554) | 810-817 |
| 2 | 113-122 | PRRARSVRYP linker 1-10 | | |
| 2 | 123-132 | EGSVRVVTTF | nsp4(8555-10054) | 207-216 |
| 2 | 133-142 | PRRARSVRYP linker 1-10 | | |
| 2 | 143-155 | QCVQLHNDILLAK | nsp7(11843-12091) | 31-43 |
| 2 | 156-165 | PRRARSVRYP linker 1-10 | | |
| 2 | 166-180 | GETFVTHSKGLYRKC | nsp2(806-2719) | 526-540 |
| 2 | 181-190 | PRRARSVRYP linker 1-10 | | |
| 2 | 191-205 | FSGYLKLTDNVYIKN | nsp3(2720-8554) | 210-224 |
| 2 | 206-215 | PRRARSVRYP linker 1-10 | | |
| 2 | 216-225 | GEVPVSIINN | endoRNAse(19621-20658) | 20-29 |
| 2 | 226-235 | PRRARSVRYP linker 1-10 | | |
| 2 | 236-260 | KVKPTVVVNAANVYLKHGGGVAGAL | nsp3(2720-8554) | 233-257 |
| 2 | 261-270 | PRRARSVRYP linker 1-10 | | |
| 2 | 271-285 | TKYLVQQESPFVMMS | nsp3(2720-8554) | 976-990 |
| 2 | 286-295 | PRRARSVRYP linker 1-10 | | |
| 2 | 286-307 | GDVVAIDYKHYT | nsp3(2720-8554) | 1147-1158 |
| 2 | 308-317 | PRRARSVRYP linker 1-10 | | |
| 2 | 318-325 | LMVVIPDY | nsp8(12092-12685) | 128-135 |
| 2 | 326-335 | PRRARSVRYP linker 1-10 | | |
| 2 | 336-352 | KHWPQIAQFAPSASAFF | nucleocapsid_phosphoprotein | 299-315 |
| | | | (28274-29533) | |
| 2 | 353-362 | PRRARSVRYP linker 1-10 | | |
| 2 | 363-376 | SVGKFCLEASFNYL | nsp3(2720-8554) | 1387-1400 |
| 2 | 377-386 | PRRARSVRYP linker 1-10 | | |
| 2 | 377-397 | ITNCVNKLCTH | nsp10(13025-13441) | 38-48 |
| 2 | 398-407 | PRRARSVRYP linker 1-10 | | |
| 2 | 408-518 | ASCCLYCRCHI | nsp10(13025-13441) | 71-81 |
| 2 | 419-428 | PRRARSVRYP linker 1-10 | | |
| 2 | 429-451 | FGGCVFSYVGCHNKCAYWVPRAS | nsp2(806-2719) | 226-248 |
| 2 | 452-461 | PRRARSVRYP linker 1-10 | | |
| 2 | 462-496 | HEFCSQHTMLVKQGDDYVYLPYPDPSR | RNA-dependent_RNA_polymerase | 810-844 |
| | | ILGAGCFV | (13442-13468 + 13468-16236) | |
| 2 | 497-506 | PRRARSVRYP linker 1-10 | | |
| 2 | 507-520 | FGTVYEKLKPVLDW | nsp2(806-2719) | 437-450 |
| 2 | 521-530 | PRRARSVRYP linker 1-10 | | |
| 2 | 531-538 | EHIVYGDF | endoRNAse(19621-20658) | 233-240 |
| 2 | 539-548 | PRRARSVRYP linker 1-10 | | |
| 2 | 549-566 | KILNNLGVDIAANTVIWD | endoRNAse(19621-20658) | 70-87 |
| 2 | 567-579 | PRRARSVRYP linker 1-10 | | |
| 2 | 577-586 | LAWPLIVTAL | nsp8(12092-12685) | 180-189 |
| 2 | 587-596 | PRRARSVRYP linker 1-10 | | |
| 2 | 597-610 | LSPVALRQMSCAAG | nsp9(12092-12685) | 4-17 |
| 2 | 611-620 | PRRARSVRYP linker 1-10 | | |
| 2 | 621-632 | YAKPFLNKVVST | nsp3(2720-8554) | 1323-1334 |
| 2 | 633-642 | PRRARSVRYP linker 1-10 | | |
| 2 | 643-650 | TFSVLACY | 3C-like_protinase(10055-10972) | 111-118 |
| 2 | 651-660 | PRRARSVRYP linker 1-10 | | |
| 2 | 661-668 | VAFNVVNK | endoRNAse(19621-20658) | 5-12 |
| 2 | 669-678 | PRRARSVRYP linker 1-10 | | |
| 2 | 679-715 | ARKHTTCCSLCHRFYRLANECAQVLSE | RNA-dependent_RNA_polymerase | 639-675 |
| | | MVMCGGSLYV | (13442-13468 + 13468-16236) | |
| 2 | 716-725 | PRRARSVRYP linker 1-10 | | |
| 2 | 726-741 | LVDFQVTIAEILLIIM | ORF6_protein(27202-27387) | 4-19 |
| 2 | 742-751 | PRRARSVRYP linker 1-10 | | |
| 2 | 752-761 | CPRHVICTSE | 3C-like_protinase(10055-10972) | 38-47 |
| 2 | 762-771 | PRRARSVRYP linker 1-10 | | |
| 2 | 772-787 | LLKDCPAVAKHDFFKF | RNA-dependent_RNA_polymerase | 89-104 |
| | | | (13442-13468 + 13468-16236) | |
| 2 | 788-797 | PRRARSVRYP linker 1-10 | | |
| 2 | 798-813 | RKIFVDGVPFVVSTGY | RNA-dependent_RNA_polymerase | 331-346 |
| | | | (13442-13468 + 13468-16236) | |
| 2 | 814-823 | PRRARSVRYP linker 1-10 | | |
| 2 | 824-843 | RAKHYVYIGDPAQLPAPRTL | helicase(16237-18039) | 392-411 |
| 2 | 844-853 | PRRARSVRYP linker 1-10 | | |
| 2 | 854-863 | TLPVNVAFEL | endoRNAse(19621-20658) | 48-57 |
| 2 | 864-873 | PRRARSVRYP linker 1-10 | | |
| 2 | 874-888 | FSVAALTNNVAFQTV | RNA-dependent_RNA_polymerase | 396-410 |
| | | | (13442-13468 + 13468-16236) | |
| 2 | 889-898 | PRRARSVRYP linker 1-10 | | |
| 2 | 899-906 | SVELKHFF | RNA-dependent_RNA_polymerase | 434-411 |
| | | | (13442-13468 + 13468-16236) | |
| 2 | 907-916 | PRRARSVRYP linker 1-10 | | |
| 2 | 917-928 | FNVYFPLQSYGF | surface_glycoprotein(21563-25384) | 486-497 |
| 2 | 929-938 | PRRARSVRYP linker 1-10 | | |
| 2 | 939-952 | FFSNVTWFHAIHVS | surface_glycoprotein(21563-25384) | 58-71 |
| 2 | 953-962 | PRRARSVRYP linker 1-10 | | |
| 2 | 963-974 | EVFAQVKQIYKT | surface_glycoprotein(21563-25384) | 780-791 |
| 2 | 975-984 | PRRARSVRYP linker 1-10 | | |
| 2 | 985-994 | YIAQVDVVNF | ORF10_protein(29558-29674) | 26-36 |
| 2 | 995-1004 | PRRARSVRYP linker 1-10 | | |
| 2 | 1005-1014 | LLKHPIVWHV | nsp3(2720-8554) | 1167-1176 |
| 2 | 1015-1024 | PRRARSVRYP linker 1-10 | | |
| 2 | 1025-1042 | YQDVNCTEVPVAIHADQL | surface_glycoprotein(21563-25384) | 612-629 |
| 2 | 1043-1052 | PRRARSVRYP linker 1-10 | | |
| 2 | 1053-1076 | SELVIGAVILRGHLRIAGHHLGRC | membrane_glycoprotein | 136-159 |
| | | | (26523-27191) | |
| 2 | 1077-1086 | PRRARSVRYP linker 1-10 | | |
| 2 | 1087-1094 | TDAVDCAL | surface_glycoprotein(21563-25384) | 286-293 |
| 2 | 1095-1104 | PRRARSVRYP linker 1-10 | | |
| 2 | 1105-1112 | SLIDLQEL | surface_glycoprotein(21563-25384) | 1196-1203 |
| 2 | 1113-1122 | PRRARSVRYP linker 1-10 | | |
| 2 | 1123-1135 | RTCCLCDRRATCF | 3′-to-5′_exonuclease(18040-19620) | 205-217 |
| 2 | 1136-1145 | PRRARSVRYP linker 1-10 | | |
| 2 | 1146-1153 | FVKATCEF | nsp2(806-2719) | 156-163 |
| 2 | 1154-1163 | PRRARSVRYP linker 1-10 | | |
| 2 | 1164-1179 | NFLVQAGNVQLRVIGH | 3C-like_protinase(10055-10972) | 65-80 |
| 2 | 1180-1189 | PRRARSVRYP linker 1-10 | | |
| 2 | 1190-1197 | EVTPSGTW | nucleocapsid_phosphoprotein | 323-330 |
| | | | (28274-29533) | |
| 2 | 1198-1207 | PRRARSVRYP linker 1-10 | | |
| 2 | 1208-1217 | FKHLIPLMYK | 3′-to-5′_exonuclease(18040-19620) | 146-155 |
| 2 | 1218-1227 | PRRARSVRYP linker 1-10 | | |
| 2 | 1228-1255 | SIPCSVCLSGLDSLDTYPSLETIQITI | nsp3(2720-8554) | 1467-1494 |
| | | S | | |
| 2 | 1256-1265 | PRRARSVRYP linker 1-10 | | |
| 2 | 1266-1273 | ISDYDYYR | RNA-dependent_RNA_polymerase | 450-457 |
| | | | (13442-13468 + 13468-16236) | |
| 2 | 1274-1283 | PRRARSVRYP linker 1-10 | | |
| 2 | 1284-1297 | FQTLLALHRSYLTP | surface_glycoprotein(21563-25384) | 238-251 |
| 2 | 1298-1307 | PRRARSVRYP linker 1-10 | | |
| 2 | 1308-1315 | NHTGVVGE | nsp2(806-2719) | 254-261 |
| 2 | 1316-1325 | PRRARSVRYP linker 1-10 | | |
| 2 | 1326-1342 | AAAYYVGYLQPRTFLLK | surface_glycoprotein(21563-25384) | 262-278 |
| 2 | 1343-1352 | PRRARSVRYP linker 1-10 | | |
| 2 | 1353-1360 | STFKCYGV | surface_glycoprotein(21563-25384) | 375-382 |
| 2 | 1364-1370 | PRRARSVRYP linker 1-10 | | |
| 2 | 1371-1385 | FKELLVYAADPAMHA | RNA-dependent_RNA_polymerase | 368-382 |
| | | | (13442-13468 + 13468-16236) | |
| 3 | 1-28 | RLCAYCCNIVNVSLVKPSFYVYSRVKN | envelope_protein(26245-26472) | 38-65 |
| | | L | | |
| 3 | 29-38 | PRRARSVRYP linker 1-10 | | |
| 3 | 39-52 | MPVCVETKAIVSTI | nsp3(2720-8554) | 571-584 |
| 3 | 53-62 | PRRARSVRYP linker 1-10 | | |
| 3 | 63-74 | KTTEVVGDIILK | nsp3(2720-8554) | 1249-1260 |
| 3 | 75-84 | PRRARSVRYP linker 1-10 | | |
| 3 | 85-102 | AFKLNIKLLGVGGKPCIK | nsp6(10973-11842) | 268-285 |
| 3 | 103-112 | PRRARSVRYP linker 1-10 | | |
| 3 | 113-120 | LLLDQALF | nsp3(2720-8554) | 1752-1759 |
| 3 | 121-130 | PRRARSVRYP linker 1-10 | | |
| 3 | 131-150 | ANSIVCRFDTRVLSNLNLPG | 3′-to-5′_exonuclease(18040-19620) | 394-413 |
| 3 | 151-160 | PRRARSVRYP linker 1-10 | | |
| 3 | 161-172 | LKKLKKSLNVAK | nsp8(12092-12685) | 35-46 |
| 3 | 173-182 | PRRARSVRYP linker 1-10 | | |
| 3 | 183-192 | KESVQTFFKL | nsp2(806-2719) | 492-501 |
| 3 | 193-202 | PRRARSVRYP linker 1-10 | | |
| 3 | 203-225 | GEAVKTQFNYYKKVDGVVQQLPE | endoRNAse(19621-20658) | 169-191 |
| 3 | 226-235 | PRRARSVRYP linker 1-10 | | |
| 3 | 236-245 | IGDCATVHTA | 2′-O-ribose_methyltransferase | 112-121 |
| | | | (20659-21552) | |
| 3 | 246-255 | PRRARSVRYP linker 1-10 | | |
| 3 | 256-265 | AQYELKHGTF | nsp3(2720-8554) | 994-1003 |
| 3 | 266-275 | PRRARSVRYP linker 1-10 | | |
| 3 | 276-286 | HNLAKHCLHVV | nsp3(2720-8554) | 290-300 |
| 3 | 287-296 | PRRARSVRYP linker 1-10 | | |
| 3 | 297-333 | GCDVTDVTQLYYLGGMSYYCKSHKPPI | helicase(16237-18039) | 54-90 |
| | | SFPLCANGQVF | | |
| 3 | 334-343 | PRRARSVRYP linker 1-10 | | |
| 3 | 344-370 | NAACRKVQHMVVKAALLADKFPVLHDI | 3′-to-5′_exonuclease(18040-19620) | 306-332 |
| 3 | 371-380 | PRRARSVRYP linker 1-10 | | |
| 3 | 381-391 | VEEVLSEARQH | leader_protein(266-805) | 35-45 |
| 3 | 392-401 | PRRARSVRYP linker 1-10 | | |
| 3 | 402-413 | IEVQGYKSVNIT | nsp3(2720-8554) | 13-24 |
| 3 | 414-423 | PRRARSVRYP linker 1-10 | | |
| 3 | 424-431 | NYQCGHYK | nsp3(2720-8554) | 1012-1019 |
| 3 | 432-441 | PRRARSVRYP linker 1-10 | | |
| 3 | 442-453 | TVNVLAWLYAAV | 3C-like_proteinase(10055-10972) | 201-212 |
| 3 | 454-463 | PRRARSVRYP linker 1-10 | | |
| 3 | 464-479 | SEFSSLPSYAAFATAQ | nsp8(12092-12685) | 4-19 |
| 3 | 480-489 | PRRARSVRYP linker 1-10 | | |
| 3 | 490-511 | LPRVFSAVGNICYTPSKLIEYT | nsp4(8555-10054) | 122-143 |
| 3 | 512-521 | PRRARSVRYP linker 1-10 | | |
| 3 | 522-536 | SVLLSMQGAVDINKL | nsp7(11843-12091) | 57-71 |
| 3 | 537-546 | PRRARSVRYP linker 1-10 | | |
| 3 | 547-560 | CGACIRRPFLCCKC | helicase(16237-18039) | 16-29 |
| 3 | 561-570 | PRRARSVRYP linker 1-10 | | |
| 3 | 571-594 | DPIHSLRVCVDTVRTNVYLAVFDK | nsp3(2720-8554) | 339-362 |
| 3 | 595-604 | PRRARSVRYP linker 1-10 | | |
| 3 | 605-615 | SEAFLIGCNYL | 2′-O-ribose_methyltransferase | 202-212 |
| | | | (20659-21552) | |
| 3 | 616-625 | PRRARSVRYP linker 1-10 | | |
| 3 | 626-641 | DQVILLNKHIDAYKTF | nucleocapsid_phosphoprotein | 348-363 |
| | | | (28274-29533) | |
| 3 | 642-651 | PRRARSVRYP linker 1-10 | | |
| 3 | 652-659 | EHVQIHTI | ORF3a_protein(25393-26220) | 242-249 |
| 3 | 660-669 | PRRARSVRYP linker 1-10 | | |
| 3 | 670-696 | AKYTQLCQYLNTLTLAVPYNMRVIHF | 2′-O-ribose_methyltransferase | 45-71 |
| | | | (20659-21552) | |
| 3 | 697-706 | PRRARSVRYP linker 1-10 | | |
| 3 | 707-726 | GYLPQNAVVKIYCPACHNSE | nsp2(806-2719) | 178-197 |
| 3 | 727-736 | PRRARSVRYP linker 1-10 | | |
| 3 | 737-753 | IVKFISTCACEIVGGQI | nsp2(806-2719) | 468-484 |
| 3 | 754-763 | PRRARSVRYP linker 1-10 | | |
| 3 | 764-775 | VPVVDSYYSLLM | RNA-dependent_RNA_polymerase | 231-242 |
| | | | (13442-13468 + 13468-16236) | |
| 3 | 776-785 | PRRARSVRYP linker 1-10 | | |
| 3 | 786-810 | DDRVILHCANFNVLFSTVFPPTSFG | RNA-dependent_RNA_polymerase | 303-327 |
| | | | (13442-13468 + 13468-16236) | |
| 3 | 811-820 | PRRARSVRYP linker 1-10 | | |
| 3 | 821-846 | QNSVRVLQKAAITILDGISQYSLRLI | nsp2(806-2719) | 376-401 |
| 3 | 847-856 | PRRARSVRYP linker 1-10 | | |
| 3 | 857-876 | VVIKVCEFQFCNDPFLGVYY | surface_glycoprotein(21563-25384) | 126-145 |
| 3 | 877-886 | PRRARSVRYP linker 1-10 | | |
| 3 | 887-897 | IEELFYSYATH | 3′-to-5′_exonuclease(18040-19620) | 363-373 |
| 3 | 898-907 | PRRARSVRYP linker 1-10 | | |
| 3 | 908-939 | VDFCGKGYHLMSFPQSAPHGVVFLHVT | surface_glycoprotein(21563-25384) | 1040-1071 |
| | | YVPAQ | | |
| 3 | 940-949 | PRRARSVRYP linker 1-10 | | |
| 3 | 950-967 | PNFVFPLNSIIKTIQPRV | nsp2(806-2719) | 91-108 |
| 3 | 968-977 | PRRARSVRYP linker 1-10 | | |
| 3 | 978-993 | NDLCFTNVYADSFVIR | surface_glycoprotein(21563-25384) | 388-403 |
| 3 | 994-1003 | PRRARSVRYP linker 1-10 | | |
| 3 | 1004-1012 | VLGSLAATV | nsp9(12686-13024) | 102-110 |
| 3 | 1013-1022 | PRRARSVRYP linker 1-10 | | |
| 3 | 1023-1032 | FEYVSQPFLM | surface_glycoprotein(21563-25384) | 168-177 |
| 3 | 1033-1042 | PRRARSVRYP linker 1-10 | | |
| 3 | 1043-1062 | KGFCDLKGKYVQIPTTCAND | nsp10(13025-13441) | 87-106 |
| 3 | 1063-1072 | PRRARSVRYP linker 1-10 | | |
| 3 | 1073-1092 | INLVRDLPQGFSALEPLVDL | surface_glycoprotein(21563-25384) | 210-229 |
| 3 | 1093-1102 | PRRARSVRYP linker 1-10 | | |
| 3 | 1103-1120 | YADVFHLYLQYIRKLHDE | RNA-dependent_RNA_polymerase | 877-894 |
| | | | (13442-13468 + 13468-16236) | |
| 3 | 1121-1130 | PRRARSVRYP linker 1-10 | | |
| 3 | 1131-1140 | KQYGDCLGDI | surface_glycoprotein(21563-25384) | 835-844 |
| 3 | 1141-1150 | PRRARSVRYP linker 1-10 | | |
| 3 | 1151-1160 | GVITHDVSSA | helicase(16237-18039) | 478-487 |
| 3 | 1161-1170 | PRRARSVRYP linker 1-10 | | |
| 3 | 1171-1180 | GLPVKYLYFI | nsp9(12686-13024) | 82-91 |
| 3 | 1181-1190 | PRRARSVRYP linker 1-10 | | |
| 3 | 1191-1201 | SPDAVTAYNGY | nsp3(2720-8554) | 661-671 |
| 3 | 1202-1211 | PRRARSVRYP linker 1-10 | | |
| 3 | 1212-1225 | ARSVASQSIIAYTM | surface_glycoprotein(21563-25384) | 684-697 |
| 3 | 1226-1235 | PRRARSVRYP linker 1-10 | | |
| 3 | 1236-1246 | VLPFNDGVYFA | surface_glycoprotein(21563-25384) | 83-93 |
| 3 | 1247-1256 | PRRARSVRYP linker 1-10 | | |
| 3 | 1257-1271 | AQSFLNRVCGVSAAR | RNA-dependent_RNA_polymerase | 4-18 |
| | | | (13442-13468 + 13468-16236) | |
| 3 | 1272-1281 | PRRARSVRYP linker 1-10 | | |
| 3 | 1282-1289 | AEVQIDRL | surface_glycoprotein(21563-25384) | 989-996 |
| 3 | 1290-1299 | PRRARSVRYP linker 1-10 | | |
| 3 | 1300-1307 | DLSVVNAR | helicase(16237-18039) | 383-390 |
| 3 | 1308-1317 | PRRARSVRYP linker 1-10 | | |
| 3 | 1318-1334 | FRELGVVHNQDVNLHSS | RNA-dependent_RNA_polymerase | 348-364 |
| | | | (13442-13468 + 13468-16236) | |
| 3 | 1335-1344 | PRRARSVRYP linker 1-10 | | |
| 3 | 1345-1356 | NFRVQPTESIVR | surface_glycoprotein(21563-25384) | 317-328 |
| 3 | 1357-1366 | PRRARSVRYP linker 1-10 | | |
| 3 | 1367-1387 | CSARHINAQVAKSHNIALIWN | nsp3(2720-8554) | 1876-1896 |
| 4 | 1-10 | EQYVFCTVNA | helicase(16237-18039) | 353-362 |
| 4 | 11-20 | PRRARSVRYP linker 1-10 | | |
| 4 | 21-32 | TRAKVGILCIMS | helicase(16237-18039) | 566-577 |
| 4 | 33-42 | PRRARSVRYP linker 1-10 | | |
| 4 | 43-54 | FKLASHMYCSFY | nsp3(2720-8554) | 96-107 |
| 4 | 55-64 | PRRARSVRYP linker 1-10 | | |
| 4 | 65-72 | SRELKVTF | nsp3(2720-8554) | 1134-1141 |
| 4 | 73-82 | PRRARSVRYP linker 1-10 | | |
| 4 | 83-100 | DKACPLIAAVITREVGFV | nsp4(8555-10054) | 85-102 |
| 4 | 101-110 | PRRARSVRYP linker 1-10 | | |
| 4 | 111-120 | LEGSVAYESL | nsp4(8555-10054) | 176-185 |
| 4 | 121-130 | PRRARSVRYP linker 1-10 | | |
| 4 | 131-140 | GTNLPLQLGF | 3′-to-5′_exonuclease(18040-19620) | 102-111 |
| 4 | 141-150 | PRRARSVRYP linker 1-10 | | |
| 4 | 151-158 | ADAVIKTL | nsp3(2720-8554) | 58-65 |
| 4 | 159-168 | PRRARSVRYP linker 1-10 | | |
| 4 | 169-177 | RSVYPVASP | nsp2(806-2719) | 121-129 |
| 4 | 178-187 | PRRARSVRYP linker 1-10 | | |
| 4 | 188-217 | SDIDITFLKKDAPYIVGDVVQEGVLTA | nsp3(2720-8554) | 454-483 |
| | | VVI | | |
| 4 | 218-227 | PRRARSVRYP linker 1-10 | | |
| 4 | 228-238 | TEISFMLWCKD | endoRNAse(19621-20658) | 325-335 |
| 4 | 239-248 | PRRARSVRYP linker 1-10 | | |
| 4 | 249-256 | ARDLSLQF | nsp3(2720-8554) | 1652-1659 |
| 4 | 257-266 | PRRARSVRYP linker 1-10 | | |
| 4 | 267-279 | KLALGGSVAIKIT | 2′-O-ribose_methyltransferase | 160-172 |
| | | | (20659-21552) | |
| 4 | 280-289 | PRRARSVRYP linker 1-10 | | |
| 4 | 290-299 | FDVVRQCSGV | 3C-like_proteinase(10055-10972) | 294-303 |
| 4 | 300-309 | PRRARSVRYP linker 1-10 | | |
| 4 | 310-319 | RHSLSHFVNL | nsp3(2720-8554) | 1697-1706 |
| 4 | 320-329 | PRRARSVRYP linker 1-10 | | |
| 4 | 330-337 | KAVFISPY | helicase(16237-18039) | 508-515 |
| 4 | 338-347 | PRRARSVRYP linker 1-10 | | |
| 4 | 348-368 | LGVLVPHVGEIPVAYRKVLLR | leader_protein(266-805) | 104-124 |
| 4 | 369-378 | PRRARSVRYP linker 1-10 | | |
| 4 | 379-394 | IKPVTYKLDGVVCTEI | nsp3(2720-8554) | 1059-1074 |
| 4 | 395-404 | PRRARSVRYP linker 1-10 | | |
| 4 | 405-412 | SLWVYKQF | 3′-to-5′_exonuclease(18040-19620) | 507-514 |
| 4 | 413-422 | PRRARSVRYP linker 1-10 | | |
| 4 | 423-436 | RLTLGVYDYLVSTQ | nsp6(10973-11842) | 236-249 |
| 4 | 437-446 | PRRARSVRYP linker 1-10 | | |
| 4 | 447-469 | ELYHYQECVRGTTVLLKEPCSSG | ORF7a_protein(27394-27759) | 16-38 |
| 4 | 470-479 | PRRARSVRYP linker 1-10 | | |
| 4 | 480-506 | FSASTSAFVETVKGLDYKAFKQIVESC | nsp2(806-2719) | 300-326 |
| 4 | 507-516 | PRRARSVRYP linker 1-10 | | |
| 4 | 517-546 | CQVHGNAHVASCSAIMTRCLAVHECFV | 3′-to-5′_exonuclease(18040-19620) | 261-290 |
| | | KRV | | |
| 4 | 547-556 | PRRARSVRYP linker 1-10 | | |
| 4 | 557-577 | VKDCVVLHSYFTSDYYQLYST | ORF3a_protein(25393-26220) | 197-217 |
| 4 | 578-587 | PRRARSVRYP linker 1-10 | | |
| 4 | 588-600 | YTELEPPCRFVTD | nsp9(12686-13024) | 66-78 |
| 4 | 601-610 | PRRARSVRYP linker 1-10 | | |
| 4 | 611-631 | QPYRVVVLSFELLHAPATVCG | surface_glycoprotein(21563-25384) | 506-526 |
| 4 | 632-641 | PRRARSVRYP linker 1-10 | | |
| 4 | 642-651 | PRWYFYYLGT | nucleocapsid_phosphoprotein | 106-115 |
| | | | (28274-29533) | |
| 4 | 652-661 | PRRARSVRYP linker 1-10 | | |
| 4 | 662-698 | ALTCFSTQFAFACPDGVKHVYQLRARS | ORF7a_protein(27394-27759) | 55-91 |
| | | VSPKLFIRQE | | |
| 4 | 699-708 | PRRARSVRYP linker 1-10 | | |
| 4 | 709-723 | VEHVTFFIYNKIVDE | ORF3a_protein(25393-26220) | 225-239 |
| 4 | 724-733 | PRRARSVRYP linker 1-10 | | |
| 4 | 734-757 | LGSLVVRCSFYEDFLEYHDVRVVL | ORF8_protein(27894-28259) | 95-118 |
| 4 | 758-767 | PRRARSVRYP linker 1-10 | | |
| 4 | 768-779 | ALGKLQDVVNQN | surface_glycoprotein(21563-25384) | 944-955 |
| 4 | 780-789 | PRRARSVRYP linker 1-10 | | |
| 4 | 790-846 | HFAIGLALYYPSARIVYTACSHAAVDA | helicase(16237-18039) | 290-346 |
| | | LCEKALKYLPIDKCSRIIPARARVECF | | |
| | | DKF | | |
| 4 | 847-856 | PRRARSVRYP linker 1-10 | | |
| 4 | 857-874 | ANSVFNICQAVTANVNAL | RNA-dependent_RNA_polymerase | 690-707 |
| | | | (13442-13468 + 13468-16236) | |
| 4 | 875-884 | PRRARSVRYP linker 1-10 | | |
| 4 | 885-900 | VREVLSDRELHLSWEV | helicase(16237-18039) | 154-169 |
| 4 | 901-910 | PRRARSVRYP linker 1-10 | | |
| 4 | 911-918 | FNSVCRLM | helicase(16237-18039) | 422-429 |
| 4 | 919-928 | PRRARSVRYP linker 1-10 | | |
| 4 | 929-936 | SASKIITL | ORF3a_protein(25393-26220) | 58-65 |
| 4 | 937-946 | PRRARSVRYP linker 1-10 | | |
| 4 | 947-959 | VEGCMVQVTCGTT | 3C-like_proteinase(10055-10972) | 13-25 |
| 4 | 960-969 | PRRARSVRYP linker 1-10 | | |
| 4 | 970-1013 | KSAFVNLKQLPFFYYSDSPCESHGKQV | 3′-to-5′_exonuclease(18040-19620) | 433-476 |
| | | VSDIDYVPLKSATCITR | | |
| 4 | 1014-1023 | PRRARSVRYP linker 1-10 | | |
| 4 | 1024-1038 | EVPANSTVLSFCAFA | nsp10(13025-13441) | 6-20 |
| 4 | 1039-1048 | PRRARSVRYP linker 1-10 | | |
| 4 | 1049-1065 | LTQYNRYLALYNKYKYF | nsp4(8555-10054) | 438-454 |
| 4 | 1066-1075 | PRRARSVRYP linker 1-10 | | |
| 4 | 1076-1083 | NNLVVMAY | nsp2(806-2719) | 413-420 |
| 4 | 1084-1093 | PRRARSVRYP linker 1-10 | | |
| 4 | 1094-1101 | CSCGSCCK | surface_glycoprotein(21563-25384) | 1248-1255 |
| 4 | 1102-1111 | PRRARSVRYP linker 1-10 | | |
| 4 | 1112-1119 | DSYFVVKR | RNA-dependent_RNA_polymerase | 67-74 |
| | | | (13442-13468 + 13468-16236) | |
| 4 | 1120-1129 | PRRARSVRYP linker 1-10 | | |
| 4 | 1130-1139 | RQALLKTVQF | RNA-dependent_RNA_polymerase | 183-192 |
| | | | (13442-13468 + 13468-16236) | |
| 4 | 1140-1149 | PRRARSVRYP linker 1-10 | | |
| 4 | 1150-1160 | AALALLLLDRL | nucleocapsid_phosphoprotein | 217-227 |
| | | | (28274-29533) | |
| 4 | 1161-1170 | PRRARSVRYP linker 1-10 | | |
| 4 | 1171-1180 | SVLNDILSRL | surface_glycoprotein(21563-25384) | 975-984 |
| 4 | 1181-1190 | PRRARSVRYP linker 1-10 | | |
| 4 | 1191-1210 | YLFQHANLDSCKRVLNVVCK | nsp3(2720-8554) | 916-935 |
| 4 | 1211-1220 | PRRARSVRYP linker 1-10 | | |
| 4 | 1221-1233 | GVNLVAVPTGYVD | 3′-to-5′_exonuclease(18040-19620) | 114-126 |
| 4 | 1234-1243 | PRRARSVRYP linker 1-10 | | |
| 4 | 1244-1251 | ISEHDYQI | ORF3a_protein(25393-26220) | 179-186 |
| 4 | 1252-1261 | PRRARSVRYP linker 1-10 | | |
| 4 | 1262-1282 | KSILSPLYAFASEAARVVRSI | nsp2(806-2719) | 347-367 |
| 4 | 1283-1292 | PRRARSVRYP linker 1-10 | | |
| 4 | 1293-1300 | FTGCVIAW | surface_glycoprotein(21563-25384) | 429-436 |
| 4 | 1301-1310 | PRRARSVRYP linker 1-10 | | |
| 4 | 1311-1322 | MPLKAPKEIIFL | nsp2(806-2719) | 551-562 |
| 4 | 1323-1332 | PRRARSVRYP linker 1-10 | | |
| 4 | 1333-1346 | WNVVRIKIVQMLSD | 3′-to-5′_exonuclease(18040-19620) | 159-172 |
| 4 | 1347-1356 | PRRARSVRYP linker 1-10 | | |
| 4 | 1357-1382 | KNPLLYDANYFLCWHTNVYDYCIPYN | ORF3a_protein(25393-26220) | 136-161 |
| 4 | 1383-1392 | PRRARSVRYP linker 1-10 | | |
| 4 | 1393-1408 | LQSLQTYVTQQLIRAA | surface_glycoprotein(21563-25384) | 1001-1016 |

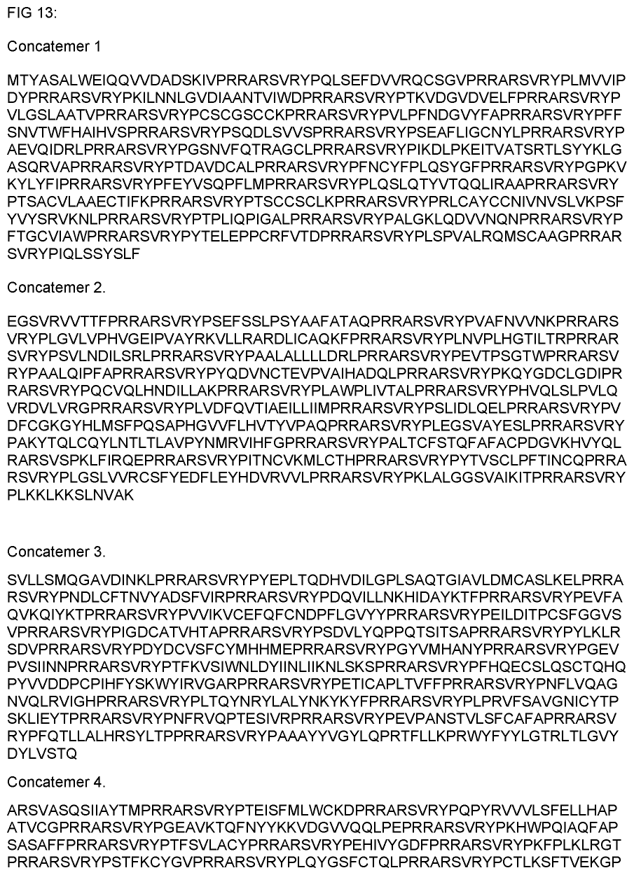

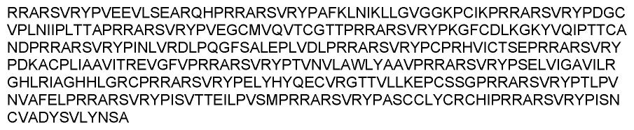

In certain embodiments, the vaccine comprises or expresses at least one of the contigs concatemers of epitopes from SARS-COV-2 with intervening linker sequences set forth in Table 2 (also see FIG. 13). In certain embodiments, the vaccine comprises or expresses at least two of the concatemers set forth below. In certain embodiments, the vaccine comprises or expresses three of the concatemers set forth below. In certain embodiments, the vaccine comprises or expresses all the concatemers set forth below.

TABLE 2

| concat- | pos_in_ | | | pos_in_ |
|---|---|---|---|---|
| emer | concatemer | epitope | gene | gene |
| 1 | 1-24 | TYASALWEIQQVVDADSKIVQLSE | nsp8(12092-12685) | 148-171 |
| 1 | 25-34 | PRRARSVRYP linker 1-10 | | |
| 1 | 35-44 | FDVVRQCSGV | 3C-like_proteinase(10055-10972) | 294-303 |
| 1 | 45-54 | PRRARSVRYP linker 1-10 | | |
| 1 | 55-62 | LMVVIPDY | nsp8(12092-12685) | 128-135 |
| 1 | 63-72 | PRRARSVRYP linker 1-10 | | |
| 1 | 73-90 | KILNNLGVDIAANTVIWD | endoRNAse(19621-20658) | 70-87 |
| 1 | 91-100 | PRRARSVRYP linker 1-10 | | |
| 1 | 101-111 | TKVDGVDVELF | endoRNAse(19621-20658) | 33-43 |
| 1 | 112-121 | PRRARSVRYP linker 1-10 | | |
| 1 | 122-130 | VLGSLAATV | nsp9(12686-13024) | 102-110 |
| 1 | 131-140 | PRRARSVRYP linker 1-10 | | |
| 1 | 141-148 | CSCGSCCK | surface_glycoprotein(21563-25384) | 1248-1255 |
| 1 | 149-158 | PRRARSVRYP linker 1-10 | | |
| 1 | 159-169 | VLPFNDGVYFA | surface_glycoprotein(21563-25384) | 83-93 |
| 1 | 170-179 | PRRARSVRYP linker 1-10 | | |
| 1 | 180-193 | FFSNVTWFHAIHVS | surface_glycoprotein(21563-25384) | |
| 1 | 194-203 | PRRARSVRYP linker 1-10 | | |
| 1 | 204-211 | SQDLSVVS | endoRNAse(19621-20658) | 308-315 |
| 1 | 212-221 | PRRARSVRYP linker 1-10 | | |
| 1 | 222-232 | SEAFLIGCNYL | 2′-O-ribose_methyltransferase | 202-212 |
| | | | (20659-21552) | |
| 1 | 233-242 | PRRARSVRYP linker 1-10 | | |
| 1 | 243-250 | AEVQIDRL | surface_glycoprotein(21563-25384) | 989-996 |
| 1 | 251-260 | PRRARSVRYP linker 1-10 | | |
| 1 | 261-272 | GSNVFQTRAGCL | surface_glycoprotein(21563-25384) | 639-650 |
| 1 | 273-282 | PRRARSVRYP linker 1-10 | | |
| 1 | 283-310 | IKDLPKEITVATSRTLSYYKLGASQRVA | membrane_glycoprotein(26523-27191) | 161-188 |
| 1 | 311-320 | PRRARSVRYP linker 1-10 | | |
| 1 | 321-328 | TDAVDCAL | surface_glycoprotein(21563-25384) | 286-293 |
| 1 | 329-338 | PRRARSVRYP linker 1-10 | | |
| 1 | 339-350 | FNCYFPLQSYGF | surface_glycoprotein(21563-25384) | 486-497 |
| 1 | 351-360 | PRRARSVRYP linker 1-10 | | |
| 1 | 361-370 | GPKVKYLYFI | nsp9(12686-13024) | 82-91 |
| 1 | 371-380 | PRRARSVRYP linker 1-10 | | |
| 1 | 381-390 | FEYVSQPFLM | surface_glycoprotein(21563-25384) | 168-177 |
| 1 | 391-400 | PRRARSVRYP linker 1-10 | | |
| 1 | 401-416 | LQSLQTYVTQQLIRAA | surface_glycoprotein(21563-25384) | 1001-1016 |
| 1 | 417-426 | PRRARSVRYP linker 1-10 | | |
| 1 | 427-440 | TSACVLAAECTIFK | nsp4(8555-10054) | 147-160 |
| 1 | 441-450 | PRRARSVRYP linker 1-10 | | |
| 1 | 451-458 | TSCCSCLK | surface_glycoprotein(21563-25384) | 1238-1245 |
| 1 | 459-468 | PRRARSVRYP linker 1-10 | | |
| 1 | 469-496 | RLCAYCCNIVNVSLVKPSFYVYSRVKNL | envelope_protein(26245-26472) | 38-65 |
| 1 | 497-506 | PRRARSVRYP linker 1-10 | | |
| 1 | 507-516 | TPLIQPIGAL | nsp4(8555-10054) | 269-278 |
| 1 | 517-526 | PRRARSVRYP linker 1-10 | | |
| 1 | 527-538 | ALGKLQDVVNQN | surface_glycoprotein(21563-25384) | 944-955 |
| 1 | 539-548 | PRRARSVRYP linker 1-10 | | |
| 1 | 549-556 | FTGCVIAW | surface_glycoprotein(21563-25384) | 429-436 |
| 1 | 557-566 | PRRARSVRYP linker 1-10 | | |
| 1 | 567-579 | YTELEPPCRFVTD | nsp9(12686-13024) | 66-78 |
| 1 | 580-589 | PRRARSVRYP linker 1-10 | | |
| 1 | 590-603 | LSPVALRQMSCAAG | nsp9(12686-13024) | 4-17 |
| 1 | 604-613 | PRRARSVRYP linker 1-10 | | |
| 1 | 614-622 | IQLSSYSLF | 2′-O-ribose_methyltransferase | 237-245 |
| | | | (20659-21552) | |
| 2 | 1-10 | EGSVRVVTTF | nsp4(8555-10054) | 207-216 |
| 2 | 11-20 | PRRARSVRYP linker 1-10 | | |
| 2 | 21-36 | SEFSSLPSYAAFATAQ | nsp8(12092-12685) | 4-19 |
| 2 | 37-46 | PRRARSVRYP linker 1-10 | | |
| 2 | 47-54 | VAFNVVNK | endoRNAse(19621-20658) | 5-12 |
| 2 | 55-64 | PRRARSVRYP linker 1-10 | | |
| 2 | 65-85 | LGVLVPHVGEIPVAYRKVLLR | leader_protein(266-805) | 104-124 |
| 2 | 86-95 | PRRARSVRYP linker 1-10 | | |
| 2 | 96-105 | ARDLICAQKF | surface_glycoprotein(21563-25384) | 846-855 |
| 2 | 106-115 | PRRARSVRYP linker 1-10 | | |
| 2 | 116-127 | LNVPLHGTILTR | membrane_glycoprotein(26523-27191) | 120-131 |
| 2 | 128-137 | PRRARSVRYP linker 1-10 | | |
| 2 | 138-147 | SVLNDILSRL | surface_glycoprotein(21563-25384) | 975-984 |
| 2 | 148-157 | PRRARSVRYP linker 1-10 | | |
| 2 | 158-168 | AALALLLLDRL | nucleocapsid_phosphoprotein | 217-227 |
| | | | (28274-29533) | |
| 2 | 169-178 | PRRARSVRYP linker 1-10 | | |
| 2 | 179-186 | EVTPSGTW | nucleocapsid_phosphoprotein | 323-330 |
| | | | (28274-29533) | |
| 2 | 187-196 | PRRARSVRYP linker 1-10 | | |
| 2 | 197-204 | AALQIPFA | surface_glycoprotein(21563-25384) | 892-899 |
| 2 | 205-214 | PRRARSVRYP linker 1-10 | | |
| 2 | 215-232 | YQDVNCTEVPVAIHADQL | surface_glycoprotein(21563-25384) | 612-629 |
| 2 | 233-242 | PRRARSVRYP linker 1-10 | | |
| 2 | 243-252 | KQYGDCLGDI | surface_glycoprotein(21563-25384) | 835-844 |
| 2 | 253-262 | PRRARSVRYP linker 1-10 | | |
| 2 | 263-275 | QCVQLHNDILLAK | nsp7(11843-12091) | 31-43 |
| 2 | 276-285 | PRRARSVRYP linker 1-10 | | |
| 2 | 286-295 | LAWPLIVTAL | nsp8(12092-12685) | 180-189 |
| 2 | 296-305 | PRRARSVRYP linker 1-10 | | |
| 2 | 306-323 | HVQLSLPVLQVRDVLVRG | leader_protein(266-805) | 13-30 |
| 2 | 324-333 | PRRARSVRYP linker 1-10 | | |
| 2 | 334-349 | LVDFQVTIAEILLIIM | ORF6_protein(27202-27387) | 4-19 |
| 2 | 350-359 | PRRARSVRYP linker 1-10 | | |
| 2 | 360-367 | SLIDLQEL | surface_glycoprotein(21563-25384) | 1196-1203 |
| 2 | 368-377 | PRRARSVRYP linker 1-10 | | |
| 2 | 378-409 | VDFCGKGYHLMSFPQSAPHGVVFLHVTY | surface_glycoprotein(21563-25384) | 1040-1071 |
| | | VPAQ | | |
| 2 | 410-419 | PRRARSVRYP linker 1-10 | | |
| 2 | 420-429 | LEGSVAYESL | nsp4(8555-10054) | 176-185 |
| 2 | 430-439 | PRRARSVRYP linker 1-10 | | |
| 2 | 440-466 | AKYTQLCQYLNTLTLAVPYNMRVIHFG | 2′-O-ribose_methyltransferase | 45-71 |
| | | | (20659-21552) | |
| 2 | 467-476 | PRRARSVRYP linker 1-10 | | |
| 2 | 477-513 | ALTCFSTQFAFACPDGVKHVYQLRARSV | ORF7a_protein(27394-27759) | 55-91 |
| | | SPKLFIRQE | | |
| 2 | 514-523 | PRRARSVRYP linker 1-10 | | |
| 2 | 524-534 | ITNCVKMLCTH | nsp10(13025-13441) | 38-48 |
| 2 | 535-544 | PRRARSVRYP linker 1-10 | | |
| 2 | 545-557 | YTVSCLPFTINCQ | ORF8_protein(27894-28259) | 79-91 |
| 2 | 558-567 | PRRARSVRYP linker 1-10 | | |
| 2 | 568-591 | LGSLVVRCSFYEDFLEYHDVRVVL | ORF8_protein(27894-28259) | 95-118 |
| 2 | 592-601 | PRRARSVRYP linker 1-10 | | |
| 2 | 602-614 | KLALGGSVAIKIT | 2′-O-ribose_methyltransferase | 160-172 |
| | | | (20659-21552) | |
| 2 | 615-624 | PRRARSVRYP linker 1-10 | | |
| 2 | 625-636 | LKKLKKSLNVAK | nsp8(12092-12685) | 35-46 |
| 3 | 1-15 | SVLLSMQGAVDINKL | nsp7(11843-12091) | 57-71 |
| 3 | 16-25 | PRRARSVRYP linker 1-10 | | |
| 3 | 26-58 | YEPLTQDHVDILGPLSAQTGIAVLDMCA | 3C-like_proteinase(10055-10972) | 239-271 |
| | | SLKEI | | |
| 3 | 59-68 | PRRARSVRYP linker 1-10 | | |
| 3 | 69-84 | NDLCFTNVYADSFVIR | surface_glycoprotein(21563-25384) | 388-403 |
| 3 | 85-94 | PRRARSVRYP linker 1-10 | | |
| 3 | 95-110 | DQVILLNKHIDAYKTF | nucleocapsid_phosphoprotein | 348-363 |
| | | | (28274-29533) | |
| 3 | 111-120 | PRRARSVRYP linker 1-10 | | |
| 3 | 121-132 | EVFAQVKQIYKT | surface_glycoprotein(21563-25384) | 780-791 |
| 3 | 133-142 | PRRARSVRYP linker 1-10 | | |
| 3 | 143-162 | VVIKVCEFQFCNDPFLGVYY | surface_glycoprotein(21563-25384) | 126-145 |
| 3 | 163-172 | PRRARSVRYP linker 1-10 | | |
| 3 | 173-187 | EILDITPCSFGGVSV | surface_glycoprotein(21563-25384) | 583-597 |
| 3 | 188-197 | PRRARSVRYP linker 1-10 | | |
| 3 | 198-207 | IGDCATVHTA | 2′-O-ribose_methyltransferase | 112-121 |
| | | | (20659-21552) | |
| 3 | 208-217 | PRRARSVRYP linker 1-10 | | |
| 3 | 218-232 | SDVLYQPPQTSITSA | nsp4(8555-10054) | 483-497 |
| 3 | 233-242 | PRRARSVRYP linker 1-10 | | |
| 3 | 243-250 | YLKLRSDV | nsp4(8555-10054) | 427-434 |
| 3 | 251-260 | PRRARSVRYP linker 1-10 | | |
| 3 | 261-274 | DYDCVSFCYMHHME | 3C-like_proteinase(10055-10972) | 153-166 |
| 3 | 275-284 | PRRARSVRYP linker 1-10 | | |
| 3 | 285-292 | GYVMHANY | 2′-O-ribose_methyltransferase | 221-228 |
| | | | (20659-21552) | |
| 3 | 293-302 | PRRARSVRYP linker 1-10 | | |
| 3 | 303-312 | GEVPVSIINN | endoRNAse(19621-20658) | 20-29 |
| 3 | 313-322 | PRRARSVRYP linker 1-10 | | |
| 3 | 323-345 | TFKVSIWNLDYIINLIIKNLSKS | ORF6_protein(27202-27387) | 21-43 |
| 3 | 346-355 | PRRARSVRYP linker 1-10 | | |
| 3 | 356-392 | FHQECSLQSCTQHQPYVVDDPCPIHFYS | ORF8_protein(27894-28259) | 16-52 |
| | | KWYIRVGAR | | |
| 3 | 393-402 | PRRARSVRYP linker 1-10 | | |
| 3 | 403-413 | ETICAPLTVFF | endoRNAse(19621-20658) | 113-123 |
| 3 | 414-423 | PRRARSVRYP linker 1-10 | | |
| 3 | 424-439 | NFLVQAGNVQLRVIGH | 3C-like_proteinase(10055-10972) | 65-80 |
| 3 | 440-449 | PRRARSVRYP linker 1-10 | | |
| 3 | 450-466 | LTQYNRYLALYNKYKYF | nsp4(8555-10054) | 438-454 |
| 3 | 467-476 | PRRARSVRYP linker 1-10 | | |
| 3 | 477-498 | LPRVFSAVGNICYTPSKLIEYT | nsp4(8555-10054) | 122-143 |
| 3 | 499-508 | PRRARSVRYP linker 1-10 | | |
| 3 | 509-520 | NFRVQPTESIVR | surface_glycoprotein(21563-25384) | 317-328 |
| 3 | 521-530 | PRRARSVRYP linker 1-10 | | |
| 3 | 531-545 | EVPANSTVLSFCAFA | nsp10(13025-13441) | 6-20 |
| 3 | 546-555 | PRRARSVRYP linker 1-10 | | |
| 3 | 556-569 | FQTLLALHRSYLTP | surface_glycoprotein(21563-25384) | 238-251 |
| 3 | 570-579 | PRRARSVRYP linker 1-10 | | |
| 3 | 580-596 | AAAYYVGYLQPRTFLLK | surface_glycoprotein(21563-25384) | 262-278 |
| 3 | 597-606 | PRRARSVRYP linker 1-10 | | |
| 3 | 607-616 | PRWYFYYLGT | nucleocapsid_phosphoprotein | 106-115 |
| | | | (28274-29533) | |
| 3 | 617-626 | PRRARSVRYP linker 1-10 | | |
| 3 | 627-640 | RLTLGVYDYLVSTQ | nsp6(10973-11842) | 236-249 |
| 4 | 1-14 | ARSVASQSIIAYTM | surface_glycoprotein(21563-25384) | 684-697 |
| 4 | 15-24 | PRRARSVRYP linker 1-10 | | |
| 4 | 25-35 | TEISFMLWCKD | endoRNAse(19621-20658) | 325-335 |
| 4 | 36-45 | PRRARSVRYP linker 1-10 | | |
| 4 | 46-66 | QPYRVVVLSFELLHAPATVCG | surface_glycoprotein(21563-25384) | 506-526 |
| 4 | 67-76 | PRRARSVRYP linker 1-10 | | |
| 4 | 77-99 | GEAVKTQFNYYKKVDGVVQQLPE | endoRNAse(19621-20658) | 169-191 |
| 4 | 100-109 | PRRARSVRYP linker 1-10 | | |
| 4 | 110-126 | KHWPQIAQFAPSASAFF | nucleocapsid_phosphoprotein | 299-315 |
| | | | (28274-29533) | |
| 4 | 127-136 | PRRARSVRYP linker 1-10 | | |
| 4 | 137-144 | TFSVLACY | 3C-like_proteinase(10055-10972) | 111-118 |
| 4 | 145-154 | PRRARSVRYP linker 1-10 | | |
| 4 | 155-162 | EHIVYGDF | endoRNAse(19621-20658) | 233-240 |
| 4 | 163-172 | PRRARSVRYP linker 1-10 | | |
| 4 | 173-181 | KFPLKLRGT | 2′-O-ribose_methyltransferase | 249-257 |
| | | | (20659-21552) | |
| 4 | 182-191 | PRRARSVRYP linker 1-10 | | |
| 4 | 192-199 | STFKCYGV | surface_glycoprotein(21563-25384) | 375-382 |
| 4 | 200-209 | PRRARSVRYP linker 1-10 | | |
| 4 | 210-219 | LQYGSFCTQL | surface_glycoprotein(21563-25384) | 754-763 |
| 4 | 220-229 | PRRARSVRYP linker 1-10 | | |
| 4 | 230-240 | CTLKSFTVEKG | surface_glycoprotein(21563-25384) | 301-311 |
| 4 | 241-250 | PRRARSVRYP linker 1-10 | | |
| 4 | 251-261 | VEEVLSEARQH | leader_protein(266-805) | 35-45 |
| 4 | 262-271 | PRRARSVRYP linker 1-10 | | |
| 4 | 272-289 | AFKLNIKLLGVGGKPCIK | nsp6(10973-11842) | 268-285 |
| 4 | 290-299 | PRRARSVRYP linker 1-10 | | |
| 4 | 300-313 | DGCVPLNIIPLTTA | nsp8(12092-12685) | 112-125 |
| 4 | 314-323 | PRRARSVRYP linker 1-10 | | |
| 4 | 324-336 | VEGCMVQVTCGTT | 3C-like_proteinase(10055-10972) | 13-25 |
| 4 | 337-346 | PRRARSVRYP linker 1-10 | | |
| 4 | 347-366 | KGFCDLKGKYVQIPTTCAND | nsp10(13025-13441) | 87-106 |
| 4 | 367-376 | PRRARSVRYP linker 1-10 | | |
| 4 | 377-396 | INLVRDLPQGFSALEPLVDL | surface_glycoprotein(21563-25384) | 210-229 |
| 4 | 397-406 | PRRARSVRYP linker 1-10 | | |
| 4 | 407-416 | CPRHVICTSE | 3C-like_proteinase(10055-10972) | 38-47 |
| 4 | 417-426 | PRRARSVRYP linker 1-10 | | |
| 4 | 427-444 | DKACPLIAAVITREVGFV | nsp4(8555-10054) | 85-102 |
| 4 | 445-454 | PRRARSVRYP linker 1-10 | | |
| 4 | 455-466 | TVNVLAWLYAAV | 3C-like_proteinase(10055-10972) | 201-212 |
| 4 | 467-476 | PRRARSVRYP linker 1-10 | | |
| 4 | 477-500 | SELVIGAVILRGHLRIAGHHLGRC | membrane_glycoprotein(26523-27191) | 136-159 |
| 4 | 501-510 | PRRARSVRYP linker 1-10 | | |
| 4 | 511-533 | ELYHYQECVRGTTVLLKEPCSSG | ORF7a_protein(27394-27759) | 16-38 |
| 4 | 534-543 | PRRARSVRYP linker 1-10 | | |
| 4 | 544-553 | TLPVNVAFEL | endoRNAse(19621-20658) | 48-57 |
| 4 | 554-563 | PRRARSVRYP linker 1-10 | | |
| 4 | 564-575 | ISVTTEILPVSM | surface_glycoprotein(21563-25384) | 720-731 |
| 4 | 576-585 | PRRARSVRYP linker 1-10 | | |
| 4 | 586-596 | ASCCLYCRCHI | nsp10(13025-13441) | 71-81 |
| 4 | 597-606 | PRRARSVRYP linker 1-10 | | |
| 4 | 607-621 | ISNCVADYSVLYNSA | surface_glycoprotein(21563-25384) | 358-372 |

In certain embodiments, the vaccine comprises or expresses at least one of the concatemers of epitopes from SARS-COV-2 with intervening linker sequences. In certain embodiments, the vaccine comprises or expresses at least two of the concatemers set forth below. In certain embodiments, the vaccine comprises or expresses three of the concatemer set forth below. In certain embodiments, the vaccine comprises or expresses all the concatemers set forth below.

TABLE 3

| concat- | pos_in_ | | | pos_in_ |
|---|---|---|---|---|
| emer | concatemer | epitope | gene | gene |
| 1 | 1-24 | TYASALWEIQQVVDADSKIVQLSE | nsp8(12092-12685) | 148-171 |
| 1 | 25-34 | PRRARSVRYP linker 1-10 | | |
| 1 | 35-47 | QCVQLHNDILLAK | nsp7(11843-12091) | 31-43 |
| 1 | 48-57 | PRRARSVRYP linker 1-10 | | |
| 1 | 58-70 | VEGCMVQVTCGTT | 3C-like_proteinase(10055-10972) | 13-25 |
| 1 | 71-80 | PRRARSVRYP linker 1-10 | | |
| 1 | 81-97 | LTQYNRYLALYNKYKYF | nsp4(8555-10054) | 438-454 |
| 1 | 98-107 | PRRARSVRYP linker 1-10 | | |
| 1 | 108-117 | TPLIQPIGAL | nsp4(8555-10054) | 269-278 |
| 1 | 118-127 | PRRARSVRYP linker 1-10 | | |
| 1 | 128-135 | LMVVIPDY | nsp8(12092-12685) | 128-135 |
| 1 | 136-145 | PRRARSVRYP linker 1-10 | | |
| 1 | 146-155 | TLPVNVAFEL | endoRNAse(19621-20658) | 48-57 |
| 1 | 156-165 | PRRARSVRYP linker 1-10 | | |
| 1 | 166-181 | DQVILLNKHIDAYKTF | nucleocapsid_phosphoprotein | 348-363 |
| | | | (28274-29533) | |
| 1 | 182-191 | PRRARSVRYP linker 1-10 | | |
| 1 | 192-203 | LKKLKKSLNVAK | nsp8(12092-12685) | 35-46 |
| 1 | 204-213 | PRRARSVRYP linker 1-10 | | |
| 1 | 214-221 | EHIVYGDF | endoRNAse(19621-20658) | 233-240 |
| 1 | 222-231 | PRRARSVRYP linker 1-10 | | |
| 1 | 232-242 | VEEVLSEARQH | leader_protein(266-805) | 35-45 |
| 1 | 243-252 | PRRARSVRYP linker 1-10 | | |
| 1 | 253-266 | DGCVPLNIIPLTTA | nsp8(12092-12685) | 112-125 |
| 1 | 267-276 | PRRARSVRYP linker 1-10 | | |
| 1 | 277-299 | ELYHYQECVRGTTVLLKEPCSSG | ORF7a_protein(27394-27759) | 16-38 |
| 1 | 300-309 | PRRARSVRYP linker 1-10 | | |
| 1 | 310-319 | PRWYFYYLGT | nucleocapsid_phosphoprotein | 106-115 |
| | | | (28274-29533) | |
| 1 | 320-329 | PRRARSVRYP linker 1-10 | | |
| 1 | 330-344 | SDVLYQPPQTSITSA | nsp4(8555-10054) | 483-497 |
| 1 | 345-354 | PRRARSVRYP linker 1-10 | | |
| 1 | 355-378 | SELVIGAVILRGHLRIAGHHLGRC | membrane_glycoprotein(26523-27191) | 136-159 |
| 1 | 379-388 | PRRARSVRYP linker 1-10 | | |
| 1 | 389-408 | KGFCDLKGKYVQIPTTCAND | nsp10(13025-13441) | 87-106 |
| 1 | 409-418 | PRRARSVRYP linker 1-10 | | |
| 1 | 419-436 | AFKLNIKLLGVGGKPCIK | nsp6(10973-11842) | 268-285 |
| 2 | 1-10 | EGSVRVVTTF | nsp4(8555-10054) | 207-216 |
| 2 | 11-20 | PRRARSVRYP linker 1-10 | | |
| 2 | 21-29 | KFPLKLRGT | 2′-O-ribose_methyltransferase | 249-257 |
| | | | (20659-21552) | |
| 2 | 30-39 | PRRARSVRYP linker 1-10 | | |
| 2 | 40-49 | CPRHVICTSE | 3C-like_proteinase(10055-10972) | 38-47 |
| 2 | 50-59 | PRRARSVRYP linker 1-10 | | |
| 2 | 60-77 | DKACPLIAAVITREVGFV | nsp4(8555-10054) | 85-102 |
| 2 | 78-87 | PRRARSVRYP linker 1-10 | | |
| 2 | 88-101 | DYDCVSFCYMHHME | 3C-like_proteinase(10055-10972) | 153-166 |
| 2 | 102-111 | PRRARSVRYP linker 1-10 | | |
| 2 | 112-127 | NFLVQAGNVQLRVIGH | 3C-like_proteinase(10055-10972) | 65-80 |
| 2 | 128-137 | PRRARSVRYP linker 1-10 | | |
| 2 | 138-146 | IQLSSYSLF | 2′-O-ribose_methyltransferase | 237-245 |
| | | | (20659-21552) | |
| 2 | 147-156 | PRRARSVRYP linker 1-10 | | |
| 2 | 157-169 | KLALGGSVAIKIT | 2′-O-ribose_methyltransferase | 160-172 |
| 2 | 170-179 | PRRARSVRYP linker 1-10 | (20659-21552) | |
| 2 | 180-197 | KILNNLGVDIAANTVIWD | endoRNAse(19621-20658) | 70-87 |
| 2 | 198-207 | PRRARSVRYP linker 1-10 | | |
| 2 | 208-235 | IKDLPKEITVATSRTLSYYKLGASQRVA | membrane_glycoprotein(26523-27191) | 161-188 |
| 2 | 236-245 | PRRARSVRYP linker 1-10 | | |
| 2 | 246-266 | LGVLVPHVGEIPVAYRKVLLR | leader_protein(266-805) | 104-124 |
| 2 | 267-276 | PRRARSVRYP linker 1-10 | | |
| 2 | 277-286 | GEVPVSIINN | endoRNAse(19621-20658) | 20-29 |
| 2 | 287-296 | PRRARSVRYP linker 1-10 | | |
| 2 | 297-306 | LAWPLIVTAL | nsp8(12092-12685) | 180-189 |
| 2 | 307-316 | PRRARSVRYP linker 1-10 | | |
| 2 | 317-328 | LNVPLHGTILTR | membrane_glycoprotein(26523-27191) | 120-131 |
| 2 | 329-338 | PRRARSVRYP linker 1-10 | | |
| 2 | 339-346 | EVTPSGTW | nucleocapsid_phosphoprotein | 323-330 |
| | | | (28274-29533) | |
| 2 | 347-356 | PRRARSVRYP linker 1-10 | | |
| 2 | 357-371 | EVPANSTVLSFCAFA | nsp10(13025-13441) | 6-20 |
| 2 | 372-381 | PRRARSVRYP linker 1-10 | | |
| 2 | 382-389 | VAFNVVNK | endoRNAse(19621-20658) | 5-12 |
| 2 | 390-399 | PRRARSVRYP linker 1-10 | | |
| 2 | 400-422 | GEAVKTQFNYYKKVDGVVQQLPE | endoRNAse(19621-20658) | 169-191 |
| 2 | 423-432 | PRRARSVRYP linker 1-10 | | |
| 2 | 433-443 | ASCCLYCRCHI | nsp10(13025-13441) | 71-81 |
| 3 | 1-15 | SVLLSMQGAVDINKL | nsp7(11843-12091) 57-71 | 57-71 |
| 3 | 16-25 | PRRARSVRYP linker 1-10 | | |
| 3 | 26-38 | YTELEPPCRFVTD | nsp9(12686-13024) | 66-78 |
| 3 | 39-48 | PRRARSVRYP linker 1-10 | | |
| 3 | 49-62 | RLTLGVYDYLVSTQ | nsp6(10973-11842) | 236-249 |
| 3 | 63-72 | PRRARSVRYP linker 1-10 | | |
| 3 | 73-86 | LSPVALRQMSCAAG | nsp9(12686-13024) | 4-17 |
| 3 | 87-96 | PRRARSVRYP linker 1-10 | | |
| 3 | 97-107 | TEISFMLWCKD | endoRNAse(19621-20658) | 325-335 |
| 3 | 108-117 | PRRARSVRYP linker 1-10 | | |
| 3 | 118-129 | TVNVLAWLYAAV | 3C-like_proteinase(10055-10972) | 201-212 |
| 3 | 130-139 | PRRARSVRYP linker 1-10 | | |
| 3 | 140-157 | HVQLSLPVLQVRDVLVRG | leader_protein(266-805) | 13-30 |
| 3 | 158-167 | PRRARSVRYP linker 1-10 | | |
| 3 | 168-180 | YTVSCLPFTINCQ | ORF8_protein(27894-28259) | 79-91 |
| 3 | 181-190 | PRRARSVRYP linker 1-10 | | |
| 3 | 191-198 | GYVMHANY | 2′-O-ribose_methyltransferase | 221-228 |
| | | | (20659-21552) | |
| 3 | 199-208 | PRRARSVRYP linker 1-10 | | |
| 3 | 209-232 | LGSLVVRCSFYEDFLEYHDVRVVL | ORF8_protein(27894-28259) | 95-118 |
| 3 | 233-242 | PRRARSVRYP linker 1-10 | | |
| 3 | 243-253 | AALALLLLDRL | nucleocapsid_phosphoprotein | 217-227 |
| | | | (28274-29533) | |
| 3 | 254-263 | PRRARSVRYP linker 1-10 | | |
| 3 | 264-271 | YLKLRSDV | nsp4(8555-10054) | 427-434 |
| 3 | 272-281 | PRRARSVRYP linker 1-10 | | |
| 3 | 282-292 | ITNCVKMLCTH | nsp10(13025-13441) | 38-48 |
| 3 | 293-302 | PRRARSVRYP linker 1-10 | | |
| 3 | 303-316 | TSACVLAAECTIFK | nsp4(8555-10054) 147-160 | 147-160 |
| 3 | 317-326 | PRRARSVRYP linker 1-10 | | |
| 3 | 327-335 | VLGSLAATV | nsp9(12686-13024) | 102-110 |
| 3 | 336-345 | PRRARSVRYP linker 1-10 | | |
| 3 | 346-353 | TFSVLACY | 3C-like_proteinase(10055-10972) | 111-118 |
| 3 | 354-363 | PRRARSVRYP linker 1-10 | | |
| 3 | 364-400 | ALTCFSTQFAFACPDGVKHVYQLRARSV | ORF7a_protein(27394-27759) | 55-91 |
| | | SPKLFIRQE | | |
| 3 | 401-410 | PRRARSVRYP linker 1-10 | | |
| 3 | 411-427 | KHWPQIAQFAPSASAFF | nucleocapsid_phosphoprotein | 299-315 |
| | | | (28274-29533) | |
| 4 | 1-10 | FDVVRQCSGV | 3C-like_proteinase(10055-10972) | 294-303 |
| 4 | 11-20 | PRRARSVRYP linker 1-10 | | |
| 4 | 21-47 | AKYTQLCQYLNTLTLAVPYNMRVIHFG | 2′-O-ribose_methyltransferase | 45-71 |
| | | | (20659-21552) | |
| 4 | 48-57 | PRRARSVRYP linker 1-10 | | |
| 4 | 58-67 | LEGSVAYESL | nsp4(8555-10054) | 176-185 |
| 4 | 68-77 | PRRARSVRYP linker 1-10 | | |
| 4 | 78-85 | SQDLSVVS | endoRNAse(19621-20658) | 308-315 |
| 4 | 86-95 | PRRARSVRYP linker 1-10 | | |
| 4 | 96-118 | TFKVSIWNLDYIINLIIKNLSKS | ORF6_protein(27202-27387) | 21-43 |
| 4 | 119-128 | PRRARSVRYP linker 1-10 | | |
| 4 | 129-139 | ETICAPLTVFF | endoRNAse(19621-20658) | 113-123 |
| 4 | 140-149 | PRRARSVRYP linker 1-10 | | |
| 4 | 150-186 | FHQECSLQSCTQHQPYVVDDPCPIHFYS | ORF8_protein(27894-28259) | 16-52 |
| | | KWYIRVGAR | | |
| 4 | 187-196 | PRRARSVRYP linker 1-10 | | |
| 4 | 197-206 | IGDCATVHTA | 2′-O-ribose_methyltransferase | 112-121 |
| | | | (20659-21552) | |
| 4 | 207-216 | PRRARSVRYP linker 1-10 | | |
| 4 | 217-227 | SEAFLIGCNYL | 2′-O-ribose_methyltransferase | 202-212 |
| | | | (20659-21552) | |
| 4 | 228-237 | PRRARSVRYP linker 1-10 | | |
| 4 | 238-253 | LVDFQVTIAEILLIIM | ORF6_protein(27202-27387) | 4-19 |
| 4 | 254-263 | PRRARSVRYP linker 1-10 | | |
| 4 | 264-274 | TKVDGVDVELF | endoRNAse(19621-20658) | 33-43 |
| 4 | 275-284 | PRRARSVRYP linker 1-10 | | |
| 4 | 285-317 | YEPLTQDHVDILGPLSAQTGIAVLDMCA | 3C-like_proteinase(10055-10972) | 239-271 |
| | | SLKEL | | |
| 4 | 318-327 | PRRARSVRYP linker 1-10 | | |
| 4 | 328-349 | LPRVFSAVGNICYTPSKLIEYT | nsp4(8555-10054) | 122-143 |
| 4 | 350-359 | PRRARSVRYP linker 1-10 | | |
| 4 | 360-375 | SEFSSLPSYAAFATAQ | nsp8(12092-12685) | 4-19 |
| 4 | 376-385 | PRRARSVRYP linker 1-10 | | |
| 4 | 386-395 | GPKVKYLYFI | nsp9(12686-13024) | 82-91 |
| 4 | 396-405 | PRRARSVRYP linker 1-10 | | |
| 4 | 406-433 | RLCAYCCNIVNVSLVKPSFYVYSRVKNL | envelope_protein(26245-26472) | 38-65 |

Depending on the actual embodiment, the number of concatemers can be different. Depending on the actual embodiment, the sequence of the linker can be different. Depending on the actual embodiment, the concatemers can include epitopes derived from viral strains circulating in a specific geographical region or otherwise defined (it should be noted that the country names listed in the following sequences are actually placeholders for viral peptides circulating in more than one country).

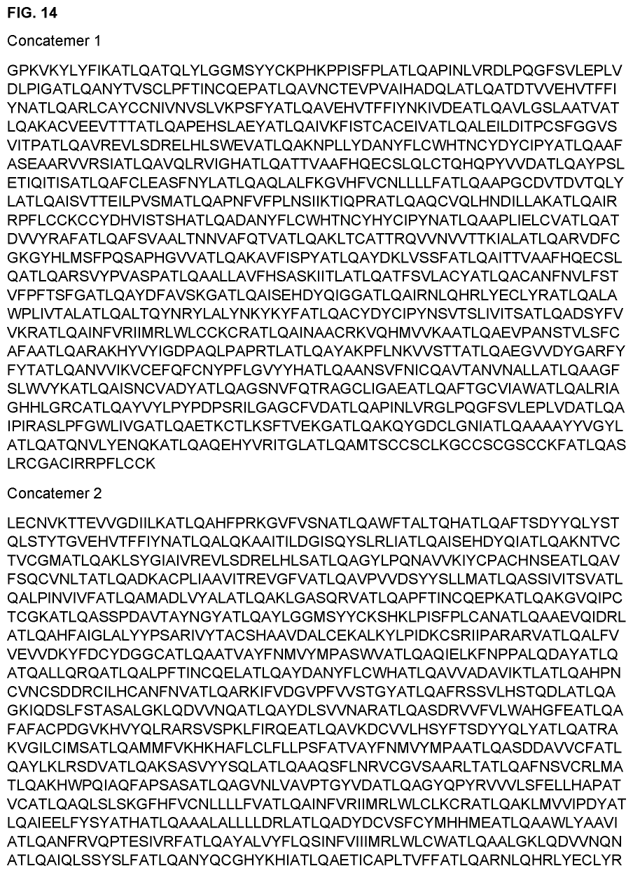

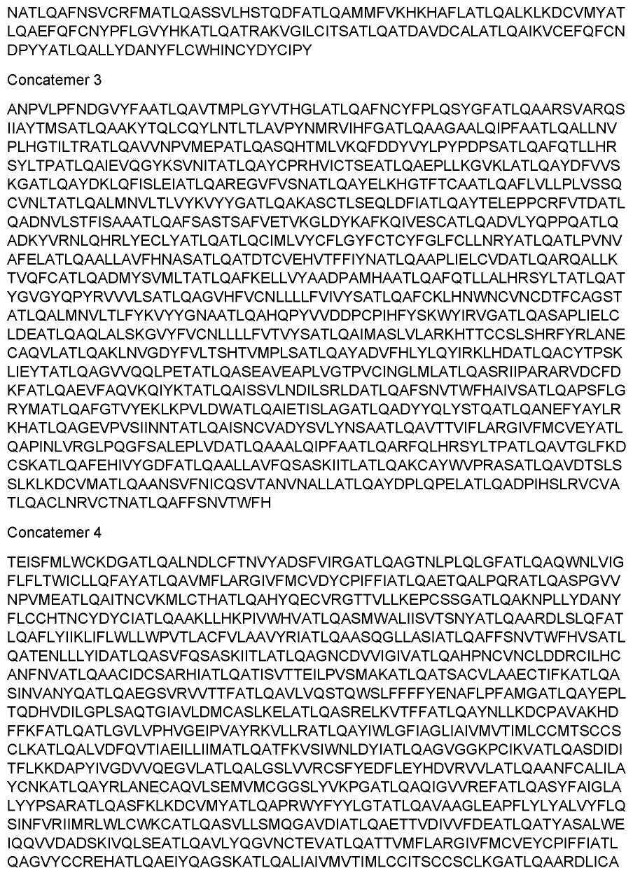

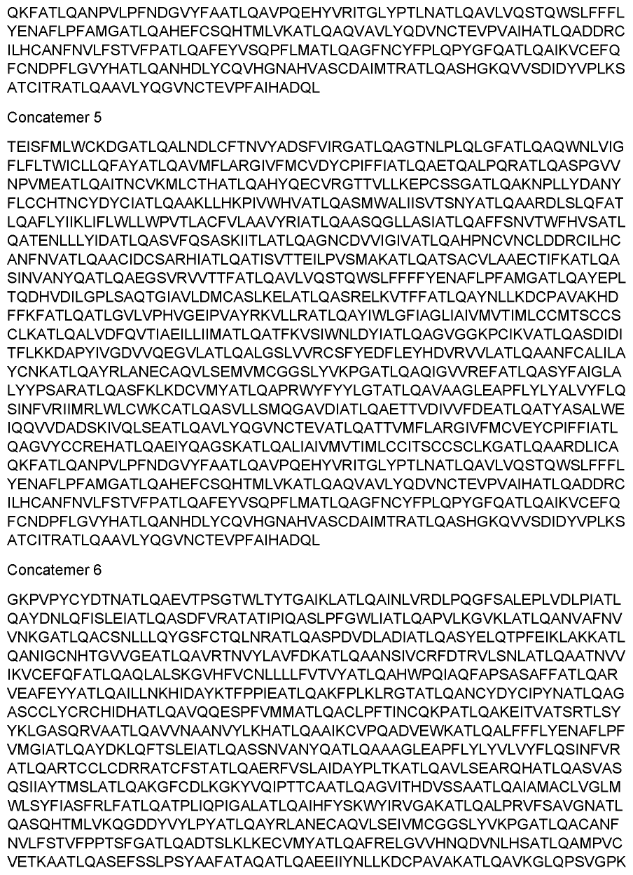

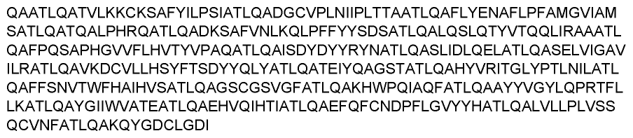

In certain embodiments, the vaccine comprises or expresses at least one of the concatemers of epitopes from SARS-COV-2 with intervening linker sequences (and as set forth in FIG. 14). In certain embodiments, the vaccine comprises or expresses at least two of the concatemers set forth below. In certain embodiments, the vaccine comprises or expresses three of the concatemers set forth below. In certain embodiments, the vaccine comprises or expresses four of the concatemers set forth below. In certain embodiments, the vaccine comprises or expresses five of the concatemers set forth below. In certain embodiments, the vaccine comprises or expresses all the concatemers set forth below.

TABLE 4

| concat- | pos_in_ | | | pos_in_ |
|---|---|---|---|---|
| emer | concatemer | epitope | gene | gene |
| 1 | 1-11 | GPKVKYLYFIK | nsp9(NC_045512: + : 12686-13024) | 82-92 |
| 1 | 12-16 | ATLQA linker 1-5 | | |
| 1 | 17-39 | TQLYLGGMSYYCKPHKPPISFPL | helicase@Iceland(NC_045512@Iceland: | 61-83 |
| | | | + : 16237-18039) | |
| 1 | 40-44 | ATLQA linker 1-5 | | |
| 1 | 45-68 | PINLVRDLPQGFSVLEPLVDLPIG | surface_glycoprotein@Belgium | 205-228 |
| | | | (NC_045512@Belgium: + : | |
| | | | 21563-25384) | |
| 1 | 69-73 | ATLQA linker 1-5 | | |
| 1 | 74-89 | NYTVSCLPFTINCQEP | ORF8_protein@Bangladesh(NC_045512@ | 78-93 |
| | | | Bangladesh: + : 27894-28259) | |
| 1 | 90-94 | ATLQA linker 1-5 | | |
| 1 | 95-109 | VNCTEVPVAIHADQL | surface_glycoprotein(NC_045512: + : | 615-629 |
| | | | 21563-25384) | |
| 1 | 110-114 | ATLQA linker 1-5 | | |
| 1 | 115-128 | TDTVVEHVTFFIYN | ORF3a_protein@CostaRica(NC_045512@ | 221-234 |
| | | | CostaRica: + : 25393-26220) | |
| 1 | 129-133 | ATLQA linker 1-5 | | |
| 1 | 134-153 | RLCAYCCNIVNVSLVKPSFY | envelope_protein(NC_045512: + : | 38-57 |
| | | | 26245-26472) | |
| 1 | 154-158 | ATLQA linker 1-5 | | |
| 1 | 159-173 | VEHVTFFIYNKIVDE | ORF3a_protein(NC_045512: + : | 225-239 |
| | | | 25393-26220) | |
| 1 | 174-178 | ATLQA linker 1-5 | | |
| 1 | 179-187 | VLGSLAATV | nsp9(NC_045512: + : 12686-13024) | 102-110 |
| 1 | 188-192 | ATLQA linker 1-5 | | |
| 1 | 193-202 | KACVEEVTTT | nsp3(NC_045512: + : 2720-8554) | 415-424 |
| 1 | 203-207 | ATLQA linker 1-5 | | |
| 1 | 208-215 | PEHSLAEY | nsp2(NC_045512: + : 806-2719) | 200-207 |
| 1 | 216-220 | ATLQA linker 1-5 | | |
| 1 | 221-233 | IVKFISTCACEIV | nsp2(NC_045512: + : 806-2719) | 468-480 |
| 1 | 234-238 | ATLQA linker 1-5 | | |
| 1 | 239-257 | LEILDITPCSFGGVSVITP | surface_glycoprotein(NC_045512: + : | 582-600 |
| | | | 21563-25384) | |
| 1 | 258-262 | ATLQA linker 1-5 | | |
| 1 | 263-278 | VREVLSDRELHLSWEV | helicase(NC_045512: + : | 154-169 |
| | | | 16237-18039) | |
| 1 | 279-283 | ATLQA linker 1-5 | | |
| 1 | 284-308 | KNPLLYDANYFLCWHTNCYDYCIPY | ORF3a_protein@Aruba(NC_045512@ | 136-160 |
| | | | Aruba: + : 25393-26220) | |
| 1 | 309-313 | ATLQA linker 1-5 | | |
| 1 | 314-326 | AFASEAARVVRSI | nsp2(NC_045512: + : 806-2719) | 355-367 |
| 1 | 327-331 | ATLQA linker 1-5 | | |
| 1 | 332-339 | VQLRVIGH | 3C-like_proteinase(NC_045512: + : | 73-80 |
| | | | 10055-10972) | |
| 1 | 340-344 | ATLQA linker 1-5 | | |
| 1 | 345-368 | TTTVAAFHQECSLQLCTQHQPYVVD | ORF8_protein@USA(NC_045512@USA: + : | 11-34 |
| | | | 27894-28259) | |
| 1 | 369-373 | ATLQA linker 1-5 | | |
| 1 | 374-385 | YPSLETIQITIS | nsp3(NC_045512: + : 2720-8554) | 582-600 |
| 1 | 386-390 | ATLQA linker 1-5 | | |
| 1 | 391-400 | FCLEASFNYL | nsp3(NC_045512: + : 2720-8554) | 1391-1400 |
| 1 | 401-405 | ATLQA linker 1-5 | | |
| 1 | 406-423 | QLALFKGVHFVCNLLLLF | ORF3a_protein@CostaRica(NC_045512@ | 70-87 |
| | | | CostaRica: + : 25393-26220) | |
| 1 | 424-428 | ATLQA linker 1-5 | | |
| 1 | 429-442 | APGCDVTDVTQLYL | helicase@Aruba(NC_045512@Aruba: + : | 52-65 |
| | | | (16237-18039) | |
| 1 | 443-447 | ATLQA linker 1-5 | | |
| 1 | 448-459 | ISVTTEILPVSM | surface_glycoprotein(NC_045512: + : | 720-731 |
| | | | 21563-25384) | |
| 1 | 460-464 | ATLQA linker 1-5 | | |
| 1 | 465-481 | PNFVFPLNSIIKTIQPR | nsp2(NC_045512: + : 806-2719) | 91-107 |
| 1 | 482-486 | ATLQA linker 1-5 | | |
| 1 | 487-499 | QCVQLHNDILLAK | nsp7(NC_045512: + : 11843-12091) | 31-43 |
| 1 | 500-504 | ATLQA linker 1-5 | | |
| 1 | 505-524 | ARRPFLCCKCCYDHVISTSH | helicase(NC_045512: + : | 20-39 |
| | | | 16237-18039) | |
| 1 | 525-529 | ATLQA linker 1-5 | | |
| 1 | 530-549 | DANYFLCWHTNCYHYCIPYN | ORF3a_protein@Indonesia(NC_045512@ | 142-161 |
| | | | Indonesia: + : 25393-26220) | |
| 1 | 550-554 | ATLQA linker 1-5 | | |
| 1 | 555-562 | APLEILCV | ORF8_protein(NC_045512: + : | 55-62 |
| | | | 27894-28259) | |
| 1 | 563-567 | ATLQA linker 1-5 | | |
| 1 | 568-575 | TDVVYRAF | RNA-dependent_RNA_polymerase@ | 28-35 |
| | | | Aruba(NC_045512@Aruba: + : | |
| | | | 13442-13468 + 13468-16236) | |
| 1 | 576-580 | ATLQA linker 1-5 | | |
| 1 | 581-595 | FSVAALTNNVAFQTV | RNA-dependent_RNA_polymerase | 396-410 |
| | | | (NC_045512: + : 13442-13468 + | |
| | | | 13468-16236) | |
| 1 | 596-600 | ATLQA linker 1-5 | | |
| 1 | 601-620 | KLTCATTRQVVNVVTTKIAL | nsp3(NC_045512: + : 2720-8554) | 1923-1942 |
| 1 | 621-625 | ATLQA linker 1-5 | | |
| 1 | 626-648 | RVDFCGKGYHLMSFPQSAPHGVV | surface_glycoprotein(NC_045512: + : | 1039-1061 |
| | | | 21563-25384) | |
| 1 | 649-653 | ATLQA linker 1-5 | | |
| 1 | 654-661 | KAVFISPY | helicase(NC_045512: + : | 508-515 |
| | | | 16237-18039) | |
| 1 | 662-666 | ATLQA linker 1-5 | | |
| 1 | 667-674 | YDKLVSSF | nsp3(NC_045512: + : 2720-8554) | 365-372 |
| 1 | 675-679 | ATLQA linker 1-5 | | |
| 1 | 680-693 | ITTVAAFHQECSLQ | ORF8_protein@Aruba(NC_045512@ | 10-23 |
| | | | Aruba: + : 27894-28259) | |
| 1 | 694-698 | ATLQA linker 1-5 | | |
| 1 | 699-707 | RSVYPVASP | nsp2(NC_045512: + : 806-2719) | 121-129 |
| 1 | 708-712 | ATLQA linker 1-5 | | |
| 1 | 713-727 | ALLAVFHSASKIITL | ORF3a_protein@Aruba(NC_045512@ | 51-65 |
| | | | Aruba: + : 25393-26220) | |
| 1 | 728-732 | ATLQA linker 1-5 | | |
| 1 | 733-740 | TFSVLACY | 3C-like_proteinase(NC_045512: + : | 111-118 |
| | | | 10055-10972) | |
| 1 | 741-745 | ATLQA linker 1-5 | | |
| 1 | 746-763 | CANFNVLFSTVFPFTSFG | RNA-dependent_RNA_polymerase@ | 310-327 |
| | | | Nigeria(NC_045512@Nigeria: + : | |
| | | | 13442-13468 + 13468-16236) | |
| 1 | 764-768 | ATLQA linker 1-5 | | |
| 1 | 769-776 | YDFAVSKG | RNA-dependent_RNA_polymerase@ | 420-427 |
| | | | Aruba(NC_045512@Aruba: + : | |
| | | | 13442-13468 + 13468-16236) | |
| 1 | 777-781 | ATLQA linker 1-5 | | |
| 1 | 782-791 | ISEHDYQIGG | ORF3a_protein@FrenchGiana | 179-188 |
| | | | (NC_045512@FrenchGiana: + : | |
| | | | 25393-26220) | |
| 1 | 792-796 | ATLQA linker 1-5 | | |
| 1 | 797-810 | IRNLQHRLYECLYR | RNA-dependent_RNA_polymerase@ | 720-733 |
| | | | Austria(NC_045512@Austria: + : | |
| | | | 13442-13468 + 13468-16236) | |
| 1 | 811-815 | ATLQA linker 1-5 | | |
| 1 | 816-825 | LAWPLIVTAL | nsp8(NC_045512: + : 12092-12685) | 180-189 |
| 1 | 826-830 | ATLQA linker 1-5 | | |
| 1 | 831-847 | LTQYNRYLALYNKYKYF | nsp4(NC_045512: + : 8555-10054) | 438-454 |
| 1 | 848-852 | ATLQA linker 1-5 | | |
| 1 | 853-871 | CYDYCIPYNSVTSLIVITS | ORF3a_protein@Netherlands | 153-171 |
| | | | (NC_045512@Netherlands: + : | |
| | | | 25393-26220) | |
| 1 | 872-876 | ATLQA linker 1-5 | | |
| 1 | 877-884 | DSYFVVKR | RNA-dependent_RNA_polymerase | 67-74 |
| | | | (NC_045512: + : 13442-13468 + | |
| | | | 13468-16236) | |
| 1 | 885-889 | ATLQA linker 1-5 | | |
| 1 | 890-906 | INFVRIIMRLWLCCKCR | ORF3a_protein@Australia | 118-134 |
| | | | (NC_045512@Australia: + : | |
| | | | 25393-26220) | |
| 1 | 907-911 | ATLQA linker 1-5 | | |
| 1 | 912-926 | INAACRKVQHMVVKA | 3′-to-5′_exonuclease(NC_045512: + : | 305-319 |
| | | | 18040-19620) | |
| 1 | 927-931 | ATLQA linker 1-5 | | |
| 1 | 932-946 | EVPANSTVLSFCAFA | nsp10(NC_045512: + : 13025-13441) | 6-20 |
| 1 | 947-951 | ATLQA linker 1-5 | | |
| 1 | 952-971 | RAKHYVYIGDPAQLPAPRTL | helicase(NC_045512: + : | 392-411 |
| | | | 16237-18039) | |
| 1 | 972-976 | ATLQA linker 1-5 | | |
| 1 | 977-989 | YAKPFLNKVVSTT | nsp3(NC_045512: + : 2720-8554) | 1323-1335 |
| 1 | 990-994 | ATLQA linker 1-5 | | |
| 1 | 995-1008 | EGVVDYGARFYFYT | nsp3(NC_045512: + : 2720-8554) | 595-608 |
| 1 | 1009-1013 | ATLQA linker 1-5 | | |
| 1 | 1014-1035 | NVVIKVCEFQFCNYPFLGVYYH | surface_glycoprotein@Brazil | 125-146 |
| | | | (NC_045512@Brazil: + : | |
| | | | 21563-25384) | |
| 1 | 1036-1040 | ATLQA linker 1-5 | | |
| 1 | 1041-1059 | ANSVFNICQAVTANVNALL | RNA-dependent_RNA_polymerase@ | 690-708 |
| | | | Aruba(NC_045512@Aruba: + : | |
| | | | 13442-13468 + 13468-16236) | |
| 1 | 1060-1064 | ATLQA linker 1-5 | | |
| 1 | 1065-1073 | AGFSLWVYK | 3′-to-5′_exonuclease(NC_045512: + : | 504-512 |
| | | | 18040-19620) | |
| 1 | 1074-1078 | ATLQA linker 1-5 | | |
| 1 | 1079-1086 | ISNCVADY | surface_glycoprotein(NC_045512: + : | 358-365 |
| | | | 21563-25384) | |
| 1 | 1087-1091 | ATLQA linker 1-5 | | |
| 1 | 1092-1107 | GSNVFQTRAGCLIGAE | surface_glycoprotein(NC_045512: + : | 639-654 |
| | | | 21563-25384) | |
| 1 | 1108-1112 | ATLQA linker 1-5 | | |
| 1 | 1113-1120 | FTGCVIAW | surface_glycoprotein(NC_045512: + : | 429-436 |
| | | | 21563-25384) | |
| 1 | 1121-1125 | ATLQA linker 1-5 | | |
| 1 | 1126-1136 | LRIAGHHLGRC | membrane_glycoprotein(NC_045512: + | 149-159 |
| | | | : 26523-27191) | |
| 1 | 1137-1141 | ATLQA linker 1-5 | | |
| 1 | 1142-1161 | YVYLPYPDPSRILGAGCFVD | RNA-dependent_RNA_polymerase | 826-845 |
| | | | (NC_045512: + : 13442-13468 + | |
| | | | 13468-16236) | |
| 1 | 1162-1166 | ATLQA linker 1-5 | | |
| 1 | 1167-1186 | PINLVRGLPQGFSVLEPLVD | surface_glycoprotein@SmallAfrica | 209-228 |
| | | | (NC_045512@SmallAfrica: + : | |
| | | | 21563-25384) | |
| 1 | 1187-1191 | ATLQA linker 1-5 | | |
| 1 | 1192-1206 | IPIRASLPFGWLIVG | ORF3a_protein@Iceland(NC_045512@ | 35-49 |
| | | | Iceland: + : 25393-26220) | |
| 1 | 1207-1211 | ATLQA linker 1-5 | | |
| 1 | 1212-1225 | ETKCTLKSFTVEKG | surface_glycoprotein@Aruba | 294-307 |
| | | | (NC_045512@Aruba: + : 21563-25384) | |
| 1 | 1226-1230 | ATLQA linker 1-5 | | |
| 1 | 1231-1240 | KQYGDCLGNI | surface_glycoprotein@Jordan | 831-840 |
| | | | (NC_045512@Jordan: + : 21563-25384) | |
| 1 | 1241-1245 | ATLQA linker 1-5 | | |
| 1 | 1246-1254 | AAAYYVGYL | surface_glycoprotein(NC_045512: + : | 262-270 |
| | | | 21563-25384) | |
| 1 | 1255-1259 | ATLQA linker 1-5 | | |
| 1 | 1260-1269 | TQNVLYENQK | surface_glycoprotein(NC_045512: + : | 912-921 |
| | | | 21563-25384) | |
| 1 | 1270-1274 | ATLQA linker 1-5 | | |
| 1 | 1275-1284 | QEHYVRITGL | helicase(NC_045512: + : | 243-252 |
| | | | 16237-18039) | |
| 1 | 1285-1289 | ATLQA linker 1-5 | | |
| 1 | 1290-1309 | MTSCCSCLKGCCSCGSCCKF | surface_glycoprotein(NC_045512: + : | 1237-1256 |
| | | | 21563-25384) | |
| 1 | 1310-1314 | ATLQA linker 1-5 | | |
| 1 | 1315-1330 | SLRCGACIRRPFLCCK | helicase@Aruba(NC_045512@Aruba: + : | 13-28 |
| | | | 16237-188039) | |
| 2 | 1-17 | LECNVKTTEVVGDIILK | nsp3(NC_045512: + : 2720-8554) | 1244-1260 |
| 2 | 18-22 | ATLQA linker 1-5 | | |
| 2 | 23-33 | HFPRKGVFVSN | surface_glycoprotein@SmallAsia | 1078-1088 |
| | | | (NC_045512@SmallAsia: + : | |
| | | | 21563-25384) | |
| 2 | 34-38 | ATLQA linker 1-5 | | |
| 2 | 39-46 | WFTALTQH | nucleocapsid_phosphoprotein | 52-59 |
| | | | (NC_045512: + : 28274-29533) | |
| 2 | 47-51 | ATLQA linker 1-5 | | |
| 2 | 52-79 | FTSDYYQLYSTQLSTYTGVEHVTFF | ORF3a_protein@Latvia(NC_045512@ | 207-234 |
| | | IYN | Latvia: + : 25393-26220) | |
| 2 | 80-84 | ATLQA linker 1-5 | | |
| 2 | 85-104 | LQKAAITILDGISQYSLRLI | nsp2(NC_045512: + : 806-2719) | 382-401 |
| 2 | 105-109 | ATLQA linker 1-5 | | |
| 2 | 110-117 | ISEHDYQI | ORF3a_protein@Aruba(NC_045512@ | 179-186 |
| | | | Aruba: + : 25393-26220) | |
| 2 | 118-122 | ATLQA linker 1-5 | | |
| 2 | 123-132 | KNTVCTVCGM | nsp10(NC_045512: + : 10325-13441) | 113-122 |
| 2 | 133-137 | ATLQA linker 1-5 | | |
| 2 | 138-158 | KLSYGIAIVREVLSDRELHLS | helicase@Colombia(NC_045512@ | 146-166 |
| | | | Colombia: + : 16237-18039) | |
| 2 | 159-163 | ATLQA linker 1-5 | | |
| 2 | 164-183 | GYLPQNAVVKIYCPACHNSE | nsp2(NC_045512: + : 806-2719) | 178-197 |
| 2 | 184-188 | ATLQA linker 1-5 | | |
| 2 | 189-197 | VFSQCVNLT | surface_glycoprotein@Egypt | 11-19 |
| | | | (NC_045512@Egypt: + : 21563-25384) | |
| 2 | 198-202 | ATLQA linker 1-5 | | |
| 2 | 203-220 | DKACPLIAAVITREVGFV | nsp4(NC_045512: + : 8555-10054) | 85-102 |
| 2 | 221-225 | ATLQA linker 1-5 | | |
| 2 | 226-237 | VPVVDSYYSLLM | RNA-dependent_RNA_polymerase | 231-242 |
| | | | (NC_045512: + : 13442-13468 + | |
| | | | 13468-16236) | |
| 2 | 238-242 | ATLQA linker 1-5 | | |
| 2 | 243-250 | SSIVITSV | ORF3a_protein@Aruba(NC_045512@ | 165-172 |
| | | | Aruba: + : 25393-26220) | |
| 2 | 251-255 | ATLQA linker 1-5 | | |
| 2 | 256-263 | LPINVIVF | nsp3(NC_045512: + : 2720-8554) | 1718-1725 |
| 2 | 264-268 | ATLQA linker 1-5 | | |
| 2 | 269-276 | MADLVYAL | RNA-dependent_RNA_polymerase | 124-131 |
| | | | (NC_045512: + : 13442-13468 + | |
| | | | 13468-16236) | |
| 2 | 277-281 | ATLQA linker 1-5 | | |
| 2 | 282-289 | KLGASQRV | membrane_glycoprotein(NC_045512: + | 180-187 |
| | | | : 26523-27191) | |
| 2 | 290-294 | ATLQA linker 1-5 | | |
| 2 | 295-304 | PFTINCQEPK | ORF8_protein@India(NC_045512@ | 85-94 |
| | | | India: + : 27894-28259) | |
| 2 | 305-309 | ATLQA linker 1-5 | | |
| 2 | 310-320 | KGVQIPCTCGK | nsp3(NC_045512: + : 2720-8554) | 963-973 |
| 2 | 321-325 | ATLQA linker 1-5 | | |
| 2 | 326-337 | SSPDAVTAYNGY | nsp3(NC_045512: + : 2720-8554) | 660-671 |
| 2 | 338-342 | ATLQA linker 1-5 | | |
| 2 | 343-365 | YLGGMSYYCKSHKLPISFPLCAN | helicase@CostaRica(NC_045512@ | 64-86 |
| | | | CostaRica: + : 16237-18039) | |
| 2 | 366-370 | ATLQA linker 1-5 | | |
| 2 | 371-378 | AEVQIDRL | surface_glycoprotein(NC_045512: + | 989-996 |
| | | | : 21563-25384) | |
| 2 | 379-383 | ATLQA linker 1-5 | | |
| 2 | 384-434 | HFAIGLALYYPSARIVYTACSHAAV | helicase(NC_045512: + : | 290-340 |
| | | DALCEKALKYLPIDKCSRIIPARAR | 16237-18039) | |
| | | V | | |
| 2 | 435-439 | ATLQA linker 1-5 | | |
| 2 | 440-457 | LFVVEVVDKYFDCYDGGC | RNA-dependent_RNA_polymerase | 470-487 |
| | | | (NC_045512: + : 13442-13468 + | |
| | | | 13468-16236) | |
| 2 | 458-462 | ATLQA linker 1-5 | | |
| 2 | 463-478 | ATVAYFNMVYMPASWV | nsp6@Aruba(NC_045512@Aruba: + : | 76-91 |
| | | | 10973-11842) | |
| 2 | 479-483 | ATLQA linker 1-5 | | |
| 2 | 484-498 | QIELKFNPPALQDAY | nsp3(NC_045512: + : 2720-8554) | 867-881 |
| 2 | 499-503 | ATLQA linker 1-5 | | |
| 2 | 504-511 | TQALLQRQ | nucleocapsid_phosphoprotein@ | 379-386 |
| | | | CostaRica(NC_045512@CostaRica: + : | |
| | | | 28274-29533) | |
| 2 | 512-516 | ATLQA linker 1-5 | | |
| 2 | 517-526 | LPFTINCQEL | ORF8_protein@Mozambique(NC_045512@ | 84-93 |
| | | | Mozambique: + : 27894-28259) | |
| 2 | 527-531 | ATLQA linker 1-5 | | |
| 2 | 532-541 | YDANYFLCWH | ORF3a_protein(NC_045512: + : | 141-150 |
| | | | 25393-26220) | |
| 2 | 542-546 | ATLQA linker 1-5 | | |
| 2 | 547-556 | VVADAVIKTL | nsp3(NC_045512: + : 2720-8554) | 56-65 |
| 2 | 557-561 | ATLQA linker 1-5 | | |
| 2 | 562-582 | HPNCVNCSDDRCILHCANFNV | RNA-dependent_RNA_polymerase@ | 295-315 |
| | | | Aruba(NC_045512@Aruba: + : | |
| | | | 13442-13468 + 13468-16236) | |
| 2 | 583-587 | ATLQA linker 1-5 | | |
| 2 | 588-603 | RKIFVDGVPFVVSTGY | RNA-dependent_RNA_polymerase | 331-346 |
| | | | (NC_045512: + : 13442-13468 + | |
| | | | 13468-16236) | |
| 2 | 604-608 | ATLQA linker 1-5 | | |
| 2 | 609-620 | FRSSVLHSTQDL | surface_glycoprotein@Aruba | 43-54 |
| | | | (NC_045512@Aruba: + : 21563-25384) | |
| 2 | 621-625 | ATLQA linker 1-5 | | |
| 2 | 626-648 | GKIQDSLFSTASALGKLQDVVNQ | surface_glycoprotein@NorthMacedonia | 928-950 |
| | | | (NC_045512@NorthMacedonia: + : | |
| | | | 21563-25384) | |
| 2 | 649-653 | ATLQA linker 1-5 | | |
| 2 | 654-662 | YDLSVVNAR | helicase(NC_045512: + : | 382-390 |
| | | | 16237-18039) | |
| 2 | 663-667 | ATLQA linker 1-5 | | |
| 2 | 668-681 | SDRVVFVLWAHGFE | 3′-to-5′_exonuclease(NC_045512: + : | 178-191 |
| | | | 18040-19620) | |
| 2 | 682-686 | ATLQA linker 1-5 | | |
| 2 | 687-715 | FAFACPDGVKHVYQLRARSVSPKLF | ORF7a_protein(NC_045512: + : | 63-91 |
| | | IRQE | 27394-27759) | |
| 2 | 716-720 | ATLQA linker 1-5 | | |
| 2 | 721-739 | VKDCVVLHSYFTSDYYQLY | ORF3a_protein@Aruba(NC_045512@ | 197-215 |
| | | | Aruba: + : 25393-26220) | |
| 2 | 740-744 | ATLQA linker 1-5 | | |
| 2 | 745-756 | TRAKVGILCIMS | helicase(NC_045512: + : | 566-577 |
| | | | 16237-18039) | |
| 2 | 757-761 | ATLQA linker 1-5 | | |
| 2 | 762-793 | MMFVKHKHAFLVLFLLPSFATVAYF | nsp6@Indonesia(NC_045512@Indonesia: | 57-88 |
| | | NMVYMPA | + : 10973-11842) | |
| 2 | 794-798 | ATLQA linker 1-5 | | |
| 2 | 799-806 | SDDAVVCF | RNA-dependent_RNA_polymerase@Aruba | 759-766 |
| | | | (NC_045512@Aruba: + : | |
| | | | 13442-13468 + 13468-16236) | |
| 2 | 807-811 | ATLQA linker 1-5 | | |
| 2 | 812-819 | YLKLRSDV | nsp4(NC_045512: + : 8555-10054) | 427-434 |
| 2 | 820-824 | ATLQA linker 1-5 | | |
| 2 | 825-834 | KSASVYYSQL | nsp3(NC_045512: + : 2720-8554) | 1737-1746 |
| 2 | 835-839 | ATLQA linker 1-5 | | |
| 2 | 840-856 | AQSFLNRVCGVSAARLT | RNA-dependent_RNA_polymerase | 4-20 |
| | | | (NC_045512: + : 13442-13468 + | |
| | | | 13468-16236) | |
| 2 | 857-861 | ATLQA linker 1-5 | | |
| 2 | 862-869 | FNSVCRLM | helicase@Aruba(NC_045512@Aruba: + : | 422-429 |
| | | | 16237-18039) | |
| 2 | 870-874 | ATLQA linker 1-5 | | |
| 2 | 875-888 | HKWPQAIQFAPSAS | nucleocapsid_phosphoprotein@ | 299-312 |
| | | | Australia(NC_045512@Australia: + : | |
| | | | 28274-29533) | |
| 2 | 889-893 | ATLQA linker 1-5 | | |
| 2 | 894-906 | GVNLVAVPTGYVD | 3′-to-5′_exonuclease(NC_045512: + : | 114-126 |
| | | | 18040-19620) | |
| 2 | 907-911 | ATLQA linker 1-5 | | |
| 2 | 912-933 | GYQPYRVVVLSFELLHAPATVC | surface_glycoprotein(NC_045512: + : | 504-525 |
| | | | 21563-25384) | |
| 2 | 934-938 | ATLQA linker 1-5 | | |
| 2 | 939-957 | QLSLSKGFHFVCNLLLLFV | ORF3a_protein@Estonia(NC_045512@ | 70-88 |
| | | | Estonia: + : 25393-26220) | |
| 2 | 958-962 | ATLQA linker 1-5 | | |
| 2 | 963-979 | INFVRIIMRLWLCLKCR | ORF3a_protein@Mozambique(NC_045512@ | 118-134 |
| | | | Mozambique: + : 25393-26220) | |
| 2 | 980-984 | ATLQA linker 1-5 | | |
| 2 | 985-993 | KLMVVIPDY | nsp8(NC_045512: + : 12092-12685) | 127-135 |
| 2 | 994-998 | ATLQA linker 1-5 | | |
| 2 | 999-1009 | IEELFYSYATH | 3′-to-5′_exonuclease(NC_045512: + : | 363-373 |
| | | | 18040-19620) | |
| 2 | 1010-1014 | ATLQA linker 1-5 | | |
| 2 | 1015-1025 | AALALLLLDRL | nucleocapsid_phosphoprotein@Aruba | 217-227 |
| | | | (NC_045512@Aruba: + : 28274-29533) | |
| 2 | 1026-1030 | ATLQA linker 1-5 | | |
| 2 | 1031-1044 | DYDCVSFCYMHHME | 3C-like_proteinase(NC_045512: + : | 153-166 |
| | | | 10055-10972) | |
| 2 | 1045-1049 | ATLQA linker 1-5 | | |
| 2 | 1050-1057 | AWLYAAVI | 3C-like_proteinase(NC_045512: + : | 206-213 |
| | | | 10055-10972) | |
| 2 | 1058-1062 | ATLQA linker 1-5 | | |
| 2 | 1063-1075 | NFRVQPTESIVRF | surface_glycoprotein(NC_045512: + : | 317-329 |
| | | | 21563-25384) | |
| 2 | 1076-1080 | ATLQA linker 1-5 | | |
| 2 | 1084-1103 | YALVYFLQSINFVIIIMRLWLCW | ORF3a_protein@Denmark(NC_045512@ | 109-131 |
| | | | Denmark: + : 25393-26220) | |
| 2 | 1104-1108 | ATLQA linker 1-5 | | |
| 2 | 1109-1120 | ALGKLQDVVNQN | surface_glycoprotein(NC_045512: + : | 944-955 |
| | | | 21563-25384) | |
| 2 | 1121-1125 | ATLQA linker 1-5 | | |
| 2 | 1126-1134 | IQLSSYSLF | 2′-O-ribose_methyltransferase | 237-245 |
| | | | (NC_045512: + : 20659-21552) | |
| 2 | 1135-1139 | ATLQA linker 1-5 | | |
| 2 | 1140-1149 | NYQCGHYKHI | nsp3(NC_045512: + : 2720-8554) | 1012-1021 |
| 2 | 1150-1154 | ATLQA linker 1-5 | | |
| 2 | 1155-1165 | ETICAPLTVFF | endoRNAse(NC_045512: + : | 113-123 |
| | | | 19621-20658) | |
| 2 | 1166-1170 | ATLQA linker 1-5 | | |
| 2 | 1171-1184 | RNLQHRLYECLYRN | RNA-dependent_RNA_polymerase | 721-734 |
| | | | (NC_045512: + : 13442-13468 + | |
| | | | 13468-16236) | |
| 2 | 1185-1189 | ATLQA linker 1-5 | | |
| 2 | 1190-1197 | FNSVCRFM | helicase@Malaysia(NC_045512@ | 422-429 |
| | | | Malaysia: + : 16237-18039) | |
| 2 | 1198-1202 | ATLQA linker 1-5 | | |
| 2 | 1203-1212 | SSVLHSTQDF | surface_glycoprotein@Norway | 45-54 |
| | | | (NC_045512@Norway: + : | |
| | | | 21563-25384) | |
| 2 | 1213-1217 | ATLQA linker 1-5 | | |
| 2 | 1218-1228 | MMFVKHKHAFL | nsp6@Aruba(NC_045512@Aruba: + : | 57-67 |
| | | | 10973-11842) | |
| 2 | 1229-1233 | ATLQA linker 1-5 | | |
| 2 | 1234-1242 | LKLKDCVMY | nsp6@Aruba(NC_045512@Aruba: + : | 105-113 |
| | | | 10973-11842) | |
| 2 | 1243-1247 | ATLQA linker 1-5 | | |
| 2 | 1248-1262 | EFQFCNYPFLGVYHK | surface_glycoprotein@Norway | 129-143 |
| | | | (NC_045512@Norway: + : | |
| | | | 21563-25384) | |
| 2 | 1263-1267 | ATLQA linker 1-5 | | |
| 2 | 1268-1279 | TRAKVGILCITS | helicase@SouthKorea(NC_045512@ | 566-577 |
| | | | SouthKorea: + : 16237-18039) | |
| 2 | 1280-1284 | ATLQA linker 1-5 | | |
| 2 | 1285-1292 | TDAVDCAL | surface_glycoprotein(NC_045512: + : | 286-293 |
| | | | 21563-25384) | |
| 2 | 1293-1297 | ATLQA linker 1-5 | | |
| 2 | 1298-1311 | IKVCEFQFCNDPYY | surface_glycoprotein@CostaRica | 128-141 |
| | | | (NC_045512@CostaRica: + : | |
| | | | 21563-25384) | |
| 2 | 1312-1316 | ATLQA linker 1-5 | | |
| 2 | 1317-1338 | LLYDANYFLCWHINCYDYCIPY | ORF3a_protein@Estonia(NC_045512@ | 139-160 |
| | | | Estonia: + : 25393-26220) | |
| 3 | 1-14 | ANPVLPFNDGVYFA | surface_glycoprotein@Austria | 77-90 |
| | | | (NC_045512@Austria: + : | |
| | | | 21563-25384) | |
| 3 | 15-19 | ATLQA linker 1-5 | | |
| 3 | 20-31 | VTMPLGYVTHGL | nsp3(NC_045512: + : 2720-8554) | 628-639 |
| 3 | 32-36 | ATLQA linker 1-5 | | |
| 3 | 37-48 | FNCYFPLQSYGF | surface_glycoprotein(NC_045512: + : | 486-497 |
| | | | 21563-25384) | |
| 3 | 49-53 | ATLQA linker 1-5 | | |
| 3 | 54-68 | ARSVARQSIIAYTMS | surface_glycoprotein@Mozambique | 680-694 |
| | | | (NC_045512@Mozambique: + : | |
| | | | 21563-25384) | |
| 3 | 69-73 | ATLQA linker 1-5 | | |
| 3 | 74-100 | AKYTQLCQYLNTLTLAVPYNMRVIH | 2′-O-ribose_methyltransferase | 45-71 |
| | | F | (NC_045512: + : 20659-21552) | |
| 3 | 101-105 | ATLQA linker 1-5 | | |
| 3 | 106-115 | AGAALQIPFA | surface_glycoprotein@Nigeria | 886-895 |
| | | | (NC_045512@Nigeria: + : | |
| | | | 21563-25384) | |
| 3 | 116-120 | ATLQA linker 1-5 | | |
| 3 | 121-133 | LLNVPLHGTILTR | membrane_glycoprotein(NC_045512: + | 119-131 |
| | | | : 26523-27191) | |
| 3 | 134-138 | ATLQA linker 1-5 | | |
| 3 | 139-146 | VVNPVMEP | ORF3a_protein(NC_045512: + : | 255-262 |
| | | | 25393-26220) | |
| 3 | 147-151 | ATLQA linker 1-5 | | |
| 3 | 152-173 | SQHTMLVKQFDDYVYLPYPDPS | RNA-dependent_RNA_polymerase@ | 814-835 |
| | | | Estonia(NC_045512@Estonia: + : | |
| | | | 13442-13468 + 13468-16236) | |
| 3 | 174-178 | ATLQA linker 1-5 | | |
| 3 | 179-190 | FQTLLHRSYLTP | surface_glycoprotein@SmallAsia | 230-241 |
| | | | (NC_045512@SmallAsia: + : | |
| | | | 21563-25384) | |
| 3 | 191-195 | ATLQA linker 1-5 | | |
| 3 | 196-207 | IEVQGYKSVNIT | nsp3(NC_045512: + : 2720-8554) | 13-24 |
| 3 | 208-212 | ATLQA linker 1-5 | | |
| 3 | 213-223 | YCPRHVICTSE | 3C-like_proteinase(NC_045512: + : | 37-47 |
| | | | 10055-10972) | |
| 3 | 224-228 | ATLQA linker 1-5 | | |
| 3 | 229-237 | EPLLKGVKL | surface_glycoprotein@Croatia | 1258-1266 |
| | | | (NC_045512@Croatia: + : | |
| | | | 21563-25384) | |
| 3 | 238-242 | ATLQA linker 1-5 | | |
| 3 | 243-250 | YDFVVSKG | RNA-dependent_RNApolymerase@Japan | 420-427 |
| | | | (NC_045512@Japan: + : | |
| | | | 13442-13468 + 13468-16236) | |
| 3 | 251-255 | ATLQA linker 1-5 | | |
| 3 | 256-266 | YDKLQFISLEI | helicase@SouthAfrica(NC_045512@ | 582-592 |
| | | | SouthAfrica: + : 16237-18039) | |
| 3 | 267-271 | ATLQA linker 1-5 | | |
| 3 | 272-279 | REGVFVSN | surface_glycoprotein@Aruba | 1087-1094 |
| | | | (NC_045512@Aruba: + : | |
| | | | 21563-25384) | |
| 3 | 280-284 | ATLQA linker 1-5 | | |
| 3 | 285-295 | YELKHGTFTCA | nsp3(NC_045512: + : 2720-8554) | 996-1006 |
| 3 | 296-300 | ATLQA linker 1-5 | | |
| 3 | 301-316 | FLVLLPLVSSQCVNLT | surface_glycoprotein@Aruba | 4-19 |
| | | | (NC_045512@Aruba: + : | |
| | | | 21563-25384) | |
| 3 | 317-321 | ATLQA linker 1-5 | | |
| 3 | 322-335 | LMNVLTLVYKVYYG | nsp6@Aruba(NC_045512@Aruba: + : | 139-152 |
| | | | 10973-11842) | |
| 3 | 336-340 | ATLQA linker 1-5 | | |
| 3 | 341-353 | KASCTLSEQLDFI | nsp2(NC_045512: + : 806-2719) | 30-42 |
| 3 | 354-358 | ATLQA linker 1-5 | | |
| 3 | 359-371 | YTELEPPCRFVTD | nsp9(NC_045512: + : 12686-13024) | 66-78 |
| 3 | 372-376 | ATLQA linker 1-5 | | |
| 3 | 377-387 | DNVLSTFISAA | nsp3(NC_045512: + : 2720-8554) | 1809-1819 |
| 3 | 388-392 | ATLQA linker 1-5 | | |
| 3 | 393-419 | FSASTSAFVETVKGLDYKAFKQIVE | nsp2(NC_045512: + : 806-2719) | 300-326 |
| | | SC | | |
| 3 | 420-424 | ATLQA linker 1-5 | | |
| 3 | 425-432 | DVLYQPPQ | nsp4(NC_045512: + : 8555-10054) | 484-491 |
| 3 | 433-437 | ATLQA linker 1-5 | | |
| 3 | 438-453 | DKYVRNLQHRLYECLY | RNA-dependent_RNA_polymerase@ | 717-732 |
| | | | Aruba(NC_045512@Aruba: + : | |
| | | | 13442-13468 + 13468-16236) | |
| 3 | 454-458 | ATLQA linker 1-5 | | |
| 3 | 459-487 | TLQCIMLVYCFLGYFCTCYFGLFCL | nsp6@Aruba(NC_045512@Aruba: + : | 203-231 |
| | | LNRY | 10973-11842) | |
| 3 | 488-492 | ATLQA linker 1-5 | | |
| 3 | 493-502 | TLPVNVAFEL | endoRNAse(NC_045512: + : | 48-57 |
| | | | 19621-20658) | |
| 3 | 503-507 | ATLQA linker 1-5 | | |
| 3 | 508-517 | ALLAVFHNAS | ORF3a_protein@SmallSouthAmerica | 51-60 |
| | | | (NC_045512@SmallSouthAmerica: + : | |
| | | | 25393-26220) | |
| 3 | 518-522 | ATLQA linker 1-5 | | |
| 3 | 523-536 | TDTCVEHVTFFIYN | ORF3a_protein@Luxemburg | 221-234 |
| | | | (NC_045512@Luxembourg: + : | |
| | | | 25393-26220) | |
| 3 | 537-541 | ATLQA linker 1-5 | | |
| 3 | 542-550 | APLIELCVD | ORF8_protein@Bangladesh(NC_04551@ | 55-63 |
| | | | Bangladesh: + : 27894-28259) | |
| 3 | 551-555 | ATLQA linker 1-5 | | |
| 3 | 556-566 | RQALLKTVQFC | RNA-dependent_RNA_polymerase@ | 183-193 |
| | | | Aruba(NC_045512@Aruba: + : | |
| | | | 13442-13468 + 13468-16236) | |
| 3 | 567-571 | ATLQA linker 1-5 | | |
| 3 | 572-579 | DMYSVMLT | RNA-dependent_RNA_polymerase@ | 901-908 |
| | | | Malaysia(NC_045512@Malaysia: + : | |
| | | | 13442-13468 + 13468-16236) | |
| 3 | 580-584 | ATLQA linker 1-5 | | |
| 3 | 585-599 | FKELLVYAADPAMHA | RNA-dependent_RNA_polymerase | 368-382 |
| | | | (NC_04551: + : 13442-13468 + | |
| | | | 13468-16236) | |
| 3 | 600-604 | ATLQA linker 1-5 | | |
| 3 | 605-617 | FQTLLALHRSYLT | surface_glycoprotein@Aruba | 234-246 |
| | | | (NC_04551@Aruba: + : | |
| | | | 21563-25384) | |
| 3 | 618-622 | ATLQA linker 1-5 | | |
| 3 | 623-637 | TYGVGYQPYRVVVLS | surface_glycoprotein@Australia | 496-510 |
| | | | (NC_045512@Australia: + : | |
| | | | 21563-25384) | |
| 3 | 638-642 | ATLQA linker 1-5 | | |
| 3 | 643-659 | GVHFVCNLLLLFVIVYS | ORF3a_protein@Malaysia | 76-92 |
| | | | (NC_045512@Malaysia: + : | |
| | | | 25393-26220) | |
| 3 | 660-664 | ATLQA linker 1-5 | | |
| 3 | 665-684 | FCKLHNWNCVNCDTFCAGST | nsp3(NC_045512: + : 2720-8554) | 1626-1645 |
| 3 | 685-689 | ATLQA linker 1-5 | | |
| 3 | 690-705 | LMNVLTLFYKVYYGNA | nsp6@Chile(NC_045512@Chile: + : | 142-157 |
| | | | 10973-11842) | |
| 3 | 706-710 | ATLQA linker 1-5 | | |
| 3 | 711-733 | HQPYVVDDPCPIHFYSKWYIRVG | ORF8_protein(NC_04551: + : | 28-50 |
| | | | 27894-28259) | |
| 3 | 734-738 | ATLQA linker 1-5 | | |
| 3 | 739-749 | SAPLIELCLDE | ORF8_protein@Russia(NC_04551@ | 54-64 |
| | | | Russia: + : 27894-28259) | |
| 3 | 750-754 | ATLQA linker 1-5 | | |
| 3 | 755-777 | QLALSKGVYFVCNLLLLFVTVYS | ORF3a_protein@Aruba | 70-92 |
| | | | (NC_045512@Aruba: + : | |
| | | | 25393-26220) | |
| 3 | 778-782 | ATLQA linker 1-5 | | |
| 3 | 783-814 | IMASLVLARKHTTCCSLSHRFYRLA | RNA-dependent_RNA_polymerase | 632-663 |
| | | NECAQVL | (NC_04551: + : 13442-13468 + | |
| | | | 13468-16236) | |
| 3 | 815-819 | ATLQA linker 1-5 | | |
| 3 | 820-838 | KLNVGDYFVLTSHTVMPLS | helicase(NC_04551: + : | 218-236 |
| | | | 16237-18039) | |
| 3 | 839-843 | ATLQA linker 1-5 | | |
| 3 | 844-860 | YADVFHLYLQYIRKLHD | RNA-dependent_RNA_polymerase | 877-893 |
| | | | (NC_04551: + : 13442-13468 + | |
| | | | 13468-16236) | |
| 3 | 861-865 | ATLQA linker 1-5 | | |
| 3 | 866-876 | CYTPSKLIEYT | nsp4(NC_045512: + : 8555-10054) | 133-143 |
| 3 | 877-881 | ATLQA linker 1-5 | | |
| 3 | 882-890 | GVVQQLPET | endoRNAse(NC_045512: + : | 184-192 |
| | | | 19621-20658) | |
| 3 | 891-895 | ATLQA linker 1-5 | | |
| 3 | 896-915 | SEAVEAPLVGTPVCNINGLML | nsp2(NC_045512: + : 806-2719) | 591-610 |
| 3 | 916-920 | ATLQA linker 1-5 | | |
| 3 | 921-936 | SRIIPARAVDCFDKF | helicase@Brazil(NC_04551@Brazil: | 331-346 |
| | | | + : 16237-18039) | |
| 3 | 937-941 | ATLQA linker 1-5 | | |
| 3 | 942-953 | EVFAQVKQIYKT | surface_glycoprotein@Aruba | 776-787 |
| | | | (NC_045512@Aruba: + : | |
| | | | 21563-25384) | |
| 3 | 954-958 | ATLQA linker 1-5 | | |
| 3 | 959-971 | ISSVLNDILSRLD | surface_glycoprotein@Bangladesh | 972-984 |
| | | | (NC_045512@Bangladesh: + : | |
| | | | 21563-25384) | |
| 3 | 972-976 | ATLQA linker 1-5 | | |
| 3 | 977-988 | FSVNTWFHAIVS | surface_glycoprotein@Ghana | 59-70 |
| | | | (NC_045512@Ghana: + : | |
| | | | 21563-25384) | |
| 3 | 989-993 | ATLQA linker 1-5 | | |
| 3 | 994-1001 | PSFLGRYM | nsp3(NC_045512: + : 2720-8554) | 822-829 |
| 3 | 1002-1006 | ATLQA linker 1-5 | | |
| 3 | 1007-1020 | FGTYEKLKPVLDW | nsp2(NC_045512: + : 806-2719) | 437-450 |
| 3 | 1021-1025 | ATLQA linker 1-5 | | |
| 3 | 1026-1033 | IETISLAG | nsp3(NC_045512: + : 2720-8554) | 684-691 |
| 3 | 1034-1038 | ATLQA linker 1-5 | | |
| 3 | 1039-1047 | DYYQLYSTQ | ORF3a_protein(NC_045512: + : | 210-218 |
| | | | 25393-26220) | |
| 3 | 1048-1052 | ATLQA linker 1-5 | | |
| 3 | 1053-1062 | NEFYAYLRKH | RNA-dependent_RNA_polymerase | 972-984 |
| | | | (NC_04551: + : 13442-13468 + | |
| | | | 13468-16236) | |
| 3 | 1063-1067 | ATLQA linker 1-5 | | |
| 3 | 1068-1078 | GEVPVSIINNT | endoRNAse(NC_045512: + : | 20-30 |
| | | | 19621-20658) | |
| 3 | 1079-1083 | ATLQA linker 1-5 | | |
| 3 | 1084-1098 | ISNCVADYSVLYNSA | surface_glycoprotein@Aruba | 354-368 |
| | | | (NC_045512@Aruba: + : | |
| | | | 21563-25384) | |
| 3 | 1099-1103 | ATLQA linker 1-5 | | |
| 3 | 1104-1121 | VTTVIFLARGIVFNCVEY | nsp6@Malaysia(NC_045512@Malaysia: | 179-196 |
| | | | + : 10973-11842) | |
| 3 | 1122-1126 | ATLQA linker 1-5 | | |
| 3 | 1127-1146 | PINLVRGLPQGFSALEPLVD | surface_glycoprotein@Austria | 205-224 |
| | | | (NC_045512@Austria: + : | |
| | | | 21563-25384) | |
| 3 | 1147-1151 | ATLQA linker 1-5 | | |
| 3 | 1152-1159 | AALQIPFA | surface_glycoprotein(NC_045512: + : | 892-899 |
| | | | 21563-25384) | |
| 3 | 1160-1164 | ATLQA linker 1-5 | | |
| 3 | 1165-1175 | RFQLHRSYLTP | surface_glycoprotein@Austria | 233-243 |
| | | | (NC_045512@Austria: + : | |
| | | | 21563-25384) | |
| 3 | 1176-1180 | ATLQA linker 1-5 | | |
| 3 | 1181-1190 | VTGLFKDCSK | 3′-to-5′_exonuclease(NC_045512: + : | 4-13 |
| | | | 18040-19620) | |
| 3 | 1191-1195 | ATLQA linker 1-5 | | |
| 3 | 1196-1204 | FEHIVYGDF | endoRNAse(NC_045512: + : | 232-240 |
| | | | 19621-20658) | |
| 3 | 1205-1209 | ATLQA linker 1-5 | | |
| 3 | 1210-1224 | ALLAVFQSASKIITL | ORF2a_protein@Bangladesh | 51-65 |
| | | | (NC_045512@Bangladesh: + : | |
| | | | 25393-26220) | |
| 3 | 1225-1229 | ATLQA linker 1-5 | | |
| 3 | 1230-1239 | KCAYWVPRAS | nsp2(NC_045512: + : 806-2719) | 239-248 |
| 3 | 1240-1244 | ATLQA linker 1-5 | | |
| 3 | 1245-1259 | VDTSLSSLKLKDCVM | nsp6@Latvia(NC_045512@Latvia: + : | 101-115 |
| | | | 10973-11842) | |
| 3 | 1260-1264 | ATLQA linker 1-5 | | |
| 3 | 1265-1283 | ANSVFNICQSVTANVNALL | RNA-dependent_RNA_polymerase@ | 690-708 |
| | | | Croatia(NC_045512@Croatia: + : | |
| | | | 13442-13468 + 13468-16236) | |
| 3 | 1284-1288 | ATLQA linker 1-5 | | |
| 3 | 1289-1296 | YDPLQPEL | surface_glycoprotein(NC_045512: + : | 1138-1145 |
| | | | 21563-25384) | |
| 3 | 1297-1301 | ATLQA linker 1-5 | | |
| 3 | 1302-1311 | DPIHSLRVCV | nsp3(NC_045512: + : 2720-8554) | 339-348 |
| 3 | 1312-1316 | ATLQA linker 1-5 | | |
| 3 | 1317-1324 | CLNRVCTN | nsp3(NC_045512: + : 2720-8554) | 1342-1349 |
| 3 | 1325-1329 | ATLQA linker 1-5 | | |
| 3 | 1330-1338 | FFSNVTWFH | surface_glycoprotein(NC_045512: + : | 58-66 |
| | | | 21563-25384) | |
| 4 | 1-12 | TEISFMLWCKDG | endoRNAse(NC_045512: + : | 325-336 |
| | | | 19621-20658) | |
| 4 | 13-17 | ATLQA linker 1-5 | | |
| 4 | 18-35 | LNDLCFTNVYADSFVIRG | surface_glycoprotein(NC_045512: + : | 387-404 |
| | | | 21563-25384) | |
| 4 | 36-40 | ATLQA linker 1-5 | | |
| 4 | 41-50 | GTNLPLQLGF | 3′-to-5′_exonuclease(NC_045512: + : | 102-111 |
| | | | 18040-19620) | |
| 4 | 51-55 | ATLQA linker 1-5 | | |
| 4 | 56-76 | QWNLVGIFLFLTWICLLQFAY | membrane_glycoprotein@Aruba | 19-39 |
| | | | (NC_045512@Aruba: + : 26523-27191) | |
| 4 | 77-81 | ATLQA linker 1-5 | | |
| 4 | 82-102 | WMFLARGIVFMCVDYCPIFFI | nsp6@Cyprus(NC_045512@Cyprus: + : | 179-199 |
| | | | 10973-11842) | |
| 4 | 103-107 | ATLQA linker 1-5 | | |
| 4 | 108-115 | ETQALPQR | nucleocapsid_phosphoprotein@ | 378-385 |
| | | | SmallNorthAmerica(NC_045512@ | |
| | | | SmallNorthAmerica: + : | |
| | | | 28274-29533) | |
| 4 | 116-120 | ATLQA linker 1-5 | | |
| 4 | 121-130 | SPGVVNPVME | ORF2a_protein@Brazil(NC_045512@ | 252-261 |
| | | | Brazil: + : 25393-26220) | |
| 4 | 131-135 | ATLQA linker 1-5 | | |
| 4 | 136-146 | ITNCVKMLCTH | nsp10(NC_045512: + : 13025-13441) | 38-48 |
| 4 | 147-151 | ATLQA linker 1-5 | | |
| 4 | 152-171 | HYQECVRGTTVLLKEPCSSG | ORF3a_protein(NC_045512: + : | 19-38 |
| | | | 25393-26220) | |
| 4 | 172-176 | ATLQA linker 1-5 | | |
| 4 | 177-199 | KNPLLYDANYFLCCHTNCYDYCI | ORF2a_protein@Senegal(NC_045512@ | 136-158 |
| | | | Senegal: + : 25393-26220) | |
| 4 | 200-204 | ATLQA linker 1-5 | | |
| 4 | 205-216 | AKLLHKPIVWHV | nsp3(NC_045512: + : 2720-8554) | 1165-1176 |
| 4 | 217-221 | ATLQA linker 1-5 | | |
| 4 | 222-234 | SMWALIISVTSNY | nsp6@Aruba(NC_045512@Aruba: + : | 160-172 |
| | | | 10973-11842) | |
| 4 | 235-239 | ATLQA linker 1-5 | | |
| 4 | 240-247 | ARDLSLQF | nsp3(NC_045512: + : 2720-8554) | |
| 4 | 248-252 | ATLQA linker 1-5 | | |
| 4 | 253-281 | FLYIIKLIFLWLLWPVTLACFVLAA | membrane_glycoprotein@Aruba | 45-73 |
| | | VYRI | (NC_045512@Aruba: + : 26523-27191) | |
| 4 | 282-286 | ATLQA linker 1-5 | | |
| 4 | 287-295 | ASQGLLASI | RNA-dependent_RNA_polymerase@ | 771-779 |
| | | | Austria(NC_045512@Austria: + : | |
| | | | 13442-13468 + 13468-16236) | |
| 4 | 296-300 | ATLQA linker 1-5 | | |
| 4 | 301-311 | FFSNVTWFHVS | surface_glycoprotein@Nigeria | 58-68 |
| | | | (NC_045512@Nigeria: + : | |
| | | | 21563-25384) | |
| 4 | 312-316 | ATLQA linker 1-5 | | |
| 4 | 317-325 | TENLLLYID | nsp3(NC_045512: + : 2720-8554) | 432-440 |
| 4 | 326-330 | ATLQA linker 1-5 | | |
| 4 | 331-342 | SVFQSASKIITL | ORF2a_protein@Iceland(NC_045512@ | 54-65 |
| | | | Iceland: + : 25393-26220) | |
| 4 | 343-347 | ATLQA linker 1-5 | | |
| 4 | 348-357 | GNCDVVIGIV | surface_glycoprotein(NC_045512: + : | 1124-1133 |
| | | | 21563-25384) | |
| 4 | 358-362 | ATLQA linker 1-5 | | |
| 4 | 363-383 | HPNCVNCLDDRCILHCANFNV | RNA-dependent_RNA_polymerase@ | 295-315 |
| | | | Australia(NC_045512@Australia: + : | |
| | | | 13442-13468 + 13468-16236) | |
| 4 | 384-388 | ATLQA linker 1-5 | | |
| 4 | 389-398 | ACIDCSARHI | nsp3(NC_045512: + : 2720-8554) | 1872-1881 |
| 4 | 399-403 | ATLQA linker 1-5 | | |
| 4 | 404-418 | TISVTTEILPVSMAK | surface_glycoprotein@Mexico | 719-733 |
| | | | (NC_045512@Mexico: + : | |
| | | | 21563-25384) | |
| 4 | 419-423 | ATLQA linker 1-5 | | |
| 4 | 424-437 | TSACVLAAECTIFK | nsp4(NC_045512: + : 8555-10054) | 147-160 |
| 4 | 438-442 | ATLQA linker 1-5 | | |
| 4 | 443-450 | SINVANYQ | helicase@Mozambique(NC_045512@ | 263-270 |
| | | | @Mozambique: + : 16237-18039) | |
| 4 | 451-455 | ATLQA linker 1-5 | | |
| 4 | 456-465 | EGSVRVVTTG | nsp4(NC_045512: + : 8555-10054) | 207-216 |
| 4 | 466-470 | ATLQA linker 1-5 | | |
| 4 | 471-495 | VLVQSTQWSLFFFFYENAFLPFAMG | nsp6@Iceland(NC_045512@ | 719-733 |
| | | | Iceland: + :10973-11842) | |
| 4 | 496-500 | ATLQA linker 1-5 | | |
| 4 | 501-533 | YEPLTQDHVDILGPLSAQTGIAVLD | 3C-like_proteinase(NC_045512: + : | 239-271 |
| | | MCASLKEL | 10055-10972) | |
| 4 | 534-538 | ATLQA linker 1-5 | | |
| 4 | 539-547 | SRELKVTFF | nsp3(NC_045512: + : 2720-8554) | 1134-1142 |
| 4 | 548-552 | ATLQA linker 1-5 | | |
| 4 | 553-570 | YNLLKDCPAVAKHDFFKF | RNA-dependent_RNA_polymerase@ | 87-104 |
| | | | Aruba(NC_045512@Aruba: + : | |
| | | | 13442-13468 + 13468-16236) | |
| 4 | 571-575 | ATLQA linker 1-5 | | |
| 4 | 576-597 | TLGVLVPHVGEIPVAYRKVLLR | leader_protein(NC_045512: + : | 103-124 |
| | | | 266-805) | |
| 4 | 598-602 | ATLQA linker 1-5 | | |
| 4 | 603-633 | YIWLGFIAGLIAIVMVTIMLCCMTS | surface_glycoprotein@Aruba | 1211-1241 |
| | | CCSCLK | (NC_045512@Aruba: + : | |
| | | | 21563-25384) | |
| 4 | 634-638 | ATLQA linker 1-5 | | |
| 4 | 639-654 | LVDFQVTIAEILLIIM | ORF6_protein(NC_045512: + : | 4-19 |
| | | | 27202-27387) | |
| 4 | 655-659 | ATLQA linker 1-5 | | |
| 4 | 660-671 | TFKVSIWNLDYI | ORF6_protein(NC_045512: + : | 21-32 |
| | | | 27202-27387) | |
| 4 | 672-676 | ATLQA linker 1-5 | | |
| 4 | 677-686 | GVGGKPCIKV | nsp6@Aruba(NC_045512@ | 274-283 |
| | | | Aruba: + :10973-11842) | |
| 4 | 687-691 | ATLQA linker 1-5 | | |
| 4 | 690-716 | SDIDITFLKKDAPYIVGDVVQEGVL | nsp3(NC_045512: + : 2720-8554) | 454-478 |
| 4 | 717-721 | ATLQA linker 1-5 | | |
| 4 | 722-745 | LGSLVVRCSFYEDFLEYHDVRVVL | ORF8_protein(NC_045512: + : | 95-118 |
| | | | 27894-28259) | |
| 4 | 746-750 | ATLQA linker 1-5 | | |
| 4 | 751-763 | ANFCALILAYCNK | nsp3(NC_045512: + : 2720-8554) | 890-902 |
| 4 | 764-768 | ATLQA linker 1-5 | | |
| 4 | 769-794 | YRLANECAQVLSEMVMCGGSLYVKP | RNA-dependent_RNA_polymerase@ | 653-678 |
| | | G | Australia(NC_045512@Australia: + : | |
| | | | 13442-13468 + 13468-16236) | |
| 4 | 795-799 | ATLQA linker 1-5 | | |
| 4 | 800-807 | QIGVVREF | helicase(NC_045512: + : | 492-499 |
| | | | 16237-18039) | |
| 4 | 808-812 | ATLQA linker 1-5 | | |
| 4 | 813-827 | SYFAIGLALYYPSAR | helicase@Austria(NC_045512@ | 289-303 |
| | | | @Austria: + : 16237-18039) | |
| 4 | 828-832 | ATLQA linker 1-5 | | |
| 4 | 833-842 | SFKLKDCVMY | nsp6@Malaysia(NC_045512@ | 107-116 |
| | | | Malaysia: + :10973-11842) | |
| 4 | 843-847 | ATLQA linker 1-5 | | |
| 4 | 848-857 | PRWYFYYLGT | nucleocapsid_phosphoprotein@Aruba | 106-115 |
| | | | (NC_045512@Aruba: + : 28274-29533) | |
| 4 | 858-862 | ATLQA linker 1-5 | | |
| 4 | 863-899 | VAAGLEAPFLYLYALVYFLQSINFV | ORF3a_protein@Aruba(NC_045512@ | 97-133 |
| | | RIIMRLWLCWKC | Aruba: + : 25393-26220) | |
| 4 | 900-904 | ATLQA linker 1-5 | | |
| 4 | 905-916 | SVLLSMQGAVDI | nsp7(NC-4551: + : 11843-12091) | 57-68 |
| 4 | 917-921 | ATLQA linker 1-5 | | |
| 4 | 922-932 | ETTVDIVVFDE | helicase@SmallAsia(NC_045512@ | 365-375 |
| | | | @SmallAsia: + : 16237-18039) | |
| 4 | 933-937 | ATLQA linker 1-5 | | |
| 4 | 938-961 | TYASALWEIQQVVDADSKIVQLSE | nsp8(NC_045512: + : 12092-12685) | 148-171 |
| 4 | 962-966 | ATLQA linker 1-5 | | |
| 4 | 967-977 | VLYQGVNCTEV | surface_glycoprotein@Aruba | 606-616 |
| | | | (NC_045512@Aruba: + : | |
| | | | 21563-25384) | |
| 4 | 978-982 | ATLQA linker 1-5 | | |
| 4 | 983-1005 | TTVMFLARGIVFMCVEYCPIFFI | nsp6@Aruba(NC_045512@ | 177-199 |
| | | | Aruba: + :10973-11842) | |
| 4 | 1006-1010 | ATLQA linker 1-5 | | |
| 4 | 1011-1018 | GVYCCREH | nsp2(NC_045512: + : 806-2719) | 47-54 |
| 4 | 1019-1023 | ATLQA linker 1-5 | | |
| 4 | 1024-1031 | EIYQAGSK | surface_glycoprotein@Mexico | 471-478 |
| | | | (NC_045512@Mexico: + : | |
| | | | 21563-25384) | |
| 4 | 1032-1036 | ATLQA linker 1-5 | | |
| 4 | 1037-1059 | LIAIVMVTIMLCCITSCCSCLKG | surface_glycoprotein@Mozambique | 1220-1242 |
| | | | (NC_045512@Mozambique: + : | |
| | | | 21563-25384) | |
| 4 | 1060-1064 | ATLQA linker 1-5 | | |
| 4 | 1065-1074 | ARDLICAQKF | surface_glycoprotein(NC_045512: + : | 846-855 |
| | | | 21563-25384) | |
| 4 | 1075-1079 | ATLQA linker 1-5 | | |
| 4 | 1080-1092 | NPVLPFNDGVYFA | surface_glycoprotein(NC_045512: + : | 81-93 |
| | | | 21563-25384) | |
| 4 | 1093-1097 | ATLQA linker 1-5 | | |
| 4 | 1098-1114 | VPQEHYVRITGLPTLN | helicase@Aruba(NC_045512@ | 241-257 |
| | | | @Aruba: + : 16237-18039) | |
| 4 | 1115-1119 | ATLQA linker 1-5 | | |
| 4 | 1120-1144 | VLVQSTQWSLFFFLYENAFLPFAMG | nsp6@Aruba(NC_045512@ | 24-48 |
| | | | Aruba: + :10973-11842) | |
| 4 | 1145-1149 | ATLQA linker 1-5 | | |
| 4 | 1150-1161 | HEFCSQHTMLVK | RNA-dependent_RNA_polymerase | 810-821 |
| | | | (NC_04551: + : 13442-13468 + | |
| | | | 13468-16236) | |
| 4 | 1162-1166 | ATLQA linker 1-5 | | |
| 4 | 1167-1185 | AVAVLYQDVNCTEVPVAIH | surface_glycoprotein@China | 607-625 |
| | | | (NC_045512@China: + : | |
| | | | 21563-25384) | |
| 4 | 1186-1190 | ATLQA linker 1-5 | | |
| 4 | 1191-1210 | DDRCILHCANFNVLFSTVFP | RNA-dependent_RNA_polymerase | 303-322 |
| | | | (NC_04551: + : 13442-13468 + | |
| | | | 13468-16236) | |
| 4 | 1211-1512 | ATLQA linker 1-5 | | |
| 4 | 1216-1225 | FEYVSQPFLM | surface_glycoprotein@Aruba | 163-173 |
| | | | (NC_045512@Aruba: + : | |
| | | | 21563-25384) | |
| 4 | 1226-1230 | ATLQA linker 1-5 | | |
| 4 | 1231-1244 | GFNCYFPLQPYGFQ | surface_glycoprotein@ | 481-494 |
| | | | SmallSouthAmerica(NC_045512@ | |
| | | | SmallSouthAmerica: + : 21563-25384) | |
| 4 | 1245-1249 | ATLQA linker 1-5 | | |
| 4 | 1250-1267 | IKVCEFQFCNFPFLGVYH | surface_glycoprotein@Aruba | 125-142 |
| | | | (NC_045512@Aruba: + : | |
| | | | 21563-25384) | |
| 4 | 1268-1272 | ATLQA linker 1-5 | | |
| 4 | 1273-1295 | NHDLYCQVHGNAHVASCDAIMTR | 3′-to-5′_exonuclease(NC_045512: + : | 256-278 |
| | | | 18040-19620) | |
| 4 | 1296-1300 | ATLQA linker 1-5 | | |
| 4 | 1301-1323 | SHGKQVVSDIDYVPLKSATCITR | 3′-to-5′_exonuclease(NC_045512: + : | 454-476 |
| | | | 18040-19620) | |
| 4 | 1324-1328 | ATLQA linker 1-5 | | |
| 4 | 1329-1349 | AVLYQGVNCTEVPFAIHADQL | surface_glycoprotein@SmallAfrica | 605-625 |
| | | | (NC_045512@SmallAfrica: + : | |
| | | | 21563-25384) | |
| 5 | 1-16 | YVRITGLYPTLNISYE | helicase@SmallNorthAmerica | 246-261 |
| | | | (NC_-45512@SmallNorthAmerica: + : | |
| | | | 16237-18039) | |
| 5 | 17-21 | ATLQA linker 1-5 | | |
| 5 | 22-35 | LEKILVTYNCCDDD | RNA-dependent_RNA_polymerase | 142-155 |
| | | | (NC_04551: + : 13442-13468 + | |
| | | | 13468-16236) | |
| 5 | 36-40 | ATLQA linker 1-5 | | |
| 5 | 41-50 | LSVNPYVCNA | helicase@Cyprus(NC_045512@Cyprus: | 43-52 |
| | | | + : 16237-18039) | |
| 5 | 51-55 | ATLQA linker 1-5 | | |
| 5 | 56-65 | ETFVTHSKGL | nsp2(NC_045512: + : 806-2719) | 527-536 |
| 5 | 66-70 | ATLQA linker 1-5 | | |
| 5 | 71-85 | TYACWHHSIGFDYVY | 3′-to-5′_exonuclease(NC_045512: + : | 223-237 |
| | | | 18040-19620) | |
| 5 | 86-90 | ATLQA linker 1-5 | | |
| 5 | 91-101 | AVHECFVKRVD | 3′-to-5′_exonuclease(NC_045512: + : | 281-291 |
| | | | 18040-19620) | |
| 5 | 102-106 | ATLQA linker 1-5 | | |
| 5 | 107-116 | SVELKHFFFA | RNA-dependent_RNA_polymerase | 434-443 |
| | | | (NC_04551: + : 13442-13468 + | |
| | | | 13468-16236) | |
| 5 | 117-121 | ATLQA linker 1-5 | | |
| 5 | 122-135 | TSGVGYQPYRVVVL | surface_glycoprotein@Aruba | 496-509 |
| | | | (NC_045512@Aruba: + : | |
| | | | 21563-25384) | |
| 5 | 136-140 | ATLQA linker 1-5 | | |
| 5 | 141-148 | EVTPSGTS | nucleocapsid_phosphoprotein | 323-330 |
| | | | (NC_045512: + : 28274-29533) | |
| 5 | 149-153 | ATLQA linker 1-5 | | |
| 5 | 154-163 | DVVAIDYKHY | nsp3(NC_045512: + : 2720-8554) | 1148-1157 |
| 5 | 164-168 | ATLQA linker 1-5 | | |
| 5 | 169-176 | NNLVVMAY | nsp2(NC_045512: + : 806-2719) | 413-420 |
| 5 | 177-181 | ATLQA linker 1-5 | | |
| 5 | 182-189 | SSIVITSG | ORF3a_protein@NorthKorea(NC_045512@ | 165-172 |
| | | | NorthKorea: + : 25393-26220) | |
| 5 | 190-194 | ATLQA linker 1-5 | | |
| 5 | 195-233 | GCDVTDVTQLYLGGMSYYCKSHKPP | helicase(NC_045512: + : | 54-92 |
| | | ISFPLCANGQVFGL | 16237-18039) | |
| 5 | 234-238 | ATLQA linker 1-5 | | |
| 5 | 239-246 | DQDVLFAY | RNA-dependent_RNA_polymerase@ | 523-530 |
| | | | FrenchGuiana(NC_045512@FrenchGuiana | |
| | | | : + :13442-13468 + 13468-16236) | |
| 5 | 247-251 | ATLQA linker 1-5 | | |
| 5 | 252-261 | RHSLSHFVNL | nsp3(NC_045512: + : 2720-8554) | 1697-1706 |
| 5 | 262-266 | ATLQA linker 1-5 | | |
| 5 | 267-274 | SVDCTMYI | surface_glycoprotein(NC_04551: + : | 735-742 |
| | | | 21563-25384) | |
| 5 | 275-279 | ATLQA linker 1-5 | | |
| 5 | 280-287 | SDFVRATA | ORF3a_protein(NC_04551: + : | 26-33 |
| | | | 25393-26220) | |
| 5 | 288-292 | ATLQA linker 1-5 | | |
| 5 | 293-304 | FRSSVLHSTRDL | surface_glycoprotein@Nigeria | 43-54 |
| | | | (NC_045512@Nigeria: + : | |
| | | | 21563-25384) | |
| 5 | 305-309 | ATLQA linker 1-5 | | |
| 5 | 310-317 | FKLASHMY | nsp3(NC_045512: + : 2720-8554) | 96-103 |
| 5 | 318-322 | ATLQA linker 1-5 | | |
| 5 | 323-336 | LSPVALRQMSCAAG | nsp9(NC_045512: + : 12686-13024) | 4-17 |
| 5 | 337-341 | ATLQA linker 1-5 | | |
| 5 | 342-360 | AIPINFTISVTTEILPVSM | surface_glycoprotein@Aruba | 709-727 |
| | | | (NC_045512@Aruba: + : | |
| | | | 21563-25384) | |
| 5 | 361-365 | ATLQA linker 1-5 | | |
| 5 | 366-375 | SVLNDILARL | surface_glycoprotein@Aruba | 971-980 |
| | | | (NC_045512@Aruba: + : | |
| | | | 21563-25384) | |
| 5 | 376-380 | ATLQA linker 1-5 | | |
| 5 | 381-394 | RLNVGDYFVLTSHT | helicase@Austria(NC_045512@Austria: | 218-231 |
| | | | + : 16237-18039) | |
| 5 | 395-399 | ATLQA linker 1-5 | | |
| 5 | 400-408 | ISEHDYHIG | ORF3a_protein@NorthMacedonia | 179-187 |
| | | | (NC_045512@NorthMacedonia: + : | |
| | | | 25393-26220) | |
| 5 | 409-413 | ATLQA linker 1-5 | | |
| 5 | 414-421 | STFKCYGV | surface_glycoprotein@Aruba | 371-378 |
| | | | (NC_045512@Aruba: + : | |
| | | | 21563-25384) | |
| 5 | 422-426 | ATLQA linker 1-5 | | |
| 5 | 427-436 | GLGLKDCVMY | nsp6@Peru(NC_045512@Peru: + : | 107-116 |
| | | | 10973-11842) | |
| 5 | 437-441 | ATLQA linker 1-5 | | |
| 5 | 442-449 | SQDLSVVS | endoRNAse(NC_045512: + : | 308-315 |
| | | | 19621-20658) | |
| 5 | 450-454 | ATLQA linker 1-5 | | |
| 5 | 455-468 | GFNCYSPLQSYGFQ | surface_glycoprotein@Peru | 485-498 |
| | | | (NC_045512@Peru: + : | |
| | | | 21563-25384) | |
| 5 | 469-473 | ATLQA linker 1-5 | | |
| 5 | 474-489 | AFKLNIKLLGVGGKPC | nsp6(NC_045512: + : 10973-11842) | 268-283 |
| 5 | 490-494 | ATLQA linker 1-5 | | |
| 5 | 495-502 | DCATVHTA | 2′-O-ribose_methyltransferase | 114-121 |
| | | | (NC_045112: + : 20659-21552) | |
| 5 | 503-507 | ATLQA linker 1-5 | | |
| 5 | 508-518 | SPGVFNPVMEP | ORF3a_protein@Colombia | 252-262 |
| | | | (NC_045512@Colombia: + : | |
| | | | 25393-26220) | |
| 5 | 519-523 | ATLQA linker 1-5 | | |
| 5 | 524-538 | VCNLLLLFVTVYSHL | ORF3a_protein@Croatia | 80-94 |
| | | | (NC_045512@Croatia: + : | |
| | | | 25393-26220) | |
| 5 | 539-543 | ATLQA linker 1-5 | | |
| 5 | 544-585 | TTVAAFHQECSLQSCTQHQPYVVDD | ORF8_protein@Bangladesh | 11-52 |
| | | PCPIHFYSKWYIRVGAR | (NC_045512@Bangladesh: + : | |
| | | | 27894-28259) | |
| 5 | 586-590 | ATLQA linker 1-5 | | |
| 5 | 591-600 | VMCGGSLYVK | RNA-dependent_RNA_polymerase | 667-676 |
| | | | (NC_04551: + : 13442-13468 + | |
| | | | 13468-16236) | |
| 5 | 601-605 | ATLQA linker 1-5 | | |
| 5 | 606-626 | ALCEKALKYFPIDKCSRIIPA | helicase@Colombia(NC_045512@ | 316-336 |
| | | | Colombia: + : 16237-18039) | |
| 5 | 627-631 | ATLQA linker 1-5 | | |
| 5 | 632-648 | KDQVILLNKHIDAYKTF | nucleocapsid_phosphoprotein@Aruba | 347-363 |
| | | | (NC_04551@Aruba: + : 28274-29533) | |
| 5 | 649-653 | ATLQA linker 1-5 | | |
| 5 | 654-666 | KLALGGSVAIKIT | 2′-O-ribose_methyltransferase | 160-172 |
| | | | (NC_045112: + : 20659-21552) | |
| 5 | 667-671 | ATLQA linker 1-5 | | |
| 5 | 672-681 | NLIIKNLSKS | ORF6_protein(NC_045512: + : | 34-43 |
| | | | 27202-27387) | |
| 5 | 682-686 | ATLQA linker 1-5 | | |
| 5 | 687-707 | ELVIGAVILRGHLRIAGHHLG | membrane_glycoprotein@Aruba | 137-157 |
| | | | (NC_045512@Aruba: + : 26523-27191) | |
| 5 | 708-712 | ATLQA linker 1-5 | | |
| 5 | 713-726 | TCFSVAALTNNVAF | RNA-dependent_RNA_polymerase@Aruba | 394-407 |
| | | | (NC_04551@Aruba: + : | |
| | | | 13442-13468 + 13468-16236) | |
| 5 | 727-731 | ATLQA linker 1-5 | | |
| 5 | 732-754 | TIATYCTGSIPCSVCLSGLDSLD | nsp3(NC_045512: + : 2720-8554) | 1459-1481 |
| 5 | 755-759 | ATLQA linker 1-5 | | |
| 5 | 760-773 | TKDVVECLKLSHQS | nsp3(NC_045512: + : 2720-8554) | 1830-1843 |
| 5 | 774-778 | ATLQA linker 1-5 | | |
| 5 | 779-796 | IKDLPKEITVATSRTLSY | membrane_glycoprotein_NC_045512: | 161-178 |
| | | | + : 26523-27191) | |
| 5 | 797-801 | ATLQA linker 1-5 | | |
| 5 | 802-817 | TGLHPTQAPTHLSVDT | 3′-to-5′_exonuclease(NC_045512: + : | 16-31 |
| | | | 18040-19620) | |
| 5 | 818-822 | ATLQA linker 1-5 | | |
| 5 | 823-851 | SMWALIISVTSNYSGVVTIVMFLAR | nsp6@SmallSouthAmerica(NC_045512@ | 160-188 |
| | | GIVF | SmallSouthAmerica: + : | |
| | | | 10973-11842) | |
| 5 | 852-856 | ATLQA linker 1-5 | | |
| 5 | 857-864 | IAANTVIW | endoRNAse(NC_045512: + : | 79-86 |
| | | | 19621-20658) | |
| 5 | 865-869 | ATLQA linker 1-5 | | |
| 5 | 870-878 | SEAFLIGCN | 2′-O-ribose_methyltransferase | 202-210 |
| | | | (NC_045112: + : 20659-21552) | |
| 5 | 879-883 | ATLQA linker 1-5 | | |
| 5 | 884-892 | VTANVNALL | RNA-dependent_RNA_polymerase | 700-708 |
| | | | (NC_04551: + : | |
| | | | 13442-13468 + 13468-16236) | |
| 5 | 893-897 | ATLQA linker 1-5 | | |
| 5 | 898-911 | ESVVKDCVVLHSYF | ORF3a_protein@CostaRica(NC_04551@ | 194-207 |
| | | | CostaRica: + : 25393-26220) | |
| 5 | 912-916 | ATLQA linker 1-5 | | |
| 5 | 917-929 | RSVASQSIIAYTM | surface_glycoprotein@Aruba | 681-693 |
| | | | (NC_045512@Aruba: + : | |
| | | | 21563-25384) | |
| 5 | 930-934 | ATLQA linker 1-5 | | |
| 5 | 935-945 | EQYVFCTVANL | helicase(NC_04551: + : | 353-363 |
| | | | 16237-18039) | |
| 5 | 946-950 | ATLQA linker 1-5 | | |
| 5 | 951-959 | ASQGLVASI | RNA-dependent_RNA_polymerase@Aruba | 771-779 |
| | | | (NC_04551@Aruba: + : | |
| | | | 13442-13468 + 13468-16236) | |
| 5 | 960-964 | ATLQA linker 1-5 | | |
| 5 | 965-980 | QLALSKGFHFVCNLLL | ORF3a_protein@Iceland(NC_04551@ | 70-85 |
| | | | Iceland: + : 25393-26220) | |
| 5 | 981-985 | ATLQA linker 1-5 | | |
| 5 | 986-999 | RLTLGVYDYLVSTQ | nsp6(NC_045512: + : 10973-11842) | 236-249 |
| 5 | 1000-1004 | ATLQA linker 1-5 | | |
| 5 | 1005-1015 | KESVQTFFKLV | nsp2(NC_04551: + : 806-2719) | 492-502 |
| 5 | 1016-1020 | ATLQA linker 1-5 | | |
| 5 | 1021-1043 | VAAGLEAPFFYLYALVYFLQSIN | ORF3a_protein@Ecuador(NC_04551@ | 97-119 |
| | | | Ecuador: + : 25393-26220) | |
| 5 | 1044-1048 | ATLQA linker 1-5 | | |
| 5 | 1049-1070 | SDFVRATATIPIRASLPFGWLI | ORF3a_protein@Belgium(NC_04551@ | 26-47 |
| | | | Belgium: + : 25393-26220) | |
| 5 | 1071-1075 | ATLQA linker 1-5 | | |
| 5 | 1076-1094 | ECAQVLSETVMCGGSLYVK | RNA-dependent_RNA_polymerase@Aruba | 658-676 |
| | | | (NC_04551@Aruba: + : | |
| | | | 13442-13468 + 13468-16236) | |
| 5 | 1095-1099 | ATLQA linker 1-5 | | |
| 5 | 1100-1111 | LKKLKKSLNVAK | nsp8(NC_045512: + : 12092-12685) | 35-46 |
| 5 | 1112-1116 | ATLQA linker 1-5 | | |
| 5 | 1117-1138 | LGGMSYYCKSHKPSISFPLCAN | helicase@NewZealand(NC_045512@ | 65-86 |
| | | | NewZealand: + : 16237-18039) | |
| 5 | 1139-1143 | ATLQA linker 1-5 | | |
| 5 | 1144-1156 | WNVVRIKIVQMLS | 3′-to-5′_exonuclease(NC_045512: + : | 159-171 |
| | | | 18040-19620) | |
| 5 | 1157-1161 | ATLQA linker 1-5 | | |
| 5 | 1162-1177 | FSNVTWFHAIHVSGTN | surface_glycoprotein@Peru | 59-74 |
| | | | (NC_045512@Peru: + : | |
| | | | 21563-25384) | |
| 5 | 1178-1182 | ATLQA linker 1-5 | | |
| 5 | 1183-1199 | SAAYYVGYLQLRTFLLK | surface_glycoprotein@Italy | 258-274 |
| | | | (NC_045512@Italy: + : | |
| | | | 21563-25384) | |
| 5 | 1200-1204 | ATLQA linker 1-5 | | |
| 5 | 1205-1220 | NYTVSCSPFTINCQEP | ORF8_protein@CostaRica(NC_045512@ | 78-93 |
| | | | CostaRica: + : 27894-28259) | |
| 5 | 1221-1225 | ATLQA linker 1-5 | | |
| 5 | 1226-1240 | YFTSDYYQLYSTQLI | ORF8_protein@Indonesia(NC_045512@ | 206-220 |
| | | | Indonesia: + : 27894-28259) | |
| 5 | 1241-1245 | ATLQA linker 1-5 | | |
| 5 | 1246-1260 | RIIPARAVECFDKF | helicase@Aruba(NC_045512@ | 332-346 |
| | | | Aruba: + : 16237-18039) | |
| 5 | 1261-1265 | ATLQA linker 1-5 | | |
| 5 | 1266-1275 | DGYVMHANYI | 2′-O-ribose_methyltransferase | 220-229 |
| | | | (NC_045112: + : 20659-21552) | |
| 5 | 1276-1280 | ATLQA linker 1-5 | | |
| 5 | 1281-1294 | SYLFQHANLDSCKR | nsp3(NC_045512: + : 2720-8554) | 915-928 |
| 5 | 1295-1299 | ATLQA linker 1-5 | | |
| 5 | 1300-1308 | EVFAQVKQI | surface_glycoprotein(NC_04551: + : | 780-788 |
| | | | 21563-25384) | |
| 5 | 1309-1313 | ATLQA linker 1-5 | | |
| 5 | 1314-1329 | FLVLLPLVSIQCVNLT | surface_glycoprotein@ | 4-19 |
| | | | SmallNorthAmerica(NC_04551@ | |
| | | | SmallNorthAmerica: + : | |
| | | | 21563-25384) | |
| 5 | 1330-1334 | ATLQA linker 1-5 | | |
| 5 | 1335-1349 | YNLLKGCPAVAKHDF | RNA-dependent_RNA_polymerase@Poland | 87-101 |
| | | | (NC_04551@Poland: + : | |
| | | | 13442-13468 + 13468-16236) | |
| 6 | 1-11 | GKPVPYCYDTN | nsp4(NC_04551: + : 8555-10054) | 164-174 |
| 6 | 12-16 | ATLQA linker 1-5 | | |
| 6 | 17-33 | EVTPSGTWLTYTGAIKL | nucleocapsid_phosphoprotein@ | 323-339 |
| | | | Bulgaria(NC_04551@Bulgaria: + : | |
| | | | 28274-29533) | |
| 6 | 34-38 | ATLQA linker 1-5 | | |
| 6 | 39-60 | INLVRDLPQGFSALEPLVDLPI | surface_glycoprotein@Aruba | 206-227 |
| | | | (NC_045512@Aruba: + : | |
| | | | 21563-25384) | |
| 6 | 61-65 | ATLQA linker 1-5 | | |
| 6 | 66-76 | YDNLQFISLEI | helicase@Austria(NC_045512@ | 582-592 |
| | | | Austria: + : 16237-18039) | |
| 6 | 77-81 | ATLQA linker 1-5 | | |
| 6 | 82-103 | SDFVRATATIPIQASLPFGWLI | ORF3a_protein@Aruba(NC_04551@ | 26-47 |
| | | | Aruba: + : 25393-26220) | |
| 6 | 104-108 | ATLQA linker 1-5 | | |
| 6 | 109-116 | PVLKGVKL | surface_glycoprotein@Aruba | 1259-1266 |
| | | | (NC_045512@Aruba: + : | |
| | | | 21563-25384) | |
| 6 | 117-121 | ATLQA linker 1-5 | | |
| 6 | 122-131 | NVAFNVVNKG | endoRNAase(NC_045512: + : | 4-13 |
| | | | 19621-20658) | |
| 6 | 132-136 | ATLQA linker 1-5 | | |
| 6 | 137-153 | CSNLLLQYGSFCTQLNR | surface_glycoprotein(NC_04551: + : | 749-765 |
| | | | 21563-25384) | |
| 6 | 154-158 | ATLQA linker 1-5 | | |
| 6 | 159-167 | SPDVDLADI | surface_glycoprotein@Chile | 1161-1169 |
| | | | (NC_045512@Chile: + : | |
| | | | 21563-25384) | |
| 6 | 168-172 | ATLQA linker 1-5 | | |
| 6 | 173-187 | SYELQTPFEIKLAKK | nsp2(NC_045512: + : 806-2719) | 68-82 |
| 6 | 188-192 | ATLQA linker 1-5 | | |
| 6 | 193-204 | NIGCNHTGVVGE | nsp2(NC_045512: + : 806-2719) | 250-261 |
| 6 | 205-209 | ATLQA linker 1-5 | | |
| 6 | 210-221 | VRTNVYLAVFDK | nsp3(NC_045512: + : 2720-8554) | 351-362 |
| 6 | 222-226 | ATLQA linker 1-5 | | |
| 6 | 227-242 | ANSIVCRFDTRVLSNL | 3′-to-5′_exonuclease(NC_045512: + : | 394-409 |
| | | | 18040-19620) | |
| 6 | 243-247 | ATLQA linker 1-5 | | |
| 6 | 248-260 | ATNVVIKVCEFQF | surface_glycoprotein(NC_04551: + : | 123-135 |
| | | | 21563-25384) | |
| 6 | 261-265 | ATLQA linker 1-5 | | |
| 6 | 266-287 | QLALSKGVHFVCNLLLLFVTVY | ORF3a_protein@Australia(NC_04551@ | 70-91 |
| | | | Australia: + : 25393-26220) | |
| 6 | 288-292 | ATLQA linker 1-5 | | |
| 6 | 293-308 | HWPQIAQFAPSASAFF | nucleocapsid_phosphoprotein@ | 300-315 |
| | | | Aruba(NC_04551@Aruba: + : | |
| | | | 28274-29533) | |
| 6 | 309-313 | ATLQA linker 1-5 | | |
| 6 | 314-321 | RVEAFEYY | nsp3(NC_045512: + : 2720-8554) | 810-817 |
| 6 | 322-326 | ATLQA linker 1-5 | | |
| 6 | 327-343 | ILLNKHIDAYKTFPPIE | nucleocapsid_phosphoprotein@ | 351-367 |
| | | | Chile(NC_04551@Chile: + : | |
| | | | 28274-29533) | |
| 6 | 344-348 | ATLQA linker 1-5 | | |
| 6 | 349-357 | KFPLKLRGT | 2′-O-ribose_methyltransferase | 249-257 |
| | | | (NC_045112: + : 20659-21552) | |
| 6 | 358-362 | ATLQA linker 1-5 | | |
| 6 | 363-372 | NCYDYCIPYN | ORF3a_protein(NC_045512: + : | 152-161 |
| | | | 25393-26220_ | |
| 6 | 373-377 | ATLQA linker 1-5 | | |
| 6 | 378-391 | GASCCLYCRCHIDH | nsp10(NC_045512: + :13025-13441) | 70-83 |
| 6 | 392-396 | ATLQA linker 1-5 | | |
| 6 | 397-406 | VQQESPFVMM | nsp3(NC_045512: + : 2720-8554) | 980-989 |
| 6 | 407-411 | ATLQA linker 1-5 | | |
| 6 | 412-422 | CLPFTINCQKP | ORF8_protein@Brazil(NC_04551@ | 83-93 |
| | | | Brazil: + : 27894-28259) | |
| 6 | 423-427 | ATLQA linker 1-5 | | |
| 6 | 428-450 | KEITVATSRTLSYYKLGASQRVA | membrane_glycoprotein@Aruba | 166-188 |
| | | | (NC_045512@Aruba: + : | |
| | | | 26523-27191) | |
| 6 | 451-455 | ATLQA linker 1-5 | | |
| 6 | 456-466 | VVNAANVYLKH | nsp3(NC_045512: + : 2720-8554) | 239-249 |
| 6 | 467-471 | ATLQA linker 1-5 | | |
| 6 | 472-484 | AIKCVPQADVEWK | 3′-to-5′_exonuclease(NC_045512: + : | 337-349 |
| | | | 18040-19620) | |
| 6 | 485-489 | ATLQA linker 1-5 | | |
| 6 | 590-506 | LFFFLYENAFLPFVMGI | nsp6@SmallSouthAmerica(NC_045512@ | 33-49 |
| | | | SmallSouthAmerica: + : | |
| | | | 10973-11842) | |
| 6 | 507-511 | ATLQA linker 1-5 | | |
| 6 | 512-522 | YDKLQFTSLEI | helicase@Aruba(NC_045512@ | 582-592 |
| | | | Aruba: + : 16237-18039) | |
| 6 | 523-527 | ATLQA linker 1-5 | | |
| 6 | 528-535 | SSNVANYQ | helicase@Austria(NC_045512@ | 263-270 |
| | | | Austria: + : 16237-18039) | |
| 6 | 536-540 | ATLQA linker 1-5 | | |
| 6 | 541-565 | AAGLEAPFLYLYVLVYFLQSINFVR | ORF3a_protein@Norway(NC_04551@ | 98-122 |
| | | | Norway: + : 25393-26220) | |
| 6 | 566-570 | ATLQA linker 1-5 | | |
| 6 | 571-585 | RTCCLCDRRATCFST | 3′-to-5′_exonuclease(NC_045512: + : | 205-219 |
| | | | 18040-19620) | |
| 6 | 586-590 | ATLQA linker 1-5 | | |
| 6 | 591-605 | ERFVSLAIDAYPLTK | RNA-dependent_RNA_polymerase | 857-871 |
| | | | (NC_04551: + : | |
| | | | 13442-13468 + 13468-16236) | |
| 6 | 606-610 | ATLQA linker 1-5 | | |
| 6 | 611-618 | VLSEARQH | leader_protein(NC_045512: + : | 38-45 |
| | | | 266-805) | |
| 6 | 619-623 | ATLQA linker 1-5 | | |
| 6 | 624-637 | SVASQSIIAYTMSL | surface_glycoprotein@Austria | 678-691 |
| | | | (NC_045512@Austria: + : | |
| | | | 21563-25384) | |
| 6 | 638-642 | ATLQA linker 1-5 | | |
| 6 | 643-660 | KGFCDLKGKYVQIPTTCA | nsp10(NC_045512: + : 13025-13441) | 87-104 |
| 6 | 661-665 | ATLQA linker 1-5 | | |
| 6 | 666-675 | GVITHDVSSA | helicase(NC_045512: + : | 478-487 |
| | | | 16237-18039) | |
| 6 | 676-680 | ATLQA linker 1-5 | | |
| 6 | 681-702 | IAMACLVGLMWLSYFIASFRLF | membrane_glycoprotein@Aruba | 82-103 |
| | | | (NC_045512@Aruba: + : | |
| | | | 26523-27191) | |
| 6 | 703-707 | ATLQA linker 1-5 | | |
| 6 | 708-717 | TPLIQPIGAL | nsp4(NC_045512: + : 8555-10054) | 269-278 |
| 6 | 718-722 | ATLQA linker 1-5 | | |
| 6 | 723-736 | IHFYSKWYIRVGAK | ORF8_protein@Mozambique(NC_04551@ | 39-52 |
| | | | Mozambique: + : 27894-28259) | |
| 6 | 737-741 | ATLQA linker 1-5 | | |
| 6 | 742-751 | LPRVFSAVGN | nsp4(NC_045512: + : 8555-10054) | 122-131 |
| 6 | 752-756 | ATLQA linker 1-5 | | |
| 6 | 757-774 | SQHTMLVKQGDDYVYLPY | RNA-dependent_RNA_polymerase@Aruba | 814-831 |
| | | | (NC_04551@Aruba: + : | |
| | | | 13442-13468 + 13468-16236) | |
| 6 | 775-779 | ATLQA linker 1-5 | | |
| 6 | 780-805 | YRLANECAQVLSEIVMCGGSLYVKP | RNA-dependent_RNA_polymerase@ | 653-678 |
| | | G | Canada(NC_04551@Canada: + : | |
| | | | 13442-13468 + 13468-16236) | |
| 6 | 806-810 | ATLQA linker 1-5 | | |
| 6 | 811-828 | CANFNVLFSTVFPPTSFG | RNA-dependent_RNA_polymerase@ | 310-327 |
| | | | China(NC_04551@China: + : | |
| | | | 13442-13468 + 13468-16236) | |
| 6 | 829-833 | ATLQA linker 1-5 | | |
| 6 | 834-845 | DTSLKLKECVMY | nsp6@SmallAsia(NC_045512@ | 102-113 |
| | | | SmallAsia: + : 10973-11842) | |
| 6 | 846-850 | ATLQA linker 1-5 | | |
| 6 | 851-866 | FRELGVVHNQDVNLHS | RNA-dependent_RNA_polymerase | 348-363 |
| | | | (NC_04551: + : | |
| | | | 13442-13468 + 13468-16236) | |
| 6 | 867-871 | ATLQA linker 1-5 | | |
| 6 | 872-880 | MPVCVETKA | nsp3(NC_045512: + : 2720-8554) | 571-579 |
| 6 | 881-885 | ATLQA linker 1-5 | | |
| 6 | 886-901 | SEFSSLPSYAAFATAQ | nsp8(NC_045512: + : 12092-12685) | 4-19 |
| 6 | 902-906 | ATLQA linker 1-5 | | |
| 6 | 907-922 | EEIIYNLLKDCPAVAK | RNA-dependent_RNA_polymerase@ | 83-98 |
| | | | Senegal(NC_04551@Senegal: + : | |
| | | | 13442-13468 + 13468-16236) | |
| 6 | 923-927 | ATLQA linker 1-5 | | |
| 6 | 928-940 | VKGLQPSVGPKQA | endoRNAse(NC_04512: + : | 148-160 |
| | | | 19621-20658) | |
| 6 | 941-645 | ATLQA linker 1-5 | | |
| 6 | 946-961 | TVLKKCKSAFYILPSI | nsp3(NC_045512: + : 2720-8554) | 626-541 |
| 6 | 962-966 | ATLQA linker 1-5 | | |
| 6 | 967-980 | DGCVPLNIIPLTTA | nsp8(NC_045512: + : 12092-12685) | 112-125 |
| 6 | 981-985 | ATLQA linker 1-5 | | |
| 6 | 986-1003 | FLYENAFPFAMGVIAMS | nsp6@Mexico(NC_045512@ | 36-53 |
| | | | Mexico: + : 10973-11842) | |
| 6 | 1004-1008 | ATLQA linker 1-5 | | |
| 6 | 1009-1016 | TQALPHRQ | nucleocapsid_phosphoprotein@ | 379-386 |
| | | | Mozambique(NC_04551@Mozambique: + : | |
| | | | 28274-29533) | |
| 6 | 1017-1021 | ATLQA linker 1-5 | | |
| 6 | 1022-1040 | DKSAFVNLKQLPFFYYSDS | 3′-to-5′_exonuclease(NC_045512: + : | 432-450 |
| | | | 18040-19620) | |
| 6 | 1041-1045 | ATLQA linker 1-5 | | |
| 6 | 1046-1061 | LQSLQTYVTQQLIRAA | surface_glycoprotein(NC_04551: + : | 1001-1016 |
| | | | 21563-25384) | |
| 6 | 1062-1066 | ATLQA linker 1-5 | | |
| 6 | 1067-1086 | FPQSAPHGVVFLHVTYVPAQ | surface_glycoprotein@Aruba | 1048-1067 |
| | | | (NC_045512@Aruba: + : | |
| | | | 21563-25384) | |
| 6 | 1087-1091 | ATLQA linker 1-5 | | |
| 6 | 1092-1101 | ISDYDYYRYN | RNA-dependent_RNA_polymerase@ | 450-459 |
| | | | Aruba(NC_04551@Aruba: + : | |
| | | | 13442-13468 + 13468-16236) | |
| 6 | 1102-1106 | ATLQA linker 1-5 | | |
| 6 | 1107-1114 | SLIDLQEL | surface_glycoprotein(NC_04551: + : | 1196-1203 |
| | | | 21563-25384) | |
| 6 | 1115-1119 | ATLQA linker 1-5 | | |
| 6 | 1120-1130 | SELVIGAVILR | membrane_glycoprotein(NC_045512: + | 136-146 |
| | | | : 26523-27191) | |
| 6 | 1131-1135 | ATLQA linker 1-5 | | |
| 6 | 1136-1154 | VKDCVLLHSYFTSDYYQLY | ORF3a_protein@Belgium(NC_04551@ | 197-215 |
| | | | Belgium: + : 25393-26220) | |
| 6 | 1155-1159 | ATLQA linker 1-5 | | |
| 6 | 1160-1168 | TEIYQAGST | surface_glycoprotein@Aruba | 466-474 |
| | | | (NC_045512@Aruba: + : | |
| | | | 21563-25384) | |
| 6 | 1169-1173 | ATLQA linker 1-5 | | |
| 6 | 1174-1188 | HYVRITGLYPTLNIL | helicase@Indonesia(NC_045512@ | 245-259 |
| | | | Indonesia: + : 16237-18039) | |
| 6 | 1189-1193 | ATLQA linker 1-5 | | |
| 6 | 1194-1207 | FFSNVTWFHAIHVS | surface_glycoprotein@Bangladesh | 58-71 |
| | | | (NC_045512@Bangladesh: + : | |
| | | | 21563-25384) | |
| 6 | 1208-1212 | ATLQA linker 1-5 | | |
| 6 | 1213-1220 | GSCGSVGF | 3C-like_proteinase(NC_045512: + : | 143-150 |
| | | | 10055-10972) | |
| 6 | 1221-1225 | ATLQA linker 1-5 | | |
| 6 | 1226-1234 | KHWPQIAQF | nucleocapsid_phosphoprotein | 299-307 |
| | | | (NC_045512: + : 28274-29533) | |
| 6 | 1235-1239 | ATLQA linker 1-5 | | |
| 6 | 1240-1254 | AYYVGYLQPRTFLLK | surface_glycoprotein@Aruba | 260-274 |
| | | | (NC_045512@Aruba: + : | |
| | | | 21563-25384) | |
| 6 | 1255-1259 | ATLQA linker 1-5 | | |
| 6 | 1260-1268 | YGIIWVATE | nucleocapsid_phosphoprotein@ | 128-136 |
| | | | SmallSouthAmerica(NC_045512@ | |
| | | | SmallSouthAmerica: + : | |
| | | | 28274-29533) | |
| 6 | 1269-1273 | ATLQA linker 1-5 | | |
| 6 | 1274-1281 | EHVQIHTI | ORF3a_protein(NC_04551: + : | 242-249 |
| | | | 25393-26220) | |
| 6 | 1282-1286 | ATLQA linker 1-5 | | |
| 6 | 1287-1301 | EFQFCNDPFLGVYYH | surface_glycoprotein@Chile | 132-146 |
| | | | (NC_045512@Chile: + : | |
| | | | 21563-25384) | |
| 6 | 1302-1306 | ATLQA linker 1-5 | | |
| 6 | 1307-1320 | LVLLPLVSSQCVNF | surface_glycoprotein@Brazil | 5-18 |
| | | | (NC_045512@Brazil: + : | |
| | | | 21563-25384) | |
| 6 | 1321-1325 | ATLQA linker 1-5 | | |
| 6 | 1326-1335 | KQYGDCLGDI | surface_glycoprotein(NC_04551: + : | 835-844 |
| | | | 21563-25384) | |

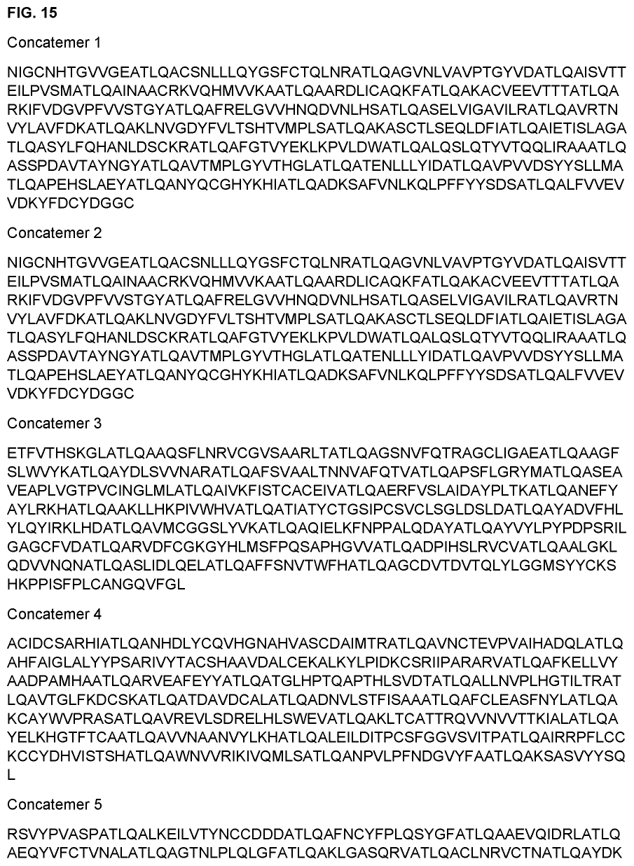

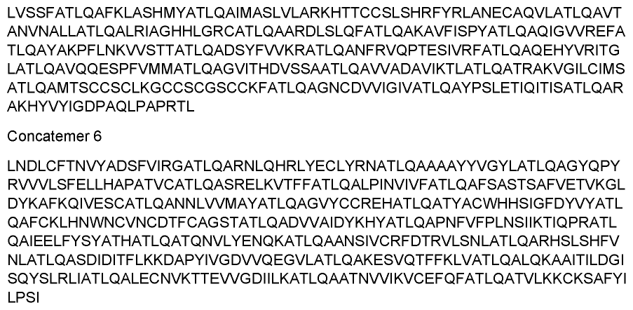

In certain embodiments, the vaccine comprises or expresses at least one of the concatemers of epitopes from SARS-COV-2 with intervening linker sequences (and as set forth in FIG. 15). In certain embodiments, the vaccine comprises or expresses at least two of the concatemers set forth below. In certain embodiments, the vaccine comprises or expresses three of the concatemers set forth below. In certain embodiments, the vaccine comprises or expresses four of the concatemers set forth below. In certain embodiments, the vaccine comprises or expresses five of the concatemers set forth below. In certain embodiments, the vaccine comprises or expresses all the concatemers set forth below.

TABLE 5

| | pos_in_ | | | pos_in_ |
|---|---|---|---|---|
| concatemers | concatemer | epitope | gene | gene |
| 1 | 1-12 | NIGCNHTGVVGE | nsp2(NC_045512:+:806-2719) | 250-261 |
| 1 | 13-17 | ATLQA | linker | 1-5 |
| 1 | 18-34 | CSNLLLQYGSFCTQLNR | surface_glycoprotein(NC_045512:+: | 749-765 |
| | | | 21563-25384) | |
| 1 | 35-39 | ATLQA | linker | 1-5 |
| 1 | 40-52 | GVNLVAVPTGYVD | 3′-to-5′_exonuclease(NC_045512:+: | 114-126 |
| | | | 18040-19620) | |
| 1 | 53-57 | ATLQA | linker | 1-5 |
| 1 | 58-69 | ISVTTEILPVSM | surface_glycoprotein(NC_045512:+: | 720-731 |
| | | | 21563-25384) | |
| 1 | 70-74 | ATLQA | linker | 1-5 |
| 1 | 75-89 | INAACRKVQHMVVKA | 3′-to-5′_exonuclease(NC_045512:+: | 305-319 |
| | | | 18040-19620) | |
| 1 | 90-94 | ATLQA | linker | 1-5 |
| 1 | 95-104 | ARDLICAQKF | surface_glycoprotein(NC_045512:+: | 846-855 |
| | | | 21563-25384) | |
| 1 | 105-109 | ATLQA | linker | 1-5 |
| 1 | 110-119 | KACVEEVTTT | nsp3(NC_045512:+:2720-8554) | 415-424 |
| 1 | 120-124 | ATLQA | linker | 1-5 |
| 1 | 125-140 | RKIFVDGVPFVVSTGY | RNA-dependent_RNA_polymerase(NC_045512:+: | 331-346 |
| | | | 13442-13468+13468-16236) | |
| 1 | 141-145 | ATLQA | linker | 1-5 |
| 1 | 146-161 | FRELGVVHNQDVNLHS | RNA-dependent_RNA_polymerase(NC_045512:+: | 348-363 |
| | | | 13442-13468+13468-16236) | |
| 1 | 162-166 | ATLQA | linker | 1-5 |
| 1 | 167-177 | SELVIGAVILR | membrane_glycoprotein(NC_045512:+: | 136-146 |
| | | | 26523-27191) | |
| 1 | 178-182 | ATLQA | linker | 1-5 |
| 1 | 183-194 | VRTNVYLAVFDK | nsp3(NC_045512:+:2720-8554) | 351-362 |
| 1 | 195-199 | ATLQA | linker | 1-5 |
| 1 | 200-218 | KLNVGDYFVLTSHTVMPLS | helicase(NC_045512:+:16237-18039) | 218-236 |
| 1 | 219-223 | ATLQA | linker | 1-5 |
| 1 | 224-236 | KASCTLSEQLDFI | nsp2(NC_045512:+:806-2719) | 30-42 |
| 1 | 237-241 | ATLQA | linker | 1-5 |
| 1 | 242-249 | IETISLAG | nsp3(NC_045512:+:2720-8554) | 684-691 |
| 1 | 250-254 | ATLQA | linker | 1-5 |
| 1 | 255-268 | SYLFQHANLDSCKR | nsp3(NC_045512:+:2720-8554) | 915-928 |
| 1 | 269-273 | ATLQA | linker | 1-5 |
| 1 | 274-287 | FGTVYEKLKPVLDW | nsp2(NC_045512:+:806-2719) | 437-450 |
| 1 | 288-292 | ATLQA | linker | 1-5 |
| 1 | 293-308 | LQSLQTYVTQQLIRAA | surface_glycoprotein(NC_045512:+: | 1001-1016 |
| | | | 21563-25384) | |
| 1 | 309-313 | ATLQA | linker | 1-5 |
| 1 | 314-325 | SSPDAVTAYNGY | nsp3(NC_045512:+:2720-8554) | 660-671 |
| 1 | 326-330 | ATLQA | linker | 1-5 |
| 1 | 331-342 | VTMPLGYVTHGL | nsp3(NC_045512:+:2720-8554) | 628-639 |
| 1 | 343-347 | ATLQA | linker | 1-5 |
| 1 | 348-356 | TENLLLYID | nsp3(NC_045512:+:2720-8554) | 432-440 |
| 1 | 357-361 | ATLQA | linker | 1-5 |
| 1 | 362-373 | VPVVDSYYSLLM | RNA-dependent_RNA_polymerase(NC_045512:+: | 231-242 |
| | | | 13442-13468+13468-16236) | |
| 1 | 374-378 | ATLQA | linker | 1-5 |
| 1 | 379-386 | PEHSLAEY | nsp2(NC_045512:+:806-2719) | 200-207 |
| 1 | 387-391 | ATLQA | linker | 1-5 |
| 1 | 392-401 | NYQCGHYKHI | nsp3(NC_045512:+:2720-8554) | 1012-1021 |
| 1 | 402-406 | ATLQA | linker | 1-5 |
| 1 | 407-425 | DKSAFVNLKQLPFFYYSDS | 3′-to-5′_exonuclease(NC_045512:+: | 432-450 |
| | | | 18040-19620) | |
| 1 | 426-430 | ATLQA | linker | 1-5 |
| 1 | 431-448 | LFVVEVVDKYFDCYDGGC | RNA-dependent_RNA_polymerase(NC_045512:+: | 470-487 |
| | | | 13442-13468+13468-16236) | |
| 2 | 1-13 | AFASEAARVVRSI | nsp2(NC_045512:+:806-2719) | 355-367 |
| 2 | 14-18 | ATLQA | linker | 1-5 |
| 2 | 19-32 | SDRVVFVLWAHGFE | 3′-to-5′_exonuclease(NC_045512:+: | 178-191 |
| | | | 18040-19620) | |
| 2 | 33-37 | ATLQA | linker | 1-5 |
| 2 | 38-45 | MADLVYAL | RNA-dependent_RNA_polymerase(NC_045512:+: | 124-131 |
| | | | 13442-13468+13468-16236) | |
| 2 | 46-50 | ATLQA | linker | 1-5 |
| 2 | 51-60 | KQYGDCLGDI | surface_glycoprotein(NC_045512:+: | 835-844 |
| | | | 21563-25384) | |
| 2 | 61-65 | ATLQA | linker | 1-5 |
| 2 | 66-73 | FTGCVIAW | surface_glycoprotein(NC_045512:+: | 429-436 |
| | | | 21563-25384) | |
| 2 | 74-78 | ATLQA | linker | 1-5 |
| 2 | 79-86 | YDPLQPEL | surface_glycoprotein(NC_045512:+: | 1138-1145 |
| | | | 21563-25384) | |
| 2 | 87-91 | ATLQA | linker | 1-5 |
| 2 | 92-111 | DDRCILHCANFNVLFSTVFP | RNA-dependent_RNA_polymerase(NC_045512:+: | 303-322 |
| | | | 13442-13468+13468-16236) | |
| 2 | 112-116 | ATLQA | linker | 1-5 |
| 2 | 117-136 | GYLPQNAVVKIYCPACHNSE | nsp2(NC_045512:+:806-2719) | 178-197 |
| 2 | 137-141 | ATLQA | linker | 1-5 |
| 2 | 142-153 | HEFCSQHTMLVK | RNA-dependent_RNA_polymerase(NC_045512:+: | 810-821 |
| | | | 13442-13468+13468-16236) | |
| 2 | 154-158 | ATLQA | linker | 1-5 |
| 2 | 159-172 | EGVVDYGARFYFYT | nsp3(NC_045512:+:2720-8554) | 595-608 |
| 2 | 173-177 | ATLQA | linker | 1-5 |
| 2 | 178-189 | IEVQGYKSVNIT | nsp3(NC_045512:+:2720-8554) | 13-24 |
| 2 | 190-194 | ATLQA | linker | 1-5 |
| 2 | 195-207 | AIKCVPQADVEWK | 3′-to-5′_exonuclease(NC_045512:+: | 337-349 |
| | | | 18040-19620) | |
| 2 | 208-212 | ATLQA | linker | 1-5 |
| 2 | 213-235 | SHGKQVVSDIDYVPLKSATCITR | 3′-to-5′_exonuclease(NC_045512:+: | 454-476 |
| | | | 18040-19620) | |
| 2 | 236-240 | ATLQA | linker | 1-5 |
| 2 | 241-253 | ANFCALILAYCNK | nsp3(NC_045512:+:2720-8554) | 890-902 |
| 2 | 254-258 | ATLQA | linker | 1-5 |
| 2 | 259-268 | SVELKHFFFA | RNA-dependent_RNA_polymerase(NC_045512:+: | 434-443 |
| | | | 13442-13468+13468-16236) | |
| 2 | 269-273 | ATLQA | linker | 1-5 |
| 2 | 274-284 | KGVQIPCTCGK | nsp3(NC_045512:+:2720-8554) | 963-973 |
| 2 | 285-289 | ATLQA | linker | 1-5 |
| 2 | 290-307 | IKDLPKEITVATSRTLSY | membrane_glycoprotein(NC_045512:+: | 161-178 |
| | | | 26523-27191) | |
| 2 | 308-312 | ATLQA | linker | 1-5 |
| 2 | 313-327 | SYELQTPFEIKLAKK | nsp2(NC_045512:+:806-2719) | 68-82 |
| 2 | 328-332 | ATLQA | linker | 1-5 |
| 2 | 333-346 | TKDVVECLKLSHQS | nsp3(NC_045512:+:2720-8554) | 1830-1843 |
| 2 | 347-351 | ATLQA | linker | 1-5 |
| 2 | 352-359 | ISNCVADY | surface_glycoprotein(NC_045512:+: | 358-365 |
| | | | 21563-25384) | |
| 2 | 360-364 | ATLQA | linker | 1-5 |
| 2 | 365-379 | RTCCLCDRRATCFST | 3′-to-5′_exonuclease(NC_045512:+: | 205-219 |
| | | | 18040-19620) | |
| 2 | 380-384 | ATLQA | linker | 1-5 |
| 2 | 385-393 | EVFAQVKQI | surface_glycoprotein(NC_045512:+: | 780-788 |
| | | | 21563-25384) | |
| 2 | 394-398 | ATLQA | linker | 1-5 |
| 2 | 399-409 | AVHECFVKRVD | 3′-to-5′_exonuclease(NC_045512:+: | 281-291 |
| | | | 18040-19620) | |
| 2 | 410-414 | ATLQA | linker | 1-5 |
| 2 | 415-423 | MPVCVETKA | nsp3(NC_045512:+:2720-8554) | 571-579 |
| 2 | 424-428 | ATLQA | linker | 1-5 |
| 2 | 429-436 | SVDCTMYI | surface_glycoprotein(NC_045512:+: | 735-742 |
| | | | 21563-25384) | |
| 2 | 437-441 | ATLQA | linker | 1-5 |
| 2 | 442-449 | AALQIPFA | surface_glycoprotein(NC_045512:+: | 892-899 |
| | | | 21563-25384) | |
| 3 | 1-10 | ETFVTHSKGL | nsp2(NC_045512:+:806-2719) | 527-536 |
| 3 | 11-15 | ATLQA | linker | 1-5 |
| 3 | 16-32 | AQSFLNRVCGVSAARLT | RNA-dependent_RNA_polymerase(NC_045512:+: | 4-20 |
| | | | 13442-13468+13468-16236) | |
| 3 | 33-37 | ATLQA | linker | 1-5 |
| 3 | 38-53 | GSNVFQTRAGCLIGAE | surface_glycoprotein(NC_045512:+: | 639-654 |
| | | | 21563-25384) | |
| 3 | 54-58 | ATLQA | linker | 1-5 |
| 3 | 59-67 | AGFSLWVYK | 3′-to-5′_exonuclease(NC_045512:+: | 504-512 |
| | | | 18040-19620) | |
| 3 | 68-72 | ATLQA | linker | 1-5 |
| 3 | 73-81 | YDLSVVNAR | helicase(NC_045512:+:16237-18039) | 382-390 |
| 3 | 82-86 | ATLQA | linker | 1-5 |
| 3 | 87-101 | FSVAALTNNVAFQTV | RNA-dependent_RNA_polymerase(NC_045512:+: | 396-410 |
| | | | 13442-13468+13468-16236) | |
| 3 | 102-106 | ATLQA | linker | 1-5 |
| 3 | 107-114 | PSFLGRYM | nsp3(NC_045512:+:2720-8554) | 822-829 |
| 3 | 115-119 | ATLQA | linker | 1-5 |
| 3 | 120-139 | SEAVEAPLVGTPVCINGLML | nsp2(NC_045512:+:806-2719) | 591-610 |
| 3 | 140-144 | ATLQA | linker | 1-5 |
| 3 | 145-157 | IVKFISTCACEIV | nsp2(NC_045512:+:806-2719) | 468-480 |
| 3 | 158-162 | ATLQA | linker | 1-5 |
| 3 | 163-177 | ERFVSLAIDAYPLTK | RNA-dependent_RNA_polymerase(NC_045512:+: | 857-871 |
| | | | 13442-13468+13468-16236) | |
| 3 | 178-182 | ATLQA | linker | 1-5 |
| 3 | 183-192 | NEFYAYLRKH | RNA-dependent_RNA_polymerase(NC_045512:+: | 743-752 |
| | | | 13442-13468+13468-16236) | |
| 3 | 193-197 | ATLQA | linker | 1-5 |
| 3 | 198-209 | AKLLHKPIVWHV | nsp3(NC_045512:+:2720-8554) | 1165-1176 |
| 3 | 210-214 | ATLQA | linker | 1-5 |
| 3 | 215-237 | TIATYCTGSIPCSVCLSGLDSLD | nsp3(NC_045512:+:2720-8554) | 1459-1481 |
| 3 | 238-242 | ATLQA | linker | 1-5 |
| 3 | 243-259 | YADVFHLYLQYIRKLHD | RNA-dependent_RNA_polymerase(NC_045512:+: | 877-893 |
| | | | 13442-13468+13468-16236) | |
| 3 | 260-264 | ATLQA | linker | 1-5 |
| 3 | 265-274 | VMCGGSLYVK | RNA-dependent_RNA_polymerase(NC_045512:+: | 667-676 |
| | | | 13442-13468+13468-16236) | |
| 3 | 275-279 | ATLQA | linker | 1-5 |
| 3 | 280-294 | QIELKFNPPALQDAY | nsp3(NC_045512:+:2720-8554) | 867-881 |
| 3 | 295-299 | ATLQA | linker | 1-5 |

| | | | | |
|---|---|---|---|---|
| 3 | 300-319 | YVYLPYPDPSRILGAGCFVD | RNA-dependent_RNA_polymerase(NC_045512:+: | 826-845 |
| | | | 13442-13468+13468-16236) | |
| 3 | 320-324 | ATLQA | linker | 1-5 |
| 3 | 325-347 | RVDFCGKGYHLMSFPQSAPHGVV | surface_glycoprotein(NC_045512:+: | 1039-1061 |
| | | | 21563-25384) | |
| 3 | 348-352 | ATLQA | linker | 1-5 |
| 3 | 353-362 | DPIHSLRVCV | nsp3(NC_045512:+:2720-8554) | 339-348 |
| 3 | 363-367 | ATLQA | linker | 1-5 |
| 3 | 368-379 | ALGKLQDVVNQN | surface_glycoprotein(NC_045512:+: | 944-955 |
| | | | 21563-25384) | |
| 3 | 380-384 | ATLQA | linker | 1-5 |
| 3 | 385-392 | SLIDLQEL | surface_glycoprotein(NC_045512:+: | 1196-1203 |
| | | | 21563-25384) | |
| 3 | 393-397 | ATLQA | linker | 1-5 |
| 3 | 398-406 | FFSNVTWFH | surface_glycoprotein(NC_045512:+: | 58-66 |
| | | | 21563-25384) | |
| 3 | 407-411 | ATLQA | linker | 1-5 |
| 3 | 412-450 | GCDVTDVTQLYLGGMSYYCKSHK | helicase(NC_045512:+:16237-18039) | 54-92 |
| | | PPISFPLCANGQVFGL | | |
| 4 | 1-10 | ACIDCSARHI | nsp3(NC_045512:+:2720-8554) | 1872-1881 |
| 4 | 11-15 | ATLQA | linker | 1-5 |
| 4 | 16-38 | NHDLYCQVHGNAHVASCDAIMTR | 3′-to-5′_exonuclease(NC_045512:+: | 256-278 |
| | | | 18040-19620) | |
| 4 | 39-43 | ATLQA | linker | 1-5 |
| 4 | 44-58 | VNCTEVPVAIHADQL | surface_glycoprotein(NC_045512:+: | 615-629 |
| | | | 21563-25384) | |
| 4 | 59-63 | ATLQA | linker | 1-5 |
| 4 | 64-114 | HFAIGLALYYPSARIVYTACSHA | helicase(NC_045512:+:16237-18039) | 290-340 |
| | | AVDALCEKALKYLPIDKCSRIIP | | |
| | | ARARV | | |
| 4 | 115-119 | ATLQA | linker | 1-5 |
| 4 | 120-134 | FKELLVYAADPAMHA | RNA-dependent_RNA_polymerase(NC_045512:+: | 368-382 |
| | | | 13442-13468+13468-16236) | |
| 4 | 135-139 | ATLQA | linker | 1-5 |
| 4 | 140-147 | RVEAFEYY | nsp3(NC_045512:+:2720-8554) | 810-817 |
| 4 | 148-152 | ATLQA | linker | 1-5 |
| 4 | 153-168 | TGLHPTQAPTHLSVDT | 3′-to-5′_exonuclease(NC_045512:+: | 16-31 |
| | | | 18040-19620) | |
| 4 | 169-173 | ATLQA | linker | 1-5 |
| 4 | 174-186 | LLNVPLHGTILTR | membrane_glycoprotein(NC_045512:+: | 119-131 |
| | | | 26523-27191) | |
| 4 | 187-191 | ATLQA | linker | 1-5 |
| 4 | 192-201 | VTGLFKDCSK | 3′-to-5′_exonuclease(NC_045512:+: | 4-13 |
| | | | 18040-19620) | |
| 4 | 202-206 | ATLQA | linker | 1-5 |
| 4 | 207-214 | TDAVDCAL | surface_glycoprotein(NC_045512:+: | 286-293 |
| | | | 21563-25384) | |
| 4 | 215-219 | ATLQA | linker | 1-5 |
| 4 | 220-230 | DNVLSTFISAA | nsp3(NC_045512:+:2720-8554) | 1809-1819 |
| 4 | 231-235 | ATLQA | linker | 1-5 |
| 4 | 236-245 | FCLEASFNYL | nsp3(NC_045512:+:2720-8554) | 1391-1400 |
| 4 | 246-250 | ATLQA | linker | 1-5 |
| 4 | 251-260 | KCAYWVPRAS | nsp2(NC_045512:+:806-2719) | 239-248 |
| 4 | 261-265 | ATLQA | linker | 1-5 |
| 4 | 266-281 | VREVLSDRELHLSWEV | helicase(NC_045512:+:16237-18039) | 154-169 |
| 4 | 282-286 | ATLQA | linker | 1-5 |
| 4 | 287-306 | KLTCATTRQVVNVVTTKIAL | nsp3(NC_045512:+:2720-8554) | 1923-1942 |
| 4 | 307-311 | ATLQA | linker | 1-5 |
| 4 | 312-322 | YELKHGTFTCA | nsp3(NC_045512:+:2720-8554) | 996-1006 |
| 4 | 323-327 | ATLQA | linker | 1-5 |
| 4 | 328-338 | VVNAANVYLKH | nsp3(NC_045512:+:2720-8554) | 239-249 |
| 4 | 339-343 | ATLQA | linker | 1-5 |
| 4 | 344-362 | LEILDITPCSFGGVSVITP | surface_glycoprotein(NC_045512:+: | 582-600 |
| | | | 21563-25384) | |
| 4 | 363-367 | ATLQA | linker | 1-5 |
| 4 | 368-387 | IRRPFLCCKCCYDHVISTSH | helicase(NC_045512:+:16237-18039) | 20-39 |
| 4 | 388-392 | ATLQA | linker | 1-5 |
| 4 | 393-405 | WNVVRIKIVQMLS | 3′-to-5′_exonuclease(NC_045512:+: | 159-171 |
| | | | 18040-19620) | |
| 4 | 406-410 | ATLQA | linker | 1-5 |
| 4 | 411-423 | NPVLPFNDGVYFA | surface_glycoprotein(NC_045512:+: | 81-93 |
| | | | 21563-25384) | |
| 4 | 424-428 | ATLQA | linker | 1-5 |
| 4 | 429-438 | KSASVYYSQL | nsp3(NC_045512:+:2720-8554) | 1737-1746 |
| 5 | 1-9 | RSVYPVASP | nsp2(NC_045512:+:806-2719) | 121-129 |
| 5 | 10-14 | ATLQA | linker | 1-5 |
| 5 | 15-28 | LKEILVTYNCCDDD | RNA-dependent_RNA_polymerase(NC_045512:+: | 142-155 |
| | | | 13442-13468+13468-16236) | |
| 5 | 29-33 | ATLQA | linker | 1-5 |
| 5 | 34-45 | FNCYFPLQSYGF | surface_glycoprotein(NC_045512:+: | 486-497 |
| | | | 21563-25384) | |
| 5 | 46-50 | ATLQA | linker | 1-5 |
| 5 | 51-58 | AEVQIDRL | surface_glycoprotein(NC_045512:+: | 989-996 |
| | | | 21563-25384) | |
| 5 | 59-63 | ATLQA | linker | 1-5 |
| 5 | 64-74 | EQYVFCTVNAL | helicase(NC_045512:+:16237-18039) | 353-363 |
| 5 | 75-79 | ATLQA | linker | 1-5 |
| 5 | 80-89 | GTNLPLQLGF | 3′-to-5′_exonuclease(NC_045512:+: | 102-111 |
| | | | 18040-19620) | |
| 5 | 90-94 | ATLQA | linker | 1-5 |
| 5 | 95-102 | KLGASQRV | membrane_glycoprotein(NC_045512:+: | 180-187 |
| | | | 26523-27191) | |
| 5 | 103-107 | ATLQA | linker | 1-5 |
| 5 | 108-115 | CLNRVCTN | nsp3(NC_045512:+:2720-8554) | 1342-1349 |
| 5 | 116-120 | ATLQA | linker | 1-5 |
| 5 | 121-128 | YDKLVSSF | nsp3(NC_045512:+:2720-8554) | 365-372 |
| 5 | 129-133 | ATLQA | linker | 1-5 |
| 5 | 134-141 | FKLASHMY | nsp3(NC_045512:+:2720-8554) | 96-103 |
| 5 | 142-146 | ATLQA | linker | 1-5 |
| 5 | 147-178 | IMASLVLARKHTTCCSLSHRFYR | RNA-dependent_RNA_polymerase(NC_045512:+: | 632-663 |
| | | LANECAQVL | 13442-13468+13468-16236) | |
| 5 | 179-183 | ATLQA | linker | 1-5 |
| 5 | 184-192 | VTANVNALL | RNA-dependent_RNA_polymerase(NC_045512:+: | 700-708 |
| | | | 13442-13468+13468-16236) | |
| 5 | 193-197 | ATLQA | linker | 1-5 |
| 5 | 198-208 | LRIAGHHLGRC | membrane_glycoprotein(NC_045512:+: | 149-159 |
| | | | 26523-27191) | |
| 5 | 209-213 | ATLQA | linker | 1-5 |
| 5 | 214-221 | ARDLSLQF | nsp3(NC_045512:+:2720-8554) | 1652-1659 |
| 5 | 222-226 | ATLQA | linker | 1-5 |
| 5 | 227-234 | KAVFISPY | helicase(NC_045512:+:16237-18039) | 508-515 |
| 5 | 235-239 | ATLQA | linker | 1-5 |
| 5 | 240-247 | QIGVVREF | helicase(NC_045512:+:16237-18039) | 492-499 |
| 5 | 248-252 | ATLQA | linker | 1-5 |
| 5 | 253-265 | YAKPFLNKVVSTT | nsp3(NC_045512:+:2720-8554) | 1323-1335 |
| 5 | 266-270 | ATLQA | linker | 1-5 |
| 5 | 271-278 | DSYFVVKR | RNA-dependent_RNA_polymerase(NC_045512:+: | 67-74 |
| | | | 13442-13468+13468-16236) | |
| 5 | 279-283 | ATLQA | linker | 1-5 |
| 5 | 284-296 | NFRVQPTESIVRF | surface_glycoprotein(NC_045512:+: | 317-329 |
| | | | 21563-25384) | |
| 5 | 297-301 | ATLQA | linker | 1-5 |
| 5 | 302-311 | QEHYVRITGL | helicase(NC_045512:+:16237-18039) | 243-252 |
| 5 | 312-316 | ATLQA | linker | 1-5 |
| 5 | 317-326 | VQQESPFVMM | nsp3(NC_045512:+:2720-8554) | 980-989 |
| 5 | 327-331 | ATLQA | linker | 1-5 |
| 5 | 332-341 | GVITHDVSSA | helicase(NC_045512:+:16237-18039) | 478-487 |
| 5 | 342-346 | ATLQA | linker | 1-5 |
| 5 | 347-356 | VVADAVIKTL | nsp3(NC_045512:+:2720-8554) | 56-65 |
| 5 | 357-361 | ATLQA | linker | 1-5 |
| 5 | 362-373 | TRAKVGILCIMS | helicase(NC_045512:+:16237-18039) | 566-577 |
| 5 | 374-378 | ATLQA | linker | 1-5 |
| 5 | 379-398 | MTSCCSCLKGCCSCGSCCKF | surface_glycoprotein(NC_045512:+: | 1237-1256 |
| | | | 21563-25384) | |
| 5 | 399-403 | ATLQA | linker | 1-5 |
| 5 | 404-413 | GNCDVVIGIV | surface_glycoprotein(NC_045512:+: | 1124-1133 |
| | | | 21563-25384) | |
| 5 | 414-418 | ATLQA | linker | 1-5 |
| 5 | 419-430 | YPSLETIQITIS | nsp3(NC_045512:+:2720-8554) | 1483-1494 |
| 5 | 431-435 | ATLQA | linker | 1-5 |
| 5 | 436-455 | RAKHYVYIGDPAQLPAPRTL | helicase(NC_045512:+:16237-18039) | 392-411 |
| 6 | 1-18 | LNDLCFTNVYADSFVIRG | surface_glycoprotein(NC_045512:+: | 387-404 |
| | | | 21563-25384) | |
| 6 | 19-23 | ATLQA | linker | 1-5 |
| 6 | 24-37 | RNLQHRLYECLYRN | RNA-dependent_RNA_polymerase(NC_045512:+: | 721-734 |
| | | | 13442-13468+13468-16236) | |
| 6 | 38-42 | ATLQA | linker | 1-5 |
| 6 | 43-51 | AAAYYVGYL | surface_glycoprotein(NC_045512:+: | 262-270 |
| | | | 21563-25384) | |
| 6 | 52-56 | ATLQA | linker | 1-5 |
| 6 | 57-78 | GYQPYRVVVLSFELLHAPATVC | surface_glycoprotein(NC_045512:+: | 504-525 |
| | | | 21563-25384) | |
| 6 | 79-83 | ATLQA | linker | 1-5 |
| 6 | 84-92 | SRELKVTFF | nsp3(NC_045512:+:2720-8554) | 1134-1142 |
| 6 | 93-97 | ATLQA | linker | 1-5 |
| 6 | 98-105 | LPINVIVF | nsp3(NC_045512:+:2720-8554) | 1718-1725 |
| 6 | 106-110 | ATLQA | linker | 1-5 |
| 6 | 111-137 | FSASTSAFVETVKGLDYKAFKQI | nsp2(NC_045512:+:806-2719) | 300-326 |
| | | VESC | | |
| 6 | 138-142 | ATLQA | linker | 1-5 |
| 6 | 143-150 | NNLVVMAY | nsp2(NC_045512:+:806-2719) | 413-420 |
| 6 | 151-155 | ATLQA | linker | 1-5 |
| 6 | 156-163 | GVYCCREH | nsp2(NC_045512:+:806-2719) | 47-54 |
| 6 | 164-168 | ATLQA | linker | 1-5 |
| 6 | 169-183 | TYACWHHSIGFDYVY | 3′-to-5′_exonuclease(NC_045512:+: | 223-237 |
| | | | 18040-19620) | |
| 6 | 184-188 | ATLQA | linker | 1-5 |
| 6 | 189-208 | FCKLHNWNCVNCDTFCAGST | nsp3(NC_045512:+:2720-8554) | 1626-1645 |
| 6 | 209-213 | ATLQA | linker | 1-5 |
| 6 | 214-223 | DVVAIDYKHY | nsp3(NC_045512:+:2720-8554) | 1148-1157 |
| 6 | 224-228 | ATLQA | linker | 1-5 |
| 6 | 229-245 | PNFVFPLNSIIKTIQPR | nsp2(NC_045512:+:806-2719) | 91-107 |
| 6 | 246-250 | ATLQA | linker | 1-5 |
| 6 | 251-261 | IEELFYSYATH | 3′-to-5′_exonuclease(NC_045512:+: | 363-373 |
| | | | 18040-19620) | |
| 6 | 262-266 | ATLQA | linker | 1-5 |
| 6 | 267-276 | TQNVLYENQK | surface_glycoprotein(NC_045512:+: | 912-921 |
| | | | 21563-25384) | |
| 6 | 277-281 | ATLQA | linker | 1-5 |
| 6 | 282-297 | ANSIVCRFDTRVLSNL | 3′-to-5′_exonuclease(NC_045512:+: | 394-409 |
| | | | 18040-19620) | |
| 6 | 298-302 | ATLQA | linker | 1-5 |
| 6 | 303-312 | RHSLSHFVNL | nsp3(NC_045512:+:2720-8554) | 1697-1706 |
| 6 | 313-317 | ATLQA | linker | 1-5 |
| 6 | 318-342 | SDIDITFLKKDAPYIVGDVVQEG | nsp3(NC_045512:+:2720-8554) | 454-478 |
| | | VL | | |
| 6 | 343-347 | ATLQA | linker | 1-5 |
| 6 | 348-358 | KESVQTFFKLV | nsp2(NC_045512:+:806-2719) | 492-502 |
| 6 | 359-363 | ATLQA | linker | 1-5 |
| 6 | 364-383 | LQKAAITILDGISQYSLRLI | nsp2(NC_045512:+:806-2719) | 382-401 |
| 6 | 384-388 | ATLQA | linker | 1-5 |
| 6 | 389-405 | LECNVKTTEVVGDIILK | nsp3(NC_045512:+:2720-8554) | 1244-1260 |
| 6 | 406-410 | ATLQA | linker | 1-5 |
| 6 | 411-423 | ATNVVIKVCEFQF | surface_glycoprotein(NC_045512:+: | 123-135 |
| | | | 21563-25384) | |
| 6 | 424-428 | ATLQA | linker | 1-5 |
| 6 | 429-444 | TVLKKCKSAFYILPSI | nsp3(NC_045512:+:2720-8554) | 526-541 |

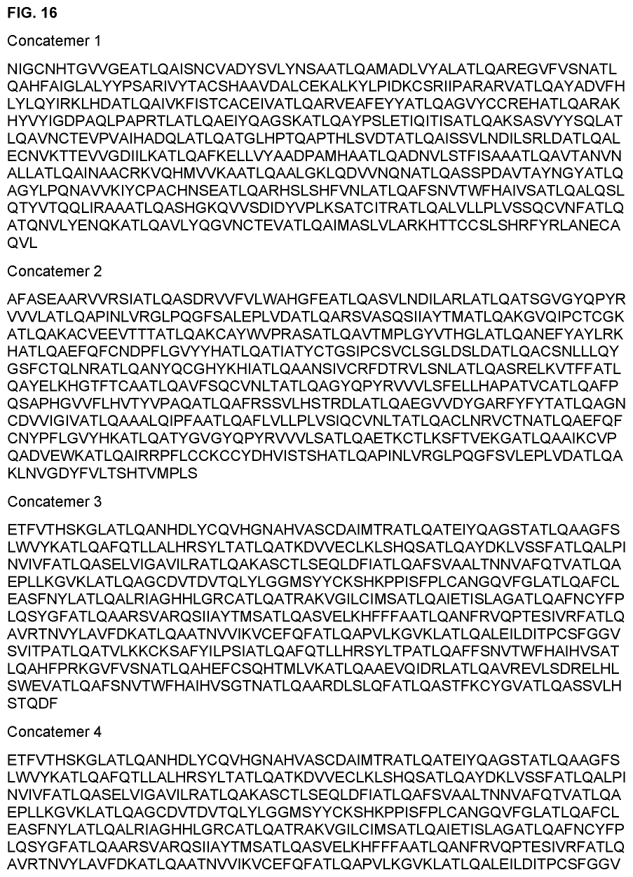

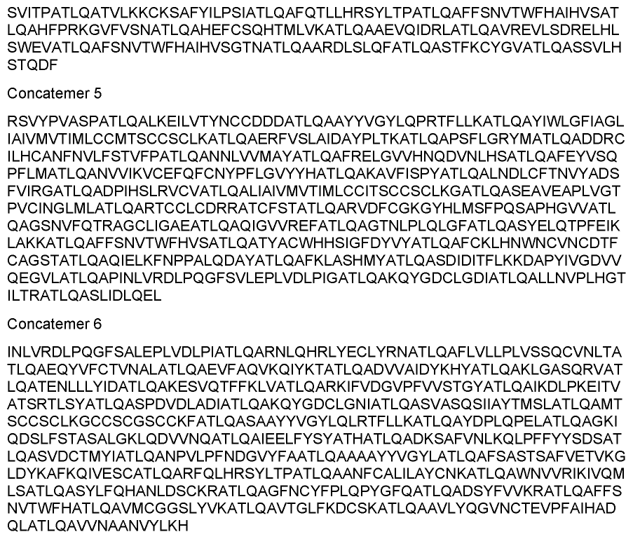

In certain embodiments, the vaccine comprises or expresses at least one of the concatemers of epitopes from SARS-COV-2 with intervening linker sequences (and as set forth in FIG. 16). In certain embodiments, the vaccine comprises or expresses at least two of the concatemers set forth below. In certain embodiments, the vaccine comprises or expresses three of the concatemers set forth below. In certain embodiments, the vaccine comprises or expresses four of the concatemers set forth below. In certain embodiments, the vaccine comprises or expresses five of the concatemers set forth below. In certain embodiments, the vaccine comprises or expresses all the concatemers set forth below.

TABLE 6

| | pos_in_ | | | pos_ |
|---|---|---|---|---|
| concatemers | concatemer | epitope | gene | in_gene |
| 1 | 1-12 | NIGCNHTGVVGE | nsp2(NC_045512:+:806-2719) | 250-261 |
| 1 | 13-17 | ATLQA | linker | 1-5 |
| 1 | 18-32 | ISNCVADYSVLYNSA | surface_glycoprotein@Aruba(NC_045512@Aruba:+: | 354-368 |
| | | | 21563-25384) | |
| 1 | 33-37 | ATLQA | linker | 1-5 |
| 1 | 38-45 | MADLVYAL | RNA-dependent_RNA_polymerase(NC_045512:+: | 124-131 |
| | | | 13442-13468+13468-16236) | |
| 1 | 46-50 | ATLQA | linker | 1-5 |
| 1 | 51-58 | REGVFVSN | surface_glycoprotein@Aruba(NC_045512@Aruba:+: | 1087-1094 |
| | | | 21563-25384) | |
| 1 | 59-63 | ATLQA | linker | 1-5 |
| 1 | 64-114 | HFAIGLALYYPSARIV | helicase(NC_045512:+:16237-18039) | 290-340 |
| | | YTACSHAAVDALCEKA | | |
| | | LKYLPIDKCSRIIPAR | | |
| | | ARV | | |
| 1 | 115-119 | ATLQA | linker | 1-5 |
| 1 | 120-136 | YADVFHLYLQYIRKLH | RNA-dependent_RNA_polymerase(NC_045512:+: | 877-893 |
| | | D | 13442-13468+13468-16236) | |
| 1 | 137-141 | ATLQA | linker | 1-5 |
| 1 | 142-154 | IVKFISTCACEIV | nsp2(NC_045512:+:806-2719) | 468-480 |
| 1 | 155-159 | ATLQA | linker | 1-5 |
| 1 | 160-167 | RVEAFEYY | nsp3(NC_045512:+:2720-8554) | 810-817 |
| 1 | 168-172 | ATLQA | linker | 1-5 |
| 1 | 173-180 | GVYCCREH | nsp2(NC_045512:+:806-2719) | 47-54 |
| 1 | 181-185 | ATLQA | linker | 1-5 |
| 1 | 186-205 | RAKHYVYIGDPAQLPA | helicase(NC_045512:+:16237-18039) | 392-411 |
| | | PRTL | | |
| 1 | 206-210 | ATLQA | linker | 1-5 |
| 1 | 211-218 | EIYQAGSK | surface_glycoprotein@Mexico(NC_045512@Mexico:+: | 471-478 |
| | | | 21563-25384) | |
| 1 | 219-223 | ATLQA | linker | 1-5 |
| 1 | 224-235 | YPSLETIQITIS | nsp3(NC_045512:+:2720-8554) | 1483-1494 |
| 1 | 236-240 | ATLQA | linker | 1-5 |
| 1 | 241-250 | KSASVYYSQL | nsp3(NC_045512:+:2720-8554) | 1737-1746 |
| 1 | 251-255 | ATLQA | linker | 1-5 |
| 1 | 256-270 | VNCTEVPVAIHADQL | surface_glycoprotein(NC_045512:+:21563-25384) | 615-629 |
| 1 | 271-275 | ATLQA | linker | 1-5 |
| 1 | 276-291 | TGLHPTQAPTHLSVDT | 3′-to-5′_exonuclease(NC_045512:+:18040-19620) | 16-31 |
| 1 | 292-296 | ATLQA | linker | 1-5 |
| 1 | 297-309 | ISSVLNDILSRLD | surface_glycoprotein@Bangladesh | 972-984 |
| | | | (NC_045512@Bangladesh:+:21563-25384) | |
| 1 | 310-314 | ATLQA | linker | 1-5 |
| 1 | 315-331 | LECNVKTTEVVGDIIL | nsp3(NC_045512:+:2720-8554) | 1244-1260 |
| | | K | | |
| 1 | 332-336 | ATLQA | linker | 1-5 |
| 1 | 337-351 | FKELLVYAADPAMHA | RNA-dependent_RNA_polymerase(NC_045512:+: | 368-382 |
| | | | 13442-13468+13468-16236) | |
| 1 | 352-356 | ATLQA | linker | 1-5 |
| 1 | 357-367 | DNVLSTFISAA | nsp3(NC_045512:+:2720-8554) | 1809-1819 |
| 1 | 368-372 | ATLQA | linker | 1-5 |
| 1 | 373-381 | VTANVNALL | RNA-dependent_RNA_polymerase(NC_045512:+: | 700-708 |
| | | | 13442-13468+13468-16236) | |
| 1 | 382-386 | ATLQA | linker | 1-5 |
| 1 | 387-401 | INAACRKVQHMVVKA | 3′-to-5′_exonuclease(NC_045512:+:18040-19620) | 305-319 |
| 1 | 402-406 | ATLQA | linker | 1-5 |
| 1 | 407-418 | ALGKLQDVVNQN | surface_glycoprotein(NC_045512:+:21563-25384) | 944-955 |
| 1 | 419-423 | ATLQA | linker | 1-5 |
| 1 | 424-435 | SSPDAVTAYNGY | nsp3(NC_045512:+:2720-8554) | 660-671 |
| 1 | 436-440 | ATLQA | linker | 1-5 |
| 1 | 441-460 | GYLPQNAVVKIYCPAC | nsp2(NC_045512:+:806-2719) | 178-197 |
| | | HNSE | | |
| 1 | 461-465 | ATLQA | linker | 1-5 |
| 1 | 466-475 | RHSLSHFVNL | nsp3(NC_045512:+:2720-8554) | 1697-1706 |
| 1 | 476-480 | ATLQA | linker | 1-5 |
| 1 | 481-492 | FSNVTWFHAIVS | surface_glycoprotein@Ghana(NC_045512@Ghana:+: | 59-70 |
| | | | 21563-25384) | |
| 1 | 493-497 | ATLQA | linker | 1-5 |
| 1 | 498-513 | LQSLQTYVTQQLIRAA | surface_glycoprotein(NC_045512:+:21563-25384) | 1001-1016 |
| 1 | 514-518 | ATLQA | linker | 1-5 |
| 1 | 519-541 | SHGKQVVSDIDYVPLK | 3′-to-5′_exonuclease(NC_045512:+:18040-19620) | 454-476 |
| | | SATCITR | | |
| 1 | 542-546 | ATLQA | linker | 1-5 |
| 1 | 547-560 | LVLLPLVSSQCVNF | surface_glycoprotein@Brazil(NC_045512@Brazil:+: | 5-18 |
| | | | 21563-25384) | |
| 1 | 561-565 | ATLQA | linker | 1-5 |
| 1 | 566-575 | TQNVLYENQK | surface_glycoprotein(NC_045512:+:21563-25384) | 912-921 |
| 1 | 576-580 | ATLQA | linker | 1-5 |
| 1 | 581-591 | VLYQGVNCTEV | surface_glycoprotein@Aruba(NC_045512@Aruba:+: | 606-616 |
| | | | 21563-25384) | |
| 1 | 592-596 | ATLQA | linker | 1-5 |
| 1 | 597-628 | IMASLVLARKHTTCCS | RNA-dependent_RNA_polymerase(NC_045512:+: | 632-663 |
| | | LSHRFYRLANECAQVL | 13442-13468+13468-16236) | |
| 2 | 1-13 | AFASEAARVVRSI | nsp2(NC_045512:+:806-2719) | 355-367 |
| 2 | 14-18 | ATLQA | linker | 1-5 |
| 2 | 19-32 | SDRVVFVLWAHGFE | 3′-to-5′_exonuclease(NC_045512:+:18040-19620) | 178-191 |
| 2 | 33-37 | ATLQA | linker | 1-5 |
| 2 | 38-47 | SVLNDILARL | surface_glycoprotein@Aruba(NC_045512@Aruba:+: | 971-980 |
| | | | 21563-25384) | |
| 2 | 48-52 | ATLQA | linker | 1-5 |
| 2 | 53-66 | TSGVGYQPYRVVVL | surface_glycoprotein@Aruba(NC_045512@Aruba:+: | 496-509 |
| | | | 21563-25384) | |
| 2 | 67-71 | ATLQA | linker | 1-5 |
| 2 | 72-91 | PINLVRGLPQGFSALE | surface_glycoprotein@Austria | 205-224 |
| | | PLVD | (NC_045512@Austria:+:21563-25384) | |
| 2 | 92-96 | ATLQA | linker | 1-5 |
| 2 | 97-109 | RSVASQSIIAYTM | surface_glycoprotein@Aruba(NC_045512@Aruba:+: | 681-693 |
| | | | 21563-25384) | |
| 2 | 110-114 | ATLQA | linker | 1-5 |
| 2 | 115-125 | KGVQIPCTCGK | nsp3(NC_045512:+:2720-8554) | 963-973 |
| 2 | 126-130 | ATLQA | linker | 1-5 |
| 2 | 131-140 | KACVEEVTTT | nsp3(NC_045512:+:2720-8554) | 415-424 |
| 2 | 141-145 | ATLQA | linker | 1-5 |
| 2 | 146-155 | KCAYWVPRAS | nsp2(NC_045512:+:806-2719) | 239-248 |
| 2 | 156-160 | ATLQA | linker | 1-5 |
| 2 | 161-172 | VTMPLGYVTHGL | nsp3(NC_045512:+:2720-8554) | 628-639 |
| 2 | 173-177 | ATLQA | linker | 1-5 |
| 2 | 178-187 | NEFYAYLRKH | RNA-dependent_RNA_polymerase(NC_045512:+: | 743-752 |
| | | | 13442-13468+13468-16236) | |
| 2 | 188-192 | ATLQA | linker | 1-5 |
| 2 | 193-207 | EFQFCNDPFLGVYYH | surface_glycoprotein@Chile(NC_045512@Chile:+: | 132-146 |
| | | | 21563-25384) | |
| 2 | 208-212 | ATLQA | linker | 1-5 |
| 2 | 213-235 | TIATYCTGSIPCSVCL | nsp3(NC_045512:+:2720-8554) | 1459-1481 |
| | | SGLDSLD | | |
| 2 | 236-240 | ATLQA | linker | 1-5 |
| 2 | 241-257 | CSNLLLQYGSFCTQLN | surface_glycoprotein(NC_045512:+:21563-25384) | 749-765 |
| | | R | | |
| 2 | 258-262 | ATLQA | linker | 1-5 |
| 2 | 263-272 | NYQCGHYKHI | nsp3(NC_045512:+:2720-8554) | 1012-1021 |
| 2 | 273-277 | ATLQA | linker | 1-5 |
| 2 | 278-293 | ANSIVCRFDTRVLSNL | 3′-to-5′_exonuclease(NC_045512:+:18040-19620) | 394-409 |
| 2 | 294-298 | ATLQA | linker | 1-5 |
| 2 | 299-307 | SRELKVTFF | nsp3(NC_045512:+:2720-8554) | 1134-1142 |
| 2 | 308-312 | ATLQA | linker | 1-5 |
| 2 | 313-323 | YELKHGTFTCA | nsp3(NC_045512:+:2720-8554) | 996-1006 |
| 2 | 324-328 | ATLQA | linker | 1-5 |
| 2 | 329-337 | VFSQCVNLT | surface_glycoprotein@Egypt(NC_045512@Egypt:+: | 11-19 |
| | | | 21563-25384) | |
| 2 | 338-342 | ATLQA | linker | 1-5 |
| 2 | 343-364 | GYQPYRVVVLSFELLH | surface_glycoprotein(NC_045512:+:21563-25384) | 504-525 |
| | | APATVC | | |
| 2 | 365-369 | ATLQA | linker | 1-5 |
| 2 | 370-389 | FPQSAPHGVVFLHVTY | surface_glycoprotein@Aruba(NC_045512@Aruba:+: | 1048-1067 |
| | | VPAQ | 21563-25384) | |
| 2 | 390-394 | ATLQA | linker | 1-5 |
| 2 | 395-406 | FRSSVLHSTRDL | surface_glycoprotein@Nigeria | 43-54 |
| | | | (NC_045512@Nigeria:+:21563-25384) | |
| 2 | 407-411 | ATLQA | linker | 1-5 |
| 2 | 412-425 | EGVVDYGARFYFYT | nsp3(NC_045512:+:2720-8554) | 595-608 |
| 2 | 426-430 | ATLQA | linker | 1-5 |
| 2 | 431-440 | GNCDVVIGIV | surface_glycoprotein(NC_045512:+:21563-25384) | 1124-1133 |
| 2 | 441-445 | ATLQA | linker | 1-5 |
| 2 | 446-453 | AALQIPFA | surface_glycoprotein(NC_045512:+:21563-25384) | 892-899 |
| 2 | 454-458 | ATLQA | linker | 1-5 |
| 2 | 459-474 | FLVLLPLVSIQCVNLT | surface_glycoprotein@SmallNorthAmerica | 4-19 |
| | | | (NC_045512@SmallNorthAmerica:+:21563-25384) | |
| 2 | 475-479 | ATLQA | linker | 1-5 |
| 2 | 480-487 | CLNRVCTN | nsp3(NC_045512:+:2720-8554) | 1342-1349 |
| 2 | 488-492 | ATLQA | linker | 1-5 |
| 2 | 493-507 | EFQFCNYPFLGVYHK | surface_glycoprotein@Norway(NC_045512@Norway:+: | 129-143 |
| | | | 21563-25384) | |
| 2 | 508-512 | ATLQA | linker | 1-5 |
| 2 | 513-527 | TYGVGYQPYRVVVLS | surface_glycoprotein@Australia | 496-510 |
| | | | (NC_045512@Australia:+:21563-25384) | |
| 2 | 528-532 | ATLQA | linker | 1-5 |
| 2 | 533-546 | ETKCTLKSFTVEKG | surface_glycoprotein@Aruba(NC_045512@Aruba:+: | 294-307 |
| | | | 21563-25384) | |
| 2 | 547-551 | ATLQA | linker | 1-5 |
| 2 | 552-564 | AIKCVPQADVEWK | 3′-to-5′_exonuclease(NC_045512:+:18040-19620) | 337-349 |
| 2 | 565-569 | ATLQA | linker | 1-5 |
| 2 | 570-589 | IRRPFLCCKCCYDHVI | helicase(NC_045512:+:16237-18039) | 20-39 |
| | | STSH | | |
| 2 | 590-594 | ATLQA | linker | 1-5 |
| 2 | 595-614 | PINLVRGLPQGFSVLE | surface_glycoprotein@SmallAfrica | 209-228 |
| | | PLVD | (NC_045512@SmallAfrica:+:21563-25384) | |
| 2 | 615-619 | ATLQA | linker | 1-5 |
| 2 | 620-638 | KLNVGDYFVLTSHTVM | helicase(NC_045512:+:16237-18039) | 218-236 |
| | | PLS | | |
| 3 | 1-10 | ETFVTHSKGL | nsp2(NC_045512:+:806-2719) | 527-536 |
| 3 | 11-15 | ATLQA | linker | 1-5 |
| 3 | 16-38 | NHDLYCQVHGNAHVAS | 3′-to-5′_exonuclease(NC_045512:+:18040-19620) | 256-278 |
| | | CDAIMTR | | |
| 3 | 39-43 | ATLQA | linker | 1-5 |
| 3 | 44-52 | TEIYQAGST | surface_glycoprotein@Aruba(NC_045512@Aruba:+: | 466-474 |
| | | | 21563-25384) | |
| 3 | 53-57 | ATLQA | linker | 1-5 |
| 3 | 58-66 | AGFSLWVYK | 3′-to-5′_exonuclease(NC_045512:+:18040-19620) | 504-512 |
| 3 | 67-71 | ATLQA | linker | 1-5 |
| 3 | 72-84 | FQTLLALHRSYLT | surface_glycoprotein@Aruba(NC_045512@Aruba:+: | 234-246 |
| | | | 21563-25384) | |
| 3 | 85-89 | ATLQA | linker | 1-5 |
| 3 | 90-103 | TKDVVECLKLSHQS | nsp3(NC_045512:+:2720-8554) | 1830-1843 |
| 3 | 104-108 | ATLQA | linker | 1-5 |
| | 109-116 | YDKLVSSF | nsp3(NC_045512:+:2720-8554) | 365-372 |
| 3 | 117-121 | ATLQA | linker | 1-5 |
| 3 | 122-129 | LPINVIVF | nsp3(NC_045512:+:2720-8554) | 1718-1725 |
| | 130-134 | ATLQA | linker | 1-5 |
| 3 | 135-145 | SELVIGAVILR | membrane_glycoprotein(NC_045512:+:26523-27191) | 136-146 |
| 3 | 146-150 | ATLQA | linker | 1-5 |
| | 151-163 | KASCTLSEQLDFI | nsp2(NC_045512:+:806-2719) | 30-42 |
| 3 | 164-168 | ATLQA | linker | 1-5 |
| 3 | 169-183 | FSVAALTNNVAFQTV | RNA-dependent_RNA_polymerase(NC_045512:+: | 396-410 |
| | | | 13442-13468+13468-16236) | |
| 3 | 184-188 | ATLQA | linker | 1-5 |
| 3 | 189-197 | EPLLKGVKL | surface_glycoprotein@Croatia | 1258-1266 |
| | | | (NC_045512@Croatia:+:21563-25384) | |
| 3 | 198-202 | ATLQA | linker | 1-5 |
| 3 | 203-241 | GCDVTDVTQLYLGGMS | helicase(NC_045512:+:16237-18039) | 54-92 |
| | | YYCKSHKPPISFPLCA | | |
| | | NGQVFGL | | |
| 3 | 242-246 | ATLQA | linker | 1-5 |
| 3 | 247-256 | FCLEASFNYL | nsp3(NC_045512:+:2720-8554) | 1391-1400 |
| 3 | 257-261 | ATLQA | linker | 1-5 |
| 3 | 262-272 | LRIAGHHLGRC | membrane_glycoprotein(NC_045512:+:26523-27191) | 149-159 |
| 3 | 273-277 | ATLQA | linker | 1-5 |
| 3 | 278-289 | TRAKVGILCIMS | helicase(NC_045512:+:16237-18039) | 566-577 |
| 3 | 290-294 | ATLQA | linker | 1-5 |
| 3 | 295-302 | IETISLAG | nsp3(NC_045512:+:2720-8554) | 684-691 |
| 3 | 303-307 | ATLQA | linker | 1-5 |
| 3 | 308-319 | FNCYFPLQSYGF | surface_glycoprotein(NC_045512:+:21563-25384) | 486-497 |
| 3 | 320-324 | ATLQA | linker | 1-5 |
| 3 | 325-339 | ARSVARQSIIAYTMS | surface_glycoprotein@Mozambique | 680-694 |
| | | | (NC_045512@Mozambique:+:21563-25384) | |
| 3 | 340-344 | ATLQA | linker | 1-5 |
| 3 | 345-354 | SVELKHFFFA | RNA-dependent_RNA_polymerase(NC_045512:+: | 434-443 |
| | | | 13442-13468+13468-16236) | |
| 3 | 355-359 | ATLQA | linker | 1-5 |
| 3 | 360-372 | NFRVQPTESIVRF | surface_glycoprotein(NC_045512:+:21563-25384) | 317-329 |
| 3 | 373-377 | ATLQA | linker | 1-5 |
| 3 | 378-389 | VRTNVYLAVFDK | nsp3(NC_045512:+:2720-8554) | 351-362 |
| 3 | 390-394 | ATLQA | linker | 1-5 |
| 3 | 395-407 | ATNVVIKVCEFQF | surface_glycoprotein(NC_045512:+:21563-25384) | 123-135 |
| 3 | 408-412 | ATLQA | linker | 1-5 |
| 3 | 413-420 | PVLKGVKL | surface_glycoprotein@Aruba(NC_045512@Aruba:+: | 1259-1266 |
| | | | 21563-25384) | |
| 3 | 421-425 | ATLQA | linker | 1-5 |
| 3 | 426-444 | LEILDITPCSFGGVSV | surface_glycoprotein(NC_045512:+:21563-25384) | 582-600 |
| | | ITP | | |
| 3 | 445-449 | ATLQA | linker | 1-5 |
| 3 | 450-465 | TVLKKCKSAFYILPSI | nsp3(NC_045512:+:2720-8554) | 526-541 |
| 3 | 466-470 | ATLQA | linker | 1-5 |
| 3 | 471-482 | FQTLLHRSYLTP | surface_glycoprotein@SmallAsia | 230-241 |
| | | | (NC_045512@SmallAsia:+:21563-25384) | |
| 3 | 483-487 | ATLQA | linker | 1-5 |
| 3 | 488-501 | FFSNVTWFHAIHVS | surface_glycoprotein@Bangladesh | 58-71 |
| | | | (NC_045512@Bangladesh:+:21563-25384) | |
| 3 | 502-506 | ATLQA | linker | 1-5 |
| 3 | 507-517 | HFPRKGVFVSN | surface_glycoprotein@SmallAsia | 1078-1088 |
| | | | (NC_045512@SmallAsia:+:21563-25384) | |
| 3 | 518-522 | ATLQA | linker | 1-5 |
| 3 | 523-534 | HEFCSQHTMLVK | RNA-dependent_RNA_polymerase(NC_045512:+: | 810-821 |
| | | | 13442-13468+13468-16236) | |
| 3 | 535-539 | ATLQA | linker | 1-5 |
| 3 | 540-547 | AEVQIDRL | surface_glycoprotein(NC_045512:+:21563-25384) | 989-996 |
| 3 | 548-552 | ATLQA | linker | 1-5 |
| 3 | 553-568 | VREVLSDRELHLSWEV | helicase(NC_045512:+:16237-18039) | 154-169 |
| 3 | 569-573 | ATLQA | linker | 1-5 |
| 3 | 574-589 | FSNVTWFHAIHVSGTN | surface_glycoprotein@Peru(NC_045512@Peru:+: | 59-74 |
| | | | 21563-25384) | |
| 3 | 590-594 | ATLQA | linker | 1-5 |
| 3 | 595-602 | ARDLSLQF | nsp3(NC_045512:+:2720-8554) | 1652-1659 |
| 3 | 603-607 | ATLQA | linker | 1-5 |
| 3 | 608-615 | STFKCYGV | surface_glycoprotein@Aruba(NC_045512@Aruba:+: | 371-378 |
| | | | 21563-25384) | |
| 3 | 616-620 | ATLQA | linker | 1-5 |
| 3 | 621-630 | SSVLHSTQDF | surface_glycoprotein@Norway(NC_045512@Norway:+: | 45-54 |
| | | | 21563-25384) | |
| 4 | 1-10 | ACIDCSARHI | nsp3(NC_045512:+:2720-8554) | 1872-1881 |
| 4 | 11-15 | ATLQA | linker | 1-5 |
| 4 | 16-32 | AQSFLNRVCGVSAARL | RNA-dependent_RNA_polymerase(NC_045512:+: | 4-20 |
| | | T | 13442-13468+13468-16236) | |
| 4 | 33-37 | ATLQA | linker | 1-5 |
| 4 | 38-50 | GVNLVAVPTGYVD | 3′-to-5′_exonuclease(NC_045512:+:18040-19620) | 114-126 |
| 4 | 51-55 | ATLQA | linker | 1-5 |
| 4 | 56-67 | FRSSVLHSTQDL | surface_glycoprotein@Aruba(NC_045512@Aruba:+: | 43-54 |
| | | | 21563-25384) | |
| 4 | 68-72 | ATLQA | linker | 1-5 |
| 4 | 73-86 | ANPVLPFNDGVYFA | surface_glycoprotein@Austria | 77-90 |
| | | | (NC_045512@Austria:+:21563-25384) | |
| 4 | 87-91 | ATLQA | linker | 1-5 |
| 4 | 92-111 | YVYLPYPDPSRILGAG | RNA-dependent_RNA_polymerase(NC_045512:+: | 826-845 |
| | | CFVD | 13442-13468+13468-16236) | |
| 4 | 112-116 | ATLQA | linker | 1-5 |
| 4 | 117-136 | LQKAAITILDGISQYS | nsp2(NC_045512:+:806-2719) | 382-401 |
| | | LRLI | | |
| 4 | 137-141 | ATLQA | linker | 1-5 |
| 4 | 142-153 | VPVVDSYYSLLM | RNA-dependent_RNA_polymerase(NC_045512:+: | 231-242 |
| | | | 13442-13468+13468-16236) | |
| 4 | 154-158 | ATLQA | linker | 1-5 |
| 4 | 159-176 | LFVVEVVDKYFDCYDG | RNA-dependent_RNA_polymerase(NC_045512:+: | 470-487 |
| | | GC | 13442-13468+13468-16236) | |
| 4 | 177-181 | ATLQA | linker | 1-5 |
| 4 | 182-192 | AVHECFVKRVD | 3′-to-5′_exonuclease(NC_045512:+:18040-19620) | 281-291 |
| 4 | 193-197 | ATLQA | linker | 1-5 |
| 4 | 198-207 | GVITHDVSSA | helicase(NC_045512:+:16237-18039) | 478-487 |
| 4 | 208-212 | ATLQA | linker | 1-5 |
| 4 | 213-224 | IEVQGYKSVNIT | nsp3(NC_045512:+:2720-8554) | 13-24 |
| 4 | 225-229 | ATLQA | linker | 1-5 |
| 4 | 230-243 | IKVCEFQFCNDPYY | surface_glycoprotein@CostaRica | 128-141 |
| | | | (NC_045512@CostaRica:+:21563-25384) | |
| 4 | 244-248 | ATLQA | linker | 1-5 |
| 4 | 249-258 | VQQESPFVMM | nsp3(NC_045512:+:2720-8554) | 980-989 |
| 4 | 259-263 | ATLQA | linker | 1-5 |
| 4 | 264-278 | TISVTTEILPVSMAK | surface_glycoprotein@Mexico(NC_045512@Mexico:+: | 719-733 |
| | | | 21563-25384) | |
| 4 | 279-283 | ATLQA | linker | 1-5 |
| 4 | 284-295 | AKLLHKPIVWHV | nsp3(NC_045512:+:2720-8554) | 1165-1176 |
| 4 | 296-300 | ATLQA | linker | 1-5 |
| 4 | 301-317 | PNFVFPLNSIIKTIQP | nsp2(NC_045512:+:806-2719) | 91-107 |
| | | R | | |
| 4 | 318-322 | ATLQA | linker | 1-5 |
| 4 | 323-331 | MPVCVETKA | nsp3(NC_045512:+:2720-8554) | 571-579 |
| 4 | 332-336 | ATLQA | linker | 1-5 |
| 4 | 337-344 | PEHSLAEY | nsp2(NC_045512:+:806-2719) | 200-207 |
| 4 | 345-349 | ATLQA | linker | 1-5 |
| 4 | 350-369 | KLTCATTRQVVNVVTT | nsp3(NC_045512:+:2720-8554) | 1923-1942 |
| | | KIAL | | |
| 4 | 370-374 | ATLQA | linker | 1-5 |
| 4 | 375-384 | ARDLICAQKF | surface_glycoprotein(NC_045512:+:21563-25384) | 846-855 |
| 4 | 385-389 | ATLQA | linker | 1-5 |
| 4 | 390-408 | AIPINFTISVTTEILP | surface_glycoprotein@Aruba(NC_045512@Aruba:+: | 709-727 |
| | | VSM | 21563-25384) | |
| 4 | 409-413 | ATLQA | linker | 1-5 |
| 4 | 414-422 | YDLSVVNAR | helicase(NC_045512:+:16237-18039) | 382-390 |
| 4 | 423-427 | ATLQA | linker | 1-5 |
| 4 | 428-441 | GFNCYSPLQSYGFQ | surface_glycoprotein@Peru(NC_045512@Peru:+: | 485-498 |
| | | | 21563-25384) | |
| 4 | 442-446 | ATLQA | linker | 1-5 |
| 4 | 447-456 | AGAALQIPFA | surface_glycoprotein@Nigeria | 886-895 |
| | | | (NC_045512@Nigeria:+:21563-25384) | |
| 4 | 457-461 | ATLQA | linker | 1-5 |
| 4 | 462-480 | QVAVLYQDVNCTEVPV | surface_glycoprotein@China(NC_045512@China:+: | 607-625 |
| | | AIH | 21563-25384) | |
| 4 | 481-485 | ATLQA | linker | 1-5 |
| 4 | 486-495 | QEHYVRITGL | helicase(NC_045512:+:16237-18039) | 243-252 |
| 4 | 496-500 | ATLQA | linker | 1-5 |
| 4 | 501-514 | FGTVYEKLKPVLDW | nsp2(NC_045512:+:806-2719) | 437-450 |
| 4 | 515-519 | ATLQA | linker | 1-5 |
| 4 | 520-529 | VVADAVIKTL | nsp3(NC_045512:+:2720-8554) | 56-65 |
| 4 | 530-534 | ATLQA | linker | 1-5 |
| 4 | 535-542 | ISNCVADY | surface_glycoprotein(NC_045512:+:21563-25384) | 358-365 |
| 4 | 543-547 | ATLQA | linker | 1-5 |
| 4 | 548-556 | EVFAQVKQI | surface_glycoprotein(NC_045512:+:21563-25384) | 780-788 |
| 4 | 557-561 | ATLQA | linker | 1-5 |
| 4 | 562-574 | YAKPFLNKVVSTT | nsp3(NC_045512:+:2720-8554) | 1323-1335 |
| 4 | 575-579 | ATLQA | linker | 1-5 |
| 4 | 580-587 | TDAVDCAL | surface_glycoprotein(NC_045512:+:21563-25384) | 286-293 |
| 4 | 588-592 | ATLQA | linker | 1-5 |
| 4 | 593-604 | ISVTTEILPVSM | surface_glycoprotein(NC_045512:+:21563-25384) | 720-731 |
| 4 | 605-609 | ATLQA | linker | 1-5 |
| 4 | 610-617 | FTGCVIAW | surface_glycoprotein(NC_045512:+:21563-25384) | 429-436 |
| 4 | 618-622 | ATLQA | linker | 1-5 |
| 4 | 623-640 | IKVCEFQFCNDPFLGV | surface_glycoprotein@Aruba(NC_045512@Aruba:+: | 125-142 |
| | | YH | 21563-25384) | |
| 5 | 1-9 | RSVYPVASP | nsp2(NC_045512:+:806-2719) | 121-129 |
| 5 | 10-14 | ATLQA | linker | 1-5 |
| 5 | 15-28 | LKEILVTYNCCDDD | RNA-dependent_RNA_polymerase(NC_045512:+: | 142-155 |
| | | | 13442-13468+13468-16236) | |
| 5 | 29-33 | ATLQA | linker | 1-5 |
| 5 | 34-48 | AYYVGYLQPRTFLLK | surface_glycoprotein@Aruba(NC_045512@Aruba:+: | 260-274 |
| | | | 21563-25384) | |
| 5 | 49-53 | ATLQA | linker | 1-5 |
| 5 | 54-84 | YIWLGFIAGLIAIVMV | surface_glycoprotein@Aruba(NC_045512@Aruba:+: | 1211-1241 |
| | | TIMLCCMTSCCSCLK | 21563-25384) | |
| 5 | 85-89 | ATLQA | linker | 1-5 |
| 5 | 90-104 | ERFVSLAIDAYPLTK | RNA-dependent_RNA_polymerase(NC_045512:+: | 857-871 |
| | | | 13442-13468+13468-16236) | |
| 5 | 105-109 | ATLQA | linker | 1-5 |
| 5 | 110-117 | PSFLGRYM | nsp3(NC_045512:+:2720-8554) | 822-829 |
| 5 | 118-122 | ATLQA | linker | 1-5 |
| 5 | 123-142 | DDRCILHCANFNVLFS | RNA-dependent_RNA_polymerase(NC_045512:+: | 303-322 |
| | | TVFP | 13442-13468+13468-16236) | |
| 5 | 143-147 | ATLQA | linker | 1-5 |
| 5 | 148-155 | NNLVVMAY | nsp2(NC_045512:+:806-2719) | 413-420 |
| 5 | 156-160 | ATLQA | linker | 1-5 |
| 5 | 161-176 | FRELGVVHNQDVNLHS | RNA-dependent_RNA_polymerase(NC_045512:+: | 348-363 |
| | | | 13442-13468+13468-16236) | |
| 5 | 177-181 | ATLQA | linker | 1-5 |
| 5 | 182-191 | FEYVSQPFLM | surface_glycoprotein@Aruba(NC_045512@Aruba:+: | 164-173 |
| | | | 21563-25384) | |
| 5 | 192-196 | ATLQA | linker | 1-5 |
| 5 | 197-218 | NVVIKVCEFQFCNYPF | surface_glycoprotein@Brazil(NC_045512@Brazil:+: | 125-146 |
| | | LGVYYH | 21563-25384) | |
| 5 | 219-223 | ATLQA | linker | 1-5 |
| 5 | 224-231 | KAVFISPY | helicase(NC_045512:+:16237-18039) | 508-515 |
| 5 | 232-236 | ATLQA | linker | 1-5 |
| 5 | 237-254 | LNDLCFTNVYADSFVI | surface_glycoprotein(NC_045512:+:21563-25384) | 387-404 |
| | | RG | | |
| 5 | 255-259 | ATLQA | linker | 1-5 |
| 5 | 260-269 | DPIHSLRVCV | nsp3(NC_045512:+:2720-8554) | 339-348 |
| 5 | 270-274 | ATLQA | linker | 1-5 |
| 5 | 275-297 | LIAIVMVTIMLCCITS | surface_glycoprotein@Mozambique | 1220-1242 |
| | | CCSCLKG | (NC_045512@Mozambique:+:21563-25384) | |
| 5 | 298-302 | ATLQA | linker | 1-5 |
| 5 | 303-322 | SEAVEAPLVGTPVCIN | nsp2(NC_045512:+:806-2719) | 591-610 |
| | | GLML | | |
| 5 | 323-327 | ATLQA | linker | 1-5 |
| 5 | 328-342 | RTCCLCDRRATCFST | 3′-to-5′_exonuclease(NC_045512:+:18040-19620) | 205-219 |
| 5 | 343-347 | ATLQA | linker | 1-5 |
| 5 | 348-370 | RVDFCGKGYHLMSFPQ | surface_glycoprotein(NC_045512:+:21563-25384) | 1039-1061 |
| | | SAPHGVV | | |
| 5 | 371-375 | ATLQA | linker | 1-5 |
| 5 | 376-391 | GSNVFQTRAGCLIGAE | surface_glycoprotein(NC_045512:+:21563-25384) | 639-654 |
| 5 | 392-396 | ATLQA | linker | 1-5 |
| 5 | 397-404 | QIGVVREF | helicase(NC_045512:+:16237-18039) | 492-499 |
| 5 | 405-409 | ATLQA | linker | 1-5 |
| 5 | 410-419 | GTNLPLQLGF | 3′-to-5′_exonuclease(NC_045512:+:18040-19620) | 102-111 |
| 5 | 420-424 | ATLQA | linker | 1-5 |
| 5 | 425-439 | SYELQTPFEIKLAKK | nsp2(NC_045512:+:806-2719) | 68-82 |
| 5 | 440-444 | ATLQA | linker | 1-5 |
| 5 | 445-455 | FFSNVTWFHVS | surface_glycoprotein@Nigeria | 58-68 |
| | | | (NC_045512@Nigeria:+:21563-25384) | |
| 5 | 456-460 | ATLQA | linker | 1-5 |
| 5 | 461-475 | TYACWHHSIGFDYVY | 3′-to-5′_exonuclease(NC_045512:+:18040-19620) | 223-237 |
| 5 | 476-480 | ATLQA | linker | 1-5 |
| 5 | 481-500 | FCKLHNWNCVNCDTFC | nsp3(NC_045512:+:2720-8554) | 1626-1645 |
| | | AGST | | |
| 5 | 501-505 | ATLQA | linker | 1-5 |
| 5 | 506-520 | QIELKFNPPALQDAY | nsp3(NC_045512:+:2720-8554) | 867-881 |
| 5 | 521-525 | ATLQA | linker | 1-5 |
| 5 | 526-533 | FKLASHMY | nsp3(NC_045512:+:2720-8554) | 96-103 |
| 5 | 534-538 | ATLQA | linker | 1-5 |
| 5 | 539-563 | SDIDITFLKKDAPYIV | nsp3(NC_045512:+:2720-8554) | 454-478 |
| | | GDVVQEGVL | | |
| 5 | 564-568 | ATLQA | linker | 1-5 |
| 5 | 569-592 | PINLVRDLPQGFSVLE | surface_glycoprotein@Belgium | 205-228 |
| | | PLVDLPIG | (NC_045512@Belgium:+:21563-25384) | |
| 5 | 593-597 | ATLQA | linker | 1-5 |
| 5 | 598-607 | KQYGDCLGDI | surface_glycoprotein(NC_045512:+:21563-25384) | 835-844 |
| 5 | 608-612 | ATLQA | linker | 1-5 |
| 5 | 613-625 | LLNVPLHGTILTR | membrane_glycoprotein(NC_045512:+:26523-27191) | 119-131 |
| 5 | 626-630 | ATLQA | linker | 1-5 |
| 5 | 631-638 | SLIDLQEL | surface_glycoprotein(NC_045512:+:21563-25384) | 1196-1203 |
| 6 | 1-22 | INLVRDLPQGFSALEP | surface_glycoprotein@Aruba(NC_045512@Aruba:+: | 206-227 |
| | | LVDLPI | 21563-25384) | |
| 6 | 23-27 | ATLQA | linker | 1-5 |
| 6 | 28-41 | RNLQHRLYECLYRN | RNA-dependent_RNA_polymerase(NC_045512:+: | 721-734 |
| | | | 13442-13468+13468-16236) | |
| 6 | 42-46 | ATLQA | linker | 1-5 |
| 6 | 47-62 | FLVLLPLVSSQCVNLT | surface_glycoprotein@Aruba(NC_045512@Aruba:+: | 4-19 |
| | | | 21563-25384) | |
| 6 | 63-67 | ATLQA | linker | 1-5 |
| 6 | 68-78 | EQYVFCTVNAL | helicase(NC_045512:+:16237-18039) | 353-363 |
| 6 | 79-83 | ATLQA | linker | 1-5 |
| 6 | 84-95 | EVFAQVKQIYKT | surface_glycoprotein@Aruba(NC_045512@Aruba:+: | 776-787 |
| | | | 21563-25384) | |
| 6 | 96-100 | ATLQA | linker | 1-5 |
| 6 | 101-110 | DVVAIDYKHY | nsp3(NC_045512:+:2720-8554) | 1148-1157 |
| 6 | 111-115 | ATLQA | linker | 1-5 |
| 6 | 116-123 | KLGASQRV | membrane_glycoprotein(NC_045512:+:26523-27191) | 180-187 |
| 6 | 124-128 | ATLQA | linker | 1-5 |
| 6 | 129-137 | TENLLLYID | nsp3(NC_045512:+:2720-8554) | 432-440 |
| 6 | 138-142 | ATLQA | linker | 1-5 |
| 6 | 143-153 | KESVQTFFKLV | nsp2(NC_045512:+:806-2719) | 492-502 |
| 6 | 154-158 | ATLQA | linker | 1-5 |
| 6 | 159-174 | RKIFVDGVPFVVSTGY | RNA-dependent_RNA_polymerase(NC_045512:+: | 331-346 |
| | | | 13442-13468+13468-16236) | |
| 6 | 175-179 | ATLQA | linker | 1-5 |
| 6 | 180-197 | IKDLPKEITVATSRTL | membrane_glycoprotein(NC_045512:+:26523-27191) | 161-178 |
| | | SY | | |
| 6 | 198-202 | ATLQA | linker | 1-5 |
| 6 | 203-211 | SPDVDLADI | surface_glycoprotein@Chile(NC_045512@Chile:+: | 1161-1169 |
| | | | 21563-25384) | |
| 6 | 212-216 | ATLQA | linker | 1-5 |
| 6 | 217-226 | KQYGDCLGNI | surface_glycoprotein@Jordan(NC_045512@Jordan:+: | 831-840 |
| | | | 21563-25384) | |
| 6 | 227-231 | ATLQA | linker | 1-5 |
| 6 | 232-245 | SVASQSIIAYTMSL | surface_glycoprotein@Austria | 678-691 |
| | | | (NC_045512@Austria:+:21563-25384) | |
| 6 | 246-250 | ATLQA | linker | 1-5 |
| 6 | 251-270 | MTSCCSCLKGCCSCGS | surface_glycoprotein(NC_045512:+:21563-25384) | 1237-1256 |
| | | CCKF | | |
| 6 | 271-275 | ATLQA | linker | 1-5 |
| 6 | 276-292 | SAAYYVGYLQLRTFLL | surface_glycoprotein@Italy(NC_045512@Italy:+: | 258-274 |
| | | K | 21563-25384) | |
| 6 | 293-297 | ATLQA | linker | 1-5 |
| 6 | 298-305 | YDPLQPEL | surface_glycoprotein(NC_045512:+:21563-25384) | 1138-1145 |
| 6 | 306-310 | ATLQA | linker | 1-5 |
| 6 | 311-333 | GKIQDSLFSTASALGK | surface_glycoprotein@NorthMacedonia | 928-950 |
| | | LQDVVNQ | (NC_045512@NorthMacedonia:+:21563-25384) | |
| 6 | 334-338 | ATLQA | linker | 1-5 |
| 6 | 339-349 | IEELFYSYATH | 3′-to-5′_exonuclease(NC_045512:+:18040-19620) | 363-373 |
| 6 | 350-354 | ATLQA | linker | 1-5 |
| 6 | 355-373 | DKSAFVNLKQLPFFYY | 3′-to-5′_exonuclease(NC_045512:+:18040-19620) | 432-450 |
| | | SDS | | |
| 6 | 374-378 | ATLQA | linker | 1-5 |
| 6 | 379-386 | SVDCTMYI | surface_glycoprotein(NC_045512:+:21563-25384) | 735-742 |
| 6 | 387-391 | ATLQA | linker | 1-5 |
| 6 | 392-404 | NPVLPFNDGVYFA | surface_glycoprotein(NC_045512:+:21563-25384) | 81-93 |
| 6 | 405-409 | ATLQA | linker | 1-5 |
| 6 | 410-418 | AAAYYVGYL | surface_glycoprotein(NC_045512:+:21563-25384) | 262-270 |
| 6 | 419-423 | ATLQA | linker | 1-5 |
| 6 | 424-450 | FSASTSAFVETVKGLD | nsp2(NC_045512:+:806-2719) | 300-326 |
| | | YKAFKQIVESC | | |
| 6 | 451-455 | ATLQA | linker | 1-5 |
| 6 | 456-466 | RFQLHRSYLTP | surface_glycoprotein@Austria | 233-243 |
| | | | (NC_045512@Austria:+:21563-25384) | |
| 6 | 467-471 | ATLQA | linker | 1-5 |
| 6 | 472-484 | ANFCALILAYCNK | nsp3(NC_045512:+:2720-8554) | 890-902 |
| 6 | 485-489 | ATLQA | linker | 1-5 |
| 6 | 490-502 | WNVVRIKIVQMLS | 3′-to-5′_exonuclease(NC_045512:+:18040-19620) | 159-171 |
| 6 | 503-507 | ATLQA | linker | 1-5 |
| 6 | 508-521 | SYLFQHANLDSCKR | nsp3(NC_045512:+:2720-8554) | 915-928 |
| 6 | 522-526 | ATLQA | linker | 1-5 |
| 6 | 527-540 | GFNCYFPLQPYGFQ | surface_glycoprotein@SmallSouthAmerica | 481-494 |
| | | | (NC_045512@SmallSouthAmerica:+:21563-25384) | |
| 6 | 541-545 | ATLQA | linker | 1-5 |
| 6 | 546-553 | DSYFVVKR | RNA-dependent_RNA_polymerase | 67-74 |
| | | | (NC_045512:+:13442-13468+13468-16236) | |
| 6 | 554-558 | ATLQA | linker | 1-5 |
| 6 | 559-567 | FFSNVTWFH | surface_glycoprotein(NC_045512:+:21563-25384) | 58-66 |
| 6 | 568-572 | ATLQA | linker | 1-5 |
| 6 | 573-582 | VMCGGSLYVK | RNA-dependent_RNA_polymerase | 667-676 |
| | | | (NC_045512:+:13442-13468+13468-16236) | |
| 6 | 583-587 | ATLQA | linker | 1-5 |
| 6 | 588-597 | VTGLFKDCSK | 3′-to-5′_exonuclease(NC_045512:+:18040-19620) | 4-13 |
| 6 | 598-602 | ATLQA | linker | 1-5 |
| 6 | 603-623 | AVLYQGVNCTEVPFAI | surface_glycoprotein@SmallAfrica | 605-625 |
| | | HADQL | (NC_045512@SmallAfrica:+:21563-25384) | |
| 6 | 624-628 | ATLQA | linker | 1-5 |
| 6 | 629-639 | VVNAANVYLKH | nsp3(NC_045512:+:2720-8554) | 239-249 |

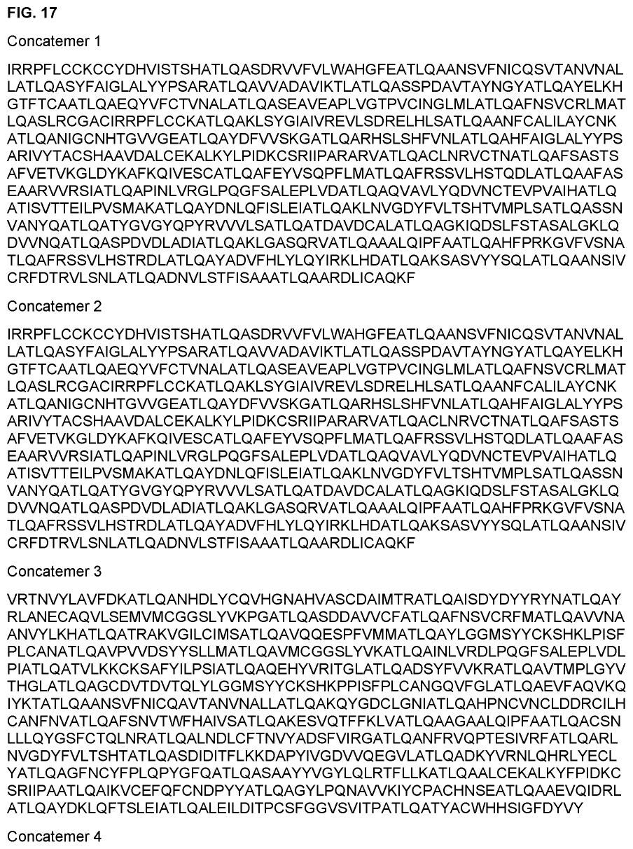

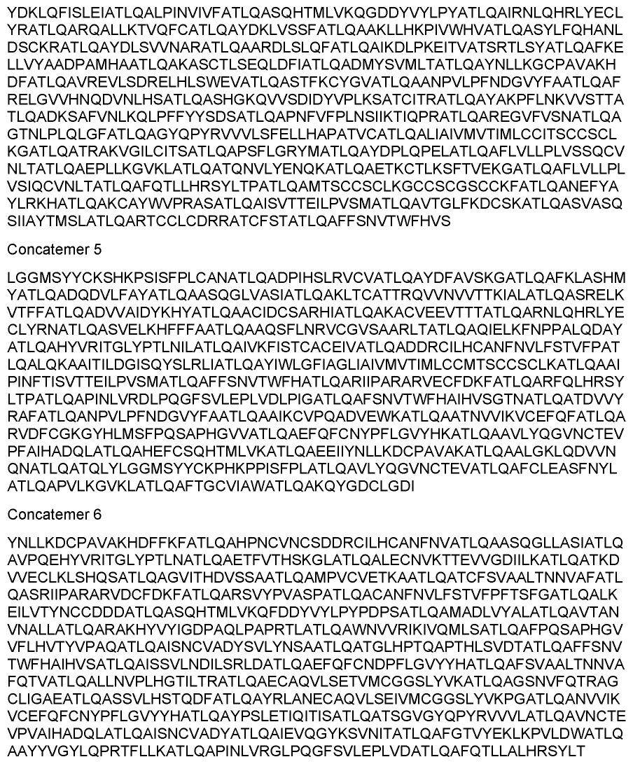

In certain embodiments, the vaccine comprises or expresses at least one of the concatemers of epitopes from SARS-COV-2 with intervening linker sequences (and as set forth in FIG. 17). In certain embodiments, the vaccine comprises or expresses at least two of the concatemers set forth below. In certain embodiments, the vaccine comprises or expresses three of the concatemers set forth below. In certain embodiments, the vaccine comprises or expresses four of the concatemers set forth below. In certain embodiments, the vaccine comprises or expresses five of the concatemers set forth below. In certain embodiments, the vaccine comprises or expresses all the concatemers set forth below.

TABLE 7

| | pos_in_ | | | pos_in_ |
|---|---|---|---|---|
| concat | concat | epitope | gene | gene |
| 1 | 1-20 | IRRPFLCCKCCYDHVISTSH | helicase(NC_045512:+:16237-18039) | 20-39 |
| 1 | 21-25 | ATLQA | linker | 1-5 |
| 1 | 26-39 | SDRVVFVLWAHGFE | 3′-to-5′_exonuclease(NC_045512:+:18040-19620) | 178-191 |
| 1 | 40-44 | ATLQA | linker | 1-5 |
| 1 | 45-63 | ANSVFNICQSVTANVNALL | RNA-dependent_RNA_polymerase@Croatia | 690-708 |
| | | | (NC_045512@Croatia:+:13442-13468+13468-16236) | |
| 1 | 64-68 | ATLQA | linker | 1-5 |
| 1 | 69-83 | SYFAIGLALYYPSAR | helicase@Austria | 289-303 |
| | | | (NC_045512@Austria:+:16237-18039) | |
| 1 | 84-88 | ATLQA | linker | 1-5 |
| 1 | 89-98 | VVADAVIKTL | nsp3(NC_045512:+:2720-8554) | 56-65 |
| 1 | 99-103 | ATLQA | linker | 1-5 |
| 1 | 104-115 | SSPDAVTAYNGY | nsp3(NC_045512:+:2720-8554) | 660-671 |
| 1 | 116-120 | ATLQA | linker | 1-5 |
| 1 | 121-131 | YELKHGTFTCA | nsp3(NC_045512:+:2720-8554) | 996-1006 |
| 1 | 132-136 | ATLQA | linker | 1-5 |
| 1 | 137-147 | EQYVFCTVNAL | helicase(NC_045512:+:16237-18039) | 353-363 |
| 1 | 148-152 | ATLQA | linker | 1-5 |
| 1 | 153-172 | SEAVEAPLVGTPVCINGLML | nsp2(NC_045512:+:806-2719) | 591-610 |
| 1 | 173-177 | ATLQA | linker | 1-5 |
| 1 | 178-185 | FNSVCRLM | helicase@Aruba(NC_045512@Aruba:+:16237-18039) | 422-429 |
| 1 | 186-190 | ATLQA | linker | 1-5 |
| 1 | 191-206 | SLRCGACIRRPFLCCK | helicase@Aruba(NC_045512@Aruba:+:16237-18039) | 13-28 |
| 1 | 207-211 | ATLQA | linker | 1-5 |
| 1 | 212-232 | KLSYGIAIVREVLSDRELHL | helicase@Colombia(NC_045512@Colombia:+: | 146-166 |
| | | S | 16237-18039) | |
| 1 | 233-237 | ATLQA | linker | 1-5 |
| 1 | 238-250 | ANFCALILAYCNK | nsp3(NC_045512:+:2720-8554) | 890-902 |
| 1 | 251-255 | ATLQA | linker | 1-5 |
| 1 | 256-267 | NIGCNHTGVVGE | nsp2(NC_045512:+:806-2719) | 250-261 |
| 1 | 268-272 | ATLQA | linker | 1-5 |
| 1 | 273-280 | YDFVVSKG | RNA-dependent_RNA_polymerase@Japan | 420-427 |
| | | | (NC_045512@Japan:+:13442-13468+13468-16236) | |
| 1 | 281-285 | ATLQA | linker | 1-5 |
| 1 | 286-295 | RHSLSHFVNL | nsp3(NC_045512:+:2720-8554) | 1697-1706 |
| 1 | 296-300 | ATLQA | linker | 1-5 |
| 1 | 301-351 | HFAIGLALYYPSARIVYTAC | helicase(NC_045512:+:16237-18039) | 290-340 |
| | | SHAAVDALCEKALKYLPIDK | | |
| | | CSRIIPARARV | | |
| 1 | 352-356 | ATLQA | linker | 1-5 |
| 1 | 357-364 | CLNRVCTN | nsp3(NC_045512:+:2720-8554) | 1342-1349 |
| 1 | 365-369 | ATLQA | linker | 1-5 |
| 1 | 370-396 | FSASTSAFVETVKGLDYKAF | nsp2(NC_045512:+:806-2719) | 300-326 |
| | | KQIVESC | | |
| 1 | 397-401 | ATLQA | linker | 1-5 |
| 1 | 402-411 | FEYVSQPFLM | surface_glycoprotein@Aruba(NC_045512@Aruba:+: | 164-173 |
| | | | 21563-25384) | |
| 1 | 412-416 | ATLQA | linker | 1-5 |
| 1 | 417-428 | FRSSVLHSTQDL | surface_glycoprotein@Aruba(NC_045512@Aruba:+: | 43-54 |
| | | | 21563-25384) | |
| 1 | 429-433 | ATLQA | linker | 1-5 |
| 1 | 434-446 | AFASEAARVVRSI | nsp2(NC_045512:+:806-2719) | 355-367 |
| 1 | 447-451 | ATLQA | linker | 1-5 |
| 1 | 452-471 | PINLVRGLPQGFSALEPLVD | surface_glycoprotein@Austria | 205-224 |
| | | | (NC_045512@Austria:+:21563-25384) | |
| 1 | 472-476 | ATLQA | linker | 1-5 |
| 1 | 477-495 | QVAVLYQDVNCTEVPVAIH | surface_glycoprotein@China | 607-625 |
| | | | (NC_045512@China:+:21563-25384) | |
| 1 | 496-500 | ATLQA | linker | 1-5 |
| 1 | 501-515 | TISVTTEILPVSMAK | surface_glycoprotein@Mexico | 719-733 |
| | | | (NC_045512@Mexico:+:21563-25384) | |
| 1 | 516-520 | ATLQA | linker | 1-5 |
| 1 | 521-531 | YDNLQFISLEI | helicase@Austria(NC_045512@Austria:+: | 582-592 |
| | | | 16237-18039) | |
| 1 | 532-536 | ATLQA | linker | 1-5 |
| 1 | 537-555 | KLNVGDYFVLTSHTVMPLS | helicase(NC_045512:+:16237-18039) | 218-236 |
| 1 | 556-560 | ATLQA | linker | 1-5 |
| 1 | 561-568 | SSNVANYQ | helicase@Austria(NC_045512@Austria:+: | 263-270 |
| | | | 16237-18039) | |
| 1 | 569-573 | ATLQA | linker | 1-5 |
| 1 | 574-588 | TYGVGYQPYRVVVLS | surface_glycoprotein@Australia | 496-510 |
| | | | (NC_045512@Australia:+:21563-25384) | |
| 1 | 589-593 | ATLQA | linker | 1-5 |
| 1 | 594-601 | TDAVDCAL | surface_glycoprotein(NC_045512:+:21563-25384) | 286-293 |
| 1 | 602-606 | ATLQA | linker | 1-5 |
| 1 | 607-629 | GKIQDSLFSTASALGKLQDV | surface_glycoprotein@NorthMacedonia | 928-950 |
| | | VNQ | (NC_045512@NorthMacedonia:+:21563-25384) | |
| 1 | 630-634 | ATLQA | linker | 1-5 |
| 1 | 635-643 | SPDVDLADI | surface_glycoprotein@Chile | 1161-1169 |
| | | | (NC_045512@Chile:+:21563-25384) | |
| 1 | 644-648 | ATLQA | linker | 1-5 |
| 1 | 649-656 | KLGASQRV | membrane_glycoprotein(NC_045512:+:26523-27191) | 180-187 |
| 1 | 657-661 | ATLQA | linker | 1-5 |
| 1 | 662-669 | AALQIPFA | surface_glycoprotein(NC_045512:+:21563-25384) | 892-899 |
| 1 | 670-674 | ATLQA | linker | 1-5 |
| 1 | 675-685 | HFPRKGVFVSN | surface_glycoprotein@SmallAsia | 1078-1088 |
| | | | (NC_045512@SmallAsia:+:21563-25384) | |
| 1 | 686-690 | ATLQA | linker | 1-5 |
| 1 | 691-702 | FRSSVLHSTRDL | surface_glycoprotein@Nigeria | 43-54 |
| | | | (NC_045512@Nigeria:+:21563-25384) | |
| 1 | 703-707 | ATLQA | linker | 1-5 |
| 1 | 708-724 | YADVFHLYLQYIRKLHD | RNA-dependent_RNA_polymerase(NC_045512:+: | 877-893 |
| | | | 13442-13468+13468-16236) | |
| 1 | 725-729 | ATLQA | linker | 1-5 |
| 1 | 730-739 | KSASVYYSQL | nsp3(NC_045512:+:2720-8554) | 1737-1746 |
| 1 | 740-744 | ATLQA | linker | 1-5 |
| 1 | 745-760 | ANSIVCRFDTRVLSNL | 3′-to-5′_exonuclease(NC_045512:+:18040-19620) | 394-409 |
| 1 | 761-765 | ATLQA | linker | 1-5 |
| 1 | 766-776 | DNVLSTFISAA | nsp3(NC_045512:+:2720-8554) | 1809-1819 |
| 1 | 777-781 | ATLQA | linker | 1-5 |
| 1 | 782-791 | ARDLICAQKF | surface_glycoprotein(NC_045512:+:21563-25384) | 846-855 |
| 2 | 1-8 | QIGVVREF | helicase(NC_045512:+:16237-18039) | 492-499 |
| 2 | 9-13 | ATLQA | linker | 1-5 |
| 2 | 14-24 | KGVQIPCTCGK | nsp3(NC_045512:+:2720-8554) | 963-973 |
| 2 | 25-29 | ATLQA | linker | 1-5 |
| 2 | 30-38 | TENLLLYID | nsp3(NC_045512:+:2720-8554) | 432-440 |
| 2 | 39-43 | ATLQA | linker | 1-5 |
| 2 | 44-56 | GVNLVAVPTGYVD | 3′-to-5′_exonuclease(NC_045512:+:18040-19620) | 114-126 |
| 2 | 57-61 | ATLQA | linker | 1-5 |
| 2 | 62-70 | AGFSLWVYK | 3′-to-5′_exonuclease(NC_045512:+:18040-19620) | 504-512 |
| 2 | 71-75 | ATLQA | linker | 1-5 |
| 2 | 76-107 | IMASLVLARKHTTCCSLSHR | RNA-dependent_RNA_polymerase(NC_045512:+: | 632-663 |
| | | FYRLANECAQVL | 13442-13468+13468-16236) | |
| 2 | 108-112 | ATLQA | linker | 1-5 |
| 2 | 113-120 | SINVANYQ | helicase@Mozambique | 263-270 |
| | | | (NC_045512@Mozambique:+:16237-18039) | |
| 2 | 121-125 | ATLQA | linker | 1-5 |
| 2 | 126-135 | LSVNPYVCNA | helicase@Cyprus(NC_045512@Cyprus:+:16237-18039) | 43-52 |
| 2 | 136-140 | ATLQA | linker | 1-5 |
| 2 | 141-148 | KAVFISPY | helicase(NC_045512:+:16237-18039) | 508-515 |
| 2 | 149-153 | ATLQA | linker | 1-5 |
| 2 | 154-163 | NYQCGHYKHI | nsp3(NC_045512:+:2720-8554) | 1012-1021 |
| 2 | 164-168 | ATLQA | linker | 1-5 |
| 2 | 169-179 | AVHECFVKRVD | 3′-to-5′_exonuclease(NC_045512:+:18040-19620) | 281-291 |
| 2 | 180-184 | ATLQA | linker | 1-5 |
| 2 | 185-200 | RKIFVDGVPFVVSTGY | RNA-dependent_RNA_polymerase(NC_045512:+: | 331-346 |
| | | | 13442-13468+13468-16236) | |
| 2 | 201-205 | ATLQA | linker | 1-5 |
| 2 | 206-220 | SYELQTPFEIKLAKK | nsp2(NC_045512:+:806-2719) | 68-82 |
| 2 | 221-225 | ATLQA | linker | 1-5 |
| 2 | 226-233 | GVYCCREH | nsp2(NC_045512:+:806-2719) | 47-54 |
| 2 | 234-238 | ATLQA | linker | 1-5 |
| 2 | 239-256 | IKVCEFQFCNDPFLGVYH | surface_glycoprotein@Aruba | 125-142 |
| | | | (NC_045512@Aruba:+:21563-25384) | |
| 2 | 257-261 | ATLQA | linker | 1-5 |
| 2 | 262-276 | ERFVSLAIDAYPLTK | RNA-dependent_RNA_polymerase(NC_045512:+: | 857-871 |
| | | | 13442-13468+13468-16236) | |
| 2 | 277-281 | ATLQA | linker | 1-5 |
| 2 | 282-301 | YVYLPYPDPSRILGAGCFVD | RNA-dependent_RNA_polymerase(NC_045512:+: | 826-845 |
| | | | 13442-13468+13468-16236) | |
| 2 | 302-306 | ATLQA | linker | 1-5 |
| 2 | 307-314 | PEHSLAEY | nsp2(NC_045512:+:806-2719) | 200-207 |
| 2 | 315-319 | ATLQA | linker | 1-5 |
| 2 | 320-330 | LRIAGHHLGRC | membrane_glycoprotein(NC_045512:+:26523-27191) | 149-159 |
| 2 | 331-335 | ATLQA | linker | 1-5 |
| 2 | 336-355 | FCKLHNWNCVNCDTFCAGST | nsp3(NC_045512:+:2720-8554) | 1626-1645 |
| 2 | 356-360 | ATLQA | linker | 1-5 |
| 2 | 361-368 | IETISLAG | nsp3(NC_045512:+:2720-8554) | 684-691 |
| 2 | 369-373 | ATLQA | linker | 1-5 |
| 2 | 374-384 | SELVIGAVILR | membrane_glycoprotein(NC_045512:+:26523-27191) | 136-146 |
| 2 | 385-389 | ATLQA | linker | 1-5 |
| 2 | 390-405 | LQSLQTYVTQQLIRAA | surface_glycoprotein(NC_045512:+:21563-25384) | 1001-1016 |
| 2 | 406-410 | ATLQA | linker | 1-5 |
| 2 | 411-423 | RSVASQSIIAYTM | surface_glycoprotein@Aruba | 681-693 |
| | | | (NC_045512@Aruba:+:21563-25384) | |
| 2 | 424-428 | ATLQA | linker | 1-5 |
| 2 | 429-437 | TEIYQAGST | surface_glycoprotein@Aruba | 466-474 |
| | | | (NC_045512@Aruba:+:21563-25384) | |
| 2 | 438-442 | ATLQA | linker | 1-5 |
| 2 | 443-456 | EGVVDYGARFYFYT | nsp3(NC_045512:+:2720-8554) | 595-608 |
| 2 | 457-461 | ATLQA | linker | 1-5 |
| 2 | 462-475 | LVLLPLVSSQCVNF | surface_glycoprotein@Brazil | 5-18 |
| | | | (NC_045512@Brazil:+:21563-25384) | |
| 2 | 476-480 | ATLQA | linker | 1-5 |
| 2 | 481-489 | AAAYYVGYL | surface_glycoprotein(NC_045512:+:21563-25384) | 262-270 |
| 2 | 490-494 | ATLQA | linker | 1-5 |
| 2 | 495-503 | VFSQCVNLT | surface_glycoprotein@Egypt | 11-19 |
| | | | (NC_045512@Egypt:+:21563-25384) | |
| 2 | 504-508 | ATLQA | linker | 1-5 |
| 2 | 509-520 | FNCYFPLQSYGF | surface_glycoprotein(NC_045512:+:21563-25384) | 486-497 |
| 2 | 521-525 | ATLQA | linker | 1-5 |
| 2 | 526-533 | SLIDLQEL | surface_glycoprotein(NC_045512:+:21563-25384) | 1196-1203 |
| 2 | 534-538 | ATLQA | linker | 1-5 |
| 2 | 539-553 | INAACRKVQHMVVKA | 3′-to-5′_exonuclease(NC_045512:+:18040-19620) | 305-319 |
| 2 | 554-558 | ATLQA | linker | 1-5 |
| 2 | 559-568 | SVLNDILARL | surface_glycoprotein@Aruba | 971-980 |
| | | | (NC_045512@Aruba:+:21563-25384) | |
| 2 | 569-573 | ATLQA | linker | 1-5 |
| 2 | 574-581 | RVEAFEYY | nsp3(NC_045512:+:2720-8554) | 810-817 |
| 2 | 582-586 | ATLQA | linker | 1-5 |
| 2 | 587-604 | CANFNVLFSTVFPPTSFG | RNA-dependent_RNA_polymerase@China | 310-327 |
| | | | (NC_045512@China:+:13442-13468+13468-16236) | |
| 2 | 605-609 | ATLQA | linker | 1-5 |
| 2 | 610-623 | GFNCYSPLQSYGFQ | surface_glycoprotein@Peru | 485-498 |
| | | | (NC_045512@Peru:+:21563-25384) | |
| 2 | 624-628 | ATLQA | linker | 1-5 |
| 2 | 629-646 | LFVVEVVDKYFDCYDGGC | RNA-dependent_RNA_polymerase(NC_045512:+: | 470-487 |
| | | | 13442-13468+13468-16236) | |
| 2 | 647-651 | ATLQA | linker | 1-5 |
| 2 | 652-661 | GNCDVVIGIV | surface_glycoprotein(NC_045512:+:21563-25384) | 1124-1133 |
| 2 | 662-666 | ATLQA | linker | 1-5 |
| 2 | 667-674 | SVDCTMYI | surface_glycoprotein(NC_045512:+:21563-25384) | 735-742 |
| 2 | 675-679 | ATLQA | linker | 1-5 |
| 2 | 680-687 | NNLVVMAY | nsp2(NC_045512:+:806-2719) | 413-420 |
| 2 | 688-692 | ATLQA | linker | 1-5 |
| 2 | 693-715 | TIATYCTGSIPCSVCLSGLD | nsp3(NC_045512:+:2720-8554) | 1459-1481 |
| | | SLD | | |
| 2 | 716-720 | ATLQA | linker | 1-5 |
| 2 | 721-735 | ARSVARQSIIAYTMS | surface_glycoprotein@Mozambique | 680-694 |
| | | | (NC_045512@Mozambique:+:21563-25384) | |
| 2 | 736-740 | ATLQA | linker | 1-5 |
| 2 | 741-751 | IEELFYSYATH | 3′-to-5′_exonuclease(NC_045512:+:18040-19620) | 363-373 |
| 2 | 752-756 | ATLQA | linker | 1-5 |
| 2 | 757-765 | EVFAQVKQI | surface_glycoprotein(NC_045512:+:21563-25384) | 780-788 |
| 2 | 766-770 | ATLQA | linker | 1-5 |
| 2 | 771-778 | EIYQAGSK | surface_glycoprotein@Mexico | 471-478 |
| | | | (NC_045512@Mexico:+:21563-25384) | |
| 2 | 779-783 | ATLQA | linker | 1-5 |
| 2 | 784-797 | APGCDVTDVTQLYL | helicase@Aruba(NC_045512@Aruba:+:16237-18039) | 52-65 |
| 3 | 1-12 | VRTNVYLAVFDK | nsp3(NC_045512:+:2720-8554) | 351-362 |
| 3 | 13-17 | ATLQA | linker | 1-5 |
| 3 | 18-40 | NHDLYCQVHGNAHVASCDAI | 3′-to-5′_exonuclease(NC_045512:+:18040-19620) | 256-278 |
| | | MTR | | |
| 3 | 41-45 | ATLQA | linker | 1-5 |
| 3 | 46-55 | ISDYDYYRYN | RNA-dependent_RNA_polymerase@Aruba | 450-459 |
| | | | (NC_045512@Aruba:+:13442-13468+13468-16236) | |
| 3 | 56-60 | ATLQA | linker | 1-5 |
| 3 | 61-86 | YRLANECAQVLSEMVMCGGS | RNA-dependent_RNA_polymerase@Australia | 653-678 |
| | | LYVKPG | (NC_045512@Australia:+:13442-13468+13468-16236) | |
| 3 | 87-91 | ATLQA | linker | 1-5 |
| 3 | 92-99 | SDDAVVCF | RNA-dependent_RNA_polymerase@Aruba | 759-766 |
| | | | (NC_045512@Aruba:+:13442-13468+13468-16236) | |
| 3 | 100-104 | ATLQA | linker | 1-5 |
| 3 | 105-112 | FNSVCRFM | helicase@Malaysia(NC_045512@Malaysia:+: | 422-429 |
| | | | 16237-18039) | |
| 3 | 113-117 | ATLQA | linker | 1-5 |
| 3 | 118-128 | VVNAANVYLKH | nsp3(NC_045512:+:2720-8554) | 239-249 |
| 3 | 129-133 | ATLQA | linker | 1-5 |
| 3 | 134-145 | TRAKVGILCIMS | helicase(NC_045512:+:16237-18039) | 566-577 |
| 3 | 146-150 | ATLQA | linker | 1-5 |
| 3 | 151-160 | VQQESPFVMM | nsp3(NC_045512:+:2720-8554) | 980-989 |
| 3 | 161-165 | ATLQA | linker | 1-5 |
| 3 | 166-188 | YLGGMSYYCKSHKLPISFPL | helicase@CostaRica(NC_045512@CostaRica:+: | 64-86 |
| | | CAN | 16237-18039) | |
| 3 | 189-193 | ATLQA | linker | 1-5 |
| 3 | 194-205 | VPVVDSYYSLLM | RNA-dependent_RNA_polymerase(NC_045512:+: | 231-242 |
| | | | 13442-13468+13468-16236) | |
| 3 | 206-210 | ATLQA | linker | 1-5 |
| 3 | 211-220 | VMCGGSLYVK | RNA-dependent_RNA_polymerase(NC_045512:+: | 667-676 |
| | | | 13442-13468+13468-16236) | |
| 3 | 221-225 | ATLQA | linker | 1-5 |
| 3 | 226-247 | INLVRDLPQGFSALEPLVDL | surface_glycoprotein@Aruba | 206-227 |
| | | PI | (NC_045512@Aruba:+:21563-25384) | |
| 3 | 248-252 | ATLQA | linker | 1-5 |
| 3 | 253-268 | TVLKKCKSAFYILPSI | nsp3(NC_045512:+:2720-8554) | 526-541 |
| 3 | 269-273 | ATLQA | linker | 1-5 |
| 3 | 274-283 | QEHYVRITGL | helicase(NC_045512:+:16237-18039) | 243-252 |
| 3 | 284-288 | ATLQA | linker | 1-5 |
| 3 | 289-296 | DSYFVVKR | RNA-dependent_RNA_polymerase(NC_045512:+: | 67-74 |
| | | | 13442-13468+13468-16236) | |
| 3 | 297-301 | ATLQA | linker | 1-5 |
| 3 | 302-313 | VTMPLGYVTHGL | nsp3(NC_045512:+:2720-8554) | 628-639 |
| 3 | 314-318 | ATLQA | linker | 1-5 |
| 3 | 319-357 | GCDVTDVTQLYLGGMSYYCK | helicase(NC_045512:+:16237-18039) | 54-92 |
| | | SHKPPISFPLCANGQVFGL | | |
| 3 | 358-362 | ATLQA | linker | 1-5 |
| 3 | 363-374 | EVFAQVKQIYKT | surface_glycoprotein@Aruba | 776-787 |
| | | | (NC_045512@Aruba:+:21563-25384) | |
| 3 | 375-379 | ATLQA | linker | 1-5 |
| 3 | 380-398 | ANSVFNICQAVTANVNALL | RNA-dependent_RNA_polymerase@Aruba | 690-708 |
| | | | (NC_045512@Aruba:+:13442-13468+13468-16236) | |
| 3 | 399-403 | ATLQA | linker | 1-5 |
| 3 | 404-413 | KQYGDCLGNI | surface_glycoprotein@Jordan | 831-840 |
| | | | (NC_045512@Jordan:+:21563-25384) | |
| 3 | 414-418 | ATLQA | linker | 1-5 |
| 3 | 419-439 | HPNCVNCLDDRCILHCANFN | RNA-dependent_RNA_polymerase@Australia | 295-315 |
| | | V | (NC_045512@Australia:+:13442-13468+13468-16236) | |
| 3 | 440-444 | ATLQA | linker | 1-5 |
| 3 | 445-456 | FSNVTWFHAIVS | surface_glycoprotein@Ghana | 59-70 |
| | | | (NC_045512@Ghana:+:21563-25384) | |
| 3 | 457-461 | ATLQA | linker | 1-5 |
| 3 | 462-472 | KESVQTFFKLV | nsp2(NC_045512:+:806-2719) | 492-502 |
| 3 | 473-477 | ATLQA | linker | 1-5 |
| 3 | 478-487 | AGAALQIPFA | surface_glycoprotein@Nigeria | 886-895 |
| | | | (NC_045512@Nigeria:+:21563-25384) | |
| 3 | 488-492 | ATLQA | linker | 1-5 |
| 3 | 493-509 | CSNLLLQYGSFCTQLNR | surface_glycoprotein(NC_045512:+:21563-25384) | 749-765 |
| 3 | 510-514 | ATLQA | linker | 1-5 |
| 3 | 515-532 | LNDLCFTNVYADSFVIRG | surface_glycoprotein(NC_045512:+:21563-25384) | 387-404 |
| 3 | 533-537 | ATLQA | linker | 1-5 |
| 3 | 538-550 | NFRVQPTESIVRF | surface_glycoprotein(NC_045512:+:21563-25384) | 317-329 |
| 3 | 551-555 | ATLQA | linker | 1-5 |
| 3 | 556-569 | RLNVGDYFVLTSHT | helicase@Austria(NC_045512@Austria:+: | 218-231 |
| | | | 16237-18039) | |
| 3 | 570-574 | ATLQA | linker | 1-5 |
| 3 | 575-599 | SDIDITFLKKDAPYIVGDVV | nsp3(NC_045512:+:2720-8554) | 454-478 |
| | | QEGVL | | |
| 3 | 600-604 | ATLQA | linker | 1-5 |
| 3 | 605-620 | DKYVRNLQHRLYECLY | RNA-dependent_RNA_polymerase@Aruba | 717-732 |
| | | | (NC_045512@Aruba:+:13442-13468+13468-16236) | |
| 3 | 621-625 | ATLQA | linker | 1-5 |
| 3 | 626-639 | GFNCYFPLQPYGFQ | surface_glycoprotein@SmallSouthAmerica | 481-494 |
| | | | (NC_045512@SmallSouthAmerica:+:21563-25384) | |
| 3 | 640-644 | ATLQA | linker | 1-5 |
| 3 | 645-661 | SAAYYVGYLQLRTFLLK | surface_glycoprotein@Italy | 258-274 |
| | | | (NC_045512@Italy:+:21563-25384) | |
| 3 | 662-666 | ATLQA | linker | 1-5 |
| 3 | 667-687 | ALCEKALKYFPIDKCSRIIP | helicase@Colombia(NC_045512@Colombia:+: | 316-336 |
| | | A | 16237-18039) | |
| 3 | 688-692 | ATLQA | linker | 1-5 |
| 3 | 693-706 | IKVCEFQFCNDPYY | surface_glycoprotein@CostaRica | 128-141 |
| | | | (NC_045512@CostaRica:+:21563-25384) | |
| 3 | 707-711 | ATLQA | linker | 1-5 |
| 3 | 712-731 | GYLPQNAVVKIYCPACHNSE | nsp2(NC_045512:+:806-2719) | 178-197 |
| 3 | 732-736 | ATLQA | linker | 1-5 |
| 3 | 737-744 | AEVQIDRL | surface_glycoprotein(NC_045512:+:21563-25384) | 989-996 |
| 3 | 745-749 | ATLQA | linker | 1-5 |
| 3 | 750-760 | YDKLQFTSLEI | helicase@Aruba(NC_045512@Aruba:+:16237-18039) | 582-592 |
| 3 | 761-765 | ATLQA | linker | 1-5 |
| 3 | 766-784 | LEILDITPCSFGGVSVITP | surface_glycoprotein(NC_045512:+:21563-25384) | 582-600 |
| 3 | 785-789 | ATLQA | linker | 1-5 |
| 3 | 790-804 | TYACWHHSIGFDYVY | 3′-to-5′_exonuclease(NC_045512:+:18040-19620) | 223-237 |
| 4 | 1-11 | YDKLQFISLEI | helicase@SouthAfrica(NC_045512@SouthAfrica:+: | 582-592 |
| | | | 16237-18039) | |
| 4 | 12-16 | ATLQA | linker | 1-5 |
| 4 | 17-24 | LPINVIVF | nsp3(NC_045512:+:2720-8554) | 1718-1725 |
| 4 | 25-29 | ATLQA | linker | 1-5 |
| 4 | 30-47 | SQHTMLVKQGDDYVYLPY | RNA-dependent_RNA_polymerase@Aruba | 814-831 |
| | | | (NC_045512@Aruba:+:13442-13468+13468-16236) | |
| 4 | 48-52 | ATLQA | linker | 1-5 |
| 4 | 53-66 | IRNLQHRLYECLYR | RNA-dependent_RNA_polymerase@Austria | 720-733 |
| | | | (NC_045512@Austria:+:13442-13468+13468-16236) | |
| 4 | 67-71 | ATLQA | linker | 1-5 |
| 4 | 72-82 | RQALLKTVQFC | RNA-dependent_RNA_polymerase@Aruba | 183-193 |
| | | | (NC_045512@Aruba:+:13442-13468+13468-16236) | |
| 4 | 83-87 | ATLQA | linker | 1-5 |
| 4 | 88-95 | YDKLVSSF | nsp3(NC_045512:+:2720-8554) | 365-372 |
| 4 | 96-100 | ATLQA | linker | 1-5 |
| 4 | 101-112 | AKLLHKPIVWHV | nsp3(NC_045512:+:2720-8554) | 1165-1176 |
| 4 | 113-117 | ATLQA | linker | 1-5 |
| 4 | 118-131 | SYLFQHANLDSCKR | nsp3(NC_045512:+:2720-8554) | 915-928 |
| 4 | 132-136 | ATLQA | linker | 1-5 |
| 4 | 137-145 | YDLSVVNAR | helicase(NC_045512:+:16237-18039) | 382-390 |
| 4 | 146-150 | ATLQA | linker | 1-5 |
| 4 | 151-158 | ARDLSLQF | nsp3(NC_045512:+:2720-8554) | 1652-1659 |
| 4 | 159-163 | ATLQA | linker | 1-5 |
| 4 | 164-181 | IKDLPKEITVATSRTLSY | membrane_glycoprotein(NC_045512:+:26523-27191) | 161-178 |
| 4 | 182-186 | ATLQA | linker | 1-5 |
| 4 | 187-201 | FKELLVYAADPAMHA | RNA-dependent_RNA_polymerase(NC_045512:+: | 368-382 |
| | | | 13442-13468+13468-16236) | |
| 4 | 202-206 | ATLQA | linker | 1-5 |
| 4 | 207-219 | KASCTLSEQLDFI | nsp2(NC_045512:+:806-2719) | 30-42 |
| 4 | 220-224 | ATLQA | linker | 1-5 |
| 4 | 225-232 | DMYSVMLT | RNA-dependent_RNA_polymerase@Malaysia | 901-908 |
| | | | (NC_045512@Malaysia:+:13442-13468+13468-16236) | |
| 4 | 233-237 | ATLQA | linker | 1-5 |
| 4 | 238-252 | YNLLKGCPAVAKHDF | RNA-dependent_RNA_polymerase@Poland | 87-101 |
| | | | (NC_045512@Poland:+:13442-13468+13468-16236) | |
| 4 | 253-257 | ATLQA | linker | 1-5 |
| 4 | 258-273 | VREVLSDRELHLSWEV | helicase(NC_045512:+:16237-18039) | 154-169 |
| 4 | 274-278 | ATLQA | linker | 1-5 |
| 4 | 279-286 | STFKCYGV | surface_glycoprotein@Aruba | 371-378 |
| | | | (NC_045512@Aruba:+:21563-25384) | |
| 4 | 287-291 | ATLQA | linker | 1-5 |
| 4 | 292-305 | ANPVLPFNDGVYFA | surface_glycoprotein@Austria | 77-90 |
| | | | (NC_045512@Austria:+:21563-25384) | |
| 4 | 306-310 | ATLQA | linker | 1-5 |
| 4 | 311-326 | FRELGVVHNQDVNLHS | RNA-dependent_RNA_polymerase(NC_045512:+: | 348-363 |
| | | | 13442-13468+13468-16236) | |
| 4 | 327-331 | ATLQA | linker | 1-5 |
| 4 | 332-354 | SHGKQVVSDIDYVPLKSATC | 3′-to-5′_exonuclease(NC_045512:+:18040-19620) | 454-476 |
| | | ITR | | |
| 4 | 355-359 | ATLQA | linker | 1-5 |
| 4 | 360-372 | YAKPFLNKVVSTT | nsp3(NC_045512:+:2720-8554) | 1323-1335 |
| 4 | 373-377 | ATLQA | linker | 1-5 |
| 4 | 378-396 | DKSAFVNLKQLPFFYYSDS | 3′-to-5′_exonuclease(NC_045512:+:18040-19620) | 432-450 |
| 4 | 397-401 | ATLQA | linker | 1-5 |
| 4 | 402-418 | PNFVFPLNSIIKTIQPR | nsp2(NC_045512:+:806-2719) | 91-107 |
| 4 | 419-423 | ATLQA | linker | 1-5 |
| 4 | 424-431 | REGVFVSN | surface_glycoprotein@Aruba | 1087-1094 |
| | | | (NC_045512@Aruba:+:21563-25384) | |
| 4 | 432-436 | ATLQA | linker | 1-5 |
| 4 | 437-446 | GTNLPLQLGF | 3′-to-5′_exonuclease(NC_045512:+:18040-19620) | 102-111 |
| 4 | 447-451 | ATLQA | linker | 1-5 |
| 4 | 452-473 | GYQPYRVVVLSFELLHAPAT | surface_glycoprotein(NC_045512:+:21563-25384) | 504-525 |
| | | VC | | |
| 4 | 474-478 | ATLQA | linker | 1-5 |
| 4 | 479-501 | LIAIVMVTIMLCCITSCCSC | surface_glycoprotein@Mozambique | 1220-1242 |
| | | LKG | (NC_045512@Mozambique:+:21563-25384) | |
| 4 | 502-506 | ATLQA | linker | 1-5 |
| 4 | 507-518 | TRAKVGILCITS | helicase@SouthKorea(NC_045512@SouthKorea:+: | 566-577 |
| | | | 16237-18039) | |
| 4 | 519-523 | ATLQA | linker | 1-5 |
| 4 | 524-531 | PSFLGRYM | nsp3(NC_045512:+:2720-8554) | 822-829 |
| 4 | 532-536 | ATLQA | linker | 1-5 |
| 4 | 537-544 | YDPLQPEL | surface_glycoprotein(NC_045512:+:21563-25384) | 1138-1145 |
| 4 | 545-549 | ATLQA | linker | 1-5 |
| 4 | 550-565 | FLVLLPLVSSQCVNLT | surface_glycoprotein@Aruba(NC_045512@Aruba:+: | 4-19 |
| | | | 21563-25384) | |
| 4 | 566-570 | ATLQA | linker | 1-5 |
| 4 | 571-579 | EPLLKGVKL | surface_glycoprotein@Croatia | 1258-1266 |
| | | | (NC_045512@Croatia:+:21563-25384) | |
| 4 | 580-584 | ATLQA | linker | 1-5 |
| 4 | 585-594 | TQNVLYENQK | surface_glycoprotein(NC_045512:+:21563-25384) | 912-921 |
| 4 | 595-599 | ATLQA | linker | 1-5 |
| 4 | 600-613 | ETKCTLKSFTVEKG | surface_glycoprotein@Aruba | 294-307 |
| | | | (NC_045512@Aruba:+:21563-25384) | |
| 4 | 614-618 | ATLQA | linker | 1-5 |
| 4 | 619-634 | FLVLLPLVSIQCVNLT | surface_glycoprotein@SmallNorthAmerica | 4-19 |
| | | | (NC_045512@SmallNorthAmerica:+:21563-25384) | |
| 4 | 635-639 | ATLQA | linker | 1-5 |
| 4 | 640-651 | FQTLLHRSYLTP | surface_glycoprotein@SmallAsia | 230-241 |
| | | | (NC_045512@SmallAsia:+:21563-25384) | |
| 4 | 652-656 | ATLQA | linker | 1-5 |
| 4 | 657-676 | MTSCCSCLKGCCSCGSCCKF | surface_glycoprotein(NC_045512:+:21563-25384) | 1237-1256 |
| 4 | 677-681 | ATLQA | linker | 1-5 |
| 4 | 682-691 | NEFYAYLRKH | RNA-dependent_RNA_polymerase | 743-752 |
| | | | (NC_045512:+:13442-13468+13468-16236) | |
| 4 | 692-696 | ATLQA | linker | 1-5 |
| 4 | 697-706 | KCAYWVPRAS | nsp2(NC_045512:+:806-2719) | 239-248 |
| 4 | 707-711 | ATLQA | linker | 1-5 |
| 4 | 712-723 | ISVTTEILPVSM | surface_glycoprotein(NC_045512:+:21563-25384) | 720-731 |
| 4 | 724-728 | ATLQA | linker | 1-5 |
| 4 | 729-738 | VTGLFKDCSK | 3′-to-5′_exonuclease(NC_045512:+:18040-19620) | 4-13 |
| 4 | 739-743 | ATLQA | linker | 1-5 |
| 4 | 744-757 | SVASQSIIAYTMSL | surface_glycoprotein@Austria | 678-691 |
| | | | (NC_045512@Austria:+:21563-25384) | |
| 4 | 758-762 | ATLQA | linker | 1-5 |
| 4 | 763-777 | RTCCLCDRRATCFST | 3′-to-5′_exonuclease(NC_045512:+:18040-19620) | 205-219 |
| 4 | 778-782 | ATLQA | linker | 1-5 |
| 4 | 783-793 | FFSNVTWFHVS | surface_glycoprotein@Nigeria | 58-68 |
| | | | (NC_045512@Nigeria:+:21563-25384) | |
| 5 | 1-22 | LGGMSYYCKSHKPSISFPLC | helicase@NewZealand(NC_045512@NewZealand:+: | 65-86 |
| | | AN | 16237-18039) | |
| 5 | 23-27 | ATLQA | linker | 1-5 |
| 5 | 28-37 | DPIHSLRVCV | nsp3(NC_045512:+:2720-8554) | 339-348 |
| 5 | 38-42 | ATLQA | linker | 1-5 |
| 5 | 43-50 | YDFAVSKG | RNA-dependent_RNA_polymerase@Aruba | 420-427 |
| | | | (NC_045512@Aruba:+:13442-13468+13468-16236) | |
| 5 | 51-55 | ATLQA | linker | 1-5 |
| 5 | 56-63 | FKLASHMY | nsp3(NC_045512:+:2720-8554) | 96-103 |
| 5 | 64-68 | ATLQA | linker | 1-5 |
| 5 | 69-76 | DQDVLFAY | RNA-dependent_RNA_polymerase@FrenchGuiana | 523-530 |
| | | | (NC_045512@FrenchGuiana:+: | |
| | | | 13442-13468+13468-16236) | |
| 5 | 77-81 | ATLQA | linker | 1-5 |
| 5 | 82-90 | ASQGLVASI | RNA-dependent_RNA_polymerase@Aruba | 771-779 |
| | | | (NC_045512@Aruba:+:13442-13468+13468-16236) | |
| 5 | 91-95 | ATLQA | linker | 1-5 |
| 5 | 96-115 | KLTCATTRQVVNVVTTKIAL | nsp3(NC_045512:+:2720-8554) | 1923-1942 |
| 5 | 116-120 | ATLQA | linker | 1-5 |
| 5 | 121-129 | SRELKVTFF | nsp3(NC_045512:+:2720-8554) | 1134-1142 |
| 5 | 130-134 | ATLQA | linker | 1-5 |
| 5 | 135-144 | DVVAIDYKHY | nsp3(NC_045512:+:2720-8554) | 1148-1157 |
| 5 | 145-149 | ATLQA | linker | 1-5 |
| 5 | 150-159 | ACIDCSARHI | nsp3(NC_045512:+:2720-8554) | 1872-1881 |
| 5 | 160-164 | ATLQA | linker | 1-5 |
| 5 | 165-174 | KACVEEVTTT | nsp3(NC_045512:+:2720-8554) | 415-424 |
| 5 | 175-179 | ATLQA | linker | 1-5 |
| 5 | 180-193 | RNLQHRLYECLYRN | RNA-dependent_RNA_polymerase(NC_045512:+: | 721-734 |
| 5 | 194-198 | ATLQA | linker13442-13468+13468-16236) | 1-5 |
| 5 | 199-208 | SVELKHFFFA | RNA-dependent_RNA_polymerase(NC_045512:+: | 434-443 |
| | | | 13442-13468+13468-16236) | |
| 5 | 209-213 | ATLQA | linker | 1-5 |
| 5 | 214-230 | AQSFLNRVCGVSAARLT | RNA-dependent_RNA_polymerase(NC_045512:+: | 4-20 |
| | | | 13442-13468+13468-16236) | |
| 5 | 231-235 | ATLQA | linker | 1-5 |
| 5 | 236-250 | QIELKFNPPALQDAY | nsp3(NC_045512:+:2720-8554) | 867-881 |
| 5 | 251-255 | ATLQA | linker | 1-5 |
| 5 | 256-270 | HYVRITGLYPTLNIL | helicase@Indonesia(NC_045512@Indonesia:+: | 245-259 |
| | | | 16237-18039) | |
| 5 | 271-275 | ATLQA | linker | 1-5 |
| 5 | 276-288 | IVKFISTCACEIV | nsp2(NC_045512:+:806-2719) | 468-480 |
| 5 | 289-293 | ATLQA | linker | 1-5 |
| 5 | 294-313 | DDRCILHCANFNVLFSTVFP | RNA-dependent_RNA_polymerase(NC_045512:+: | 303-322 |
| | | | 13442-13468+13468-16236) | |
| 5 | 314-318 | ATLQA | linker | 1-5 |
| 5 | 319-338 | LQKAAITILDGISQYSLRLI | nsp2(NC_045512:+:806-2719) | 382-401 |
| 5 | 339-343 | ATLQA | linker | 1-5 |
| 5 | 344-374 | YIWLGFIAGLIAIVMVTIML | surface_glycoprotein@Aruba(NC_045512@Aruba:+: | 1211-1241 |
| | | CCMTSCCSCLK | 21563-25384) | |
| 5 | 375-379 | ATLQA | linker | 1-5 |
| 5 | 380-398 | AIPINFTISVTTEILPVSM | surface_glycoprotein@Aruba(NC_045512@Aruba:+: | 709-727 |
| | | | 21563-25384) | |
| 5 | 399-403 | ATLQA | linker | 1-5 |
| 5 | 404-412 | FFSNVTWFH | surface_glycoprotein(NC_045512:+:21563-25384) | 58-66 |
| 5 | 413-417 | ATLQA | linker | 1-5 |
| 5 | 418-432 | RIIPARARVECFDKF | helicase@Aruba(NC_045512@Aruba:+:16237-18039) | 332-346 |
| 5 | 433-437 | ATLQA | linker | 1-5 |
| 5 | 438-448 | RFQLHRSYLTP | surface_glycoprotein@Austria | 233-243 |
| | | | (NC_045512@Austria:+:21563-25384) | |
| 5 | 449-453 | ATLQA | linker | 1-5 |
| 5 | 454-477 | PINLVRDLPQGFSVLEPLVD | surface_glycoprotein@Belgium | 205-228 |
| | | LPIG | (NC_045512@Belgium:+:21563-25384) | |
| 5 | 478-482 | ATLQA | linker | 1-5 |
| 5 | 483-498 | FSNVTWFHAIHVSGTN | surface_glycoprotein@Peru(NC_045512@Peru:+: | 59-74 |
| | | | 21563-25384) | |
| 5 | 499-503 | ATLQA | linker | 1-5 |
| 5 | 504-511 | TDVVYRAF | RNA-dependent_RNA_polymerase@Aruba | 28-35 |
| | | | (NC_045512@Aruba:+:13442-13468+13468-16236) | |
| 5 | 512-516 | ATLQA | linker | 1-5 |
| 5 | 517-529 | NPVLPFNDGVYFA | surface_glycoprotein(NC_045512:+:21563-25384) | 81-93 |
| 5 | 530-534 | ATLQA | linker | 1-5 |
| 5 | 535-547 | AIKCVPQADVEWK | 3′-to-5′_exonuclease(NC_045512:+:18040-19620) | 337-349 |
| 5 | 548-552 | ATLQA | linker | 1-5 |
| 5 | 553-565 | ATNVVIKVCEFQF | surface_glycoprotein(NC_045512:+:21563-25384) | 123-135 |
| 5 | 566-570 | ATLQA | linker | 1-5 |
| 5 | 571-593 | RVDFCGKGYHLMSFPQSAPH | surface_glycoprotein(NC_045512:+:21563-25384) | 1039-1061 |
| | | GVV | | |
| 5 | 594-598 | ATLQA | linker | 1-5 |
| 5 | 599-613 | EFQFCNYPFLGVYHK | surface_glycoprotein@Norway | 129-143 |
| | | | (NC_045512@Norway:+:21563-25384) | |
| 5 | 614-618 | ATLQA | linker | 1-5 |
| 5 | 619-639 | AVLYQGVNCTEVPFAIHADQ | surface_glycoprotein@SmallAfrica | 605-625 |
| | | L | (NC_045512@SmallAfrica:+:21563-25384) | |
| 5 | 640-644 | ATLQA | linker | 1-5 |
| 5 | 645-656 | HEFCSQHTMLVK | RNA-dependent_RNA_polymerase(NC_045512:+: | 810-821 |
| | | | 13442-13468+13468-16236) | |
| 5 | 657-661 | ATLQA | linker | 1-5 |
| 5 | 662-677 | EEIIYNLLKDCPAVAK | RNA-dependent_RNA_polymerase@Senegal | 83-98 |
| | | | (NC_045512@Senegal:+:13442-13468+13468-16236) | |
| 5 | 678-682 | ATLQA | linker | 1-5 |
| 5 | 683-694 | ALGKLQDVVNQN | surface_glycoprotein(NC_045512:+:21563-25384) | 944-955 |
| 5 | 695-699 | ATLQA | linker | 1-5 |
| 5 | 700-722 | TQLYLGGMSYYCKPHKPPIS | helicase@Iceland(NC_045512@Iceland:+: | 61-83 |
| | | FPL | 16237-18039) | |
| 5 | 723-727 | ATLQA | linker | 1-5 |
| 5 | 728-738 | VLYQGVNCTEV | surface_glycoprotein@Aruba | 606-616 |
| | | | (NC_045512@Aruba:+:21563-25384) | |
| 5 | 739-743 | ATLQA | linker | 1-5 |
| 5 | 744-753 | FCLEASFNYL | nsp3(NC_045512:+:2720-8554) | 1391-1400 |
| 5 | 754-758 | ATLQA | linker | 1-5 |
| 5 | 759-766 | PVLKGVKL | surface_glycoprotein@Aruba | 1259-1266 |
| | | | (NC_045512@Aruba:+:21563-25384) | |
| 5 | 767-771 | ATLQA | linker | 1-5 |
| 5 | 772-779 | FTGCVIAW | surface_glycoprotein(NC_045512:+:21563-25384) | 429-436 |
| 5 | 780-784 | ATLQA | linker | 1-5 |
| 5 | 785-794 | KQYGDCLGDI | surface_glycoprotein(NC_045512:+:21563-25384) | 835-844 |
| 6 | 1-18 | YNLLKDCPAVAKHDFFKF | RNA-dependent_RNA_polymerase@Aruba | 87-104 |
| | | | (NC_045512@Aruba:+:13442-13468+13468-16236) | |
| 6 | 19-23 | ATLQA | linker | 1-5 |
| 6 | 24-44 | HPNCVNCSDDRCILHCANFN | RNA-dependent_RNA_polymerase@Aruba | 295-315 |
| | | V | (NC_045512@Aruba:+:13442-13468+13468-16236) | |
| 6 | 45-49 | ATLQA | linker | 1-5 |
| 6 | 50-58 | ASQGLLASI | RNA-dependent_RNA_polymerase@Austria | 771-779 |
| | | | (NC_045512@Austria:+:13442-13468+13468-16236) | |
| 6 | 59-63 | ATLQA | linker | 1-5 |
| 6 | 64-80 | VPQEHYVRITGLYPTLN | helicase@Aruba(NC_045512@Aruba:+:16237-18039) | 241-257 |
| 6 | 81-85 | ATLQA | linker | 1-5 |
| 6 | 86-95 | ETFVTHSKGL | nsp2(NC_045512:+:806-2719) | 527-536 |
| 6 | 96-100 | ATLQA | linker | 1-5 |
| 6 | 101-117 | LECNVKTTEVVGDIILK | nsp3(NC_045512:+:2720-8554) | 1244-1260 |
| 6 | 118-122 | ATLQA | linker | 1-5 |
| 6 | 123-136 | TKDVVECLKLSHQS | nsp3(NC_045512:+:2720-8554) | 1830-1843 |
| 6 | 137-141 | ATLQA | linker | 1-5 |
| 6 | 142-151 | GVITHDVSSA | helicase(NC_045512:+:16237-18039) | 478-487 |
| 6 | 152-156 | ATLQA | linker | 1-5 |
| 6 | 157-165 | MPVCVETKA | nsp3(NC_045512:+:2720-8554) | 571-579 |
| 6 | 166-170 | ATLQA | linker | 1-5 |
| 6 | 171-184 | TCFSVAALTNNVAF | RNA-dependent_RNA_polymerase@Aruba | 394-407 |
| | | | (NC_045512@Aruba:+:13442-13468+13468-16236) | |
| 6 | 185-189 | ATLQA | linker | 1-5 |
| 6 | 190-205 | SRIIPARARVDCFDKF | helicase@Brazil(NC_045512@Brazil:+:16237-18039) | 331-346 |
| 6 | 206-210 | ATLQA | linker | 1-5 |
| 6 | 211-219 | RSVYPVASP | nsp2(NC_045512:+:806-2719) | 121-129 |
| 6 | 220-224 | ATLQA | linker | 1-5 |
| 6 | 225-242 | CANFNVLFSTVFPFTSFG | RNA-dependent_RNA_polymerase@Nigeria | 310-327 |
| | | | (NC_045512@Nigeria:+:13442-13468+13468-16236) | |
| 6 | 243-247 | ATLQA | linker | 1-5 |
| 6 | 248-261 | LKEILVTYNCCDDD | RNA-dependent_RNA_polymerase(NC_045512:+: | 142-155 |
| | | | 13442-13468+13468-16236) | |
| 6 | 262-266 | ATLQA | linker | 1-5 |
| 6 | 267-288 | SQHTMLVKQFDDYVYLPYPD | RNA-dependent_RNA_polymerase@Estonia | 814-835 |
| | | PS | (NC_045512@Estonia:+:13442-13468+13468-16236) | |
| 6 | 289-293 | ATLQA | linker | 1-5 |
| 6 | 294-301 | MADLVYAL | RNA-dependent_RNA_polymerase(NC_045512:+: | 124-131 |
| | | | 13442-13468+13468-16236) | |
| 6 | 302-306 | ATLQA | linker | 1-5 |
| 6 | 307-315 | VTANVNALL | RNA-dependent_RNA_polymerase(NC_045512:+: | 700-708 |
| | | | 13442-13468+13468-16236) | |
| 6 | 316-320 | ATLQA | linker | 1-5 |
| 6 | 321-340 | RAKHYVYIGDPAQLPAPRTL | helicase(NC_045512:+:16237-18039) | 392-411 |
| 6 | 341-345 | ATLQA | linker | 1-5 |
| 6 | 346-358 | WNVVRIKIVQMLS | 3′-to-5′_exonuclease(NC_045512:+:18040-19620) | 159-171 |
| 6 | 359-363 | ATLQA | linker | 1-5 |
| 6 | 364-383 | FPQSAPHGVVFLHVTYVPAQ | surface_glycoprotein@Aruba(NC_045512@Aruba:+: | 1048-1067 |
| | | | 21563-25384) | |
| 6 | 384-388 | ATLQA | linker | 1-5 |
| 6 | 389-403 | ISNCVADYSVLYNSA | surface_glycoprotein@Aruba(NC_045512@Aruba:+: | 354-368 |
| | | | 21563-25384) | |
| 6 | 404-408 | ATLQA | linker | 1-5 |
| 6 | 409-424 | TGLHPTQAPTHLSVDT | 3′-to-5′_exonuclease(NC_045512:+:18040-19620) | 16-31 |
| 6 | 425-429 | ATLQA | linker | 1-5 |
| 6 | 430-443 | FFSNVTWFHAIHVS | surface_glycoprotein@Bangladesh | 58-71 |
| | | | (NC_045512@Bangladesh:+:21563-25384) | |
| 6 | 444-448 | ATLQA | linker | 1-5 |
| 6 | 449-461 | ISSVLNDILSRLD | surface_glycoprotein@Bangladesh | 972-984 |
| | | | (NC_045512@Bangladesh:+:21563-25384) | |
| 6 | 462-466 | ATLQA | linker | 1-5 |
| 6 | 467-481 | EFQFCNDPFLGVYYH | surface_glycoprotein@Chile | 132-146 |
| | | | (NC_045512@Chile:+:21563-25384) | |
| 6 | 482-486 | ATLQA | linker | 1-5 |
| 6 | 487-501 | FSVAALTNNVAFQTV | RNA-dependent_RNA_polymerase(NC_045512:+: | 396-410 |
| | | | 13442-13468+13468-16236) | |
| 6 | 502-506 | ATLQA | linker | 1-5 |
| 6 | 507-519 | LLNVPLHGTILTR | membrane_glycoprotein(NC_045512:+:26523-27191) | 119-131 |
| 6 | 520-524 | ATLQA | linker | 1-5 |
| 6 | 525-543 | ECAQVLSETVMCGGSLYVK | RNA-dependent_RNA_polymerase@Aruba | 658-676 |
| | | | (NC_045512@Aruba:+:13442-13468+13468-16236) | |
| 6 | 544-548 | ATLQA | linker | 1-5 |
| 6 | 549-564 | GSNVFQTRAGCLIGAE | surface_glycoprotein(NC_045512:+:21563-25384) | 639-654 |
| 6 | 565-569 | ATLQA | linker | 1-5 |
| 6 | 570-579 | SSVLHSTQDF | surface_glycoprotein@Norway | 45-54 |
| | | | (NC_045512@Norway:+:21563-25384) | |
| 6 | 580-584 | ATLQA | linker | 1-5 |
| 6 | 585-610 | YRLANECAQVLSEIVMCGGS | RNA-dependent_RNA_polymerase@Canada | 653-678 |
| | | LYVKPG | (NC_045512@Canada:+:13442-13468+13468-16236) | |
| 6 | 611-615 | ATLQA | linker | 1-5 |
| 6 | 616-637 | NVVIKVCEFQFCNYPFLGVY | surface_glycoprotein@Brazil | 125-146 |
| | | YH | (NC_045512@Brazil:+:21563-25384) | |
| 6 | 638-642 | ATLQA | linker | 1-5 |
| 6 | 643-654 | YPSLETIQITIS | nsp3(NC_045512:+:2720-8554) | 1483-1494 |
| 6 | 655-659 | ATLQA | linker | 1-5 |
| 6 | 660-673 | TSGVGYQPYRVVVL | surface_glycoprotein@Aruba | 496-509 |
| | | | (NC_045512@Aruba:+:21563-25384) | |
| 6 | 674-678 | ATLQA | linker | 1-5 |
| 6 | 679-693 | VNCTEVPVAIHADQL | surface_glycoprotein(NC_045512:+:21563-25384) | 615-629 |
| 6 | 694-698 | ATLQA | linker | 1-5 |
| 6 | 699-706 | ISNCVADY | surface_glycoprotein(NC_045512:+:21563-25384) | 358-365 |
| 6 | 707-711 | ATLQA | linker | 1-5 |
| 6 | 712-723 | IEVQGYKSVNIT | nsp3(NC_045512:+:2720-8554) | 13-24 |
| 6 | 724-728 | ATLQA | linker | 1-5 |
| 6 | 729-742 | FGTVYEKLKPVLDW | nsp2(NC_045512:+:806-2719) | 437-450 |
| 6 | 743-747 | ATLQA | linker | 1-5 |
| 6 | 748-762 | AYYVGYLQPRTFLLK | surface_glycoprotein@Aruba | 260-274 |
| | | | (NC_045512@Aruba:+:21563-25384) | |
| 6 | 763-767 | ATLQA | linker | 1-5 |
| 6 | 768-787 | PINLVRGLPQGFSVLEPLVD | surface_glycoprotein@SmallAfrica | 209-228 |
| | | | (NC_045512@SmallAfrica:+:21563-25384) | |
| 6 | 788-792 | ATLQA | linker | 1-5 |
| 6 | 793-805 | FQTLLALHRSYLT | surface_glycoprotein@Aruba | 234-246 |
| | | | (NC_045512@Aruba:+:21563-25384) | |

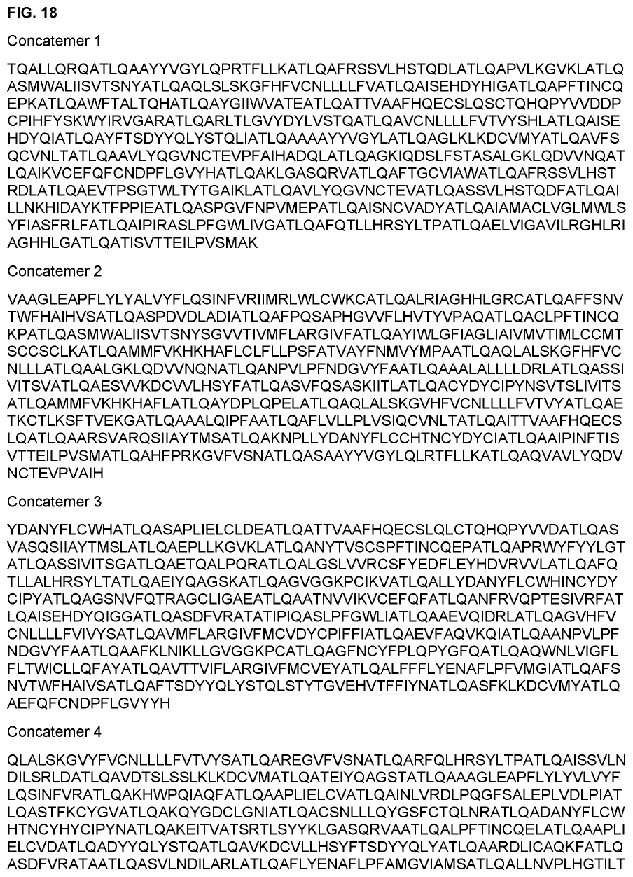

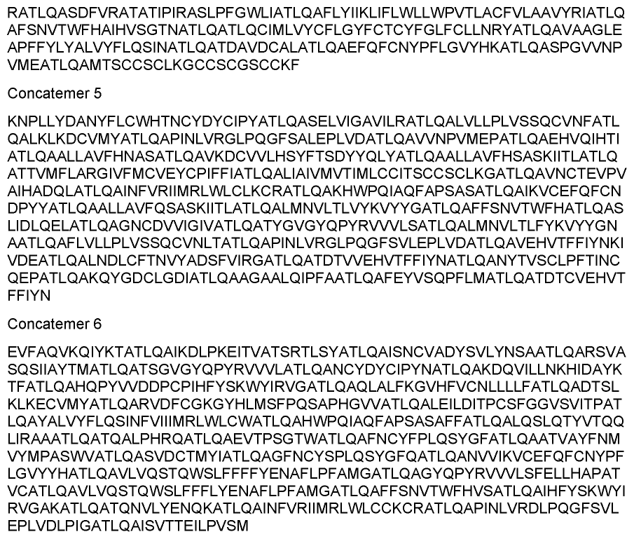

In certain embodiments, the vaccine comprises or expresses at least one of the concatemers of epitopes from SARS-COV-2 with intervening linker sequences (and as set forth in FIG. 18). In certain embodiments, the vaccine comprises or expresses at least two of the concatemers set forth below. In certain embodiments, the vaccine comprises or expresses three of the concatemers set forth below. In certain embodiments, the vaccine comprises or expresses four of the concatemers set forth below. In certain embodiments, the vaccine comprises or expresses five of the concatemers set forth below. In certain embodiments, the vaccine comprises or expresses all the concatemers set forth below.

TABLE 8

| concatemer | pos_in_concatemer | epitope | gebe | pos_in_gene |
|---|---|---|---|---|
| 1 | 1-8 | TQALLQRQ | nucleocapsid_phosphoprotein@CostaRica(NC_045512@CostaRica: + : | 379-386 |
| | | | 28274-29533) | |
| 1 | 9-13 | ATLQA linker 1-5 | | |
| 1 | 14-28 | AYYVGYLQPRTFLLK | surface_glycoprotein@Aruba(NC_045512@Aruba: + : 21563-25384) | 260-274 |
| 1 | 29-33 | ATLQA linker 1-5 | | |
| 1 | 34-45 | FRSSVLHSTQDL | surface_glycoprotein@Aruba(NC_045512@Aruba: + : 21563-25384) | 43-54 |
| 1 | 46-50 | ATLQA linker 1-5 | | |
| 1 | 51-58 | PVLKGVKL | surface_glycoprotein@Aruba(NC_045512@Aruba: + : 21563-25384) | 1259-1266 |
| 1 | 59-63 | ATLQA linker 1-5 | | |
| 1 | 64-76 | SMWALIISVTSNY | nsp6@Aruba(NC_045512@Aruba: + : 10973-11842) | 160-172 |
| 1 | 77-81 | ATLQA linker 1-5 | | |
| 1 | 82-100 | QLSLSKGFHFVCNLLLFV | ORF3a_protein@Estonia(NC_045512@Estonia: + : 25393-26220) | 70-88 |
| 1 | 101-105 | ATLQA linker 1-5 | | |
| 1 | 106-114 | ISEHDYHIG | ORF3a_protein@NorthMacedonia(NC_045512@NorthMacedonia: + : 25393-26220) | 179-187 |
| 1 | 115-119 | ATLQA linker 1-5 | | |
| 1 | 120-129 | PFTINCQEPK | ORF8_protein@India(NC_045512@India: + : 27894-28259) | 85-94 |
| 1 | 130-134 | ATLQA linker 1-5 | | |
| 1 | 135-142 | WFTALTQH | nucleocapsid_phosphoprotein(NC_045512: + : 28274-29533) | 52-59 |
| 1 | 143-147 | ATLQA linker 1-5 | | |
| 1 | 148-156 | YGIIWVATE | nucleocapsid_phosphoprotein@SmallSouthAmerica(NC_045512@ | 128-136 |
| | | | SmallSouthAmerica: + : 28274-29533) | |
| 1 | 157-161 | ATLQA linker 1-5 | | |
| 1 | 162-203 | TTVAAFHQECSLQSCTQHQPYVVDDPCPIHFY | ORF8_protein@Bangladesh(NC_045512@Bangladesh: + : 27894-28259) | 11-52 |
| | | SKWYIRVGAR | | |
| 1 | 204-208 | ATLQA linker 1-5 | | |
| 1 | 209-222 | RLTLGVYDYLVSTQ | nsp6(NC_045512: + : 10973-11842) | 236-249 |
| 1 | 223-227 | ATLQA linker 1-5 | | |
| 1 | 228-242 | VCNLLLLFVTVYSHL | ORF3a_protein@Croatia(NC_045512@Croatia: + : 25393-26220) | 80-94 |
| 1 | 243-247 | ATLQA linker 1-5 | | |
| 1 | 248-255 | ISEHDYQI | ORF3a_protein@Aruba(NC_045512@Aruba: + : 25393-26220) | 179-186 |
| 1 | 256-260 | ATLQA linker 1-5 | | |
| 1 | 261-275 | YFTSDYYQLYSTQLI | ORF3a_protein@Indonesia(NC_045512@Indonesia: + : 25393-26220) | 206-220 |
| 1 | 276-280 | ATLQA linker 1-5 | | |
| 1 | 281-289 | AAAYYVGYL | surface_glycoprotein(NC_045512: + : 21563-25384) | 262-270 |
| 1 | 290-294 | ATLQA linker 1-5 | | |
| 1 | 295-304 | GLKLKDCVMY | nsp6@Peru(NC_045512@Peru: + : 10973-11842) | 107-116 |
| 1 | 305-309 | ATLQA linker 1-5 | | |
| 1 | 310-318 | VFSQCVNLT | surface_glycoprotein@Egypt(NC_045512@Egypt: + : 21563-25384) | 11-19 |
| 1 | 319-323 | ATLQA linker 1-5 | | |
| 1 | 324-344 | AVLYQGVNCTEVPFAIHADQL | surface_glycoprotein@SmallAfrica(NC_045512@SmallAfrica: + : 21563-25384) | 605-625 |
| 1 | 345-349 | ATLQA linker 1-5 | | |
| 1 | 350-372 | GKIQDSLFSTASALGKLQDVVNQ | surface_glycoprotein@NorthMacedonia(NC_045512@NorthMacedonia: + : | 928-950 |
| | | | 21563-25384) | |
| 1 | 373-377 | ATLQA linker 1-5 | | |
| 1 | 378-395 | IKVCEFQFCNDPFLGVYH | surface_glycoprotein@Aruba(NC_045512@Aruba: + : 21563-25384) | 125-142 |
| 1 | 396-400 | ATLQA linker 1-5 | | |
| 1 | 401-408 | KLGASQRV | membrane_glycoprotein(NC_045512: + : 26523-27191) | 180-187 |
| 1 | 409-413 | ATLQA linker 1-5 | | |
| 1 | 414-421 | FTGCVIAW | surface_glycoprotein(NC_045512: + : 21563-25384) | 429-436 |
| 1 | 422-426 | ATLQA linker 1-5 | | |
| 1 | 427-438 | FRSSVLHSTRDL | surface_glycoprotein@Nigeria(NC_045512@Nigeria: + : 21563-25384) | 43-54 |
| 1 | 439-443 | ATLQA linker 1-5 | | |
| 1 | 444-460 | EVTPSGTWLTYTGAIKL | nucleocapsid_phosphoprotein@Bulgaria(NC_045512@Bulgaria: + : | 323-339 |
| | | | 28274-29533) | |
| 1 | 461-465 | ATLQA linker 1-5 | | |
| 1 | 466-476 | VLYQGVNCTEV | surface_glycoprotein@Aruba(NC_045512@Aruba: + : 21563-25384) | 606-616 |
| 1 | 477-481 | ATLQA linker 1-5 | | |
| 1 | 482-491 | SSVLHSTQDF | surface_glycoprotein@Norway(NC_045512@Norway: + : 21563-25384) | 45-54 |
| 1 | 492-496 | ATLQA linker 1-5 | | |
| 1 | 497-513 | ILLNKHIDAYKTFPPIE | nucleocapsid_phosphoprotein@Chile(NC_045512@Chile: + : 28274-29533) | 351-367 |
| 1 | 514-518 | ATLQA linker 1-5 | | |
| 1 | 519-529 | SPGVFNPVMEP | ORF3a_protein@Colombia(NC_045512@Colombia: + : 25393-26220) | 252-262 |
| 1 | 530-534 | ATLQA linker 1-5 | | |
| 1 | 535-542 | ISNCVADY | surface_glycoprotein(NC_045512: + : 21563-25384) | 358-365 |
| 1 | 543-547 | ATLQA linker 1-5 | | |
| 1 | 548-569 | IAMACLVGLMWLSYFIASFRLF | membrane_glycoprotein@Aruba(NC_045512@Aruba: + : 26523-27191) | 82-103 |
| 1 | 570-574 | ATLQA linker 1-5 | | |
| 1 | 575-589 | IPIRASLPFGWLIVG | ORF3a_protein@Iceland(NC_045512@Iceland: + : 25393-26220) | 35-49 |
| 1 | 590-594 | ATLQA linker 1-5 | | |
| 1 | 595-606 | FQTLLHRSYLTP | surface_glycoprotein@SmallAsia(NC_045512@SmallAsia: + : 21563-25384) | 230-241 |
| 1 | 607-611 | ATLQA linker 1-5 | | |
| 1 | 612-632 | ELVIGAVILRGHLRIAGHHLG | membrane_glycoprotein@Aruba(NC_045512@Aruba: + : 26523-27191) | 137-157 |
| 1 | 633-637 | ATLQA linker 1-5 | | |
| 1 | 638-652 | TISVTTEILPVSMAK | membrane_glycoprotein@Mexico(NC_045512@Mexico: + : 26523-27191) | 719-733 |
| 2 | 1-37 | VAAGLEAPFLYLYALVYFLQSINFVRIIMRLW | ORF3a_protein@Aruba(NC_045512@Aruba: + : 25393-26220) | 97-133 |
| | | LCWKC | | |
| 2 | 38-42 | ATLQA linker 1-5 | | |
| 2 | 43-53 | LRIAGHHLGRC | membrane_glycoprotein(NC_045512: + : 26523-27191) | 149-159 |
| 2 | 54-58 | ATLQA linker 1-5 | | |
| 2 | 59-72 | FFSNVTWFHAIHVS | surface_glycoprotein@Bangledesh(NC_045512@Bangledesh: + : 21563-25384) | 58-71 |
| 2 | 73-77 | ATLQA linker 1-5 | | |
| 2 | 78-86 | SPDVDLADI | surface_glycoprotein@Chile(NC_045512@Chile: + : 21563-25384) | 1161-1169 |
| 2 | 87-91 | ATLQA linker 1-5 | | |
| 2 | 92-111 | FPQSAPHGVVFLHVTYVPAQ | surface_glycoprotein@Aruba(NC_045512@Aruba: + : 21563-25384) | 1048-1067 |
| 2 | 112-116 | ATLQA linker 1-5 | | |
| 2 | 117-127 | CLPFTINCQKP | ORF8_protein@Brazil(NC_045512@Brazil: + : 27894-28259) | 83-93 |
| 2 | 128-132 | ATLQA linker 1-5 | | |
| 2 | 133-161 | MWALIISVTSNYSGVVTIVMFLARGIVF | nsp6@SmallSouthAmerica(NC_045512@SmallSouthAmerical: + : | 160-188 |
| 2 | 162-166 | ATLQA linker 1-5 | | |
| 2 | 167-197 | YIWLGFIAGLIAIVMVTIMLCCMTSCCSCLK | surface_glycoprotein@Aruba(NC_045512@Aruba: + : 21563-25384) | 1121-1241 |
| 2 | 198-202 | ATLQA linker 1-5 | | |
| 2 | 203-234 | MMFVKHKHAFLCLFLLPSFATVAYFNMVYMPA | nsp6@Indonesia(NC_045512@Indonesia: + : 10973-11842) | 57-88 |
| 2 | 235-239 | ATLQA linker 1-5 | | |
| 2 | 240-255 | QLALSKGFHFVCNLLL | ORF3a_protein@Iceland(NC_045512@Iceland: + : 25393-26220) | 70-85 |
| 2 | 256-260 | ATLQA linker 1-5 | | |
| 2 | 261-272 | ALGKLQDVVNQN | surface_glycoprotein(NC_045512: + : 21563-25384) | 944-955 |
| 2 | 273-277 | ATLQA linker 1-5 | | |
| 2 | 278-290 | NPVLPFNDGVYFA | surface_glycoprotein(NC_045512: + : 21563-25384) | 81-93 |
| 2 | 291-295 | ATLQA linker 1-5 | | |
| 2 | 296-306 | AALALLLLDRL | nucleocapsid_phosphoprotein@Aruba(NC_045512@Aruba: + : 28274-29533) | 217-227 |
| 2 | 307-311 | ATLQA linker 1-5 | | |
| 2 | 312-319 | SSIVITSV | ORF3a_protein@Aruba(NC_045512@Aruba: + : 25393-26220) 165-172 | |
| 2 | 320-324 | ATLQA linker 1-5 | 1-5 | |
| 2 | 325-338 | ESVVKDCVVLHSYF | ORF3a_protein@CostaRica(NC_045512@CostaRica: + : 25393-26220) | 194-207 |
| 2 | 339-343 | ATLQA linker 1-5 | | |
| 2 | 344-355 | SVFQSASKIITL | ORF3a_protein@Iceland(NC_045512@Iceland: + : 25393-26220) | 54-65 |
| 2 | 356-360 | ATLQA linker 1-5 | | |
| 2 | 361-379 | CYDYCIPYNSVTSLIVITS | ORF3a_protein@Netherlands(NC_045512@Netherlands: + : 25393-26220) | 153-171 |
| 2 | 380-384 | ATLQA linker 1-5 | | |
| 2 | 385-395 | MMFVKHKHAFL | nsp6@Aruba(NC_045512@Aruba: + : 10973-11842) | 57-67 |
| 2 | 396-400 | ATLQA linker 1-5 | | |
| 2 | 401-408 | YDPLQPEL | surface_glycoprotein(NC_045512: + : 21563-25384) | 1138-1145 |
| 2 | 409-413 | ATLQA linker 1-5 | | |
| 2 | 414-435 | QLALSKGVHFVCNLLLLFVTVY | ORF3a_protein@Australia(NC_045512@Australia: + : 25393-26220) | 70-91 |
| 2 | 436-440 | ATLQA linker 1-5 | | |
| 2 | 441-454 | ETKCTLKSFTVEKG | surface_glycoprotein@Aruba(NC_045512@Aruba: + : 21563-25384) | 294-307 |
| 2 | 455-459 | ATLQA linker 1-5 | | |
| 2 | 460-467 | AALQIPFA | surface_glycoprotein(NC_045512: + : 21563-25384) | 892-899 |
| 2 | 468-472 | ATLQA linker 1-5 | | |
| 2 | 473-488 | FLVLLPLVSIQCVNLT | surface_glycoprotein@SmallNorthAmerica(NC_045512@SmallNorthAmerica: + : | 4-19 |
| | | | 21563-25384) | |
| 2 | 494-507 | ITTVAAFHQECSLQ | ORF8_protein@Aruba(NC_045512@Aruba: + : 27894-28259) | 10-23 |
| 2 | 508-512 | ATLQA linker 1-5 | | |
| 2 | 513-527 | ARSVARQSIIAYTMS | surface_glycoprotein@Mozambique(NC_045512@Mozambique: + : 21563-25384) | 680-694 |
| 2 | 528-532 | ATLQA linker 1-5 | | |
| 2 | 533-555 | KNPLLYDANYFLCCHTNCYDYCI | ORF3a_protein@Senegal(NC_045512@Senegal: + : 25393-26220) | 136-158 |
| 2 | 556-560 | ATLQA linker 1-5 | | |
| 2 | 561-579 | AIPINFTISVTTEILPVSM | surface_glycoprotein@Aruba(NC_045512@Aruba: + : 21563-25384) | 709-727 |
| 2 | 580-584 | ATLQA linker 1-5 | | |
| 2 | 585-595 | HFPRKGVFVSN | surface_glycoprotein@SmallAsia(NC_045512@SmallAsia: + : 21563-25384) | 1078- |
| 2 | 596-600 | ATLQA linker 1-5 | | |
| 2 | 601-617 | SAAYYVGYLQLRTFLLK | surface_glycoprotein@Italy(NC_045512@Italy: + : 21563-25384) | 258-274 |
| 2 | 618-622 | ATLQA linker 1-5 | | |
| 2 | 623-641 | QVAVLYQDVNCTEVPVAIH | surface_glycoprotein@China(NC_045512@China: + : 21563-25384) | 607-625 |
| 3 | 1-10 | YDANYFLCWH | ORF3a_protein(NC_045512: + : 25393-26220) | 141-150 |
| 3 | 11-15 | ATLQA linker 1-5 | | |
| 3 | 16-26 | SAPLIELCLDE | ORF8_protein@Russia(NC_045512@Russia: + : 27894-28259) | 54-64 |
| 3 | 27-31 | ATLQA linker 1-5 | | |
| 3 | 32-55 | TTVAAFHQECSLQLCTQHQPYVVD | ORF8_protein@USA(NC_045512@USA: + : 27894-28259) | 11-34 |
| 3 | 56-60 | ATLQA linker 1-5 | | |
| 3 | 61-74 | SVASQSIIAYTMSL | surface_glycoprotein@Austria(NC_045512@Austria: + : 21563-25384) | 678-691 |
| 3 | 75-79 | ATLQA linker 1-5 | | |
| 3 | 80-88 | EPLLKGVKL | surface_glycoprotein@Croatia(NC_045512@Croatia: + : 21563-25384) | 1258-1266 |
| 3 | 89-93 | ATLQA linker 1-5 | | |
| 3 | 94-109 | NYTVSCSPFTINCQEP | ORF8_protein@CostaRica(NC_045512@CostaRica: + : 27894-28259) | 78-93 |
| 3 | 110-114 | ATLQA linker 1-5 | | |
| 3 | 115-124 | PRWYFYYLGT | nucleocapsid_phosphoprotein@Aruba(NC_045512@Aruba: + : 28274-29533) | 106-115 |
| 3 | 125-129 | ATLQA linker 1-5 | | |
| 3 | 130-137 | SSIVITSG | ORF3a_protein@SouthKorea(NC_045512@SouthKorea: + : 25393-26220) | 165-172 |
| 3 | 138-142 | ATLQA linker 1-5 | | |
| 3 | 143-150 | ETQALPQR | nucleocapsid_phosphoprotein@SmallNorthAmerica(NC_04551@ | 378-385 |
| 3 | 151-155 | ATLQA linker 1-5 | | |
| 3 | 156-179 | LGSLVVRCSFYEDFLEYHDVRVVL | ORF8_protein(NC_045512: + : 27894-28259) | 95-118 |
| 3 | 180-184 | ATLQA linker 1-5 | | |
| 3 | 185-197 | FQTLLALHRSYLT | surface_glycoprotein@Aruba(NC_045512@Aruba: + : 21563-25384) | 234-246 |
| 3 | 198-202 | ATLQA linker 1-5 | | |
| 3 | 203-210 | EIYQAGSK | surface_glycoprotein@Mexico(NC_045512@Mexico: + : 21563-25384) | 471-478 |
| 3 | 211-215 | ATLQA linker 1-5 | | |
| 3 | 216-225 | GVGGKPCIKV | nsp6@Aruba(NC_045512@Aruba: + : 10973-11842) | 274-283 |
| 3 | 226-230 | ATLQA linker 1-5 | | |
| 3 | 231-252 | LLYDANYFLCWHINCYDYCIPY | ORF3a_protein@Estonia(NC_045512@Estonia: + : 25393-26220) | 139-160 |
| 3 | 253-257 | ATLQA linker 1-5 | | |
| 3 | 258-273 | GSNVFQTRAGCLIGAE | surface_glycoprotein(NC_045512: + : 21563-25384) | 639-654 |
| 3 | 274-278 | ATLQA linker 1-5 | | |
| 3 | 279-291 | ATNVVIKVCEFQF | surface_glycoprotein(NC_045512: + : 21563-25384) | 123-135 |
| 3 | 292-296 | ATLQA linker 1-5 | | |
| 3 | 297-309 | NFRVQPTESIVRF | surface_glycoprotein(NC_045512: + : 21563-25384) | 317-329 |
| 3 | 310-314 | ATLQA linker 1-5 | | |
| 3 | 315-324 | ISEHDYQIGG | ORF3a_protein@FrenchGuiana(NC_045512@FrenchGuiana: + : 25393-26220) | 179-188 |
| 3 | 325-329 | ATLQA linker 1-5 | | |
| 3 | 330-351 | SDFVRATATIPIQASLPFGWL | ORF3a_protein@Aruba(NC_045512@Aruba: + : 25393-26220) | 26-47 |
| 3 | 352-356 | ATLQA linker 1-5 | | |
| 3 | 357-364 | AEVQIDRL | surface_glycoprotein(NC_045512: + : 21563-25384) | 989-996 |
| 3 | 365-369 | ATLQA linker 1-5 | | |
| 3 | 370-386 | GVHFVCNLLLLFVIVYS | ORF3a_protein@Malaysia(NC_045512@Malaysia: + : 25393-26220) | 76-92 |
| 3 | 387-391 | ATLQA linker 1-5 | | |
| 3 | 392-412 | VMFLARGIVFMCVDYCPIFFI | nsp6@Cyprus(NC_045512@Cyprus: + : 10973-11842) | 179-199 |
| 3 | 413-417 | ATLQA linker 1-5 | | |
| 3 | 418-426 | EVFAQVKQI | surface_glycoprotein(NC_045512: + : 21563-25384) | 780-788 |
| 3 | 427-431 | ATLQA linker 1-5 | | |
| 3 | 432-445 | ANPVLPFNDGVYFA | surface_glycoprotein@Austria(NC_045512@Austria: + : 21563-25384) | 77-90 |
| 3 | 446-450 | ATLQA linker 1-5 | | |
| 3 | 451-466 | AFKLNIKLLGVGGKPC | nsp6(NC_045512: + : 10973-11842) | 268-283 |
| 3 | 467-471 | ATLQA linker 1-5 | | |
| 3 | 472-485 | GFNCYFPLQPYGFQ | surface_glycoprotein@SmallSouthAmerica(NC_045512@SmallSouthAmerica: + : | 481-494 |
| 3 | 486-490 | ATLQA linker 1-5 | | |
| 3 | 491-511 | QWNLVIGFLFLTWICLLQFAY | membrane_glycoprotein@Aruba(NC_045512@Aruba: + : 26523-27191) | 19-39 |
| 3 | 512-516 | ATLQA linker 1-5 | | |
| 3 | 517-534 | VTTVIFLARGIVFMCVEY | nsp6@Malaysia(NC_045512@Malaysia: + : 10973-11842) | 179-196 |
| 3 | 535-539 | ATLQA linker 1-5 | | |
| 3 | 540-556 | LFFFLYENAFLPFVMGI | nsp6@SmallSouthAmerica(NC_045512@SmallSouthAmerica: + : 10973-11842) | 33-49 |
| 3 | 557-561 | ATLQA linker 1-5 | | |
| 3 | 562-573 | FSNVTWFHAIVS | surface_glycoprotein@Ghana(NC_045512@Ghana: + : 21563-25384) | 59-70 |
| 3 | 574-578 | ATLQA linker 1-5 | | |
| 3 | 579-606 | FTSDYYQLYSTQLSTYTGVEHVTFFIYN | ORF3a_protein@Latvia(NC_045512@Latvia: + : 25393-26220) | 207-234 |
| 3 | 607-611 | ATLQA linker 1-5 | | |
| 3 | 612-621 | SFKLKDCVMY | nsp6@Malaysia(NC_045512@Malaysia: + : 10973-11842) | 107-116 |
| 3 | 622-626 | ATLQA linker 1-5 | | |
| 3 | 627-641 | EFQFCNDPFLGVYYH | surface_glycoprotein@Chile(NC_045512@Chile: + : 21563-25384) | 132-146 |
| 4 | 1-23 | QLALSKGVYFVCNLLLLFVTVYS | ORF3a_protein@Aruba(NC_045512@Aruba: + : 25393-26220) | 70-92 |
| 4 | 24-28 | ATLQA inker | | |
| 4 | 29-36 | REGVFVSN | surface_glycoprotein@Aruba(NC_045512@Aruba: + : 21563-25384) | 1087-1094 |
| 4 | 37-41 | ATLQA linker 1-5 | | |
| 4 | 42-52 | RFQLHRSYLTP | surface_glycoprotein@Austria(NC_045512@Austria: + : 21563-25384) | 233-243 |
| 4 | 53-57 | ATLQA linker 1-5 | | |
| 4 | 58-70 | ISSVLNDILSRLD | surface_glycoprotein@Bangladesh(NC_045512@Bangladesh: + : 21563-25384) | 972-984 |
| 4 | 71-75 | ATLQA linker 1-5 | | |
| 4 | 76-90 | VDTSLSSLKLKDCVM | nsp6@Latvia(NC_045512@Latvia: + : 10973-11842) | 101-115 |
| 4 | 91-95 | ATLQA linker 1-5 | | |
| 4 | 96-104 | TEIYQAGST | surface_glycoprotein@Aruba(NC_045512@Aruba: + : 21563-25384) | 466-474 |
| 4 | 105-109 | ATLQA linker 1-5 | | |
| 4 | 110-134 | AAGLEAPFLYLYVLVYFLQSINFVR | ORF3a_protein@Norway(NC_045512@Norway: + : 25393-26220) | 98-122 |
| 4 | 135-139 | ATLQA linker 1-5 | | |
| 4 | 140-148 | KHWPQIAQF | nucleocapsid_phosphoprotein(NC_045512: + : 28274-29533) | 299-307 |
| 4 | 149-153 | ATLQA linker 1-5 | | |
| 4 | 154-161 | APLIELCV | ORF8_protien(NC_045512: + : 27894-28259) | 55-62 |
| 4 | 162-166 | ATLQA linker 1-5 | | |
| 4 | 167-188 | INLVRDLPQGFSALEPLVDLPI | surface_glycoprotein@Aruba(NC_045512@Aruba: + : 21563-25384) | 206-227 |
| 4 | 189-193 | ATLQA linker 1-5 | | |
| 4 | 194-201 | STFKCYGV | surface_glycoprotein@Aruba(NC_045512@Aruba: + : 21563-25384) | 371-378 |
| 4 | 202-206 | ATLQA linker 1-5 | | |
| 4 | 207-216 | KQYGDCLGNI | surface_glycoprotein@Jordan(NC_045512@Jordan: + : 21563-25384) | 831-840 |
| 4 | 217-221 | ATLQA linker 1-5 | | |
| 4 | 222-238 | CSNLLLQYGSFCTQLNR | surface_glycoprotein(NC_045512: + : 21563-25384) | 749-765 |
| 4 | 239-243 | ATLQA linker 1-5 | | |
| 4 | 244-263 | DANYFLCWHTNCYHYCIPYN | ORF3a_protein@Indonesia(NC_045512@Indonesia: + : 25393-26220) | 142-161 |
| 4 | 264-268 | ATLQA linker 1-5 | | |
| 4 | 269-291 | KEITVATSRTLSYYKLGASQRVA | membrane_glycoprotein@Aruba(NC_045512@Aruba: + : 26523-27191) | 166-188 |
| 4 | 292-296 | ATLQA linker 1-5 | | |
| 4 | 297-306 | LPFTINCQEL | ORF8_protien@Mozambique(NC_045512@Mozambique: + : 27894-28259) | 84-93 |
| 4 | 307-311 | ATLQA linker 1-5 | | |
| 4 | 312-320 | APLEILCVD | ORF8_protien@Bangladesh(NC_045512@Bangladesh: + : 27894-28259) | 55-63 |
| 4 | 321-325 | ATLQA linker 1-5 | | |
| 4 | 326-334 | DYYQLYSTQ | ORF3a_protein(NC_045512: + : 25393-26220) | 210-218 |
| 4 | 335-339 | ATLQA linker 1-5 | | |
| 4 | 340-358 | VKDCVLLHSYFTSDYYQLY | ORF3a_protein@Belgium(NC_045512@Belgium: + : 25393-26220) | 197-215 |
| 4 | 359-363 | ATLQA linker 1-5 | | |
| 4 | 364-373 | ARDLICAQKF | surface_glycoprotein(NC_045512: + : 21563-25384) | 846-855 |
| 4 | 374-378 | ATLQA linker 1-5 | | |
| 4 | 379-386 | SDFVRATA | ORF3a_protein(NC_045512: + : 25393-26220) | 26-33 |
| 4 | 387-391 | ATLQA linker 1-5 | | |
| 4 | 392-401 | SVLNDILARL | surface_glycoprotein@Aruba(NC_045512@Aruba: + : 21563-25384) | 971-980 |
| 4 | 402-406 | ATLQA linker 1-5 | | |
| 4 | 407-424 | FLYENAFLPFAMGVIAMS | nsp6@Mexico(NC_045512@Mexico: + : 10973-11842) | 36-53 |
| 4 | 425-429 | ATLQA linker 1-5 | | |
| 4 | 430-442 | LLNVPLHGTILTR | membrane_glycoprotein(NC_045512: + : 26523-27191) | 119-131 |
| 4 | 443-447 | ATLQA linker 1-5 | | |
| 4 | 448-469 | SDFVRATATIPIRALSPFGWLI | ORF3a_protein@Belgium(NC_045512@Belgium: + : 25393-26220) | 26-47 |
| 4 | 470-474 | ATLQA linker 1-5 | | |
| 4 | 475-503 | FLYIIKLIFLWLLWPVTLACFVLAAVYRI | membrane_glycoprotein@Aruba(NC_045512@Aruba: + : 26523-27191) | 45-73 |
| 4 | 504-508 | ATLQA linker 1-5 | | |
| 4 | 509-524 | FSNVTWFHAIHVSGTN | surface_glycoprotein@Peru(NC_045512@Peru: + : 21563-25384) | 59-74 |
| 4 | 525-529 | ATLQA linker 1-5 | | |
| 4 | 530-558 | TLQCIMLVYCFGYFCTCYFGLFCLLNRY | nsp6@Aruba(NC_045512@Aruba: + : 10973-11842) | 203-231 |
| 4 | 559-563 | ATLQA linker 1-5 | | |
| 4 | 564-586 | VAAGLEAPFFYLYALVYFLQSIN | ORF3a_protein@Ecuador(NC_045512@Ecuador: + : 25393-26220) | 97-119 |
| 4 | 587-591 | ATLQA linker 1-5 | | |
| 4 | 592-599 | TDAVDCAL | surface_glycoprotein(NC_045512: + : 21563-25384) | 286-293 |
| 4 | 600-604 | ATLQA linker 1-5 | | |
| 4 | 605-619 | EFQFCNUPFLGVYHK | surface_glycoprotein@Norway(NC_045512@Norway: + : 21563-25384) | 129-143 |
| 4 | 620-624 | ATLQA linker 1-5 | | |
| 4 | 625-634 | SPGVVNPVME | ORF3a_protein@Brazil(NC_045512@Brazil: + : 25393-26220) | 252-261 |
| 4 | 635-639 | ATLQA linker 1-5 | | |
| 4 | 640-659 | MTSCCSCLKGCCSCGSCCKF | surface_glycoprotein(NC_045512: + : 21563-25384) | 1237-1256 |
| 5 | 1-25 | KNPLLYDANYFLCWHTNCYDYCIPY | ORF3a_protein@Aruba(NC_045512@Aruba: + : 25393-26220) | 136-160 |
| 5 | 26-30 | ATLQA linker 1-5 | | |
| 5 | 31-41 | SELVIGAVILR | membrane_glycoprotein(NC_045512: + : 26523-27191) | 136-146 |
| 5 | 42-46 | ATLQA linker 1-5 | | |
| 5 | 47-60 | LVLLPLVSSQCVNF | surface_glycoprotein@Brazil(NC_045512@Brazil: + : 21563-25384) | 5-18 |
| 5 | 61-65 | ATLQA linker 1-5 | | |
| 5 | 66-74 | LKLKDCVMY | nsp6@Aruba(NC_045512@Aruba: + : 10973-11842) | 105-113 |
| 5 | 75-79 | ATLQA linker 1-5 | | |
| 5 | 80-99 | PINLVRGLPQGFSALEPLVD | surface_glycoprotein@Austria(NC_045512@Austria: + : 21563-25384) | 205-224 |
| 5 | 100-104 | ATLQA linker 1-5 | | |
| 5 | 105-112 | VVNPVMEP | ORF3a_protein(NC_045512: + : 25393-26220) | 255-262 |
| 5 | 113-117 | ATLQA linker 1-5 | | |
| 5 | 118-125 | EHVQIHTI | ORF3a_protein(NC_045512: + : 25393-26220) | 242-249 |
| 5 | 126-130 | ATLQA linker 1-5 | | |
| 5 | 131-140 | ALAAVFHNAS | ORF3a_protein@SmallSouthAmerica(NC_045512@SmallSouthAmerica: + : | 51-60 |
| | | | 25393-26220) | |
| 5 | 141-145 | ATLQA linker 1-5 | | |
| 5 | 146-164 | VKDCVVLHSYFTSDYYQLY | ORF3a_protein@Aruba(NC_045512@Aruba: + : 25393-26220) | 197-215 |
| 5 | 165-169 | ATLQA linker 1-5 | | |
| 5 | 170-184 | ALLAVFHSASKIITL | ORF3a_protein@Aruba(NC_045512@Aruba: + : 25393-26220) | 51-65 |
| 5 | 185-189 | ATLQA linker 1-5 | | |
| 5 | 190-212 | TTVMFLARGIVFMCVEYCPIFFI | nsp6@Aruba(NC_045512@Aruba: + : 10973-11842) | 177-199 |
| 5 | 213-217 | ATLQA linker 1-5 | | |
| 5 | 218-240 | LIAIVMVTIMLCCITSCCSCLKG | surface_glycoprotein@Mozambique(NC_045512@Mozambique: + : 21563-25384) | 1220-1242 |
| 5 | 241-245 | ATLQA linker 1-5 | | |
| 5 | 246-260 | VNCTEVPVAIHADQL | surface_glycoprotein(NC_045512: + : 21563-25384) | 615-629 |
| 5 | 261-265 | ATLQA linker 1-5 | | |
| 5 | 266-282 | INFVRIIMRLWLCLKCR | ORF3a_protein@Mozambique(NC_045512@Mozambique: + : 25393-26220) | 118-134 |
| 5 | 283-287 | ATLQA linker 1-5 | | |
| 5 | 288-301 | KHWPQAIQFAPSAS | nucleocapsid_phosphoprotein@Australia(NC_045512@Australia: + : | 299-312 |
| | | | 28274-29533) | |
| 5 | 302-306 | ATLQA linker 1-5 | | |
| 5 | 307-320 | IKVCEFQFCNDPYY | surface_glycoprotein@CostaRica(NC_045512@CostaRica: + : 21563-25384) | 128-141 |
| 5 | 321-325 | ATLQA linker 1-5 | | |
| 5 | 326-340 | ALLAVFQSASKIITL | ORF3a_protein@Bangladesh(NC_045512@Bangladesh: + : 25393-26220) | 51-65 |
| 5 | 341-345 | ATLQA linker 1-5 | | |
| 5 | 346-359 | LMNVLTLVYKVYYG | nsp6@Aruba(NC_045512@Aruba: + : 10973-11842) | 139-152 |
| 5 | 360-364 | ATLQA linker 1-5 | | |
| 5 | 365-373 | FFSNVTWFH | surface_glycoprotein(NC_045512: + : 21563-25384) | 58-66 |
| 5 | 374-378 | ATLQA linker 1-5 | | |
| 5 | 379-386 | SLIDLQEL | surface_glycoprotein(NC_045512: + : 21563-25384) | 1196-1203 |
| 5 | 387-391 | ATLQA linker 1-5 | | |
| 5 | 392-401 | GNCDVVIGIV | surface_glycoprotein(NC_045512: + : 21563-25384) | 1124-1133 |
| 5 | 402-406 | ATLQA linker 1-5 | | |
| 5 | 407-421 | TYGVGYQPYRVVVLS | surface_glycoprotein@Australia(NC_045512@Australia: + : 21563-25384) | 496-510 |
| 5 | 422-426 | ATLQA linker 1-5 | | |
| 5 | 427-442 | LMNVLTLFYKVYYGNA | nsp6@Chile(NC_045512@Chile: + : 10973-11842) | 142-157 |
| 5 | 443-447 | ATLQA linker 1-5 | | |
| 5 | 448-463 | FLVLLPLVSSQCVNLT | surface_glycoprotein@Aruba(NC_045512@Aruba: + : 21563-25384) | 4-19 |
| 5 | 464-468 | ATLQA linker 1-5 | | |
| 5 | 469-488 | PINLVRGLPQGFSVLEPLVD | surface_glycoprotein@SmallAfrica(NC_045512@SmallAfrica: + : 21563-25384) | 209-228 |
| 5 | 489-493 | ATLQA linker 1-5 | | |
| 5 | 494-508 | VEHVTFFIYNKIVDE | ORF3a_protein(NC_045512: + : 25393-26220) | 225-239 |
| 5 | 509-513 | ATLQA linker 1-5 | | |
| 5 | 514-531 | LNDLCFTNVYADSFVIRG | surface_glycoprotein(NC_045512: + : 21563-25384) | 387-404 |
| 5 | 532-536 | ATLQA linker 1-5 | | |
| 5 | 537-550 | TDTVVEHVTFFIYN | ORF3a_protein@CostaRica(NC_045512@CostaRica: + : 25393-26220) | 221-234 |
| 5 | 551-555 | ATLQA linker 1-5 | | |
| 5 | 556-571 | NYTVSCLPFTINCQEP | ORF8_protein@Bangladesh(NC_045512@Bangladesh: + : 27894-28259) | 78-93 |
| 5 | 572-576 | ATLQA linker 1-5 | | |
| 5 | 577-586 | KQYGDCLGDI | surface_glycoprotein(NC_045512: + : 21563-25384) | 835-844 |
| 5 | 587-591 | ATLQA linker 1-5 | | |
| 5 | 592-601 | AGAALQIPFA | surface_glycoprotein@Nigeria(NC_045512@Nigeria: + : 21563-25384) | 886-895 |
| 5 | 602-606 | ATLQA linker 1-5 | | |
| 5 | 607-616 | FEYVSQPFLM | surface_glycoprotein@Aruba(NC_045512@Aruba: + : 21563-25384) | 164-173 |
| 5 | 617-621 | ATLQA linker 1-5 | | |
| 5 | 622-635 | TDTCVEHVTFFIYN | ORF3a_protein@Luxembourg(NC_045512@Luxembourg: + : 25393-26220) | 221-234 |
| 6 | 1-12 | EVFAQVKQIYKT | surface_glycoprotein@Aruba(NC_045512@Aruba: + : 21563-25384) | 776-787 |
| 6 | 13-17 | ATLQA linker 1-5 | | |
| 6 | 18-35 | IKDLPKEITVATSRTLSY | membrane_glycoprotein(NC_045512: + : 26523-27191) | 161-178 |
| 6 | 36-40 | ATLQA linker 1-5 | | |
| 6 | 41-55 | ISNCVADYSVLYNSA | surface_glycoprotein@Aruba(NC_045512@Aruba: + : 21563-25384) | 354-368 |
| 6 | 56-60 | ATLQA linker 1-5 | | |
| 6 | 61-73 | RSVASQSIIAYTM | surface_glycoprotein@Aruba(NC_045512@Aruba: + : 21563-25384) | 681-693 |
| 6 | 74-78 | ATLQA linker 1-5 | | |
| 6 | 79-92 | TSGVGYQPYRVVVL | surface_glycoprotein@Aruba(NC_045512@Aruba: + : 21563-25384) | 496-509 |
| 6 | 93-97 | ATLQA linker 1-5 | | |
| 6 | 98-107 | NCYDYCIPYN | ORF3a_protein(NC_045512: + : 25393-26220) | 152-161 |
| 6 | 108-112 | ATLQA linker 1-5 | | |
| 6 | 113-129 | KDQVILLNKHIDAYKTF | nucleocapsid_phosphoprotein@Aruba(NC_045512@Aruba: + : 28274-29533) | 347-363 |
| 6 | 130-134 | ATLQA linker 1-5 | | |
| 6 | 135-157 | HQPYVVDDPCPIHFYSKWYIRVG | ORF8_protein(NC_045512: + : 27894-28259) | 28-50 |
| 6 | 158-162 | ATLQA linker 1-5 | | |
| 6 | 163-180 | QLALFKGVHFVCNLLLLF | ORF3a_protein@CostaRica(NC_045512@CostaRica: + : 25393-26220) | 70-87 |
| 6 | 181-185 | ATLQA linker 1-5 | | |
| 6 | 186-197 | DTSLKLKECVMY | nsp6@SmallAsia(NC_045512@SmallAsia: + : 10973-11842) | 102-113 |
| 6 | 198-202 | ATLQA linker 1-5 | | |
| 6 | 203-225 | RVDFCGKGYHLMSFPQSAPHGVV | surface_glycoprotein(NC_045512: + : 21563-25384) | 1039-1061 |
| 6 | 226-230 | ATLQA linker 1-5 | | |
| 6 | 231-249 | LEILDITPCSFGGVSVITP | surface_glycoprotein(NC_045512: + : 21563-25384) | 582-600 |
| 6 | 250-254 | ATLQA linker 1-5 | | |
| 6 | 255-277 | YALVYFLQSINFVIIIMRLWLCW | ORF3a_protein@Denmark(NC_045512@Denmark: + : 25393-26220) | 109-131 |
| 6 | 278-282 | ATLQA linker 1-5 | | |
| 6 | 283-298 | HWPQIAQFAPSASAFF | nucleocapsid_phosphoprotein@Aruba(NC_045512@Aruba: + : 28274-29533) | 300-315 |
| 6 | 299-303 | ATLQA linker 1-5 | | |
| 6 | 304-319 | LQSLQTYVTQQLIRAA | surface_glycoprotein(NC_045512: + : 21563-25384) | 1001-1016 |
| 6 | 320-324 | ATLQA linker 1-5 | | |
| 6 | 325-332 | TQALPHRQ | nucleocapsid_phosphoprotein@Mozambique(NC_045512@Mozambique: + : | 379-386 |
| | | | 28274-29533) | |
| 6 | 333-337 | ATLQA linker 1-5 | | |
| 6 | 338-345 | EVTPSGTW | nucleocapsid_phosphoprotein(NC_045512: + : 28274-29533) | 323-330 |
| 6 | 346-350 | ATLQA linker 1-5 | | |
| 6 | 351-362 | FNCYFPLQSYGF | surface_glycoprotein(NC_045512: + : 21563-25384) | 486-497 |
| 6 | 363-367 | ATLQA linker 1-5 | | |
| 6 | 368-383 | ATVAYFNMVYMPASWV | nsp6@Aruba(NC_045512@Aruba: + : 10973-11842) | 76-91 |
| 6 | 384-388 | ATLQA linker 1-5 | | |
| 6 | 389-396 | SVDCTMYI | surface_glycoprotein(NC_045512: + : 21563-25384) | 735-742 |
| 6 | 397-401 | ATLQA linker 1-5 | | |
| 6 | 402-415 | GFNCYSPLQSYGFQ | surface_glycoprotein@Peru(NC_045512@Peru: + : 21563-25384) | 485-498 |
| 6 | 416-420 | ATLQA linker 1-5 | | |
| 6 | 421-442 | NVVIKVCEFQFCNYPFLGVYYH | surface_glycoprotein@Brazil(NC_045512@Brazil: + : 21563-25384) | 125-146 |
| 6 | 443-447 | ATLQA linker 1-5 | | |
| 6 | 448-472 | VLVQSTQWSLFFFFYENAFLPFAMG | nsp6@Iceland(NC_045512@Iceland: + : 10973-11842) | 24-48 |
| 6 | 473-477 | ATLQA linker 1-5 | | |
| 6 | 478-499 | GYQPYRVVVLSFELLHAPATVC | surface_glycoprotein(NC_045512: + : 21563-25384) | 504-525 |
| 6 | 500-504 | ATLQA linker 1-5 | | |
| 6 | 505-529 | VLVQSTQWSLFFFLYENAFLPFAMG | nsp6@Aruba(NC_045512@Aruba: + : 10973-11842) | 24-48 |
| 6 | 530-534 | ATLQA linker 1-5 | | |
| 6 | 535-545 | FFSNVTWFHVS | surface_glycoprotein@Nigeria(NC_045512@Nigeria: + : 21563-25384) | 58-68 |
| 6 | 546-550 | ATLQA linker 1-5 | | |
| 6 | 551-564 | IHFYSKWYIRVGAK | ORF8_protein@Mozambique(NC_045512@Mozambique: + : 27894-28259) | 39-52 |
| 6 | 565-569 | ATLQA linker 1-5 | | |
| 6 | 570-579 | TQNVLYENQK | surface_glycoprotein(NC_045512: + : 21563-25384) | 912-921 |
| 6 | 580-584 | ATLQA linker 1-5 | | |
| 6 | 585-601 | INFVRIIMRLWLCCKCR | ORF3a_protein@Australia(NC_045512@Australia: + : 25393-26220) | 118-134 |
| 6 | 602-606 | ATLQA linker 1-5 | | |
| 6 | 607-630 | PINLVRDLPQGFSVLEPLVDLPIG | surface_glycoprotein@Belgium(NC_045512@Belgium: + : 21563-25384) | 205-228 |
| 6 | 631-635 | ATLQA linker 1-5 | | |
| 6 | 636-647 | ISVTTEILPVSM | surface_glycoprotein(NC_045512: + : 21563-25384) | 720-731 |

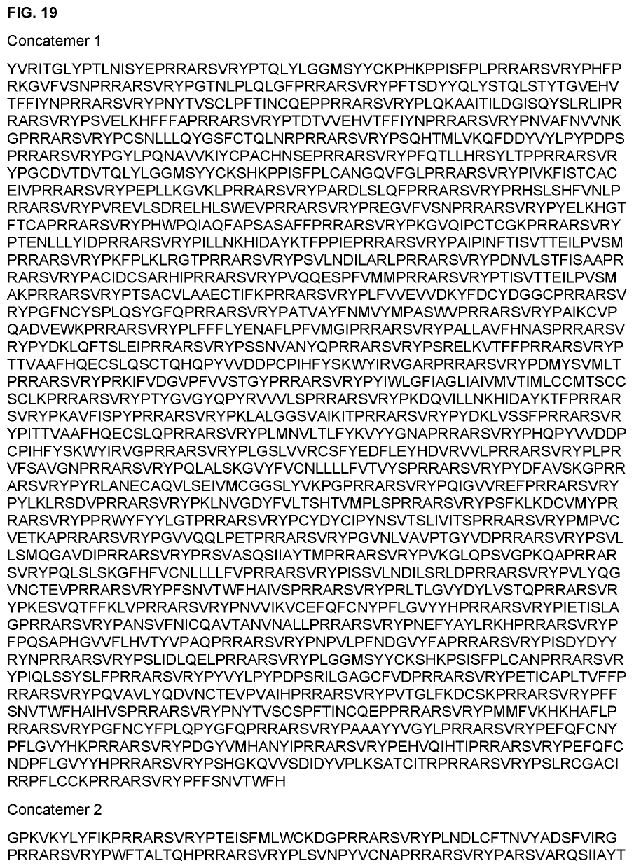

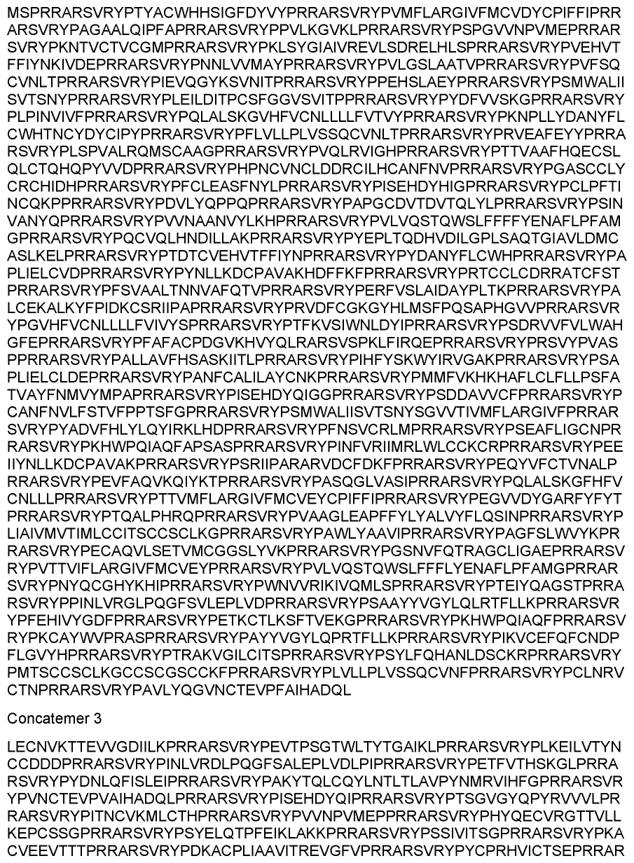

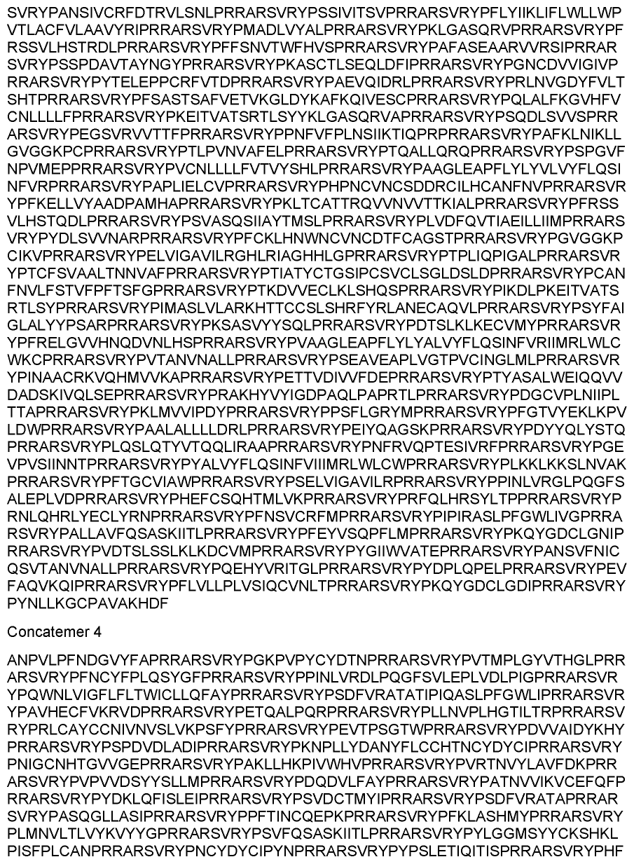

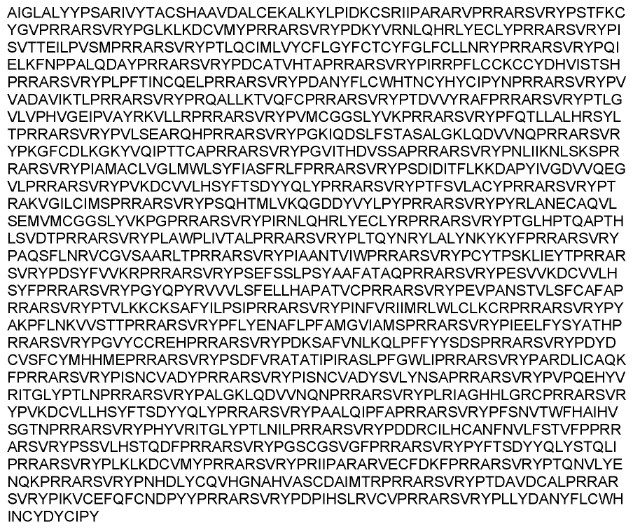

In certain embodiments, the vaccine comprises or expresses at least one of the concatemers of epitopes with intervening linker sequences (and as set forth in FIG. 19). In certain embodiments, the vaccine comprises or expresses at least two of the concatemers set forth below. In certain embodiments, the vaccine comprises or expresses three of the concatemers set forth below. In certain embodiments, the vaccine comprises or expresses all the concatemers set forth below.

TABLE 9

| concatemer | pos_in_concatemer | epitope | gene | pos_in_gene |
|---|---|---|---|---|
| 1 | 1-16 | YVRITGLYPTLNISYE | helicase@SmallNorthAmerical(NC_045512@SmallNorthAmerica: + : 16237-18039) | 246-261 |
| 1 | 17-26 | PRRARSVRYP linker 1-10 | | |
| 1 | 27-49 | TQLYLGGMSYYCKPHKPPISFPL | helicase@Iceland(NC_045512@Iceland: + : 16237-18039) | 61-83 |
| 1 | 50-59 | PRRARSVRYP linker 1-10 | | |
| 1 | 60-70 | HFPRKGVFVSN | surface_glycoprotein@SmallAsia(NC_045512@SmallAsia: + : 21563-25384) | 1078-1088 |
| 1 | 71-80 | PRRARSVRYP linker 1-10 | | |
| 1 | 81-90 | GTNLPLQLGF | 3′-to-5′_exonuclease(NC_045512: + : 10840-19620) | 102-111 |
| 1 | 91-100 | PRRARSVRYP linker 1-10 | | |
| 1 | 101-128 | FTSDYYQLYSTQLSTYTGVEHVTFFIYN | ORF3a_protein@Lativa(NC_045512@Lativa: + : 25393-26220) | 207-234 |
| 1 | 129-138 | PRRARSVRYP linker 1-10 | | |
| 1 | 139-154 | NYTVSCLPFTINCQEP | ORF8_protein@Bangladesh(NC_045512@Bangladesh: + : 27894-28259) | 78-93 |
| 1 | 155-164 | PRRARSVRYP linker 1-10 | | |
| 1 | 165-186 | LQKAAITILDGISQYSLRLI | nsp2(NC_045512: + : 806-2719) | 382-401 |
| 1 | 185-194 | PRRARSVRYP linker 1-10 | | |
| 1 | 195-204 | SVELKHFFFA | RNA-dependent_RNA-polymerase(NC_045512: + : 13442-13468 + 13468-16236) | 434-443 |
| 1 | 205-214 | PRRARSVRYP linker 1-10 | | |
| 1 | 215-228 | TDTVVEHVTFFIYN | ORF3a_protein@CostaRica(NC_045512@CostaRica: + : 25393-26220) | 221-234 |
| 1 | 229-238 | PRRARSVRYP linker 1-10 | | |
| 1 | 239-248 | NVAFNVVNKG | endoRNAse(NC_045512: + : 19621-20658) | 4-13 |
| 1 | 249-258 | PRRARSVRYP linker 1-10 | | |
| 1 | 259-275 | CSNLLLQYGSFCTQLNR | surface_glycoprotein(NC_045512: + : 21563-25384) | 749-765 |
| 1 | 276-285 | PRRARSVRYP linker 1-10 | | |
| 1 | 286-307 | SQHTMLVKQFDDYVYLPYPDPS | RNA-dependent_RNA_polymerase@Estonia(NC_045512@Estonia: + : | 814-835 |
| | | | 13442-13468 + 13468-16236) | |
| 1 | 308-317 | PRRARSVRYP linker 1-10 | | |
| 1 | 318-337 | GYLPQNAVVKIYCPACHNSE | nsp2(NC_045512: + : 806-2719) | 178-197 |
| 1 | 338-347 | PRRARSVRYP linker 1-10 | | |
| 1 | 348-359 | FQTLLHRSYLTP | surface_glycoprotein@SmallAsia(NC_045512@SmallAsia: + : 21563-25384) | 230-241 |
| 1 | 360-369 | PRRARSVRYP linker 1-10 | | |
| 1 | 370-408 | GCDVTDVTQLYLGGMSYYCKSHKPPISFPLCAN | helicase(NC_045512: + : 16237-18039) | 54-92 |
| | | GQVFGL | | |
| 1 | 409-418 | PRRARSVRYP linker 1-10 | | |
| 1 | 419-431 | IVKFISTCACEIV | nsp2(NC_045512: + : 806-2719) | 468-480 |
| 1 | 432-441 | PRRARSVRYP linker 1-10 | | |
| 1 | 442-450 | EPLLKGVKL | surface_glycoprotein@Croatia(NC_045512@Croatia: + : 21563-25384) | 1258-1266 |
| 1 | 451-460 | PRRARSVRYP linker 1-10 | | |
| 1 | 461-468 | ARDLSLQF | nsp3(NC_045512: + : 2720-8554) | 1652-1659 |
| 1 | 469-478 | PRRARSVRYP linker 1-10 | | |
| 1 | 479-488 | RHSLSHFVNL | nsp3(NC_045512: + : 2720-8554) | 1697-1706 |
| 1 | 489-498 | PRRARSVRYP linker 1-10 | | |
| 1 | 499-514 | VREVLSDRELHLSWEV | helicase(NC_045512: + : 16237-18039) | 154-169 |
| 1 | 515-524 | PRRARSVRYP linker 1-10 | | |
| 1 | 525-532 | REGVFVSN | surface_glycoprotein@Aruba(NC_045512@Aruba: + : 21563-25384) | 1087-1094 |
| 1 | 533-542 | PRRARSVRYP linker 1-10 | | |
| 1 | 543-553 | YELKHGTFTCA | nsp3(NC_045512: + : 2720-8554) | 996-1006 |
| 1 | 554-563 | PRRARSVRYP linker 1-10 | | |
| 1 | 564-579 | HWPQIAQFAPSASAFF | nucleocapsid_phosphoprotein@Aruba(NC_045512@Aruba: + : 28274-29533) | 300-315 |
| 1 | 580-589 | PRRARSVRYP linker 1-10 | | |
| 1 | 590-600 | KGVQIPCTCGK | nsp3(NC_045512: + : 2720-8554) | 963-973 |
| 1 | 601-610 | PRRARSVRYP linker 1-10 | | |
| 1 | 611-619 | TENLLLID | nsp3(NC_045512: + : 2720-8554) | 432-440 |
| 1 | 620-629 | PRRARSVRYP linker 1-10 | | |
| 1 | 630-646 | ILLNKHIDAYKTFPPIE | nucleocapsid_phosphoprotein@Chile(NC_045512@Chile: + : 28274-29533) | 351-367 |
| 1 | 647-656 | PRRARSVRYP linker 1-10 | | |
| 1 | 657-675 | AIPINFTISVTTEILPVSM | surface_glycoprotein@Aruba(NC_045512@Aruba: + : 21563-25384) | 709-727 |
| 1 | 676-685 | PRRARSVRYP linker 1-10 | | |
| 1 | 686-694 | KFPLKLRGT | 2′-O-ribose_methyltransferase(NC_045512: + : 20659-21552) | 249-257 |
| 1 | 695-704 | PRRARSVRYP linker 1-10 | | |
| 1 | 705-714 | SVLNDILARL | surface_glycoprotein@Aruba(NC_045512@Aruba: + : 21563-25384) | 971-980 |
| 1 | 715-724 | PRRARSVRYP linker 1-10 | | |
| 1 | 725-735 | DNVLSTFISAA | nsp3(NC_045512: + : 2720-8554) | 1809-1819 |
| 1 | 736-745 | PRRARSVRYP linker 1-10 | | |
| 1 | 746-755 | ACIDCSARHI | nsp3(NC_045512: + : 2720-8554) | 1872-1881 |
| 1 | 756-765 | PRRARSVRYP linker 1-10 | | |
| 1 | 766-775 | VQQESPFVMM | nsp3(NC_045512: + : 2720-8554) | 980-989 |
| 1 | 776-785 | PRRARSVRYP linker 1-10 | | |
| 1 | 786-800 | TISVTTEILPVSMAK | surface_glycoprotein@Mexico(NC_045512@Mexico: + : 21563-25384) | 719-733 |
| 1 | 801-810 | PRRARSVRYP linker 1-10 | | |
| 1 | 811-824 | TSACLVLAAECTIFK | nsp4(NC_045512: + : 8555-10054) | 147-160 |
| 1 | 825-834 | PRRARSVRYP linker 1-10 | | |
| 1 | 835-852 | LFVVEVVDKYFDCYDGGC | RNA-dependent_RNA_polymerase(NC_045512: + : 13442-13468 + 13468-16236) | 470-487 |
| 1 | 853-862 | PRRARSVRYP linker 1-10 | | |
| 1 | 863-876 | GFNCYSPLQSYGFQ | surface_glycoprotein@Peru(NC_045512@Peru: + : 21563-25384) | 485-498 |
| 1 | 877-886 | PRRARSVRYP linker 1-10 | | |
| 1 | 887-902 | ATVAYFNMVYMPASWV | nsp6@Aruba(NC_04551@Aruba: + : 10973-11842) | 76-91 |
| 1 | 903-912 | PRRARSVRYP linker 1-10 | | |
| 1 | 913-925 | AIKCVPQADVEWK | 3′-to-5′_exonuclease(NC_045512: + : 10840-19620) | 337-349 |
| 1 | 926-935 | PRRARSVRYP linker 1-10 | | |
| 1 | 936-952 | LFFFLYENAFLPFVMGI | nsp6@SmallSouthAmerica(NC_045512@SmallSouthAmerica: + : 10973-11842) | 33-49 |
| 1 | 953-962 | PRRARSVRYP linker 1-10 | | |
| 1 | 963-972 | ALLAVFHNAS | ORF3a_protein@SmallSouthAmerica(NC_045512@SmallSouthAmerica: + : | 51-60 |
| | | | 25393-26220) | |
| 1 | 973-982 | PRRARSVRYP linker 1-10 | | |
| 1 | 983-993 | YDKLQFTSLEI | helicase@Aruba(NC_045512@Aruba: + : 16237-18039) | 582-592 |
| 1 | 994-1003 | PRRARSVRYP linker 1-10 | | |
| 1 | 1004-1011 | SSNVANYQ | helicase@Austria(NC_045512@Austria: + : 16237-18039) | 263-270 |
| 1 | 1012-1021 | PRRARSVRYP linker 1-10 | | |
| 1 | 1022-1030 | SRELKVTFF | nsp3(NC_045512: + : 2720-8554) | 1134-1142 |
| 1 | 1031-1040 | PRRARSVRYP linker 1-10 | | |
| 1 | 1041-1082 | TTVAAFHQECSLQSCTQHQPYVVDDPCPIHFYS | ORF8_protein@Bangladesh(NC_045512@Bangladesh: + : 27894-28259) | 11-52 |
| | | KWYIRVGAR | | |
| 1 | 1083-1092 | PRRARSVRYP linker 1-10 | | |
| 1 | 1093-1100 | DMYSVMLT | RNA-dependent_RNA_polymerase@Malaysia(NC_045512@Malaysia: + : | 901-908 |
| | | | 13442-13468 + 13468-16236) | |
| 1 | 1101-1110 | PRRARSVRYP linker 1-10 | | |
| 1 | 1111-1126 | RKIFVDGVPFVVSTGY | RNA-dependent_RNA_polymerase(NC_045512: + : 13442-13468 + 13468-16236) | 331-346 |
| 1 | 1127-1136 | PRRARSVRYP linker 1-10 | | |
| 1 | 1137-1167 | YIWLGFIAGLIAIVMVTIMLCCMTSCCSCLK | surface_glycoprotein@Aruba(NC_045512@Aruba: + : 21563-25384) | 1211-1241 |
| 1 | 1168-1177 | PRRARSVRYP linker 1-10 | | |
| 1 | 1178-1192 | TYGVGYQPYRVVVLS | surface_glycoprotein@Australia(NC_045512@Australia: + : 21563-25384) | 496-510 |
| 1 | 1193-1202 | PRRARSVRYP linker 1-10 | | |
| 1 | 1203-1219 | KDQVILLNKHIDAYKTF | nucleocapsid_phosphoprotein@Aruba(NC_045512@Aruba: + : 28274-29533) | 347-363 |
| 1 | 1220-1229 | PRRARSVRYP linker 1-10 | | |
| 1 | 1230-1237 | KAVFISPY | helicase(NC_045512: + : 16237-18039) | 508-515 |
| 1 | 1238-1247 | PRRARSVRYP linker 1-10 | | |
| 1 | 1248-1260 | KLALGGSVAIKIT | 2′-O-ribose_methyltransferase(NC_045512: + : 20659-21552) | 160-172 |
| 1 | 1261-1270 | PRRARSVRYP linker 1-10 | | |
| 1 | 1271-1278 | YDKLVSSF | nsp3(NC_045512: + : 2720-8554) | 365-372 |
| 1 | 1279-1288 | PRRARSVRYP linker 1-10 | | |
| 1 | 1289-1302 | ITTVAAFHQECSLQ | ORF8_protein@Aruba(NC_045512@Aruba: + : 27894-28259) | 10-23 |
| 1 | 1303-1312 | PRRARSVRYP linker 1-10 | | |
| 1 | 1313-1328 | LMNVLTLFYKVYYGNA | nsp6@Chile(NC_045512@Chile: + : 10973-11842) | 142-157 |
| 1 | 1329-1338 | PRRARSVRYP linker 1-10 | | |
| 1 | 1339-1361 | HQPYVVDDPCPIHFYSKWYIRVG | ORF8_protein(NC_045512: + : 27894-28259) | 28-50 |
| 1 | 1362-1371 | PRRARSVRYP linker 1-10 | | |
| 1 | 1372-1395 | LGSLVVRCSFYEDFLEYHDVRVVL | ORF8_protein(NC_045512: + : 27894-28259) | 95-118 |
| 1 | 1396-1405 | PRRARSVRYP linker 1-10 | | |
| 1 | 1406-1415 | LPRVFSAVGN | nsp4(NC_045512: + : 8555-10054) | 122-131 |
| 1 | 1416-1425 | PRRARSVRYP linker 1-10 | | |
| 1 | 1426-1448 | QLALSKGVYFVCNLLLLFVTVYS | ORF3a_protein@Aruba(NC_045512@Aruba: + : 25393-26220) | 70-92 |
| 1 | 1449-1458 | PRRARSVRYP linker 1-10 | | |
| 1 | 1459-1466 | YDFAVSKG | RNA-dependent_RNA_polymerase@Aruba(NC_045512@Aruba: + : | 420-427 |
| | | | 13442-13468 + 13468-16236) | |
| 1 | 1467-1476 | PRRARSVRYP linker 1-10 | | |
| 1 | 1477-1502 | YRLANECAQVLSEIVMCGGSLYVKPG | RNA-dependent_RNA_polymerase@Canada(NC_045512@Canada: + : | 653-678 |
| | | | 13442-13468 + 13468-16236) | |
| 1 | 1503-1512 | PRRARSVRYP linker 1-10 | | |
| 1 | 1513-1520 | QIGVVREF | helicase(NC_045512: + : 16237-18039) | 492-499 |
| 1 | 1521-1530 | PRRARSVRYP linker 1-10 | | |
| 1 | 1531-1538 | YLKLRSDV | nsp4(NC_045512: + : 8555-10054) | 427-434 |
| 1 | 1539-1547 | PRRARSVRYP linker 1-10 | | |
| 1 | 1547-1567 | KLNVGDYFVLTSHTVMPLS | helicase(NC_045512: + : 16237-18039) | 218-236 |
| 1 | 1568-1577 | PRRARSVRYP linker 1-10 | | |
| 1 | 1578-1587 | SFLKLDCVMY | nsp6@Malaysia(NC_04551@Malaysia: + : 10973-11842) | 107-116 |
| 1 | 1588-1597 | PRRARSVRYP linker 1-10 | | |
| 1 | 1598-1607 | PRWYFYYLGT | nucleocapsid_phosphoprotein@Aruba(NC_045512@Aruba: + : 28274-29533) | 106-115 |
| 1 | 1608-1617 | PRRARSVRYP linker 1-10 | | |
| 1 | 1618-1636 | CYDYCIPYNSVTSLIVITS | ORF3a_protein@Netherlands(NC_045512@Netherlands: + : 25393-26220) | 153-171 |
| 1 | 1637-1646 | PRRARSVRYP linker 1-10 | | |
| 1 | 1647-1655 | MPVCVETKA | nsp3(NC_045512: + : 2720-8554) | 571-579 |
| 1 | 1656-1665 | PRRARSVRYP linker 1-10 | | |
| 1 | 1666-1674 | GVVQQLPET | endoRNAase(NC_045512: + : 184-192) | |
| 1 | 1675-1684 | PRRARSVRYP linker 1-10 | | |
| 1 | 1685-1697 | GVNLVAVPTGYVD | 3′-to-5′_exonuclease(NC_045512: + : 10840-19620) | 114-126 |
| 1 | 1698-1707 | PRRARSVRYP linker 1-10 | | |
| 1 | 1708-1719 | SVLLSMQGAVDI | nsp7(NC_045512: + : 11843-12091) | 57-68 |
| 1 | 1720-1729 | PRRARSVRYP linker 1-10 | | |
| 1 | 1730-1742 | RSVASQSIIAYTM | surface_glycoprotein@Aruba(NC_045512@Aruba: + : 21563-25384) | 681-693 |
| 1 | 1743-1752 | PRRARSVRYP linker 1-10 | | |
| 1 | 1753-1765 | VKGLQPSVGPKQA | endoRNAase(NC_045512: + : 184-192) | 148-160 |
| 1 | 1766-1775 | PRRARSVRYP linker 1-10 | | |
| 1 | 1776-1794 | QLSLSKGFHFVCNLLLLFV | ORF3a_protein@Estonia(NC_045512@Estonia: + : 25393-26220) | 70-88 |
| 1 | 1795-1804 | PRRARSVRYP linker 1-10 | | |
| 1 | 1805-1817 | ISSVLNDILSRLD | surface_glycoprotein@Bangladesh(NC_045512@Bangladesh: + : 21563-25384) | 972-984 |
| 1 | 1818-1827 | PRRARSVRYP linker 1-10 | | |
| 1 | 1828-1838 | VLYQGVNCTEV | surface_glycoprotein@Aruba(NC_045512@Aruba: + : 21563-25384) | 606-616 |
| 1 | 1839-1848 | PRRARSVRYP linker 1-10 | | |
| 1 | 1849-1860 | FSNVTWFHAIVS | surface_glycoprotein@Ghana(NC_045512@Ghana: + : 21563-25384) | 59-70 |
| 1 | 1861-1870 | PRRARSVRYP linker 1-10 | | |
| 1 | 1871-1884 | RLTLGVYDYLVSTQ | nsp6(NC_045512: + : 10973-11842) | 236-249 |
| 1 | 1885-1894 | PRRARSVRYP linker 1-10 | | |
| 1 | 1895-1905 | KESVQTFFKLV | nsp2(NC_045512: + : 806-2719) | 492-502 |
| 1 | 1906-1915 | PRRARSVRYP linker 1-10 | | |
| 1 | 1916-1937 | NVVIKVCEFQFCNYPFLGVYYH | surface_glycoprotein@Brazil(NC_045512@Brazil: + : 21563-25384) | 125-146 |
| 1 | 1938-1947 | PRRARSVRYP linker 1-10 | | |
| 1 | 1948-1955 | IETISLAG | nsp3(NC_045512: + : 2720-8554) | 684-691 |
| 1 | 1956-1965 | PRRARSVRYP linker 1-10 | | |
| 1 | 1966-1984 | ANSVFNICQAVTANVNALL | RNA-dependent_RNA_polymerase@Aruba(NC_045512@Aruba: + : | 690-708 |
| | | | 13442-13468 + 13468-16236) | |
| 1 | 1985-1994 | PRRARSVRYP linker 1-10 | | |
| 1 | 1995-2004 | NEVYAYLRKH | RNA-dependent_RNA_polymerase(NC_045512: + : 13442-13468 + 13468-16236) | 743-752 |
| 1 | 2005-2014 | PRRARSVRYP linker 1-10 | | |
| 1 | 2015-2034 | FPQSAPHGVVFLHVTYVPAQ | surface_glycoprotein@Aruba(NC_045512@Aruba: + : 21563-25384) | 1048-1067 |
| 1 | 2035-2044 | PRRARSVRYP linker 1-10 | | |
| 1 | 2045-2057 | NPVLPFNDGVYFA | surface_glycoprotein(NC_045512: + : 21563-25384) | 81-93 |
| 1 | 2058-2067 | PRRARSVRYP linker 1-10 | | |
| 1 | 2068-2077 | ISDYDYYRYN | RNA-dependent_RNA_polymerase@Aruba(NC_045512@Aruba: + : | 450-459 |
| | | | 13442-13468 + 13468-16236) | |
| 1 | 2078-2087 | PRRARSVRYP linker 1-10 | | |
| 1 | 2088-2095 | SLIDLQEL | surface_glycoprotein(NC_045512: + : 21563-25384) | 1196-1203 |
| 1 | 2096-2105 | PRRARSVRYP linker 1-10 | | |
| 1 | 2106-2127 | LGGMSYYCKSHKPSISFPLCAN | helicase@NewZealand(NC-045512@NewZealand: + : 16237-18039) | 65-86 |
| 1 | 2128-2137 | PRRARSVRYP linker 1-10 | | |
| 1 | 2138-2146 | IQLSSYSLF | 2′-O-ribose_methyltransferase(NC_045512: + : 20659-21552) | 237-245 |
| 1 | 2147-2156 | PRRARSVRYP linker 1-10 | | |
| 1 | 2157-2176 | YVYLPYPDPSRILGAGCFVD | RNA-dependent_RNA_polymerase(NC_045512: + : 13442-13468 + 13468-16236) | 826-845 |
| 1 | 2177-2186 | PRRARSVRYP linker 1-10 | | |
| 1 | 2187-2197 | ETICAPLTVFF | endoRNAase(NC_045512: + : 184-192) | 113-123 |
| 1 | 2198-2207 | PRRARSVRYP linker 1-10 | | |
| 1 | 2208-2226 | QVAVLYQDVNCTEVPVAIH | surface_glycoprotein@China(NC_045512@China: + : 21563-25384) | 607-625 |
| 1 | 2227-2236 | PRRARSVRYP linker 1-10 | | |
| 1 | 2239-2246 | VTGLFKDCSK | 3′-to-5′_exonuclease(NC_045512: + : 10840-19620) | 4-13 |
| 1 | 2247-2256 | PRRARSVRYP linker 1-10 | | |
| 1 | 2257-2270 | FFSNVTWFHAIHVS | surface_glycoprotein@Bangladehs(NC_045512@Bangladesh: + : 21563-25384) | 58-71 |
| 1 | 2271-2280 | PRRARSVRYP linker 1-10 | | |
| 1 | 2281-2296 | NYTVSCSPFTINCQEP | ORF8_protein@CostaRica(NC_045512@CostaRica: + : 27894-28259) | 78-93 |
| 1 | 2297-2306 | PRRARSVRYP linker 1-10 | | |
| 1 | 2307-2317 | MMFVKHKHAFL | nsp6@Aruba(NC_04551@Aruba: + : 10973-11842) | 57-67 |
| 1 | 2318-2327 | PRRARSVRYP linker 1-10 | | |
| 1 | 2328-2341 | GFNCYFPLQPYGFQ | surface_glycoprotein@SmallSouthAmerica(NC_045512@SmallSouthAmerica: + : | 481-494 |
| | | | 21563-25384) | |
| 1 | 2342-2351 | PRRARSVRYP linker 1-10 | | |
| 1 | 2352-2360 | AAAYYVGYL | surface_glycoprotein(NC_045512: + : 21563-25384) | 262-270 |
| 1 | 2361-2370 | PRRARSVRYP linker 1-10 | | |
| 1 | 2371-2385 | EFQFCNYPFLGVYHK | surface_glycoprotein@Norway(NC_045512@Norway: + : 21563-25384) | 129-143 |
| 1 | 2386-2395 | PRRARSVRYP linker 1-10 | | |
| 1 | 2396-2405 | DGYVMHANYI | 2′-O-ribose_methyltransferase(NC_045512: + : 20659-21552) | 220-229 |
| 1 | 2406-2415 | PRRARSVRYP linker 1-10 | | |
| 1 | 2416-2423 | EHVQIHTI | ORF3a_protein(NC_045512: + : 25393-26220) | 242-249 |
| 1 | 2424-2433 | PRRARSVRYP linker 1-10 | | |
| 1 | 2434-2448 | EFQFCNDPFLGVYYH | surface_glycoprotein@Chile(NC_045512@Chile: + : 21563-25384) | 132-146 |
| 1 | 2449-2458 | PRRARSVRYP linker 1-10 | | |
| 1 | 2459-2481 | SHGKQVVSDIDYVPLKSATCITR | 3′-to-5′_exonuclease(NC_045512: + : 10840-19620) | 454-476 |
| 1 | 2482-2491 | PRRARSVRYP linker 1-10 | | |
| 1 | 2492-2507 | SLRCGACIRRPFLCCK | helicase@Aruba(NC-045512@Aruba: + : 16237-18039) | 13-28 |
| 1 | 2508-2517 | PRRARSVRYP linker 1-10 | | |
| 1 | 2518-2526 | FFSNVTWFH | surface_glycoprotein@Chile(NC_045512@Chile: + : 21563-25384) | 58-66 |
| 2 | 1-11 | GPKVKYLYFIK | nsp9(NC_045512: + : 12686-13024) | 82-92 |
| 2 | 12-21 | PRRARSVRYP linker 1-10 | | |
| 2 | 22-33 | TEISFMLWCKDG | endoRNAase(NC_045512: + : 184-192) | 325-336 |
| 2 | 34-43 | PRRARSVRYP linker 1-10 | | |
| 2 | 44-61 | LNDLCFTNVYADSFVIRG | surface_glycoprotein(NC_045512: + : 21563-25384) | 387-404 |
| 2 | 62-71 | PRRARSVRYP linker 1-10 | | |
| 2 | 72-79 | WFTALTQH | nucleocapsid_phosphoprotein(NC_045512: + : 28274-29533) | 52-59 |
| 2 | 80-89 | PRRARSVRYP linker 1-10 | | |
| 2 | 90-99 | LSVNPYVCNA | helicase@Cyprus(NC-045512@Cyprus: + : 16237-18039) | 43-52 |
| 2 | 100-109 | PRRARSVRYP linker 1-10 | | |
| 2 | 110-124 | ARSVARQSIIAYTMS | surface_glycoprotein@Mozambiqye(NC_045512@Mozambique: + : 21563-25384) | 680-694 |
| 2 | 125-134 | PRRARSVRYP linker 1-10 | | |
| 2 | 135-149 | TYACWHHSIGFDYVY | 3′-to-5′_exonuclease(NC_045512: + : 10840-19620) | 223-237 |
| 2 | 150-159 | PRRARSVRYP linker 1-10 | | |
| 2 | 160-180 | VMFLARGIVFMCVDYCPIFFI | nsp6@Cyprus(NC_04551@Cyprus: + : 10973-11842) | 179-199 |
| 2 | 181-190 | PRRARSVRYP linker 1-10 | | |
| 2 | 191-200 | AGAALQIPFA | surface_glycoprotein@Nigeria(NC_045512@Nigeria: + : 21563-25384) | 886-895 |
| 2 | 201-210 | PRRARSVRYP linker 1-10 | | |
| 2 | 211-218 | PVLKGVKL | surface_glycoprotein@Aruba(NC_045512@Aruba: + : 21563-25384) | 1259-1266 |
| 2 | 219-228 | PRRARSVRYP linker 1-10 | | |
| 2 | 229-238 | SPGVVNPVME | ORF3a_protein@Brazil(NC_045512@Brazil: + : 25393-26220) | 252-261 |
| 2 | 239-248 | PRRARSVRYP linker 1-10 | | |
| 2 | 249-258 | KNTVCTVCGM | nsp10(NC_045512: + : 13025-13441) | 113-122 |
| 2 | 259-268 | PRRARSVRYP linker 1-10 | | |
| 2 | 269-289 | KLSYGIAIVREVLSDRELHLS | helicase@Colombia(NC-045512@Colombia: + : 16237-18039) | 146-166 |
| 2 | 290-299 | PRRARSVRYP linker 1-10 | | |
| 2 | 300-314 | VEHVTFFIYNKIVDE | ORF3a_protein(NC_045512: + : 25393-26220) | 225-239 |
| 2 | 315-324 | PRRARSVRYP linker 1-10 | | |
| 2 | 325-332 | NNLVVMAY | nsp2(NC_045512: + : 806-2719) | 413-420 |
| 2 | 333-342 | PRRARSVRYP linker 1-10 | | |
| 2 | 343-351 | VLGSLAATV | nsp9(NC_045512: + : 12686-13024) | 102-110 |
| 2 | 352-361 | PRRARSVRYP linker 1-10 | | |
| 2 | 362-370 | VFSQCVNLT | surface_glycoprotein@Egypt(NC_045512@Egypt: + : 21563-25384) | 11-19 |
| 2 | 371-380 | PRRARSVRYP linker 1-10 | | |
| 2 | 381-392 | IEVQGYKSVNIT | nsp3(NC_045512: + : 2720-8554) | 13-24 |
| 2 | 393-402 | PRRARSVRYP linker 1-10 | | |
| 2 | 403-410 | PEHSLAEY | nsp2(NC_045512: + : 806-2719) | 200-207 |
| 2 | 411-420 | PRRARSVRYP linker 1-10 | | |
| 2 | 421-433 | SMWALIISVTSNY | nsp6@Aruba(NC_04551@Aruba: + : 10973-11842) | 160-172 |
| 2 | 434-443 | PRRARSVRYP linker 1-10 | | |
| 2 | 444-462 | LEILDITPCSFGGVSVITP | surface_glycoprotein(NC_045512: + : 21563-25384) | 582-600 |
| 2 | 463-472 | PRRARSVRYP linker 1-10 | | |
| 2 | 473-480 | YDFVVSKG | RNA-dependent_RNA_polymerase@Japan(NC_045512@Japan: + : | 420-427 |
| | | | 13442-13468 + 13468-16236) | |
| 2 | 481-490 | PRRARSVRYP linker 1-10 | | |
| 2 | 491-498 | LPINVIVF | nsp3(NC_045512: + : 2720-8554) | 1718-1725 |
| 2 | 499-508 | PRRARSVRYP linker 1-10 | | |
| 2 | 509-530 | QLALSKGVHFVCNLLLLFVTVY | ORF3a_protein@Australia(NC_045512@Australia: + : 25393-26220) | 70-91 |
| 2 | 531-540 | PRRARSVRYP linker 1-10 | | |
| 2 | 541-565 | KNPLLYDANYFLCWHTNCYDYCIPY | ORF3a_protein@Aruba(NC_045512@Aruba: + : 25393-26220) | 136-160 |
| 2 | 566-575 | PRRARSVRYP linker 1-10 | | |
| 2 | 576-591 | FLVLLPLVSSQCVNLT | surface_glycoprotein@Aruba(NC_045512@Aruba: + : 21563-25384) | 4-19 |
| 2 | 592-601 | PRRARSVRYP linker 1-10 | | |
| 2 | 602-609 | RVEAFEYY | nsp3(NC_045512: + : 2720-8554) | 810-817 |
| 2 | 610-619 | PRRARSVRYP linker 1-10 | | |
| 2 | 620-633 | LSPVALRQMSCAAAG | nsp9(NC_045512: + : 12686-13024) | 4-17 |
| 2 | 634-643 | PRRARSVRYP linker 1-10 | | |
| 2 | 644-651 | VQLRVIGH | 3C-like_proteinase(NC_045512: + : 10055-10972) | 73-80 |
| 2 | 652-661 | PRRARSVRYP linker 1-10 | | |
| 2 | 662-685 | TTVAAFHQECSLQLCTQHQPYVVD | ORF8_protein@USA(NC_045512@USA: + : 27894-28259) | 11-34 |
| 2 | 686-695 | PRRARSVRYP linker 1-10 | | |
| 2 | 696-716 | HPNCVNCLDDRCILHCANFNV | RNA-dependent_RNA_polymerase@Australia(NC_045512@Australia: + : | 295-315 |
| | | | 13442-13468 + 13468-16236) | |
| 2 | 717-726 | PRRARSVRYP linker 1-10 | | |
| 2 | 727-740 | GASCCLYCRCHIDH | nsp10(NC_045512: + : 13025-13441) | 70-83 |
| 2 | 741-750 | PRRARSVRYP linker 1-10 | | |
| 2 | 751-760 | FCLEASFNYL | nsp3(NC_045512: + : 2720-8554) | 1391-1400 |
| 2 | 761-770 | PRRARSVRYP linker 1-10 | | |
| 2 | 771-779 | ISEHDYHIG | ORF3a_protein@NorthMacedonia(NC_045512@NorthMacedonia: + : 25393-26220) | 179-187 |
| 2 | 780-789 | PRRARSVRYP linker 1-10 | | |
| 2 | 790-800 | CLPFTINCQKP | ORF8_protein@Brazil(NC_045512@Brazil: + : 27894-28259) | 83-93 |
| 2 | 801-810 | PRRARSVRYP linker 1-10 | | |
| 2 | 811-818 | DVLYQPPQ | nsp4(NC_045512: + : 8555-10054) | 484-491 |
| 2 | 819-828 | PRRARSVRYP linker 1-10 | | |
| 2 | 829-842 | APGCDVTDVTQLYL | helicase@Aruba(NC-045512@Aruba: + : 16237-18039) | 52-65 |
| 2 | 843-852 | PRRARSVRYP linker 1-10 | | |
| 2 | 853-860 | SINVANYQ | helicase@Mozambique(NC-045512@Mozambique: + : 16237-18039) | 263-270 |
| 2 | 861-870 | PRRARSVRYP linker 1-10 | | |
| 2 | 871-881 | VVNAANVYLKH | nsp3(NC_045512: + : 2720-8554) | 239-249 |
| 2 | 882-891 | PRRARSVRYP linker 1-10 | | |
| 2 | 892-916 | VLVQSTQWSLFFFFYENAFLPFAMG | nsp6@Iceland(NC_04551@Iceland: + : 10973-11842) | 24-48 |
| 2 | 917-926 | PRRARSVRYP linker 1-10 | | |
| 2 | 927-939 | QCVQLHNDILLAK | nsp7(NC_045512: + : 11843-12091) | 31-43 |
| 2 | 940-949 | PRRARSVRYP linker 1-10 | | |
| 2 | 950-982 | YEPLTQDHVDILGPLSAQTGIAVLDMCASLKEL | 3C-like_proteinase(NC_045512: + : 10055-10972) | 239-271 |
| 2 | 983-992 | PRRARSVRYP linker 1-10 | | |
| 2 | 993-1006 | TDTCVEHVTFFIYN | ORF3a_protein@Luxembourg(NC_045512@Luxembourg: + : 25393-26220) | 221-234 |
| 2 | 1007-1016 | PRRARSVRYP linker 1-10 | | |
| 2 | 1017-1026 | YDANYFLCWH | ORF3a_protein(NC_045512: + : 25393-26220) | 141-150 |
| 2 | 1027-1036 | PRRARSVRYP linker 1-10 | | |
| 2 | 1037-1045 | APLIELCVD | ORF8_protein@Bangladesh(NC_045512@Bangladesh: + : 27894-28259) | 55-63 |
| 2 | 1046-1055 | PRRARSVRYP linker 1-10 | | |
| 2 | 1056-1073 | YNLLKDCPAVAKHDFFKV | RNA-dependent_RNA_polymerase@Aruba(NC_045512@Aruba: + : | 87-104 |
| | | | 13442-13468 + 13468-16236) | |
| 2 | 1074-1083 | PRRARSVRYP linker 1-10 | | |
| 2 | 1084-1098 | RTCCLCDRRATCFST | 3′-to-5′_exonuclease(NC_045512: + : 18040-19620) | 205-219 |
| 2 | 1099-1108 | PRRARSVRYP linker 1-10 | | |
| 2 | 1109-1123 | FSVAALTNNVAFQTV | RNA-dependent_RNA_polymerase(NC_045512: + : 13442-13468 + 13468-16236) | 396-410 |
| 2 | 1124-1133 | PRRARSVRYP linker 1-10 | | |
| 2 | 1134-1148 | ERFVSLAIDAYPLTK | RNA-dependent_RNA_polymerase(NC_045512: + : 13442-13468 + 13468-16236) | 857-871 |
| 2 | 1149-1159 | PRRARSVRYP linker 1-10 | | |
| 2 | 1159-1179 | ALCEKALKYFPIDKCSRIIPA | helicase@Colombia(NC-045512@Colombia: + : 16237-18039) | 316-336 |
| 2 | 1180-1189 | PRRARSVRYP linker 1-10 | | |
| 2 | 1190-1212 | RVDFCGKGYHLMSFPQSAPHGVV | surface_glycoprotein(NC_045512: + : 21563-25384) | 1039-1061 |
| 2 | 1213-1222 | PRRARSVRYP linker 1-10 | | |
| 2 | 1223-1239 | GVHFVCNLLLLFVIVYS | ORF3a_protein@Malaysia(NC_045512@Malaysia: + : 25393-26220) | 76-92 |
| 2 | 1240-1249 | PRRARSVRYP linker 1-10 | | |
| 2 | 1250-1261 | TFKVSIWNLDYI | ORF6_protein(NC_045512: + : 27202-27387) | 21-32 |
| 2 | 1262-1271 | PRRARSVRYP linker 1-10 | | |
| 2 | 1272-1285 | SDRVVFVLWAHGFE | 3′-to-5′_exonuclease(NC_045512: + : 18040-19620) | 178-191 |
| 2 | 1286-1295 | PRRARSVRYP linker 1-10 | | |
| 2 | 1296-1324 | FAFACPDGVKHVYQLRARSVSPKLFIRQE | ORF7a_protein(NC_045512: + : 27394-27759) | 63-91 |
| 2 | 1325-1334 | PRRARSVRYP linker 1-10 | | |
| 2 | 1335-1343 | RSVYPVASP | nsp2(NC_045512: + : 806-2719) | 121-129 |
| 2 | 1344-1353 | PRRARSVRYP linker 1-10 | | |
| 2 | 1354-1368 | ALLAVFHSASKIITL | ORF3a_protein@Aruba(NC_045512@Aruba: + : 25393-26220) | 51-65 |
| 2 | 1369-1378 | PRRARSVRYP linker 1-10 | | |
| 2 | 1379-1392 | IHFYSKWYIRVGAK | ORF8_protein@Mozambique(NC_045512@Mozambique: + : 27894-28259) | 39-52 |
| 2 | 1393-1402 | PRRARSVRYP linker 1-10 | | |
| 2 | 1403-1413 | SAPLIELCLDE | ORF8_protein@Russia(NC_045512@Russia: + : 27894-28259) | 54-64 |
| 2 | 1414-1423 | PRRARSVRYP linker 1-10 | | |
| 2 | 1424-1436 | ANFCALILAYCNK | nsp3(NC_045512: + : 2720-8554) | 890-902 |
| 2 | 1437-1446 | PRRARSVRYP linker 1-10 | | |
| 2 | 1447-1478 | MMFVKHKHAFLCLFLLPSFATVAYFNMVYMPA | nsp6@Indonesia(NC_04551@Indonesia: + : 10973-11842) | 57-88 |
| 2 | 1479-1488 | PRRARSVRYP linker 1-10 | | |
| 2 | 1489-1498 | ISEHDYQIGG | ORF3a_protein@FrenchGuiana(NC_045512@FrenchGuiana: + : 25393-26220) | 179-188 |
| 2 | 1499-1508 | PRRARSVRYP linker 1-10 | | |
| 2 | 1509-1516 | SDDAVVCF | RNA-dependent_RNA_polymerase@Aruba(NC_045512@Aruba: + : | 759-766 |
| | | | 13442-13468 + 13468-16236) | |
| 2 | 1517-1526 | PRRARSVRYP linker 1-10 | | |
| 2 | 1527-1544 | CANFNVLFSTVFPPTSFG | RNA-dependent_RNA_polymerase@China(NC_045512@China: + : | 310-327 |
| | | | 13442-13468 + 13468-16236) | |
| 2 | 1545-1554 | PRRARSVRYP linker 1-10 | | |
| 2 | 1555-1583 | SMWALIISVTSNYSGVVTIVMFLARGIVF | nsp6@SmallSouthAmerica(NC_04551@SmallSouthAmerica: + : 10973-11842) | 160-188 |
| 2 | 1584-1593 | PRRARSVRYP linker 1-10 | | |
| 2 | 1594-1610 | YADVFHLYLQYIRKLHD | RNA-dependent_RNA_polymerase(NC_045512: + : 13442-13468 + 13468-16236) | 877-893 |
| 2 | 1611-1620 | PRRARSVRYP linker 1-10 | | |
| 2 | 1621-1628 | FNSVCRLM | helicase@Aruba(NC-045512@Aruba: + : 16237-18039) | 422-429 |
| 2 | 1629-1638 | PRRARSVRYP linker 1-10 | | |
| 2 | 1639-1647 | SEAFLIGCN | 2′-O-ribose_methyltransferase(NC_045512: + : 20659-21552) | 202-210 |
| 2 | 1648-1657 | PRRARSVRYP linker 1-10 | | |
| 2 | 1658-1671 | KHWPQIAQFAPSAS | nucleocapsid_phosphoprotein@Australia(NC_045512@Australia: + : | 299-312 |
| | | | 28274-29533) | |
| 2 | 1672-1681 | PRRARSVRYP linker 1-10 | | |
| 2 | 1682-1698 | INFVRIIMRLWLCCKCR | ORF3a_protein@Australia(NC_045512@Australia: + : 25393-26220) | 118-134 |
| 2 | 1699-1708 | PRRARSVRYP linker 1-10 | | |
| 2 | 1709-1724 | EEIIYNLLKDCPAVAK | RNA-dependent_RNA_polymerase@Senegal(NC_045512@Senegal: + : | 83-98 |
| | | | 13442-13468 + 13468-16236) | |
| 2 | 1725-1734 | PRRARSVRYP linker 1-10 | | |
| 2 | 1735-1750 | SRIIPARAVDCFDKF | helicase@Brazil(NC-045512@Brazil: + : 16237-18039) | 331-346 |
| 2 | 1751-1760 | PRRARSVRYP linker 1-10 | | |
| 2 | 1761-1771 | EQYVFCTVNAL | helicase(NC-045512: + : 16237-18039) | 353-363 |
| 2 | 1772-1781 | PRRARSVRYP linker 1-10 | | |
| 2 | 1782-1793 | EVFAQVKQIYKT | surface_glycoprotein@Aruba(NC_045512@Aruba: + : 21563-25384) | 776-787 |
| 2 | 1794-1803 | PRRARSVRYP linker 1-10 | | |
| 2 | 1804-1812 | ASQGLVASI | RNA-dependent_RNA_polymerase@Aruba(NC_045512@Aruba: + : | 771-779 |
| | | | 13442-13468 + 13468-16236) | |
| 2 | 1813-1822 | PRRARSVRYP linker 1-10 | | |
| 2 | 1823-1838 | QLALSKGFHFVCNLLL | ORF3a_protein@Iceland(NC_045512@Iceland: + : 25393-26220) | 70-85 |
| 2 | 1839-1848 | PRRARSVRYP linker 1-10 | | |
| 2 | 1849-1871 | TTVMFLARGIVFMCVEYCPIFFI | nsp6@Aruba(NC_04551@Aruba: + : 10973-11842) | 177-199 |
| 2 | 1872-1881 | PRRARSVRYP linker 1-10 | | |
| 2 | 1882-1895 | EGVVDYGARFYFYT | nsp3(NC_045512: + : 2720-8554) | 595-608 |
| 2 | 1896-1905 | PRRARSVRYP linker 1-10 | | |
| 2 | 1906-1913 | TQALPHRQ | nucleocapsid_phosphoprotein@Mozambique(NC_045512@Mozambique: + : | 379-386 |
| | | | 28274-29533) | |
| 2 | 1914-1923 | PRRARSVRYP linker 1-10 | | |
| 2 | 1924-1946 | VAAGLEAPFFYLYALVYFLQSIN | ORF3a_protein@Ecuador(NC_045512@Ecuador: + : 25393-26220) | 97-119 |
| 2 | 1947-1956 | PRRARSVRYP linker 1-10 | | |
| 2 | 1957-1979 | LIAIVMVTIMLCCITSCCSCLKG | surface_glycoprotein@Mozambique(NC_045512@Mozambique: + : 21563-25384) | 1220-1242 |
| 2 | 1980-1989 | PRRARSVRYP linker 1-10 | | |
| 2 | 1990-1997 | AWLYAAVI | 3C-like_proteinase(NC_045512: + : 10055-10972) | 206-213 |
| 2 | 1998-2007 | PRRARSVRYP linker 1-10 | | |
| 2 | 2008-2016 | AGFSLWVYK | 3′-to-5′_exonuclease(NC_045512: + : 18040-19620) | 504-512 |
| 2 | 2017-2026 | PRRARSVRYP linker 1-10 | | |
| 2 | 2027-2045 | ECAQVLSETVMCGGSLYVK | RNA-dependent_RNA_polymerase@Aruba(NC_045512@Aruba: + : | 658-676 |
| | | | 13442-13468 + 13468-16236) | |
| 2 | 2046-2055 | PRRARSVRYP linker 1-10 | | |
| 2 | 2056-2071 | GSNVFQTRAGCLIGAE | surface_glycoprotein(NC_045512: + : 21563-25384) | 639-654 |
| 2 | 2072-2081 | PRRARSVRYP linker 1-10 | | |
| 2 | 2082-2099 | VTTVIFLARGIVFMCVEY | nsp6@Malaysia(NC_04551@Malaysia: + : 10973-11842) | 179-196 |
| 2 | 2100-2109 | PRRARSVRYP linker 1-10 | | |
| 2 | 2110-2134 | VLVQSTQWSLFFFLYENAFLPFAMG | nsp6@Aruba(NC_04551@Aruba: + : 10973-11842) | 24-48 |
| 2 | 2135-2144 | PRRARSVRYP linker 1-10 | | |
| 2 | 2145-2154 | NYQCGHYKHI | nsp3(NC_045512: + : 2720-8554) | 1012-1021 |
| 2 | 2155-2164 | PRRARSVRYP linker 1-10 | | |
| 2 | 2165-2177 | WNVVRIKIVQMLS | 3′-to-5′_exonuclease(NC_045512: + : 18040-19620) | 159-171 |
| 2 | 2178-2187 | PRRARSVRYP linker 1-10 | | |
| 2 | 2188-2196 | TEIYQAGST | surface_glycoprotein@Aruba(NC_045512@Aruba: + : 21563-25384) | 466-474 |
| 2 | 2197-2206 | PRRARSVRYP linker 1-10 | | |
| 2 | 2207-2226 | PINLVRGLPQGFSVLEPLVD | surface_glycoprotein@SmallAfrica(NC_045512@SmallAfrica: + : 21563-25384) | 209-228 |
| 2 | 2227-2236 | PRRARSVRYP linker 1-10 | | |
| 2 | 2237-2253 | SAAYYVGYLQLRTFLLK | surface_glycoprotein@Italy(NC_045512@Italy: + : 21563-25384) | 258-274 |
| 2 | 2254-2263 | PRRARSVRYP linker 1-10 | | |
| 2 | 2264-2272 | FEHIVYGDF | endoRNAse(NC_045512: + : 19621-20658) | 232-240 |
| 2 | 2273-2282 | PRRARSVRYP linker 1-10 | | |
| 2 | 2283-2296 | ETKCTLKSFTVEKG | surface_glycoprotein@Aruba(NC_045512@Aruba: + : 21563-25384) | 294-307 |
| 2 | 2297-2306 | PRRARSVRYP linker 1-10 | | |
| 2 | 2307-2315 | KHWPQIAQF | nucleocapsid_phosphoprotein(NC_045512: + : 28274-29533) | 299-307 |
| 2 | 2316-2325 | PRRARSVRYP linker 1-10 | | |
| 2 | 2326-2335 | KCAYWVPRAS | nsp2(NC_045512: + : 806-2719) | 239-248 |
| 2 | 2336-2345 | PRRARSVRYP linker 1-10 | | |
| 2 | 2346-2360 | AYYVGYLQPRTFLLK | surface_glycoprotein@Aruba(NC_045512@Aruba: + : 21563-25384) | 260-274 |
| 2 | 2361-2370 | PRRARSVRYP linker 1-10 | | |
| 2 | 2371-2388 | IKVCEFQFCNDPFLGVHY | surface_glycoprotein@Aruba(NC_045512@Aruba: + : 21563-25384) | 125-142 |
| 2 | 2389-2398 | PRRARSVRYP linker 1-10 | | |
| 2 | 2399-2410 | TRAKVGILCITS | helicase@SouthKorea(NC-045512@SouthKorea: + : 16237-18039) | 566-577 |
| 2 | 2411-2420 | PRRARSVRYP linker 1-10 | | |
| 2 | 2421-2434 | SYLFQHANLDSCKR | nsp3(NC_045512: + : 2720-8554) | 915-928 |
| 2 | 2435-2444 | PRRARSVRYP linker 1-10 | | |
| 2 | 2445-2464 | MTSCCSCLKGCCSCGSCCKF | surface_glycoprotein(NC_045512: + : 21563-25384) | 1237-1256 |
| 2 | 2465-2474 | PRRARSVRYP linker 1-10 | | |
| 2 | 2475-2488 | LVLLPLVSSQCVNF | surface_glycoprotein@Brazil(NC_045512@Brazil: + : 21563-25384) | 5-18 |
| 2 | 2489-2498 | PRRARSVRYP linker 1-10 | | |
| 2 | 2499-2506 | CLNRVCTN | nsp3(NC_045512: + : 2720-8554) | 1342-1349 |
| 2 | 2507-2516 | PRRARSVRYP linker 1-10 | | |
| 2 | 2517-2537 | AVLYQGVNCTEVPFAIHADQL | surface_glycoprotein@SmallAfrica(NC_045512@SmallAfrica: + : | 506-625 |
| | | | 21563-25384) | |
| 3 | 1-17 | LECNVKTTEVVGDIILK | nsp3(NC_045512: + : 2720-8554) | 1244-1260 |
| 3 | 18-27 | PRRARSVRYP linker 1-10 | | |
| 3 | 28-44 | EVTPSGTWLTYTGAIKL | nucleocapsid_phosphoprotein@Bulgaria(NC_045512@Bulgaria: + : | 323-339 |
| | | | 28274-29533) | |
| 3 | 45-54 | PRRARSVRYP linker 1-10 | | |
| 3 | 55-68 | LKEILVTYNCCDDD | RNA-dependent_RNA_polymerase(NC_045512: + : 13442-13468 + 13468-16236) | 142-155 |
| 3 | 69-78 | PRRARSVRYP linker 1-10 | | |
| 3 | 79-100 | INLVRDLPQGFSALEPLVDLPI | surface_glycoprotein@Aruba(NC_045512@Aruba: + : 21563-25384) | 206-227 |
| 3 | 101-110 | PRRARSVRYP linker 1-10 | | |
| 3 | 111-120 | ETFVTHSKGL | nsp2(NC_045512: + : 806-2719) | 527-536 |
| 3 | 121-130 | PRRARSVRYP linker 1-10 | | |
| 3 | 131-141 | YDNLQFISLEI | helicase@Austria(NC-045512@Austria: + : 16237-18039) | 582-592 |
| 3 | 142-151 | PRRARSVRYP linker 1-10 | | |
| 3 | 152-178 | AKYTQLCQYLNTLTLAVPYNMRVIHFG | 2′-O-ribose_methyltransferase(NC_045512: + : 20659-21552) | 45-71 |
| 3 | 179-188 | PRRARSVRYP linker 1-10 | | |
| 3 | 189-203 | VNCTEVPVAIHADQL | surface_glycoprotein(NC_045512: + : 21563-25384) | 615-629 |
| 3 | 204-213 | PRRARSVRYP linker 1-10 | | |
| 3 | 214-221 | ISEHDYQI | ORF3a_protein@Aruba(NC_045512@Aruba: + : 25393-26220) | 179-186 |
| 3 | 222-231 | PRRARSVRYP linker 1-10 | | |
| 3 | 232-245 | TSGVGYQPYRVVVL | surface_glycoprotein@Aruba(NC_045512@Aruba: + : 21563-25384) | 496-509 |
| 3 | 246-255 | PRRARSVRYP linker 1-10 | | |
| 3 | 256-266 | ITNCVKMLCTH | nsp10(NC_045512: + : 13025-13441) | 38-48 |
| 3 | 267-276 | PRRARSVRYP linker 1-10 | | |
| 3 | 277-284 | VVNPVMEP | ORF3a_protein(NC_045512: + : 25393-26220) | 255-262 |
| 3 | 285-294 | PRRARSVRYP linker 1-10 | | |
| 3 | 295-314 | JYQECVRGTTVLLKEPCSSG | ORF7a_protein(NC_045512: + : 27394-27759) | 19-38 |
| 3 | 315-324 | PRRARSVRYP linker 1-10 | | |
| 3 | 325-339 | SYELQTPFEIKLAKK | nsp2(NC_045512: + : 806-2719) | 68-82 |
| 3 | 340-349 | PRRARSVRYP linker 1-10 | | |
| 3 | 350-357 | SSIVITSG | ORF3a_protein@SouthKorea(NC_045512@SouthKorea: + : 25393-26220) | 165-172 |
| 3 | 358-367 | PRRARSVRYP linker 1-10 | | |
| 3 | 368-377 | KACVEEVTTT | nsp3(NC_045512: + : 2720-8554) | 415-424 |
| 3 | 378-387 | PRRARSVRYP linker 1-10 | | |
| 3 | 388-405 | DKACPLIAAVITREVGFV | nsp4(NC_045512: + : 8555-10054) | 85-102 |
| 3 | 406-415 | PRRARSVRYP linker 1-10 | | |
| 3 | 416-426 | YCPRHVICTSE | 3C-like_proteinase(NC_045512: + : 10055-10972) | 37-47 |
| 3 | 427-436 | PRRARSVRYP linker 1-10 | | |
| 3 | 437-452 | ANSIVCRFDTRVLSNL | 3′-to-5′_exonuclease(NC_045512: + : 18040-19620) | 394-409 |
| 3 | 453-462 | PRRARSVRYP linker 1-10 | | |
| 3 | 463-470 | SSIVITSV | ORF3a_protein@Aruba(NC_045512@Aruba: + : 25393-26220) | 165-172 |
| 3 | 471-480 | PRRARSVRYP linker 1-10 | | |
| 3 | 481-509 | FLYIIKLIFLWLLWPVTLACFVLAAVYRI | membrane_glycoprotein@Aruba(NC_045512@Aruba: + : 26523-27191) | 45-73 |
| 3 | 510-519 | PRRARSVRYP linker 1-10 | | |
| 3 | 520-527 | MADLVYAL | RNA-dependent_RNA_polymerase(NC_045512: + : 13442-13468 + 13468-16236) | 124-131 |
| 3 | 528-537 | PRRARSVRYP linker 1-10 | | |
| 3 | 538-545 | KLGASQRV | membrane_glycoprotein(NC_045512: + : 26523-27191) | 180-187 |
| 3 | 546-555 | PRRARSVRYP linker 1-10 | | |
| 3 | 556-567 | FRSSVLHSTRDL | surface_glycoprotein@Nigeria(NC_045512@Nigeria: + : 21563-25384) | 43-54 |
| 3 | 568-577 | PRRARSVRYP linker 1-10 | | |
| 3 | 578-588 | FFSNVTWFHVS | surface_glycoprotein@Nigeria(NC_045512@Nigeria: + : 21563-25384) | 58-68 |
| 3 | 589-598 | PRRARSVRYP linker 1-10 | | |
| 3 | 599-611 | AFASEAARVVRSI | nsp2(NC_045512: + : 806-2719) | 355-367 |
| 3 | 612-621 | PRRARSVRYP linker 1-10 | | |
| 3 | 622-633 | SSPDAVTAYNGY | nsp3(NC_045512: + : 2720-8554) | 660-671 |
| 3 | 634-643 | PRRARSVRYP linker 1-10 | | |
| 3 | 644-656 | KASCTLSEQLDFI | nsp2(NC_045512: + : 806-2719) | 30-42 |
| 3 | 657-666 | PRRARSVRYP linker 1-10 | | |
| 3 | 667-676 | GNCDVVIGIV | surface_glycoprotein(NC_045512: + : 21563-25384) | 1124-1133 |
| 3 | 677-686 | PRRARSVRYP linker 1-10 | | |
| 3 | 687-699 | YTELEPPCRFVTD | nsp9(NC_045512: + : 12686-13024) | 66-78 |
| 3 | 700-709 | PRRARSVRYP linker 1-10 | | |
| 3 | 710-717 | AEVQIDRL | surface_glycoprotein(NC_045512: + : 21563-25384) | 989-996 |
| 3 | 718-727 | PRRARSVRYP linker 1-10 | | |
| 3 | 728-741 | RLNVGDYFVLTSHT | helicase@Austria(NC-045512@Austria: + : 16237-18039) | 218-231 |
| 3 | 442-751 | PRRARSVRYP linker 1-10 | | |
| 3 | 752-778 | FSASTSAFVETVKGLDYKAFKQIVESC | nsp2(NC_045512: + : 806-2719) | 300-326 |
| 3 | 779-788 | PRRARSVRYP linker 1-10 | | |
| 3 | 789-806 | QLALFKGVHFVCNLLLLF | ORF3a_protein@CostaRica(NC_045512@CostaRica: + : 25393-26220) | 70-87 |
| 3 | 807-816 | PRRARSVRYP linker 1-10 | | |
| 3 | 817-839 | KEITVATSRTLSYYKLGASQRVA | membrane_glycoprotein@Aruba(NC_045512@Aruba: + : 26523-27191) | 166-188 |
| 3 | 840-849 | PRRARSVRYP linker 1-10 | | |
| 3 | 850-857 | SQDLSVVS | endoRNAse(NC_045512: + : 19621-20658) | 308-315 |
| 3 | 858-867 | PRRARSVRYP linker 1-10 | | |
| 3 | 868-877 | EGSVRVVTTF | nsp4(NC_045512: + : 8555-10054) | 207-216 |
| 3 | 878-887 | PRRARSVRYP linker 1-10 | | |
| 3 | 888-904 | PNFVFPLNSIIKTIQPR | nsp2(NC_045512: + : 806-2719) | 91-107 |
| 3 | 905-914 | PRRARSVRYP linker 1-10 | | |
| 3 | 915-930 | AFKLNIKLLGVGGKPC | nsp6(NC_045512: + : 10973-11842) | 268-283 |
| 3 | 931-940 | PRRARSVRYP linker 1-10 | | |
| 3 | 941-950 | TLPVNVAFEL | endoRNAse(NC_045512: + : 19621-20658) | 48-57 |
| 3 | 951-960 | PRRARSVRYP linker 1-10 | | |
| 3 | 961-968 | TQALLQRQ | nucleocapsid_phosphoprotein@CostaRica(NC_045512@CostaRica: + : | 379-386 |
| | | | 28274-29533) | |
| 3 | 969-978 | PRRARSVRYP linker 1-10 | | |
| 3 | 979-989 | SPGVFNPVMEP | ORF3a_protein@Colombia(NC_045512@Colombia: + : 25393-26220) | 252-262 |
| 3 | 990-999 | PRRARSVRYP linker 1-10 | | |
| 3 | 1000-1014 | VCNLLLLFVTVYSHL | ORF3a_protein@Croatia(NC_045512@Croatia: + : 25393-26220) | 80-94 |
| 3 | 1015-1024 | PRRARSVRYP linker 1-10 | | |
| 3 | 1025-1049 | AAGLEAPFLYLYVLVYFLQSINFVR | ORF3a_protein@Norway(NC_045512@Norway: + : 25393-26220) | 98-122 |
| 3 | 1050-1059 | PRRARSVRYP linker 1-10 | | |
| 3 | 1060-1067 | APLEILCV | ORF8_protein(NC_045512: + : 27894-28259) | 55-62 |
| 3 | 1068-1077 | PRRARSVRYP linker 1-10 | | |
| 3 | 1078-1098 | HPNCVNCSDDRCILHCANFNV | RNA-dependent_RNA_polymerase@Aruba(NC_045512@Aruba: + : | 295-315 |
| | | | 13442-13468 + 13468-16236) | |
| 3 | 1099-1108 | PRRARSVRYP linker 1-10 | | |
| 3 | 1109-1123 | FKELLVYAADPAMHA | RNA-dependent_RNA_polymerase(NC_045512: + : 13442-13468 + 13468-16236) | 368-382 |
| 3 | 1124-1133 | PRRARSVRYP linker 1-10 | | |
| 3 | 1134-1153 | KLTCATTRQVVNVVTTKIAL | nsp3(NC_045512: + : 2720-8554) | 1923-1942 |
| 3 | 1154-1163 | PRRARSVRYP linker 1-10 | | |
| 3 | 1164-1175 | FRSSVLHSTQDL | surface_glycoprotein@Aruba(NC_045512@Aruba: + : 21563-25384) | 43-54 |
| 3 | 1176-1185 | PRRARSVRYP linker 1-10 | | |
| 3 | 1186-1199 | SVASQSIIAYTMSL | surface_glycoprotein@Aruba(NC_045512@Aruba: + : 21563-25384) | 678-691 |
| 3 | 1200-1209 | PRRARSVRYP linker 1-10 | | |
| 3 | 1210-1225 | LVDFQVTIAEILLIIM | ORF6_protein(NC_045512: + : 27202-27387) | 4-19 |
| 3 | 1226-1235 | PRRARSVRYP linker 1-10 | | |
| 3 | 1236-1244 | YDLSVVNAR | helicase(NC_045512: + : 16237-18039) | 382-390 |
| 3 | 1245-1254 | PRRARSVRYP linker 1-10 | | |
| 3 | 1255-1274 | FCKLHNWNCVNCDTFCAGST | nsp3(NC_045512: + : 2720-8554) | 1626-1645 |
| 3 | 1275-1284 | PRRARSVRYP linker 1-10 | | |
| 3 | 1285-1294 | GVGGKPCIKV | nsp6@Aruba(NC_045512@Aruba: + : 10973-11842) | 274-283 |
| 3 | 1295-1304 | PRRARSVRYP linker 1-10 | | |
| 3 | 1305-1325 | ELVIGAVILRGHLRIAGHHLG | membrane_glycoprotein@Aruba(NC_045512@Aruba: + : 26523-27191) | 137-157 |
| 3 | 1326-1335 | PRRARSVRYP linker 1-10 | | |
| 3 | 1336-1345 | TPLIQPIGAL | nsp4(NC_045512: + : 8555-10054) | 269-278 |
| 3 | 1346-1355 | PRRARSVRYP linker 1-10 | | |
| 3 | 1356-1369 | TCFSVAATNNVAF | RNA-dependent_RNA_polymerase@Aruba(NC_045512@Aruba: + : | 394-407 |
| | | | 13442-13468 + 13468-16236) | |
| 3 | 1370-1379 | PRRARSVRYP linker 1-10 | | |
| 3 | 1380-1402 | TIATYCTGSIPCSVCLSGLDSLD | nsp3(NC_045512: + : 2720-8554) | 1459-1481 |
| 3 | 1403-1412 | PRRARSVRYP linker 1-10 | | |
| 3 | 1413-1430 | CANFNVLFSTVFPFTSFG | RNA-dependent_RNA_polymerase@Nigeria(NC_045512@Nigeria: + : | 310-327 |
| | | | 13442-13468 + 13468-16236) | |
| 3 | 1431-1440 | PRRARSVRYP linker 1-10 | | |
| 3 | 1441-1454 | TKDVVECLKLSHQS | nsp3(NC_045512: + : 2720-8554) | 1830-1843 |
| 3 | 1455-1464 | PRRARSVRYP linker 1-10 | | |
| 3 | 1465-1482 | IKDLPKEITVATSRTLSY | membrane_glycoprotein(NC_045512: + : 26523-27191) | 161-178 |
| 3 | 1483-1492 | PRRARSVRYP linker 1-10 | | |
| 3 | 1493-1524 | IMASLVLARKHTTCCSLSHRFYRLANECAQVL | RNA-dependent_RNA_polymerase(NC_045512: + : 13442-13468 + 13468-16236) | 632-663 |
| 3 | 1525-1534 | PRRARSVRYP linker 1-10 | | |
| 3 | 1535-1549 | SYFAIGLALYYPSAR | helicase@Austria(NC-045512@Austria: + : 16237-18039) | 289-303 |
| 3 | 1550-1559 | PRRARSVRYP linker 1-10 | | |
| 3 | 1560-1569 | KSASVYYSQL | nsp3(NC_045512: + : 2720-8554) | 1737-1746 |
| 3 | 1570-1579 | PRRARSVRYP linker 1-10 | | |
| 3 | 1580-1591 | DTSLKLKECVMY | nsp6@SmallAsia(NC_045512@SmallAsia: + : 10973-11842) | 102-113 |
| 3 | 1592-1601 | PRRARSVRYP linker 1-10 | | |
| 3 | 1602-1617 | FRELGVVHNQDVNLHS | RNA-dependent_RNA_polymerase(NC_045512: + : 13442-13468 + 13468-16236) | 348-363 |
| 3 | 1618-1627 | PRRARSVRYP linker 1-10 | | |
| 3 | 1628-1664 | VAAGLEAPFLYLYALVYFLQSINFVRIIMRLW | ORF3a_protein@Aruba(NC_045512@Aruba: + : 25393-26220) | 97-133 |
| | | LCWKC | | |
| 3 | 1665-1674 | PRRARSVRYP linker 1-10 | | |
| 3 | 1675-1683 | VTANVNALL | RNA-dependent_RNA_polymerase(NC_045512: + : 13442-13468 + 13468-16236) | 700-708 |
| 3 | 1684-1693 | PRRARSVRYP linker 1-10 | | |
| 3 | 1694-1713 | SEAVEAPLVGTPVCINGLML | nsp2(NC_045512: + : 806-2719) | 591-610 |
| 3 | 1714-1723 | PRRARSVRYP linker 1-10 | | |
| 3 | 1724-1738 | INAACRKVQHMVVKA | 3′-to-5′_exonuclease(NC_045512: + : 18040-19620) | 305-319 |
| 3 | 1739-1748 | PRRARSVRYP linker 1-10 | | |
| 3 | 1749-1759 | ETTVDIVVFDE | helicase@SmallAsia(NC-045512@SmallAsia: + : 16237-18039) | 365-375 |
| 3 | 1760-1769 | PRRARSVRYP linker 1-10 | | |
| 3 | 1770-1793 | TYASALWEIQQVVDADSKIVQLSE | nsp8(NC_045512: + : 12092-12685) | 148-171 |
| 3 | 1794-1803 | PRRARSVRYP linker 1-10 | | |
| 3 | 1804-1823 | RAKHYVYIGDPAQLPAPRTL | helicase(NC_045512: + : 16237-18039) | 392-411 |
| 3 | 1824-1833 | PRRARSVRYP linker 1-10 | | |
| 3 | 1834-1847 | DGCVPLNIIPLTTA | nsp8(NC_045512: + : 12092-12685) | 112-125 |
| 3 | 1848-1857 | PRRARSVRYP linker 1-10 | | |
| 3 | 1858-1866 | KLMVVIPDY | nsp8(NC_045512: + : 12092-12685) | 127-135 |
| 3 | 1867-1876 | PRRARSVRYP linker 1-10 | | |
| 3 | 1877-1884 | PSFLGRYM | nsp3(NC_045512: + : 2720-8554) | 822-829 |
| 3 | 1885-1894 | PRRARSVRYP linker 1-10 | | |
| 3 | 1895-1908 | FGTVYEKLKPVLDW | nsp2(NC_045512: + : 806-2719) | 437-450 |
| 3 | 1909-1918 | PRRARSVRYP linker 1-10 | | |
| 3 | 1919-1929 | AALALLLLDRL | nucleocapsid_phosphoprotein@Aruba(NC_045512@Aruba: + : 28274-29533) | 217-227 |
| 3 | 1930-1939 | PRRARSVRYP linker 1-10 | | |
| 3 | 1940-1947 | EIYQAGSK | surface_glycoprotein@Mexico(NC_045512@Mexico: + : 21563-25384) | 471-478 |
| 3 | 1948-1957 | PRRARSVRYP linker 1-10 | | |
| 3 | 1958-1966 | DYYQLYSTQ | ORF3a_protein(NC_045512: + : 25393-26220) | 210-218 |
| 3 | 1967-1976 | PRRARSVRYP linker 1-10 | | |
| 3 | 1977-1992 | LQSLQTYVTQQLIRAA | surface_glycoprotein(NC_045512: + : 21563-25384) | 1001-1016 |
| 3 | 1993-2002 | PRRARSVRYP linker 1-10 | | |
| 3 | 2003-2015 | NFRVQPTESIVRF | surface_glycoprotein(NC_045512: + : 21563-25384) | 317-329 |
| 3 | 2016-2025 | PRRARSVRYP linker 1-10 | | |
| 3 | 2026-2036 | GEVPVSIINNT | endoRNAse(NC_045512: + : 19621-20658) | 20-30 |
| 3 | 2037-2046 | PRRARSVRYP linker 1-10 | | |
| 3 | 2047-2069 | YALVYFLQSINFVIIIMRLWLCW | ORF3a_protein@Denmark(NC_045512@Denmark: + : 25393-26220) | 109-131 |
| 3 | 2070-2079 | PRRARSVRYP linker 1-10 | | |
| 3 | 2080-2091 | LKKLKKSLNVAK | nsp8(NC_045512: + : 12092-12685) | 35-46 |
| 3 | 2092-2101 | PRRARSVRYP linker 1-10 | | |
| 3 | 2102-2109 | FTGCVIAW | surface_glycoprotein(NC_045512: + : 21563-25384) | 429-436 |
| 3 | 2110-2119 | PRRARSVRYP linker 1-10 | | |
| 3 | 2121-2130 | SELVIGAVILR | membrane_glycoprotein(NC_045512: + : 26523-27191) | 136-146 |
| 3 | 2131-2140 | PRRARSVRYP linker 1-10 | | |
| 3 | 2141-2160 | PINLVRGLPQGFSALEPLVD | surface_glycoprotein@Austria(NC_045512@Austria: + : 21563-25384) | 205-224 |
| 3 | 2161-2170 | PRRARSVRYP linker 1-10 | | |
| 3 | 2171-2182 | HEFCSQHTMLVK | RNA-dependent_RNA_polymerase(NC_045512: + : 13442-13468 + 13468-16236) | 810-821 |
| 3 | 2183-2192 | PRRARSVRYP linker 1-10 | | |
| 3 | 2193-2203 | RFQLHRSYLTP | surface_glycoprotein@Austria(NC_045512@Austria: + : 21563-25384) | 233-243 |
| 3 | 2204-2213 | PRRARSVRYP linker 1-10 | | |
| 3 | 2214-2227 | RNLQHRLYECLYRN | RNA-dependent_RNA_polymerase(NC_045512: + : 13442-13468 + 13468-16236) | 721-734 |
| 3 | 2228-2237 | PRRARSVRYP linker 1-10 | | |
| 3 | 2238-2245 | FNSVCRFM | helicase@Malaysia(NC_045512@Malaysia: + : 16237-18039) | 422-429 |
| 3 | 2246-2255 | PRRARSVRYP linker 1-10 | | |
| 3 | 2256-2270 | IPIRASLPFWGLIVG | ORF3a_protein@Iceland(NC_045512@Iceland: + : 25393-26220) | 35-49 |
| 3 | 2271-2280 | PRRARSVRYP linker 1-10 | | |
| 3 | 2281-2295 | ALLAVFQSASKIITL | ORF3a_protein@Bangladesh(NC_045512@Bangladesh: + : 25393-26220) | 51-65 |
| 3 | 2296-2305 | PRRARSVRYP linker 1-10 | | |
| 3 | 2306-2315 | FEYVSQPFLM | surface_glycoprotein@Aruba(NC_045512@Aruba: + : 21563-25384) | 164-173 |
| 3 | 2316-2325 | PRRARSVRYP linker 1-10 | | |
| 3 | 2326-2335 | KQYGDCLGNI | surface_glycoprotein@Jordan(NC_045512@Jordan: + : 21563-25384) | 831-840 |
| 3 | 2336-2345 | PRRARSVRYP linker 1-10 | | |
| 3 | 2346-2360 | VDTSLSSLKLKDCVM | nsp6@Latvia(NC_045512@Latvia: + : 10973-11842) | 101-115 |
| 3 | 2361-2370 | PRRARSVRYP linker 1-10 | | |
| 3 | 2371-2379 | YGIIWVATE | nucleocapsid_phosphoprotein@SmallSouthAmerica(NC_045512@ | 128-136 |
| | | | SmallSouthAmterica: + : 28274-29533) | |
| 3 | 2380-2389 | PRRARSVRYP linker 1-10 | | |
| 3 | 2390-2408 | ANSVFNICQSVTANVNALL | RNA-dependent_RNA_polymerase@Croatia(NC_045512@Croatia: + : | 690-708 |
| | | | 13442-13468 + 13468-16236) | |
| 3 | 2409-2418 | PRRARSVRYP linker 1-10 | | |
| 3 | 2419-2428 | QEHYVRITGL | helicase(NC_045512: + : 16237-18039) | 243-252 |
| 3 | 2429-2438 | PRRARSVRYP linker 1-10 | | |
| 3 | 2439-2446 | YDPLQPEL | surface_glycoprotein(NC_045512: + : 21563-25384) | 1138-1145 |
| 3 | 2447-2456 | PRRARSVRYP linker 1-10 | | |
| 3 | 2457-2465 | EVFAQVKQI | surface_glycoprotein(NC_045512: + : 21563-25384) | 780-788 |
| 3 | 2466-2475 | PRRARSVRYP linker 1-10 | | |
| 3 | 2476-2491 | FLVLLPLVSIQCVNLT | surface_glycoprotein@SmallNorthAmerica(NC_045512@SmallNorthAmerica: + : | 4-19 |
| | | | 21563-25384) | |
| 3 | 2492-2501 | PRRARSVRYP linker 1-10 | | |
| 3 | 2502-2511 | KQYGDCLGDI | surface_glycoprotein(NC_045512: + : 21563-25384) | 835-844 |
| 3 | 2512-2521 | PRRARSVRYP linker 1-10 | | |
| 3 | 2522-2536 | YNLLKGCPAVAKHDF | RNA-dependent_RNA_polymerase@Poland(NC_045512@Poland: + : | 87-101 |
| | | | 13442-13468 + 13468-16236) | |
| 4 | 1-14 | ANPVLPFNDGVYFA | surface_glycoprotein@Austria(NC_045512@Austria: + : 21563-25384) | 77-90 |
| 4 | 15-24 | PRRARSVRYP linker 1-10 | | |
| 4 | 25-35 | GKPVPYCYDTN | nsp4(NC_045512: + : 8555-10054) | 164-174 |
| 4 | 36-45 | PRRARSVRYP linker 1-10 | | |
| 4 | 46-57 | VTMPLGYVTHGL | nsp3(NC_045512: + : 2720-8554) | 628-639 |
| 4 | 58-67 | PRRARSVRYP linker 1-10 | | |
| 4 | 68-79 | FNCYFPLQSYGF | surface_glycoprotein(NC_045512: + : 21563-25384) | 486-497 |
| 4 | 80-89 | PRRARSVRYP linker 1-10 | | |
| 4 | 90-113 | PINLVRDLPQGFSVLEPLVDLPIG | surface_glycoprotein@Belgium(NC_045512@Belgium: + : 21563-25384) | 205-228 |
| 4 | 114-123 | PRRARSVRYP linker 1-10 | | |
| 4 | 124-144 | QWNLVIGFLFLTWICLLQFAY | membrane_glycoprotein@Aruba(NC_045512@Aruba: + : 26523-27191) | 19-39 |
| 4 | 145-154 | PRRARSVRYP linker 1-10 | | |
| 4 | 155-176 | SDFVRATATIPIQASLPFGWLI | ORF3a_protein@Aruba(NC_045512@Aruba: + : 25393-26220) | 26-47 |
| 4 | 177-186 | PRRARSVRYP linker 1-10 | | |
| 4 | 187-197 | AVHECFVKRVD | 3′-to-5′_exonuclease(NC_045512: + : 18040-19620) | 281-291 |
| 4 | 198-207 | PRRARSVRYP linker 1-10 | | |
| 4 | 208-215 | ETQALPQR | nucleocapsid_phosphoprotein@SmallNorthAmerica(NC_045512@ | 378-385 |
| | | | SmallNorthAmterica: + : 28274-29533) | |
| 4 | 216-225 | PRRARSVRYP linker 1-10 | | |
| 4 | 226-238 | LLNVPLHGTILTR | membrane_glycoprotein(NC_045512: + : 26523-27191) | 119-131 |
| 4 | 239-248 | PRRARSVRYP linker 1-10 | | |
| 4 | 249-268 | RLCAYCCNIVNVSLVKPSFY | envelope_protein(NC_045512: + : 26245-26472) | 38-57 |
| 4 | 269-278 | PRRARSVRYP linker 1-10 | | |
| 4 | 279-286 | EVTPSGTW | nucleocapsid_phosphoprotein(NC_045512: + : 28274-29533) | 323-330 |
| 4 | 287-296 | PRRARSVRYP linker 1-10 | | |
| 4 | 297-306 | DVVAIDYKHY | nsp3(NC_045512: + : 2720-8554) | 1148-1157 |
| 4 | 307-316 | PRRARSVRYP linker 1-10 | | |
| 4 | 317-325 | SPDVDLADI | surface_glycoprotein@Chile(NC_045512@Chile: + : 21563-25384) | 1161-1169 |
| 4 | 326-335 | PRRARSVRYP linker 1-10 | | |
| 4 | 336-358 | KNPLLYDANYFLCCHTNCYDYCI | ORF3a_protein@Senegal(NC_045512@Senegal: + : 25393-26220) | 136-158 |
| 4 | 359-368 | PRRARSVRYP linker 1-10 | | |
| 4 | 369-380 | NIGCNHTGVVGE | nsp2(NC_045512: + : 806-2719) | 250-261 |
| 4 | 381-390 | PRRARSVRYP linker 1-10 | | |
| 4 | 391-402 | AKLLHKPIVWHV | nsp3(NC_045512: + : 2720-8554) | 1165-1176 |
| 4 | 403-412 | PRRARSVRYP linker 1-10 | | |
| 4 | 413-424 | VRTNVYLAVFDK | nsp3(NC_045512: + : 2720-8554) | 351-362 |
| 4 | 425-434 | PRRARSVRYP linker 1-10 | | |
| 4 | 435-446 | VPVVDSYYSLLM | RNA-dependent_RNA_polymerase(NC_045512: + : 13442-13468 + 13468-16236) | 231-242 |
| 4 | 447-456 | PRRARSVRYP linker 1-10 | | |
| 4 | 457-464 | DQDVLFAY | RNA-dependent_RNA_polymerase@FrenchGuiana(NC_045512@ | 523-530 |
| | | | FrenchGuiana: + :13442-13468 + 13468-16236) | |
| 4 | 465-474 | PRRARSVRYP linker 1-10 | | |
| 4 | 475-487 | ATNVVIKVCEFQF | surface_glycoprotein(NC_045512: + : 21563-25384) | 123-135 |
| 4 | 488-497 | PRRARSVRYP linker 1-10 | | |
| 4 | 498-508 | YDKLQFISLEI | helicase@SouthAfrica(NC_045512@SouthAfrica: + : 16237-18039) | 582-592 |
| 4 | 509-518 | PRRARSVRYP linker 1-10 | | |
| 4 | 519-526 | SVDCTMYI | surface_glycoprotein(NC_045512: + : 21563-25384) | 735-742 |
| 4 | 528-536 | PRRARSVRYP linker 1-10 | | |
| 4 | 537-544 | SDFVRATA | ORF3a_protein(NC_045512: + : 25393-26220) | 26-33 |
| 4 | 545-554 | PRRARSVRYP linker 1-10 | | |
| 4 | 555-563 | ASQGLLASI | RNA-dependent_RNA_polymerase@Austria(NC_045512@Austria: + : | 771-779 |
| | | | 13442-13468 + 13468-16236) | |
| 4 | 564-573 | PRRARSVRYP linker 1-10 | | |
| 4 | 574-583 | PFTINVQEPK | ORF8_protein@India(NC_04551@India: + : 27894-28259) | 85-94 |
| 4 | 584-593 | PRRARSVRYP linker 1-10 | | |
| 4 | 594-601 | FKLASHMY | nsp3(NC_045512: + : 2720-8554) | 96-103 |
| 4 | 602-611 | PRRARSVRYP linker 1-10 | | |
| 4 | 612-625 | LMNVLTLVYKVYYG | nsp6@Aruba(NC_045512@Aruba: + : 10973-11842) | 139-152 |
| 4 | 626-635 | PRRARSVRYP linker 1-10 | | |
| 4 | 636-647 | SVFQSASKIITL | ORF3a_protein@Iceland(NC_045512@Iceland: + : 25393-26220) | 54-65 |
| 4 | 648-657 | PRRARSVRYP linker 1-10 | | |
| 4 | 658-680 | YLGGMSYYCKSHKLPSIFPLCAN | helicase@CostaRica(NC_045512@CostaRica: + : 16237-18039) | 64-86 |
| 4 | 681-690 | PRRARSVRYP linker 1-10 | | |
| 4 | 691-700 | NCYDYCIPYN | RF3a_protein(NC_045512: + : 25393-26220) | 152-161 |
| 4 | 701-710 | PRRARSVRYP linker 1-10 | | |
| 4 | 711-722 | YPSLETIQITIS | nsp3(NC_045512: + : 2720-8554) | 1483-1494 |
| 4 | 723-732 | PRRARSVRYP linker 1-10 | | |
| 4 | 733-783 | HFAIGLALYYPSARIVYTACSHAAVDALCEKY | helicase(NC_045512: + : 16237-18039) | 290-340 |
| | | LPIDKCSRIIPARARV | | |
| 4 | 784-793 | PRRARSVRYP linker 1-10 | | |
| 4 | 794-801 | STFKCYGV | surface_glycoprotein@Aruba(NC_045512@Aruba: + : 21563-25384) | 371-378 |
| 4 | 802-811 | PRRARSVRYP linker 1-10 | | |
| 4 | 812-821 | GLKLKDCVMY | nsp6@Peru(NC_045512@Peru: + : 10973-11842) | 107-116 |
| 4 | 822-831 | PRRARSVRYP linker 1-10 | | |
| 4 | 832-847 | DKYVRNLQHRLYECLY | RNA-dependent_RNA_polymerase@Aruba(NC_045512@Aruba: + : | 717-732 |
| | | | 13442-13468 + 13468-16236) | |
| 4 | 848-857 | PRRARSVRYP linker 1-10 | | |
| 4 | 858-869 | ISVTTEILPVSM | surface_glycoprotein(NC_045512: + : 21563-25384) | 720-731 |
| 4 | 870-879 | PRRARSVRYP linker 1-10 | | |
| 4 | 880-908 | TLQCIMLVYCFLGYGCTCYFGLFCLLNRY | nsp6@Aruba(NC_045512@Aruba: + : 10973-11842) | 203-231 |
| 4 | 909-918 | PRRARSVRYP linker 1-10 | | |
| 4 | 919-933 | QIELKFNPPALQDAY | nsp3(NC_045512: + : 2720-8554) | 867-881 |
| 4 | 934-943 | PRRARSVRYP linker 1-10 | | |
| 4 | 944-951 | DCATVHTA | 2′-O-ribose_methyltransferase(NC_04551: + : 20659-21552) | 114-121 |
| 4 | 952-961 | PRRARSVRYP linker 1-10 | | |
| 4 | 962-981 | IRRPFLCCKCCYDHVISTSH | helicase(NC_045512: + : 16237-18039) | 20-39 |
| 4 | 982-990 | PRRARSVRYP linker 1-10 | | |
| 4 | 992-1001 | LPFTINCQEL | ORF8_protein@Mozambique(NC_04551@Mozambique: + : 27894-28259) | 84-93 |
| 4 | 1002-1011 | PRRARSVRYP linker 1-10 | | |
| 4 | 1012-1031 | DANYFLCWHTNCYHYCIPYN | ORF3a_protein@Indonesia(NC_045512@Indonesia: + : 25393-26220) | 142-161 |
| 4 | 1032-1041 | PRRARSVRYP linker 1-10 | | |
| 4 | 1042-1051 | VVADAVIKTL | nsp3(NC_045512: + : 2720-8554) | 56-65 |
| 4 | 1052-1061 | PRRARSVRYP linker 1-10 | | |
| 4 | 1062-1072 | RQALLKTVQFC | RNA-dependent_RNA_polymerase@Aruba(NC_045512@Aruba: + : | 183-193 |
| | | | 13442-13468 + 13468-16236) | |
| 4 | 1073-1082 | PRRARSVRYP linker 1-10 | | |
| 4 | 1083-1090 | TDVVYRAF | RNA-dependent_RNA_polymerase@Aruba(NC_045512@Aruba: + : | 28-35 |
| | | | 13442-13468 + 13468-16236) | |
| 4 | 1091-1100 | PRRARSVRYP linker 1-10 | | |
| 4 | 1101-1122 | TLGVLPHVGEIPVAYRKVLLR | leader_protein(NC_045512: + : 266-805) | 103-124 |
| 4 | 1123-1132 | PRRARSVRYP linker 1-10 | | |
| 4 | 1133-1142 | VMCGGSLYVK | RNA-dependent_RNA_polymerase(NC_045512: + : 13442-13468 + 13468-16236) | 667-676 |
| 4 | 1143-1152 | PRRARSVRYP linker 1-10 | | |
| 4 | 1153-1165 | FQTLLALHRSYLT | surface_glycoprotein@Aruba(NC_045512@Aruba: + : 21563-25384) | 234-246 |
| 4 | 1166-1175 | PRRARSVRYP linker 1-10 | | |
| 4 | 1176-1183 | VLSEARQH | leader_protein(NC_045512: + : 266-805) | 38-45 |
| 4 | 1184-1193 | PRRARSVRYP linker 1-10 | | |
| 4 | 1194-1216 | GKIQDSLFSTASALGKLQDVVNQ | surface_glycoprotein@NorthMacedonia(NC_045512@NorthMacedonia: + : | 928-950 |
| | | | 21563-25384) | |
| 4 | 1217-1226 | PRRARSVRYP linker 1-10 | | |
| 4 | 1227-1244 | KGFCDLKGKYVQIPTTCA | nsp10(NC_045512: + : 13025-13441) | 87-104 |
| 4 | 1245-1254 | PRRARSVRYP linker 1-10 | | |
| 4 | 1255-1264 | GVITHDVSSA | helicase(NC_045512: + : 16237-18039) | 478-487 |
| 4 | 1265-1274 | PRRARSVRYP linker 1-10 | | |
| 4 | 1275-1284 | NLIIKNLSKS | ORF6_protein(NC_045512: + : 27202-27387) | 34-43 |
| 4 | 1285-1291 | PRRARSVRYP linker 1-10 | | |
| 4 | 1295-1316 | IAMACLVGLMWLSYFIASFRLF | membrane_glycoprotein@Aruba(NC_045512@Aruba: + : 26523-27191) | 82-103 |
| 4 | 1317-1326 | PRRARSVRYP linker 1-10 | | |
| 4 | 1327-1351 | SDIDITFLKKDAPYIVGDVVQEGVL | nsp3(NC_045512: + : 2720-8554) | 454-478 |
| 4 | 1352-1361 | PRRARSVRYP linker 1-10 | | |
| 4 | 1362-1380 | VKDCVVLHSYFTSDYYQLY | ORF3a_protein@Aruba(NC_045512@Aruba: + : 25393-26220) | 197-215 |
| 4 | 1381-1390 | PRRARSVRYP linker 1-10 | | |
| 4 | 1391-1398 | TFSVLACY | 3C-like_proteinase(NC_045512: + : 10055-10972) | 111-118 |
| 4 | 1399-1408 | PRRARSVRYP linker 1-10 | | |
| 4 | 1409-1420 | TRAKVGILCIMS | helicase(NC_045512: + : 16237-18039) | 566-577 |
| 4 | 1421-1430 | PRRARSVRYP linker 1-10 | | |
| 4 | 1431-1448 | SQHTMLVKQGDDYVYLPY | RNA-dependent_RNA_polymerase@Aruba(NC_045512@Aruba: + : | 814-831 |
| | | | 13442-13468 + 13468-16236) | |
| 4 | 1449-1458 | PRRARSVRYP linker 1-10 | | |
| 4 | 1459-1484 | YRLANECAQVLSEMVMCGGSLYVKPG | RNA-dependent_RNA_polymerase@Australia(NC_045512@Australia: + : | 653-678 |
| | | | 13442-13468 + 13468-16236) | |
| 4 | 1485-1494 | PRRARSVRYP linker 1-10 | | |
| 4 | 1495-1508 | IRNLQHRLYECLYR | RNA-dependent_RNA_polymerase@Austria(NC_045512@Austria: + : | 720-733 |
| | | | 13442-13468 + 13468-16236) | |
| 4 | 1509-1518 | PRRARSVRYP linker 1-10 | | |
| 4 | 1519-1534 | TGLHPTQAPTHLSVDT | 3′-to-5′_exonuclease(NC_045512: + : 18040-19620) | 16-31 |
| 4 | 1535-1544 | PRRARSVRYP linker 1-10 | | |
| 4 | 1545-1554 | LAWPLIVTAL | nsp8(NC_045512: + : 12092-12685) | 180-189 |
| 4 | 1555-1564 | PRRARSVRYP linker 1-10 | | |
| 4 | 1565-1581 | LTQYNRYLALYNKYKYF | nsp4(NC_045512: + : 8555-10054) | 438-454 |
| 4 | 1582-1591 | PRRARSVRYP linker 1-10 | | |
| 4 | 1592-1608 | AQSFLNRVCGVSAARLT | RNA-dependent_RNA_polymerase(NC_045512: + : 13442-13468 + 13468-16236) | 4-20 |
| 4 | 1609-1618 | PRRARSVRYP linker 1-10 | | |
| 4 | 1619-1626 | IAANTVIW | endoRNAse(NC_045512: + : 19621-20658) | 79-86 |
| 4 | 1627-1636 | PRRARSVRYP linker 1-10 | | |
| 4 | 1637-1647 | CYTPSLKLIEYT | nsp4(NC_045512: + : 8555-10054) | 133-143 |
| 4 | 1648-1657 | PRRARSVRYP linker 1-10 | | |
| 4 | 1658-1665 | DSYFVVKR | RNA-dependent_RNA_polymerase(NC_045512: + : 13442-13468 + 13468-16236) | 67-74 |
| 4 | 1666-1675 | PRRARSVRYP linker 1-10 | | |
| 4 | 1676-1691 | SEFSSLPSYAAFATAQ | nsp8(NC_045512: + : 12092-12685) | 4-19 |
| 4 | 1692-1701 | PRRARSVRYP linker 1-10 | | |
| 4 | 1702-1715 | ESVVKDCVVLHSYF | ORF3a_protein@CostaRica(NC_045512@CostaRica: + : 25393-26220) | 194-207 |
| 4 | 1716-1725 | PRRARSVRYP linker 1-10 | | |
| 4 | 1726-1747 | GYQPYRVVVLSFELLHAPATVC | surface_glycoprotein(NC_045512: + : 21563-25384) | 504-525 |
| 4 | 1748-1757 | PRRARSVRYP linker 1-10 | | |
| 4 | 1758-1772 | EVPANSTVLSFCAFA | nsp10(NC_045512: + : 13025-13441) | 6-20 |
| 4 | 1773-1782 | PRRARSVRYP linker 1-10 | | |
| 4 | 1783-1798 | TVLKKCKSAFYILPSI | nsp3(NC_045512: + : 2720-8554) | 526-541 |
| 4 | 1799-1808 | PRRARSVRYP linker 1-10 | | |
| 4 | 1809-1825 | INFVRIIMRLWLCLKCR | ORF3a_protein@Mozambique(NC_045512@Mozambique: + : 25393-26220) | 118-134 |
| 4 | 1826-1835 | PRRARSVRYP linker 1-10 | | |
| 4 | 1836-1848 | YAKPFLNKVVSTT | nsp3(NC_045512: + : 2720-8554) | 1323-1335 |
| 4 | 1849-1858 | PRRARSVRYP linker 1-10 | | |
| 4 | 1859-1876 | FLYENAFLPFAMGVIAMS | nsp6@Mexico(NC_045512@Mexico: + : 10973-11842) | 36-53 |
| 4 | 1877-1886 | PRRARSVRYP linker 1-10 | | |
| 4 | 1887-1897 | IEELFYSYATH | 3′-to-5′_exonuclease(NC_045512: + : 18040-19620) | 363-373 |
| 4 | 1898-1907 | PRRARSVRYP linker 1-10 | | |
| 4 | 1908-1915 | GVYCCREH | nsp2(NC_045512: + : 806-2719) | 47-54 |
| 4 | 1916-1925 | PRRARSVRYP linker 1-10 | | |
| 4 | 1926-1944 | DKSAFVNLKQLPFFYYSDS | 3′-to-5′_exonuclease(NC_045512: + : 18040-19620) | 432-450 |
| 4 | 1945-1954 | PRRARSVRYP linker 1-10 | | |
| 4 | 1955-1968 | DYDCVSFCYMHHME | 3C-like_proteinase(NC_045512: + : 10055-10972) | 153-166 |
| 4 | 1969-1978 | PRRARSVRYP linker 1-10 | | |
| 4 | 1979-2000 | SDFVRATATIPIRASLPFGWLI | ORF3a_protein@Belgium(NC_045512@Belgium: + : 25393-26220) | 26-47 |
| 4 | 2001-2010 | PRRARSVRYP linker 1-10 | | |
| 4 | 2011-2020 | ARDLICAQKF | surface_glycoprotein(NC_045512: + : 21563-25384) | 846-855 |
| 4 | 2021-2030 | PRRARSVRYP linker 1-10 | | |
| 4 | 2031-2038 | ISNCVADY | surface_glycoprotein(NC_045512: + : 21563-25384) | 358-365 |
| 4 | 2039-2057 | PRRARSVRYP linker 1-10 | | |
| 4 | 2049-2063 | ISNCVADYSVLYNSA | surface_glycoprotein@Aruba(NC_045512@Aruba: + : 21563-25384) | 354-368 |
| 4 | 2064-2073 | PRRARSVRYP linker 1-10 | | |
| 4 | 2074-2090 | VPQEHYVRITGLYPTLN | helicase@Aruba(NC_045512@Aruba: + : 16237-18039) | 241-257 |
| 4 | 2091-2100 | PRRARSVRYP linker 1-10 | | |
| 4 | 2101-2112 | ALGKLQDVVNQN | surface_glycoprotein(NC_045512: + : 21563-25384) | 944-955 |
| 4 | 2113-2122 | PRRARSVRYP linker 1-10 | | |
| 4 | 2123-2133 | LRIAGHHLGRC | membrane_glycoprotein(NC_045512: + : 26523-27191) | 149-159 |
| 4 | 2134-2143 | PRRARSVRYP linker 1-10 | | |
| 4 | 2144-2162 | VKDCVLLHSYFTSDYYQLY | ORF3a_protein@Belgium(NC_045512@Belgium: + : 25393-26220) | 197-215 |
| 4 | 2163-2172 | PRRARSVRYP linker 1-10 | | |
| 4 | 2173-2180 | AALQIPFA | surface_glycoprotein(NC_045512: + : 21563-25384) | 892-899 |
| 4 | 2181-2190 | PRRARSVRYP linker 1-10 | | |
| 4 | 2191-2206 | FSNVTWFHAIHVSGTN | surface_glycoprotein@Peru(NC_045512@Peru: + : 21563-25384) | 59-74 |
| 4 | 2207-2216 | PRRARSVRYP linker 1-10 | | |
| 4 | 2217-2231 | HYVRITGLYPTLNIL | helicase@Indonesia(NC_045512@Indonesia: + : 16237-18039) | 245-259 |
| 4 | 2232-2241 | PRRARSVRYP linker 1-10 | | |
| 4 | 2242-2261 | DDRCILHCANFNVLFSTVFP | RNA-dependent_RNA_polymerase(NC_045512: + : 13442-13468 + 13468-16236) | 303-322 |
| 4 | 2262-2271 | PRRARSVRYP linker 1-10 | | |
| 4 | 2272-2281 | SSVLHSTQDF | surface_glycoprotein@Norway(NC_045512@Norway: + : 21563-25384) | 45-54 |
| 4 | 2282-2291 | PRRARSVRYP linker 1-10 | | |
| 4 | 2292-2299 | GSCGSVGF | 3C-like_proteinase(NC_045512: + : 10055-10972) | 143-150 |
| 4 | 2300-2309 | PRRARSVRYP linker 1-10 | | |
| 4 | 2310-2324 | YFTSDYYQLYSTQLI | ORF3a_protein@Indonesia(NC_045512@Indonesia: + : 25393-26220) | 206-220 |
| 4 | 2325-2334 | PRRARSVRYP linker 1-10 | | |
| 4 | 2335-2343 | LKLKDCVMY | nsp6@Aruba(NC_045512@Aruba: + : 10973-11842) | 105-113 |
| 4 | 2344-2353 | PRRARSVRYP linker 1-10 | | |
| 4 | 2354-2368 | RIIPARARVECFDKF | helicase@Aruba(NC_045512@Aruba: + : 16237-18039) | 332-346 |
| 4 | 2369-2378 | PRRARSVRYP linker 1-10 | | |
| 4 | 2379-2388 | TQNLYENQK | surface_glycoprotein(NC_045512: + : 21563-25384) | 912-921 |
| 4 | 2389-2398 | PRRARSVRYP linker 1-10 | | |
| 4 | 2399-2421 | NHDLYCQVHGNAHVASCDAIMTR | 3′-to-5′_exonuclease(NC_045512: + : 18040-19620) | 256-278 |
| 4 | 2422-2431 | PRRARSVRYP linker 1-10 | | |
| 4 | 2432-2439 | TDAVDCAL | surface_glycoprotein(NC_045512: + : 21563-25384) | 286-293 |
| 4 | 2440-2449 | PRRARSVRYP linker 1-10 | | |
| 4 | 2450-2463 | IKVCEFQFCNDPYY | surface_glycoprotein@CostaRica(NC_045512@CostaRica: + : 21563-25384) | 128-141 |
| 4 | 2464-2473 | PRRARSVRYP linker 1-10 | | |
| 4 | 2474-2483 | DPIHSLRVCV | nsp3(NC_045512: + : 2720-8554) | 339-348 |
| 4 | 2484-2493 | PRRARSVRYP linker 1-10 | | |
| 4 | 2494-2515 | LLYDANYFLCWHINCYDYCIPY | ORF3a_protein@Estonai(NC_045512@Estonia: + : 25393-26220) | 139-160 |

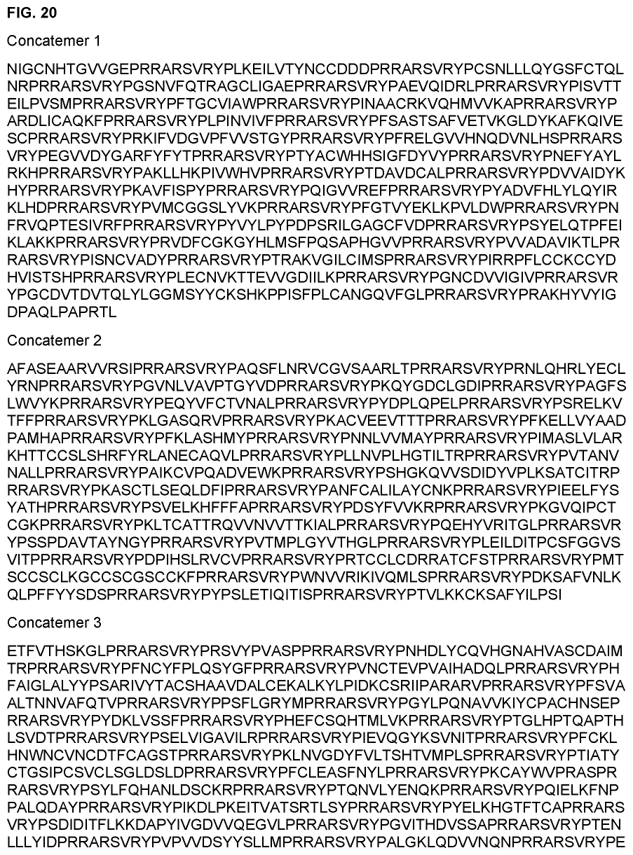

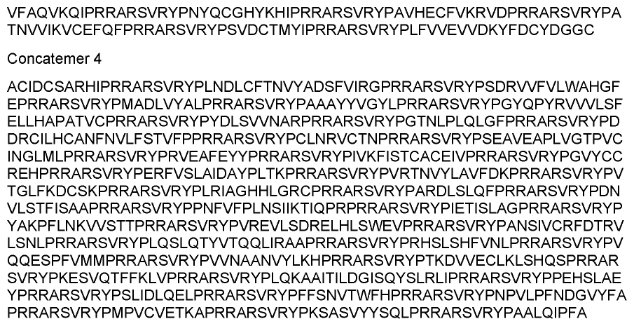

In certain embodiments, the vaccine comprises or expresses at least one of the concatemers of epitopes with intervening linker sequences (and as set forth in FIG. 20). In certain embodiments, the vaccine comprises or expresses at least two of the concatemers set forth below. In certain embodiments, the vaccine comprises or expresses three of the concatemers set forth below. In certain embodiments, the vaccine comprises or expresses all the concatemers set forth below.

TABLE 10

| concatemer | pos_in_concatemer | epitope | gene | pos_in_gene |
|---|---|---|---|---|
| 1 | 1-12 | NIGCNHTGVVGE | nsp2(NC_045512: + : 806-2719) | 250-261 |
| 1 | 13-22 | PRRARSVRYP linker 1-10 | | |
| 1 | 23-36 | LKEILVTYNCCDDD | RNA-dependent_RNA_polymerase(NC_045512: + : 13442-13468 + 13468-16236) | 142-155 |
| 1 | 37-46 | PRRARSVRYP linker 1-10 | | |
| 1 | 47-63 | CSNLLLQYGSFCTQLNR | surface_glycoprotein(NC_045512: + : 21563-25384) | 749-765 |
| 1 | 64-73 | PRRARSVRYP linker 1-10 | | |
| 1 | 74-89 | GSNVFQTRAGCLIGAE | surface_glycoprotein(NC_045512: + : 21563-25384) | 639-654 |
| 1 | 90-99 | PRRARSVRYP linker 1-10 | | |
| 1 | 100-107 | AEVQIDRL | surface_glycoprotein(NC_045512: + : 21563-25384) | 989-996 |
| 1 | 108-117 | PRRARSVRYP linker 1-10 | | |
| 1 | 118-129 | ISVTTEILPVSM | surface_glycoprotein(NC_045512: + : 21563-25384) | 720-731 |
| 1 | 130-139 | PRRARSVRYP linker 1-10 | | |
| 1 | 140-147 | FTGCVIAW | surface_glycoprotein(NC_045512: + : 21563-25384) | 429-436 |
| 1 | 148-157 | PRRARSVRYP linker 1-10 | | |
| 1 | 158-172 | INAACRKVQHMVVKA | 3′-to-5′_exonuclease(NC_045512: + : 18040-19620) | 305-319 |
| 1 | 173-182 | PRRARSVRYP linker 1-10 | | |
| 1 | 183-192 | ARDLICAQKF | surface_glycoprotein(NC_045512: + : 21563-25384) | 846-855 |
| 1 | 193-202 | PRRARSVRYP linker 1-10 | | |
| 1 | 203-210 | LPINVIVF | nsp3(NC_045512: + : 2720-8554) | 1718-1725 |
| 1 | 211-220 | PRRARSVRYP linker 1-10 | | |
| 1 | 221-247 | FSASTSAFVETVKGLDYKAFKQIVESC | nsp2(NC_045512: + : 806-2719) | 300-326 |
| 1 | 248-257 | PRRARSVRYP linker 1-10 | | |
| 1 | 258-273 | RKIFVDGVPFVVSTGY | RNA-dependent_RNA_polymerase(NC_045512: + : 13442-13468 + 13468-16236) | 331-346 |
| 1 | 274-283 | PRRARSVRYP linker 1-10 | | |
| 1 | 284-299 | FRELGVVHNQDVNLHS | RNA-dependent_RNA_polymerase(NC_045512: + : 13442-13468 + 13468-16236) | 348-363 |
| 1 | 300-309 | PRRARSVRYP linker 1-10 | | |
| 1 | 310-323 | EGVVDYGARFYFYT | nsp3(NC_045512: + : 2720-8554) | 595-608 |
| 1 | 324-333 | PRRARSVRYP linker 1-10 | | |
| 1 | 334-348 | TYACWHHSIGFDYVY | 3′-to-5′_exonuclease(NC_045512: + : 18040-19620) | 223-237 |
| 1 | 349-358 | PRRARSVRYP linker 1-10 | | |
| 1 | 359-368 | NEFYAYLRKH | RNA-dependent_RNA_polymerase(NC_045512: + : 13442-13468 + 13468-16236) | 743-752 |
| 1 | 369-378 | PRRARSVRYP linker 1-10 | | |
| 1 | 379-390 | AKLLHKPIVWHV | nsp3(NC_045512: + : 2720-8554) | 1165-1176 |
| 1 | 391-400 | PRRARSVRYP linker 1-10 | | |
| 1 | 401-408 | TDAVDCAL | surface_glycoprotein(NC_045512: + : 21563-25384) | 286-293 |
| 1 | 409-418 | PRRARSVRYP linker 1-10 | | |
| 1 | 419-428 | DVVAIDYKHY | nsp3(NC_045512: + : 2720-8554) | 1148-1157 |
| 1 | 429-438 | PRRARSVRYP linker 1-10 | | |
| 1 | 439-446 | KAVFISPY | helicase(NC_045512: + : 16237-18039) | 508-515 |
| 1 | 447-456 | PRRARSVRYP linker 1-10 | | |
| 1 | 457-464 | QIGVVREF | helicase(NC_045512: + : 16237-18039) | 492-499 |
| 1 | 465-474 | PRRARSVRYP linker 1-10 | | |
| 1 | 475-491 | YUADVFHLYLQYRIKLHD | RNA-dependent_RNA_polymerase(NC_045512: + : 13442-13468 + 13468-16236) | 877-893 |
| 1 | 492-501 | PRRARSVRYP linker 1-10 | | |
| 1 | 502-511 | VMCGGSLYVK | RNA-dependent_RNA_polymerase(NC_045512: + : 13442-13468 + 13468-16236) | 667-676 |
| 1 | 512-521 | PRRARSVRYP linker 1-10 | | |
| 1 | 522-535 | FGTVYEKLKPVLDW | nsp2(NC_045512: + : 806-2719) | 437-450 |
| 1 | 536-545 | PRRARSVRYP linker 1-10 | | |
| 1 | 546-558 | VFRVQPTESIVRF | surface_glycoprotein(NC_045512: + : 21563-25384) | 317-329 |
| 1 | 559-568 | PRRARSVRYP linker 1-10 | | |
| 1 | 569-588 | YVYLPYPDPSRILGAGCFVD | RNA-dependent_RNA_polymerase(NC_045512: + : 13442-13468 + 13468-16236) | 826-845 |
| 1 | 589-598 | PRRARSVRYP linker 1-10 | | |
| 1 | 599-613 | SYELQTPFEIKLAKK | nsp2(NC_045512: + : 806-2719) | 68-82 |
| 1 | 614-623 | PRRARSVRYP linker 1-10 | | |
| 1 | 624-646 | RVDFCGKGYHLMSFPQSAPHGVV | surface_glycoprotein(NC_045512: + : 21563-25384) | 1039-1061 |
| 1 | 647-656 | PRRARSVRYP linker 1-10 | | |
| 1 | 657-666 | VVADAVIKTL | nsp3(NC_045512: + : 2720-8554) | 56-65 |
| 1 | 667-676 | PRRARSVRYP linker 1-10 | | |
| 1 | 677-684 | ISNCVADY | surface_glycoprotein(NC_045512: + : 21563-25384) | 358-365 |
| 1 | 685-694 | PRRARSVRYP linker 1-10 | | |
| 1 | 695-706 | TRAKVGILCIMS | helicase(NC_045512: + : 16237-18039) | 566-577 |
| 1 | 707-716 | PRRARSVRYP linker 1-10 | | |
| 1 | 717-736 | IRRPFLCCKCCYDHVISTSH | helicase(NC_045512: + : 16237-18039) | 20-39 |
| 1 | 737-746 | PRRARSVRYP linker 1-10 | | |
| 1 | 747-763 | LECNVKTTEVVGDIILK | nsp3(NC_045512: + : 2720-8554) | 1244-1260 |
| 1 | 764-773 | PRRARSVRYP linker 1-10 | | |
| 1 | 774-783 | GNCDVVIGIV | surface_glycoprotein(NC_045512: + : 21563-25384) | 1124-1133 |
| 1 | 784-793 | PRRARSVRYP linker 1-10 | | |
| 1 | 794-832 | GCDVTDVTQLYLGGMSYYCKSHKPPISFPLCAN | helicase(NC_045512: + : 16237-18039) | 54-92 |
| | | GQVFGL | | |
| 1 | 833-842 | PRRARSVRYP linker 1-10 | | |
| 1 | 843-862 | RAKHYVYIGDPAQLPAPRTL | helicase(NC_045512: + : 16237-18039) | 392-411 |
| 2 | 1-13 | AFASEAARVVRSI | nsp2(NC_045512: + : 806-2719) | 355-367 |
| 2 | 14-23 | PRRARSVRYP linker 1-10 | | |
| 2 | 24-40 | AQSFLNRVCGVSAARLT | RNA-dependent_RNA_polymerase(NC_045512: + : 13442-13468 + 13468-16236) | 4-20 |
| 2 | 41-50 | PRRARSVRYP linker 1-10 | | |
| 2 | 51-64 | RNLQHRLYECLYRN | RNA-dependent_RNA_polymerase(NC_045512: + : 13442-13468 + 13468-16236) | 721-734 |
| 2 | 65-74 | PRRARSVRYP linker 1-10 | | |
| 2 | 75-87 | GVNLVAVPTGYVD | 3′-to-5′_exonuclease(NC_045512: + : 18040-19620) | 114-126 |
| 2 | 88-97 | PRRARSVRYP linker 1-10 | | |
| 2 | 98-107 | KQYGDCLGDI | surface_glycoprotein(NC_045512: + : 21563-25384) | 835-844 |
| 2 | 108-117 | PRRARSVRYP linker 1-10 | | |
| 2 | 118-126 | AGFSLWVYK | 3′-to-5′_exonuclease(NC_045512: + : 18040-19620) | 504-512 |
| 2 | 127-136 | PRRARSVRYP linker 1-10 | | |
| 2 | 137-147 | EQYVFCTVNAL | helicase(NC_045512: + : 16237-18039) | 353-363 |
| 2 | 148-157 | PRRARSVRYP linker 1-10 | | |
| 2 | 158-165 | YDPLQPEL | surface_glycoprotein(NC_045512: + : 21563-25384) | 1138-1145 |
| 2 | 166-175 | PRRARSVRYP linker 1-10 | | |
| 2 | 176-184 | SRELKVTFF | nsp3(NC_045512: + : 2720-8554) | 1134-1142 |
| 2 | 185-194 | PRRARSVRYP linker 1-10 | | |
| 2 | 195-202 | KLGASQRV | membrane_glycoprotein(NC_045512: + : 26523-27191) | 180-187 |
| 2 | 203-212 | PRRARSVRYP linker 1-10 | | |
| 2 | 213-222 | KACVEEVTTT | nsp3(NC_045512: + : 2720-8554) | 415-424 |
| 2 | 223-232 | PRRARSVRYP linker 1-10 | | |
| 2 | 233-247 | FKELLVYAADPAMHA | RNA-dependent_RNA_polymerase(NC_045512: + : 13442-13468 + 13468-16236) | 368-382 |
| 2 | 248-257 | PRRARSVRYP linker 1-10 | | |
| 2 | 258-265 | FKLASHMY | nsp3(NC_045512: + : 2720-8554) | 96-103 |
| 2 | 266-275 | PRRARSVRYP linker 1-10 | | |
| 2 | 276-283 | NNLVVMAY | nsp2(NC_045512: + : 806-2719) | 413-420 |
| 2 | 284-293 | PRRARSVRYP linker 1-10 | | |
| 2 | 294-325 | IMASLVLARKHTTCCSLSHRFYRLANECAQVL | RNA-dependent_RNA_polymerase(NC_045512: + : 13442-13468 + 13468-16236) | 632-663 |
| 2 | 326-335 | PRRARSVRYP linker 1-10 | | |
| 2 | 336-348 | LLNVPLHGTILTR | membrane_glycoprotein(NC_045512: + : 26523-27191) | 119-131 |
| 2 | 349-358 | PRRARSVRYP linker 1-10 | | |
| 2 | 359-367 | VTANVNALL | RNA-dependent_RNA_polymerase(NC_045512: + : 13442-13468 + 13468-16236) | 700-706 |
| 2 | 368-377 | PRRARSVRYP linker 1-10 | | |
| 2 | 378-390 | AIKCVPQADVEWK | 3′-to-5′_exonuclease(NC_045512: + : 18040-19620) | 337-349 |
| 2 | 391-400 | PRRARSVRYP linker 1-10 | | |
| 2 | 401-423 | SHGKQVVSDIDYVPLKSATCITR | 3′-to-5′_exonuclease(NC_045512: + : 18040-19620) | 454-476 |
| 2 | 424-433 | PRRARSVRYP linker 1-10 | | |
| 2 | 434-446 | KASCTLSEQLDFI | nsp2(NC_045512: + : 806-2719) | 30-42 |
| 2 | 447-456 | PRRARSVRYP linker 1-10 | | |
| 2 | 457-469 | ANFCALILAYCNK | nsp3(NC_045512: + : 2720-8554) | 890-902 |
| 2 | 470-479 | PRRARSVRYP linker 1-10 | | |
| 2 | 480-490 | IEELFYSYATH | 3′-to-5′_exonuclease(NC_045512: + : 18040-19620) | 363-373 |
| 2 | 491-500 | PRRARSVRYP linker 1-10 | | |
| 2 | 501-510 | SVELKHFFFA | RNA-dependent_RNA_polymerase(NC_045512: + : 13442-13468+ 13468-16236) | 434-443 |
| 2 | 511-520 | PRRARSVRYP linker 1-10 | | |
| 2 | 521-528 | DSYFVVKR | RNA-dependent_RNA_polymerase(NC_045512: + : 13442-13468+ 13468-16236) | 67-74 |
| 2 | 529-538 | PRRARSVRYP linker 1-10 | | |
| 2 | 539-549 | KGVQIPCTCGK | nsp3(NC_045512: + : 2720-8554) | 963-973 |
| 2 | 550-559 | PRRARSVRYP linker 1-10 | | |
| 2 | 560-579 | KLTCATTRQVVNVVTTKIAL | nsp3(NC_045512: + : 2720-8554) | 1923-1942 |
| 2 | 580-589 | PRRARSVRYP linker 1-10 | | |
| 2 | 590-599 | QEHYVRITGL | helicase(NC_045512: + : 16237-18039) | 243-252 |
| 2 | 600-609 | PRRARSVRYP linker 1-10 | | |
| 2 | 610-621 | SSPDAVTAYNGY | nsp3(NC_045512: + : 2720-8554) | 660-671 |
| 2 | 622-631 | PRRARSVRYP linker 1-10 | | |
| 2 | 632-643 | VTMPLGYVTHGL | nsp3(NC_045512: + : 2720-8554) | 628-639 |
| 2 | 644-653 | PRRARSVRYP linker 1-10 | | |
| 2 | 654-672 | LEILDITPCSFGGVSVITP | surface_glycoprotein(NC_045512: + : 21563-25384) | 582-600 |
| 2 | 673-682 | PRRARSVRYP linker 1-10 | | |
| 2 | 683-692 | DPIHSLRVCV | nsp3(NC_045512: + : 2720-8554) | 339-348 |
| 2 | 693-702 | PRRARSVRYP linker 1-10 | | |
| 2 | 703-717 | RTCCLCDRRATCFST | 3′-to-5′_exonuclease(NC_045512: + : 18040-19620) | 205-219 |
| 2 | 718-727 | PRRARSVRYP linker 1-10 | | |
| 2 | 728-747 | MTSCCSCLKGCCSCGSCCKF | surface_glycoprotein(NC_045512: + : 21563-25384) | 1237-1256 |
| 2 | 748-757 | PRRARSVRYP linker 1-10 | | |
| 2 | 758-770 | WNVVRIKIVQMLS | 3′-to-5′_exonuclease(NC_045512: + : 18040-19620) | 159-171 |
| 2 | 771-780 | PRRARSVRYP linker 1-10 | | |
| 2 | 781-799 | DKSAFVNLKQLPFFYYSDS | 3′-to-5′_exonuclease(NC_045512: + : 18040-19620) | 432-450 |
| 2 | 800-809 | PRRARSVRYP linker 1-10 | | |
| 2 | 810-821 | YPSLETIQITIS | nsp3(NC_045512: + : 2720-8554) | 1483-1494 |
| 2 | 822-831 | PRRARSVRYP linker 1-10 | | |
| 2 | 832-847 | TVLKKCKSAFYILPSI | nsp3(NC_045512: + : 2720-8554) | 526-541 |
| 3 | 1-10 | ETFVTHSKGL | nsp2(NC_045512: + : 806-2719) | 527-536 |
| 3 | 11-20 | PRRARSVRYP linker 1-10 | | |
| 3 | 21-29 | RSVYPVASP | nsp2(NC_045512: + : 806-2719) | 121-129 |
| 3 | 30-39 | PRRARSVRYP linker 1-10 | | |
| 3 | 40-62 | NHDLYCQVHGNAHVASCDAIMTR | 3′-to-5′_exonuclease(NC_045512: + : 18040-19620) | 256-278 |
| 3 | 63-72 | PRRARSVRYP linker 1-10 | | |
| 3 | 73-84 | FNCYFPLQSYGF | surface_glycoprotein(NC_045512: + : 21563-25384) | 486-497 |
| 3 | 85-94 | PRRARSVRYP linker 1-10 | | |
| 3 | 95-109 | VNCTEVPVAIHADQL | surface_glycoprotein(NC_045512: + : 21563-25384) | 615-629 |
| 3 | 110-119 | PRRARSVRYP linker 1-10 | | |
| 3 | 120-170 | HFAIGLALYYPSARIVYTACSHAAVDALCEKAL | helicase(NC_045512: + : 16237-18039) | 290-340 |
| | | KYLPIDKCSRIIPARARV | | |
| 3 | 171-180 | PRRARSVRYP linker 1-10 | | |
| 3 | 181-195 | FSVAALTNNVAFQTV | RNA-dependent_RNA_polymerase(NC_045512: + : 13442-13468+ 13468-16236) | 396-410 |
| 3 | 196-205 | PRRARSVRYP linker 1-10 | | |
| 3 | 206-213 | PSFLGRYM | nsp3(NC_045512: + : 2720-8554) | 822-829 |
| 3 | 214-223 | PRRARSVRYP linker 1-10 | | |
| 3 | 224-243 | GYLPQNAVVKIYCPACHNSE | nsp2(NC_045512: + : 806-2719) | 178-197 |
| 3 | 244-253 | PRRARSVRYP linker 1-10 | | |
| 3 | 254-261 | YDKLVSSF | nsp3(NC_045512: + : 2720-8554) | 365-372 |
| 3 | 262-271 | PRRARSVRYP linker 1-10 | | |
| 3 | 272-283 | HEFCSQHTMLVK | RNA-dependent_RNA_polymerase(NC_045512: + : 13442-13468+ 13468-16236) | 821-821 |
| 3 | 284-293 | PRRARSVRYP linker 1-10 | | |
| 3 | 294-309 | TGLHPTQAPTHLSVDT | 3′-to-5′_exonuclease(NC_045512: + : 18040-19620) | 16-31 |
| 3 | 310-319 | PRRARSVRYP linker 1-10 | | |
| 3 | 320-330 | SELVIGAVILR | membrane_glycoprotein(NC_045512: + : 26523-27191) | 136-146 |
| 3 | 331-340 | PRRARSVRYP linker 1-10 | | |
| 3 | 341-352 | IEVQGYKSVNIT | nsp3(NC_045512: + : 2720-8554) | 13-24 |
| 3 | 353-362 | PRRARSVRYP linker 1-10 | | |
| 3 | 363-382 | FCKLHNWNCVNCDTFCAGST | nsp3(NC_045512: + : 2720-8554) | 1626-1645 |
| 3 | 383-392 | PRRARSVRYP linker 1-10 | | |
| 3 | 393-411 | KLNVGDYFVLTSHTVMPLS | helicase(NC_045512: + : 16237-18039) | 218-236 |
| 3 | 412-421 | PRRARSVRYP linker 1-10 | | |
| 3 | 422-444 | TIATYCTGSIPCSVCLSGLDSLD | nsp3(NC_045512: + : 2720-8554) | 1459-1481 |
| 3 | 445-454 | PRRARSVRYP linker 1-10 | | |
| 3 | 455-464 | FCLEASFNYL | nsp3(NC_045512: + : 2720-8554) | 1931-1400 |
| 3 | 465-474 | PRRARSVRYP linker 1-10 | | |
| 3 | 475-484 | KCAYWVPRAS | nsp2(NC_045512: + : 806-2719) | 239-248 |
| 3 | 485-494 | PRRARSVRYP linker 1-10 | | |
| 3 | 495-508 | SYLFQHANLDSCKR | nsp3(NC_045512: + : 2720-8554) | 915-928 |
| 3 | 509-518 | PRRARSVRYP linker 1-10 | | |
| 3 | 519-528 | TQNVLYENQK | surface_glycoprotein(NC_045512: + : 21563-25384) | 912-921 |
| 3 | 529-538 | PRRARSVRYP linker 1-10 | | |
| 3 | 539-553 | QIELKFNPPALQDAY | nsp3(NC_045512: + : 2720-8554) | 867-881 |
| 3 | 554-563 | PRRARSVRYP linker 1-10 | | |
| 3 | 564-581 | IKDLPKEITVATSRTLSY | membrane_glycoprotein(NC_045512: + : 26523-27191) | 161-178 |
| 3 | 582-591 | PRRARSVRYP linker 1-10 | | |
| 3 | 592-602 | YELKHGTFTCA | nsp3(NC_045512: + : 2720-8554) | 996-1006 |
| 3 | 603-612 | PRRARSVRYP linker 1-10 | | |
| 3 | 613-637 | SDIDITFLKKDAPYIVGDVVQEGVL | nsp3(NC_045512: + : 2720-8554) | 454-478 |
| 3 | 638-647 | PRRARSVRYP linker 1-10 | | |
| 3 | 648-657 | GVITHDVSSA | helicase(NC_045512: + : 16237-18039) | 478-487 |
| 3 | 658-667 | PRRARSVRYP linker 1-10 | | |
| 3 | 668-676 | TENLLLYID | nsp3(NC_045512: + : 2720-8554) | 432-440 |
| 3 | 677-686 | PRRARSVRYP linker 1-10 | | |
| 3 | 687-698 | VPVVDAYYSLLM | RNA-dependent_RNA_polymerase(NC_045512: + : 13442-13468+ 13468-16236) | 231-242 |
| 3 | 699-708 | PRRARSVRYP linker 1-10 | | |
| 3 | 709-720 | ALGKLQDVVNQN | surface_glycoprotein(NC_045512: + : 21563-25384) | 944-955 |
| 3 | 721-730 | PRRARSVRYP linker 1-10 | | |
| 3 | 731-739 | EVFAQVKQI | surface_glycoprotein(NC_045512: + : 21563-25384) | 780-788 |
| 3 | 740-749 | PRRARSVRYP linker 1-10 | | |
| 3 | 750-759 | NYQCGHYKHI | nsp3(NC_045512: + : 2720-8554) | 1012-1021 |
| 3 | 760-769 | PRRARSVRYP linker 1-10 | | |
| 3 | 770-780 | AVHECFVKRVD | 3′-to-5′_exonuclease(NC_045512: + : 18040-19620) | 281-291 |
| 3 | 781-790 | PRRARSVRYP linker 1-10 | | |
| 3 | 791-803 | ATNVVIKVCEFQF | surface_glycoprotein(NC_045512: + : 21563-25384) | 123-135 |
| 3 | 804-813 | PRRARSVRYP linker 1-10 | | |
| 3 | 814-821 | SVDCTMYI | surface_glycoprotein(NC_045512: + : 21563-25384) | 735-742 |
| 3 | 822-831 | PRRARSVRYP linker 1-10 | | |
| 3 | 832-849 | LFVVEVVDKYFDCYDGGC | RNA-dependent_RNA_polymerase(NC_045512: + : 13442-13468+ 13468-16236) | 470-487 |
| 4 | 1-10 | ACIDCSARHI | nsp3(NC_045512: + : 2720-8554) | 1872-1881 |
| 4 | 11-20 | PRRARSVRYP linker 1-10 | | |
| 4 | 21-38 | LNDLCFTNVYADSFVIRG | surface_glycoprotein(NC_045512: + : 21563-25384) | 387-404 |
| 4 | 39-48 | PRRARSVRYP linker 1-10 | | |
| 4 | 49-62 | SDRVVFVLWAHGFE | 3′-to-5′_exonuclease(NC_045512: + : 18040-19620) | 178-191 |
| 4 | 63-72 | PRRARSVRYP linker 1-10 | | |
| 4 | 73-80 | MADLVYAL | RNA-dependent_RNA_polymerase(NC_045512: + : 13442-13468+ 13468-16236) | 124-131 |
| 4 | 81-90 | PRRARSVRYP linker 1-10 | | |
| 4 | 91-99 | AAYYVGYL | surface_glycoprotein(NC_045512: + : 21563-25384) | 262-270 |
| 4 | 100-109 | PRRARSVRYP linker 1-10 | | |
| 4 | 110-131 | GYQPYRVVVLSFELLHAPATVC | surface_glycoprotein(NC_045512: + : 21563-25384) | 504-525 |
| 4 | 132-141 | PRRARSVRYP linker 1-10 | | |
| 4 | 142-150 | YDLSVVNAR | helicase(NC_045512: + : 16237-18039) | 382-390 |
| 4 | 151-160 | PRRARSVRYP linker 1-10 | | |
| 4 | 161-170 | GTNLPLQLGF | 3′-to-5′_exonuclease(NC_045512: + : 18040-19620) | 102-111 |
| 4 | 171-180 | PRRARSVRYP linker 1-10 | | |
| 4 | 181-200 | DDRVILHCANFNVLFSTVFP | RNA-dependent_RNA_polymerase(NC_045512: + : 13442-13468+ 13468-16236) | 303-322 |
| 4 | 201-210 | PRRARSVRYP linker 1-10 | | |
| 4 | 211-218 | CLNRVCTN | nsp3(NC_045512: + : 2720-8554) | 1342-1349 |
| 4 | 219-228 | PRRARSVRYP linker 1-10 | | |
| 4 | 229-248 | SEAVEAPLVGTPVCINGLML | nsp2(NC_045512: + : 806-2719) | 591-610 |
| 4 | 249-258 | PRRARSVRYP linker 1-10 | | |
| 4 | 259-266 | RVEAFEYY | nsp3(NC_045512: + : 2720-8554) | 810-817 |
| 4 | 267-276 | PRRARSVRYP linker 1-10 | | |
| 4 | 277-289 | IVKFISTCACEIV | nsp2(NC_045512: + : 806-2719) | 468-480 |
| 4 | 290-299 | PRRARSVRYP linker 1-10 | | |
| 4 | 300-307 | GVYCCREH | nsp2(NC_045512: + : 806-2719) | 47-54 |
| 4 | 308-317 | PRRARSVRYP linker 1-10 | | |
| 4 | 318-332 | ERFVSLAIDAYPLTK | RNA-dependent_RNA_polymerase(NC_045512: + : 13442-13468+ 13468-16236) | 857-871 |
| 4 | 333-342 | PRRARSVRYP linker 1-10 | | |
| 4 | 343-354 | VRTNVYLAVFDK | nsp3(NC_045512: + : 2720-8554) | |
| 4 | 355-364 | PRRARSVRYP linker 1-10 | | 351-362 |
| 4 | 365-374 | VTGLFKDCSK | 3′-to-5′_exonuclease(NC_045512: + : 18040-19620) | |
| 4 | 375-384 | PRRARSVRYP linker 1-10 | | 4-13 |
| 4 | 385-395 | LRIAGHHLGRC | membrane_glycoprotein(NC_045512: + : 26523-27191) | 149-159 |
| 4 | 396-405 | PRRARSVRYP linker 1-10 | | |
| 4 | 406-413 | ARDLSLQF | nsp3(NC_045512: + : 2720-8554) | |
| 4 | 414-423 | PRRARSVRYP linker 1-10 | | 1652-1659 |
| 4 | 424-434 | DNVLSTFISAA | nsp3(NC_045512: + : 2720-8554) | |
| 4 | 435-444 | PRRARSVRYP linker 1-10 | | 1809-1819 |
| 4 | 445-461 | PNFVFPLNSIIKTIQPR | nsp2(NC_045512: + : 806-2719) | 91-107 |
| 4 | 462-471 | PRRARSVRYP linker 1-10 | | |
| 4 | 472-479 | IETISLAG | nsp3(NC_045512: + : 2720-8554) | 684-691 |
| 4 | 480-489 | PRRARSVRYP linker 1-10 | | |
| 4 | 490-502 | YAKPFLNKVVSTT | nsp3(NC_045512: + : 2720-8554) | |
| 4 | 503-512 | PRRARSVRYP linker 1-10 | | 1323-1335 |
| 4 | 513-528 | VREVLSDRELHLSWEV | helicase(NC_045512: + : 16237-18039) | 154-169 |
| 4 | 529-538 | PRRARSVRYP linker | | |
| 4 | 539-554 | ANSIVCRFDTRVLSNL | 3′-to-5′_exonuclease(NC_045512: + : 18040-19620) | 394-409 |
| 4 | 555-564 | PRRARSVRYP linker | | |
| 4 | 565-580 | LQSLQTYVTQQLIRAA | surface_glycoprotein(NC_045512: + : 21563-25384) | 1001-1016 |
| 4 | 581-590 | PRRARSVRYP linker 1-10 | | |
| 4 | 591-600 | RHSLSHFVNL | nsp3(NC_045512: + : 2720-8554) | 1697-1706 |
| 4 | 601-610 | PRRARSVRYP linker 1-10 | | |
| 4 | 611-620 | VQQESPFVMM | nsp3(NC_045512: + : 2720-8554) | 980-989 |
| 4 | 621-630 | PRRARSVRYP linker 1-10 | | |
| 4 | 631-641 | VVNAANVYLKH | nsp3(NC_045512: + : 2720-8554) | 239-249 |
| 4 | 642-651 | PRRARSVRYP linker 1-10 | | |
| 4 | 652-665 | TKDVVECLKLSHQS | nsp3(NC_045512: + : 2720-8554) | 1830-1843 |
| 4 | 666-675 | PRRARSVRYP linker 1-10 | | |
| 4 | 676-686 | KESVQTFFKLV | nsp2(NC_045512: + : 806-2719) | 492-502 |
| 4 | 687-696 | PRRARSVRYP linker 1-10 | | |
| 4 | 697-716 | LQKAAITILDGISQYSLRLI | nsp2(NC_045512: + : 806-2719) | 382-401 |
| 4 | 717-726 | PRRARSVRYP linker 1-10 | | |
| 4 | 727-734 | PEHSLAEY | nsp2(NC_045512: + : 806-2719) | 200-207 |
| 4 | 735-744 | PRRARSVRYP linker 1-10 | | |
| 4 | 745-752 | SLIDLQEL | surface_glycoprotein(NC_045512: + : 21563-25384) | 1196-1203 |
| 4 | 753-762 | PRRARSVRYP linker 1-10 | | |
| 4 | 763-771 | FFSNVTWFH | surface_glycoprotein(NC_045512: + : 21563-25384) | 58-66 |
| 4 | 772-781 | PRRARSVRYP linker 1-10 | | |
| 4 | 782-794 | NPVLPFNDGVYFA | surface_glycoprotein(NC_045512: + : 21563-25384) | |
| 4 | 795-804 | PRRARSVRYP linker 1-10 | | 81-93 |
| 4 | 805-813 | MPVCVETKA | nsp3(NC_045512: + : 2720-8554) | 571-579 |
| 4 | 814-823 | PRRARSVRYP linker 1-10 | | |
| 4 | 824-833 | KSASVYYSQL | nsp3(NC_045512: + : 2720-8554) | 1737-1746 |
| 4 | 834-843 | PRRARSVRYP linker 1-10 | | |
| 4 | 844-851 | AALQIPFA | surface_glycoprotein(NC_045512: + : 21563-25384) | 892-899 |

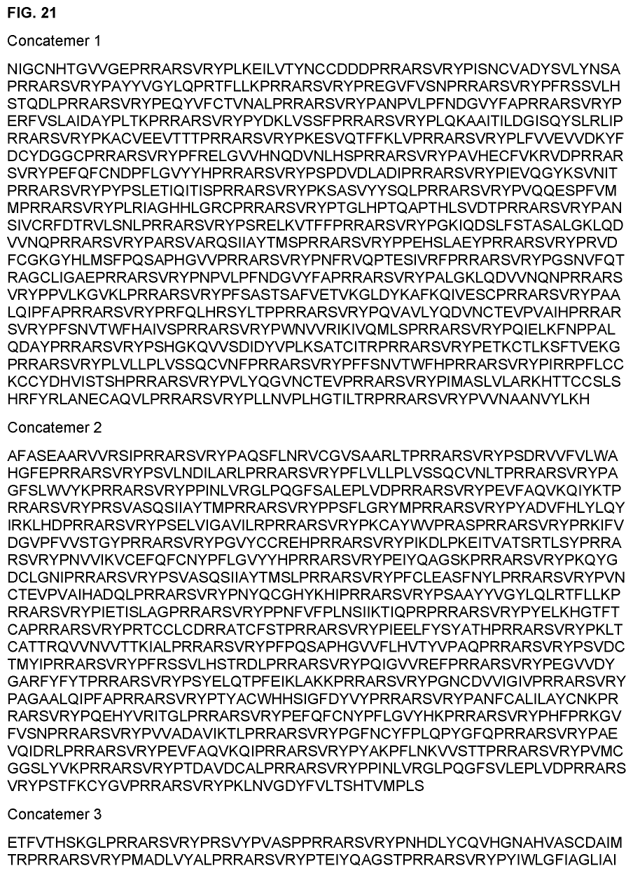

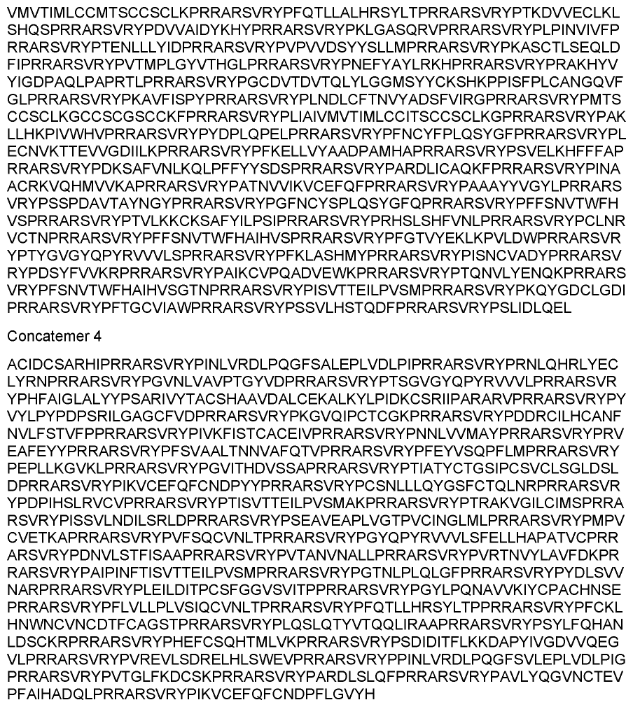

In certain embodiments, the vaccine comprises or expresses at least one of the concatemers of epitopes with intervening linker sequences (and as set forth in FIG. 21). In certain embodiments, the vaccine comprises or expresses at least two of the concatemers set forth below. In certain embodiments, the vaccine comprises or expresses three of the concatemers set forth below. In certain embodiments, the vaccine comprises or expresses all the concatemers set forth below.

TABLE 11

| | pos_ | | | |
|---|---|---|---|---|
| | in_ | | | pos_in_ |
| concatemer | concat | epitope | gene | gene |
| 1 | 1-12 | NIGCNHTGVVGE | nsp2(NC_045512: + : 806-2719) | 250-261 |
| 1 | 13-22 | PRRARSVRYP | linker | 1-10 |
| 1 | 23-36 | LKEILVTYNCCDDD | RNA-dependent_RNA_polymerase(NC_045512: + : 13442-13468 + 13468-16236) | 142-155 |
| 1 | 37-46 | PRRARSVRYP | linker | 1-10 |
| 1 | 47-61 | ISNCVADYSVLYNSA | surface_glycoprotein@Aruba(NC_045512@Aruba: + : 21563-25384) | 354-368 |
| 1 | 62-71 | PRRARSVRYP | linker | 1-10 |
| 1 | 72-86 | AYYVGYLQPRTFLLK | surface_glycoprotein@Aruba(NC_045512@Aruba: + : 21563-25384) | 260-274 |
| 1 | 87-96 | PRRARSVRYP | linker | 1-10 |
| 1 | 97-104 | REGVFVSN | surface_glycoprotein@Aruba(NC_045512@Aruba: + : 21563-25384) | 1087-1094 |
| 1 | 105-114 | PRRARSVRYP | linker | 1-10 |
| 1 | 115-126 | FRSSVLHSTQDL | surface_glycoprotein@Aruba(NC_045512@Aruba: + : 21563-25384) | 43-54 |
| 1 | 127-136 | PRRARSVRYP | linker | 1-10 |
| 1 | 137-147 | EQYVFCTVNAL | helicase(NC_045512: + : 16237-18039) | 353-363 |
| 1 | 148-157 | PRRARSVRYP | linker | 1-10 |
| 1 | 158-171 | ANPVLPFNDGVYFA | surface_glycoprotein@Austria(NC_045512@Austria: + : 21563-25384) | 77-90 |
| 1 | 172-181 | PRRARSVRYP | linker | 1-10 |
| 1 | 182-196 | ERFVSLAIDAYPLTK | RNA-dependent_RNA_polymerase(NC_045512: + : 13442-13468 + 13468-16236) | 857-871 |
| 1 | 197-206 | PRRARSVRYP | linker | 1-10 |
| 1 | 207-214 | YDKLVSSF | nsp3(NC_045512: + : 2720-8554) | 365-372 |
| 1 | 215-224 | PRRARSVRYP | linker | 1-10 |
| 1 | 225-244 | LQKAAITILDGISQYSLRLI | nsp2(NC_045512: + : 806-2719) | 382-401 |
| 1 | 245-254 | PRRARSVRYP | linker | 1-10 |
| 1 | 255-264 | KACVEEVTTT | nsp3(NC_045512: + : 2720-8554) | 415-424 |
| 1 | 265-274 | PRRARSVRYP | linker | 1-10 |
| 1 | 275-285 | KESVQTFFKLV | nsp2(NC_045512: + : 806-2719) | 492-502 |
| 1 | 286-295 | PRRARSVRYP | linker | 1-10 |
| 1 | 296-313 | LFVVEVVDKYFDCYDGGC | RNA-dependent_RNA_polymerase(NC_045512: + : 13442-13468 + 13468-16236) | 470-487 |
| 1 | 314-323 | PRRARSVRYP | linker | 1-10 |
| 1 | 324-339 | FRELGVVHNQDVNLHS | RNA-dependent_RNA_polymerase(NC_045512: + : 13442-13468 + 13468-16236) | 348-363 |
| 1 | 340-349 | PRRARSVRYP | linker | 1-10 |
| 1 | 350-360 | AVHECFVKRVD | 3′-to-5′_exonuclease(NC_045512: + : 18040-19620) | 281-291 |
| 1 | 361-370 | PRRARSVRYP | linker | 1-10 |
| 1 | 371-385 | EFQFCNDPFLGVYYH | surface_glycoprotein@Chile(NC_045512@Chile: + : 21563-25384) | 132-146 |
| 1 | 386-395 | PRRARSVRYP | linker | 1-10 |
| 1 | 396-404 | SPDVDLADI | surface_glycoprotein@Chile(NC_045512@Chile: + : 21563-25384) | 1161-1169 |
| 1 | 405-414 | PRRARSVRYP | linker | 1-10 |
| 1 | 415-426 | IEVQGYKSVNIT | nsp3(NC_045512: + : 2720-8554) | 13-24 |
| 1 | 427-436 | PRRARSVRYP | linker | 1-10 |
| 1 | 437-448 | YPSLETIQITIS | nsp3(NC_045512: + : 2720-8554) | 1483-1494 |
| 1 | 449-458 | PRRARSVRYP | linker | 1-10 |
| 1 | 459-468 | KSASVYYSQL | nsp3(NC_045512: + : 2720-8554) | 1737-1746 |
| 1 | 469-478 | PRRARSVRYP | linker | 1-10 |
| 1 | 479-488 | VQQESPFVMM | nsp3(NC_045512: + : 2720-8554) | 980-989 |
| 1 | 489-498 | PRRARSVRYP | linker | 1-10 |
| 1 | 499-509 | LRIAGHHLGRC | membrane_glycoprotein(NC_045512: + : 26523-27191) | 149-159 |
| 1 | 510-519 | PRRARSVRYP | linker | 1-10 |
| 1 | 520-535 | TGLHPTQAPTHLSVDT | 3′-to-5′_exonuclease(NC_045512: + : 18040-19620) | 16-31 |
| 1 | 536-545 | PRRARSVRYP | linker | 1-10 |
| 1 | 546-561 | ANSIVCRFDTRVLSNL | 3′-to-5′_exonuclease(NC_045512: + : 18040-19620) | 394-409 |
| 1 | 562-571 | PRRARSVRYP | linker | 1-10 |
| 1 | 572-580 | SRELKVTFF | nsp3(NC_045512: + : 2720-8554) | 1134-1142 |
| 1 | 581-590 | PRRARSVRYP | linker | 1-10 |
| 1 | 591-613 | GKIQDSLFSTASALGKLQDVVNQ | surface_glycoprotein@NorthMacedonia(NC_045512@NorthMacedonia: + : 21563-25384) | 928-950 |
| 1 | 614-623 | PRRARSVRYP | linker | 1-10 |
| 1 | 624-638 | ARSVARQSIIAYTMS | surface_glycoprotein@Mozambique(NC_045512@Mozambique: + : 21563-25384) | 680-694 |
| 1 | 639-648 | PRRARSVRYP | linker | 1-10 |
| 1 | 649-656 | PEHSLAEY | nsp2(NC_045512: + : 806-2719) | 200-207 |
| 1 | 657-666 | PRRARSVRYP | linker | 1-10 |
| 1 | 667-689 | RVDFCGKGYHLMSFPQSAPHGVV | surface_glycoprotein(NC_045512: + : 21563-25384) | 1039-1061 |
| 1 | 690-699 | PRRARSVRYP | linker | 1-10 |
| 1 | 700-712 | NFRVQPTESIVRF | surface_glycoprotein(NC_045512: + : 21563-25384) | 317-329 |
| 1 | 713-722 | PRRARSVRYP | linker | 1-10 |
| 1 | 723-738 | GSNVFQTRAGCLIGAE | surface_glycoprotein(NC_045512: + : 21563-25384) | 639-654 |
| 1 | 739-748 | PRRARSVRYP | linker | 1-10 |
| 1 | 749-761 | NPVLPFNDGVYFA | surface_glycoprotein(NC_045512: + : 21563-25384) | 81-93 |
| 1 | 762-771 | PRRARSVRYP | linker | 1-10 |
| 1 | 772-783 | ALGKLQDVVNQN | surface_glycoprotein(NC_045512: + : 21563-25384) | 944-955 |
| 1 | 784-793 | PRRARSVRYP | linker | 1-10 |
| 1 | 794-801 | PVLKGVKL | surface_glycoprotein@Aruba(NC_045512@Aruba: + : 21563-25384) | 1259-1266 |
| 1 | 802-811 | PRRARSVRYP | linker | 1-10 |
| 1 | 812-838 | FSASTSAFVETVKGLDYKAFKQIVESC | nsp2(NC_045512: + : 806-2719) | 300-326 |
| 1 | 839-848 | PRRARSVRYP | linker | 1-10 |
| 1 | 849-856 | AALQIPFA | surface_glycoprotein(NC_045512: + : 21563-25384) | 892-899 |
| 1 | 857-866 | PRRARSVRYP | linker | 1-10 |
| 1 | 867-877 | RFQLHRSYLTP | surface_glycoprotein@Austria(NC_045512@Austria: + : 21563-25384) | 233-243 |
| 1 | 878-887 | PRRARSVRYP | linker | 1-10 |
| 1 | 888-906 | QVAVLYQDVNCTEVPVAIH | surface_glycoprotein@China(NC_045512@China: + : 21563-25384) | 607-625 |
| 1 | 907-916 | PRRARSVRYP | linker | 1-10 |
| 1 | 917-928 | FSNVTWFHAIVS | surface_glycoprotein@Ghana(NC_045512@Ghana: + : 21563-25384) | 59-70 |
| 1 | 929-938 | PRRARSVRYP | linker | 1-10 |
| 1 | 939-951 | WNVVRIKIVQMLS | 3′-to-5′_exonuclease(NC_045512: + : 18040-19620) | 159-171 |
| 1 | 952-961 | PRRARSVRYP | linker | 1-10 |
| 1 | 962-976 | QIELKFNPPALQDAY | nsp3(NC_045512: + : 2720-8554) | 867-881 |
| 1 | 977-986 | PRRARSVRYP | linker | 1-10 |
| 1 | 987-1009 | SHGKQVVSDIDYVPLKSATCITR | 3′-to-5′_exonuclease(NC_045512: + : 18040-19620) | 454-476 |
| 1 | 1010-1019 | PRRARSVRYP | linker | 1-10 |
| 1 | 1020-1033 | ETKCTLKSFTVEKG | surface_glycoprotein@Aruba(NC_045512@Aruba: + : 21563-25384) | 294-307 |
| 1 | 1034-1043 | PRRARSVRYP | linker | 1-10 |
| 1 | 1044-1057 | LVLLPLVSSQCVNF | surface_glycoprotein@Brazil(NC_045512@Brazil: + : 21563-25384) | 5-18 |
| 1 | 1058-1067 | PRRARSVRYP | linker | 1-10 |
| 1 | 1068-1076 | FFSNVTWFH | surface_glycoprotein(NC_045512: + : 21563-25384) | 58-66 |
| 1 | 1077-1086 | PRRARSVRYP | linker | 1-10 |
| 1 | 1087-1106 | IRRPFLCCKCCYDHVISTSH | helicase(NC_045512: + : 16237-18039) | 20-39 |
| 1 | 1107-1116 | PRRARSVRYP | linker | 1-10 |
| 1 | 1117-1127 | VLYQGVNCTEV | surface_glycoprotein@Aruba(NC_045512@Aruba: + : 21563-25384) | 606-616 |
| 1 | 1128-1137 | PRRARSVRYP | linker | 1-10 |
| 1 | 1138-1169 | IMASLVLARKHTTCCSLSHRF | RNA-dependent_RNA_polymerase(NC_045512: + : 13442-13468 + 13468-16236) | 632-663 |
| | | YRLANECAQVL | | |
| 1 | 1170-1179 | PRRARSVRYP | linker | 1-10 |
| 1 | 1180-1192 | LLNVPLHGTILTR | membrane_glycoprotein(NC_045512: + : 26523-27191) | 119-131 |
| 1 | 1193-1202 | PRRARSVRYP | linker | 1-10 |
| 1 | 1203-1213 | VVNAANVYLKH | nsp3(NC_045512: + : 2720-8554) | 239-249 |
| 2 | 1-13 | AFASEAARVVRSI | nsp2(NC_045512: + : 806-2719) | 355-367 |
| 2 | 14-23 | PRRARSVRYP | linker | 1-10 |
| 2 | 24-40 | AQSFLNRVCGVSAARLT | RNA-dependent_RNA_polymerase(NC_045512: + : 13442-13468 + 13468-16236) | 4-20 |
| 2 | 41-50 | PRRARSVRYP | linker | 1-10 |
| 2 | 51-64 | SDRVVFVLWAHGFE | 3′-to-5′_exonuclease(NC_045512: + : 18040-19620) | 178-191 |
| 2 | 65-74 | PRRARSVRYP | linker | 1-10 |
| 2 | 75-84 | SVLNDILARL | surface_glycoprotein@Aruba(NC_045512@Aruba: + : 21563-25384) | 971-980 |
| 2 | 85-94 | PRRARSVRYP | linker | 1-10 |
| 2 | 95-110 | FLVLLPLVSSQCVNLT | surface_glycoprotein@Aruba(NC_045512@Aruba: + : 21563-25384) | 4-19 |
| 2 | 111-120 | PRRARSVRYP | linker | 1-10 |
| 2 | 121-129 | AGFSLWVYK | 3′-to-5′_exonuclease(NC_045512: + : 18040-19620) | 504-512 |
| 2 | 130-139 | PRRARSVRYP | linker | 1-10 |
| 2 | 140-159 | PINLVRGLPQGFSALEPLVD | surface_glycoprotein@Austria(NC_045512@Austria: + : 21563-25384) | 205-224 |
| 2 | 160-169 | PRRARSVRYP | linker | 1-10 |
| 2 | 170-181 | EVFAQVKQIYKT | surface_glycoprotein@Aruba(NC_045512@Aruba: + : 21563-25384) | 776-787 |
| 2 | 182-191 | PRRARSVRYP | linker | 1-10 |
| 2 | 192-204 | RSVASQSIIAYTM | surface_glycoprotein@Aruba(NC_045512@Aruba: + : 21563-25384) | 681-693 |
| 2 | 205-214 | PRRARSVRYP | linker | 1-10 |
| 2 | 215-222 | PSFLGRYM | nsp3(NC_045512: + : 2720-8554) | 822-829 |
| 2 | 223-232 | PRRARSVRYP | linker | 1-10 |
| 2 | 233-249 | YADVFHLYLQYIRKLHD | RNA-dependent_RNA_polymerase(NC_045512: + : 13442-13468 + 13468-16236) | 877-893 |
| 2 | 250-259 | PRRARSVRYP | linker | 1-10 |
| 2 | 260-270 | SELVIGAVILR | membrane_glycoprotein(NC_045512: + : 26523-27191) | 136-146 |
| 2 | 271-280 | PRRARSVRYP | linker | 1-10 |
| 2 | 281-290 | KCAYWVPRAS | nsp2(NC_045512: + : 806-2719) | 239-248 |
| 2 | 291-300 | PRRARSVRYP | linker | 1-10 |
| 2 | 301-316 | RKIFVDGVPFVVSTGY | RNA-dependent_RNA_polymerase(NC_045512: + : 13442-13468 + 13468-16236) | 331-346 |
| 2 | 317-326 | PRRARSVRYP | linker | 1-10 |
| 2 | 327-334 | GVYCCREH | nsp2(NC_045512: + : 806-2719) | 47-54 |
| 2 | 335-344 | PRRARSVRYP | linker | 1-10 |
| 2 | 345-362 | IKDLPKEITVATSRTLSY | membrane_glycoprotein(NC_045512: + : 26523-27191) | 161-178 |
| 2 | 363-372 | PRRARSVRYP | linker | 1-10 |
| 2 | 373-394 | NVVIKVCEFQFCNYPFLGVYYH | surface_glycoprotein@Brazil(NC_045512@Brazil: + : 21563-25384) | 125-146 |
| 2 | 395-404 | PRRARSVRYP | linker | 1-10 |
| 2 | 405-412 | EIYQAGSK | surface_glycoprotein@Mexico(NC_045512@Mexico: + : 21563-25384) | 471-478 |
| 2 | 413-422 | PRRARSVRYP | linker | 1-10 |
| 2 | 423-432 | KQYGDCLGNI | surface_glycoprotein@Jordan(NC_045512@Jordan: + : 21563-25384) | 831-840 |
| 2 | 433-442 | PRRARSVRYP | linker | 1-10 |
| 2 | 443-456 | SVASQSIIAYTMSL | surface_glycoprotein@Austria(NC_045512@Austria: + : 21563-25384) | 678-691 |
| 2 | 457-466 | PRRARSVRYP | linker | 1-10 |
| 2 | 467-476 | FCLEASFNYL | nsp3(NC_045512: + : 2720-8554) | 1391-1400 |
| 2 | 477-486 | PRRARSVRYP | linker | 1-10 |
| 2 | 487-501 | VNCTEVPVAIHADQL | surface_glycoprotein(NC_045512: + : 21563-25384) | 615-629 |
| 2 | 502-511 | PRRARSVRYP | linker | 1-10 |
| 2 | 512-521 | NYQCGHYKHI | nsp3(NC_045512: + : 2720-8554) | 1012-1021 |
| 2 | 522-531 | PRRARSVRYP | linker | 1-10 |
| 2 | 532-548 | SAAYYVGYLQLRTFLLK | surface_glycoprotein@Italy(NC_045512@Italy: + : 21563-25384) | 258-274 |
| 2 | 549-558 | PRRARSVRYP | linker | 1-10 |
| 2 | 559-566 | IETISLAG | nsp3(NC_045512: + : 2720-8554) | 684-691 |
| 2 | 567-576 | PRRARSVRYP | linker | 1-10 |
| 2 | 577-593 | PNFVFPLNSIIKTIQPR | nsp2(NC_045512: + : 806-2719) | 9 1-107 |
| 2 | 594-603 | PRRARSVRYP | linker | 1-10 |
| 2 | 604-614 | YELKHGTFTCA | nsp3(NC_045512: + : 2720-8554) | 996-1006 |
| 2 | 615-624 | PRRARSVRYP | linker | 1-10 |
| 2 | 625-639 | RTCCLCDRRATCFST | 3′-to-5′_exonuclease(NC_045512: + : 18040-19620) | 205-219 |
| 2 | 640-649 | PRRARSVRYP | linker | 1-10 |
| 2 | 650-660 | IEELFYSYATH | 3′-to-5′_exonuclease(NC_045512: + : 18040-19620) | 363-373 |
| 2 | 661-670 | PRRARSVRYP | linker | 1-10 |
| 2 | 671-690 | KLTCATTRQVVNVVTTKIAL | nsp3(NC_045512: + : 2720-8554) | 1923-1942 |
| 2 | 691-700 | PRRARSVRYP | linker | 1-10 |
| 2 | 701-720 | FPQSAPHGVVFLHVTYVPAQ | surface_glycoprotein@Aruba(NC_045512@Aruba: + : 21563-25384) | 1048-1067 |
| 2 | 721-730 | PRRARSVRYP | linker | 1-10 |
| 2 | 731-738 | SVDCTMYI | surface_glycoprotein(NC_045512: + : 21563-25384) | 735-742 |
| 2 | 739-748 | PRRARSVRYP | linker | 1-10 |
| 2 | 749-760 | FRSSVLHSTRDL | surface_glycoprotein@Nigeria(NC_045512@Nigeria: + : 21563-25384) | 43-54 |
| 2 | 761-770 | PRRARSVRYP | linker | 1-10 |
| 2 | 771-778 | QIGVVREF | helicase(NC_045512: + : 16237-18039) | 492-499 |
| 2 | 779-788 | PRRARSVRYP | linker | 1-10 |
| 2 | 789-802 | EGVVDYGARFYFYT | nsp3(NC_045512: + : 2720-8554) | 595-608 |
| 2 | 803-812 | PRRARSVRYP | linker | 1-10 |
| 2 | 813-827 | SYELQTPFEIKLAKK | nsp2(NC_045512: + : 806-2719) | 68-82 |
| 2 | 828-837 | PRRARSVRYP | linker | 1-10 |
| 2 | 838-847 | GNCDVVIGIV | surface_glycoprotein(NC_045512: + : 21563-25384) | 1124-1133 |
| 2 | 848-857 | PRRARSVRYP | linker | 1-10 |
| 2 | 858-867 | AGAALQIPFA | surface_glycoprotein@Nigeria(NC_045512@Nigeria: + : 21563-25384) | 886-895 |
| 2 | 868-877 | PRRARSVRYP | linker | 1-10 |
| 2 | 878-892 | TYACWHHSIGFDYVY | 3′-to-5′_exonuclease(NC_045512: + : 18040-19620) | 223-237 |
| 2 | 893-902 | PRRARSVRYP | linker | 1-10 |
| 2 | 903-915 | ANFCALILAYCNK | nsp3(NC_045512: + : 2720-8554) | 890-902 |
| 2 | 916-925 | PRRARSVRYP | linker | 1-10 |
| 2 | 926-935 | QEHYVRITGL | helicase(NC_045512: + : 16237-18039) | 243-252 |
| 2 | 936-945 | PRRARSVRYP | linker | 1-10 |
| 2 | 946-960 | EFQFCNYPFLGVYHK | surface_glycoprotein@Norway(NC_045512@Norway: + : 21563-25384) | 129-143 |
| 2 | 961-970 | PRRARSVRYP | linker | 1-10 |
| 2 | 971-981 | HFPRKGVFVSN | surface_glycoprotein@SmallAsia(NC_045512@SmallAsia: + : 21563-25384) | 1078-1088 |
| 2 | 982-991 | PRRARSVRYP | linker | 1-10 |
| 2 | 992-1001 | VVADAVIKTL | nsp3(NC_045512: + : 2720-8554) | 56-65 |
| 2 | 1002-1011 | PRRARSVRYP | linker | 1-10 |
| 2 | 1012-1025 | GFNCYFPLQPYGFQ | surface_glycoprotein@SmallSouthAmerica(NC_045512@ | 481-494 |
| | | | SmallSouthAmerica: + : 21563-25384) | |
| 2 | 1026-1035 | PRRARSVRYP | linker | 1-10 |
| 2 | 1036-1043 | AEVQIDRL | surface_glycoprotein(NC_045512: + : 21563-25384) | 989-996 |
| 2 | 1044-1053 | PRRARSVRYP | linker | 1-10 |
| 2 | 1054-1062 | EVFAQVKQI | surface_glycoprotein(NC_045512: + : 21563-25384) | 780-788 |
| 2 | 1063-1072 | PRRARSVRYP | linker | 1-10 |
| 2 | 1073-1085 | YAKPFLNKVVSTT | nsp3(NC_045512: + : 2720-8554) | 1323-1335 |
| 2 | 1086-1095 | PRRARSVRYP | linker | 1-10 |
| 2 | 1096-1105 | VMCGGSLYVK | RNA-dependent_RNA_polymerase(NC_045512: + : 13442-13468 + 13468-16236) | 667-676 |
| 2 | 1106-1115 | PRRARSVRYP | linker | 1-10 |
| 2 | 1116-1123 | TDAVDCAL | surface_glycoprotein(NC_045512: + : 21563-25384) | 286-293 |
| 2 | 1124-1133 | PRRARSVRYP | linker | 1-10 |
| 2 | 1134-1153 | PINLVRGLPQGFSVLEPLVD | surface_glycoprotein@SmallAfrica(NC_045512@SmallAfrica: + : 21563-25384) | 209-228 |
| 2 | 1154-1163 | PRRARSVRYP | linker | 1-10 |
| 2 | 1164-1171 | STFKCYGV | surface_glycoprotein@Aruba(NC_045512@Aruba: + : 21563-25384) | 371-378 |
| 2 | 1172-1181 | PRRARSVRYP | linker | 1-10 |
| 2 | 1182-1200 | KLNVGDYFVLTSHTVMPLS | helicase(NC_045512: + : 16237-18039) | 218-236 |
| 3 | 1-10 | ETFVTHSKGL | nsp2(NC_045512: + : 806-2719) | 527-536 |
| 3 | 11-20 | PRRARSVRYP | linker | 1-10 |
| 3 | 21-29 | RSVYPVASP | nsp2(NC_045512: + : 806-2719) | 121-129 |
| 3 | 30-39 | PRRARSVRYP | linker | 1-10 |
| 3 | 40-62 | NHDLYCQVHGNAHVASCDAIMTR | 3′-to-5′_exonuclease(NC_045512: + : 18040-19620) | 256-278 |
| 3 | 63-72 | PRRARSVRYP | linker | 1-10 |
| 3 | 73-80 | MADLVYAL | RNA-dependent_RNA_polymerase(NC_045512: + : 13442-13468 + 13468-16236) | 124-131 |
| 3 | 81-90 | PRRARSVRYP | linker | 1-10 |
| 3 | 91-99 | TEIYQAGST | surface_glycoprotein@Aruba(NC_045512@Aruba: + : 21563-25384) | 466-474 |
| 3 | 100-109 | PRRARSVRYP | linker | 1-10 |
| 3 | 110-140 | YIWLGFIAGLIAIVMVTIMLCC | surface_glycoprotein@Aruba(NC_045512@Aruba: + : 21563-25384) | 1211-1241 |
| | | MTSCCSCLK | | |
| 3 | 141-150 | PRRARSVRYP | linker | 1-10 |
| 3 | 151-163 | FQTLLALHRSYLT | surface_glycoprotein@Aruba(NC_045512@Aruba: + : 21563-25384) | 234-246 |
| 3 | 164-173 | PRRARSVRYP | linker | 1-10 |
| 3 | 174-187 | TKDVVECLKLSHQS | nsp3(NC_045512: + : 2720-8554) | 1830-1843 |
| 3 | 188-197 | PRRARSVRYP | linker | 1-10 |
| 3 | 198-207 | DVVAIDYKHY | nsp3(NC_045512: + : 2720-8554) | 1148-1157 |
| 3 | 208-217 | PRRARSVRYP | linker | 1-10 |
| 3 | 218-225 | KLGASQRV | membrane_glycoprotein(NC_045512: + : 26523-27191) | 180-187 |
| 3 | 226-235 | PRRARSVRYP | linker | 1-10 |
| 3 | 236-243 | LPINVIVF | nsp3(NC_045512: + : 2720-8554) | 1718-1725 |
| 3 | 244-253 | PRRARSVRYP | linker | 1-10 |
| 3 | 254-262 | TENLLLYID | nsp3(NC_045512: + : 2720-8554) | 432-440 |
| 3 | 263-272 | PRRARSVRYP | linker | 1-10 |
| 3 | 273-284 | VPVVDSYYSLLM | RNA-dependent_RNA_polymerase(NC_045512: + : 13442-13468 + 13468-16236) | 231-242 |
| 3 | 285-294 | PRRARSVRYP | linker | 1-10 |
| 3 | 295-307 | KASCTLSEQLDFI | nsp2(NC_045512: + : 806-2719) | 30-42 |
| 3 | 308-317 | PRRARSVRYP | linker | 1-10 |
| 3 | 318-329 | VTMPLGYVTHGL | nsp3(NC_045512: + : 2720-8554) | 628-639 |
| 3 | 330-339 | PRRARSVRYP | linker | 1-10 |
| 3 | 340-349 | NEFYAYLRKH | RNA-dependent_RNA_polymerase(NC_045512: + : 13442-13468 + 13468-16236) | 743-752 |
| 3 | 350-359 | PRRARSVRYP | linker | 1-10 |
| 3 | 360-379 | RAKHYVYIGDPAQLPAPRTL | helicase(NC_045512: + : 16237-18039) | 392-411 |
| 3 | 380-389 | PRRARSVRYP | linker | 1-10 |
| 3 | 390-428 | GCDVTDVTQLYLGGMSYYC | helicase(NC_045512: + : 16237-18039) | 54-92 |
| | | KSHKPPISFPLCANGQVFGL | | |
| 3 | 429-438 | PRRARSVRYP | linker | 1-10 |
| 3 | 439-446 | KAVFISPY | helicase(NC_045512: + : 16237-18039) | 508-515 |
| 3 | 447-456 | PRRARSVRYP | linker | 1-10 |
| 3 | 457-474 | LNDLCFTNVYADSFVIRG | surface_glycoprotein(NC_045512: + : 21563-25384) | 387-404 |
| 3 | 475-484 | PRRARSVRYP | linker | 1-10 |
| 3 | 485-504 | MTSCCSCLKGCCSCGSCCKF | surface_glycoprotein(NC_045512: + : 21563-25384) | 1237-1256 |
| 3 | 505-514 | PRRARSVRYP | linker | 1-10 |
| 3 | 515-537 | LIAIVMVTIMLCCITSCCSCLKG | surface_glycoprotein@Mozambique(NC_045512@Mozambique: + : 21563-25384) | 1220-1242 |
| 3 | 538-547 | PRRARSVRYP | linker | 1-10 |
| 3 | 548-559 | AKLLHKPIVWHV | nsp3(NC_045512: + : 2720-8554) | 1165-1176 |
| 3 | 560-569 | PRRARSVRYP | linker | 1-10 |
| 3 | 570-577 | YDPLQPEL | surface_glycoprotein(NC_045512: + : 21563-25384) | 1138-1145 |
| 3 | 578-587 | PRRARSVRYP | linker | 1-10 |
| 3 | 588-599 | FNCYFPLQSYGF | surface_glycoprotein(NC_045512: + : 21563-25384) | 486-497 |
| 3 | 600-609 | PRRARSVRYP | linker | 1-10 |
| 3 | 610-626 | LECNVKTTEVVGDIILK | nsp3(NC_045512: + : 2720-8554) | 1244-1260 |
| 3 | 627-636 | PRRARSVRYP | linker | 1-10 |
| 3 | 637-651 | FKELLVYAADPAMHA | RNA-dependent_RNA_polymerase(NC_045512: + : 13442-13468 + 13468-16236) | 368-382 |
| 3 | 652-661 | PRRARSVRYP | linker | 1-10 |
| 3 | 662-671 | SVELKHFFFA | RNA-dependent_RNA_polymerase(NC_045512: + : 13442-13468 + 13468-16236) | 434-443 |
| 3 | 672-681 | PRRARSVRYP | linker | 1-10 |
| 3 | 682-700 | DKSAFVNLKQLPFFYYSDS | 3′-to-5′_exonuclease(NC_045512: + : 18040-19620) | 432-450 |
| 3 | 701-710 | PRRARSVRYP | linker | 1-10 |
| 3 | 711-720 | ARDLICAQKF | surface_glycoprotein(NC_045512: + : 21563-25384) | 846-855 |
| 3 | 721-730 | PRRARSVRYP | linker | 1-10 |
| 3 | 731-745 | INAACRKVQHMVVKA | 3′-to-5′_exonuclease(NC_045512: + : 18040-19620) | 305-319 |
| 3 | 746-755 | PRRARSVRYP | linker | 1-10 |
| 3 | 756-768 | ATNVVIKVCEFQF | surface_glycoprotein(NC_045512: + : 21563-25384) | 123-135 |
| 3 | 769-778 | PRRARSVRYP | linker | 1-10 |
| 3 | 779-787 | AAAYYVGYL | surface_glycoprotein(NC_045512: + : 21563-25384) | 262-270 |
| 3 | 788-797 | PRRARSVRYP | linker | 1-10 |
| 3 | 798-809 | SSPDAVTAYNGY | nsp3(NC_045512: + : 2720-8554) | 660-671 |
| 3 | 810-819 | PRRARSVRYP | linker | 1-10 |
| 3 | 820-833 | GFNCYSPLQSYGFQ | surface_glycoprotein@Peru(NC_045512@Peru: + : 21563-25384) | 485-498 |
| 3 | 834-843 | PRRARSVRYP | linker | 1-10 |
| 3 | 844-854 | FFSNVTWFHVS | surface_glycoprotein@Nigeria(NC_045512@Nigeria: + : 21563-25384) | 58-68 |
| 3 | 855-864 | PRRARSVRYP | linker | 1-10 |
| 3 | 865-880 | TVLKKCKSAFYILPSI | nsp3(NC_045512: + : 2720-8554) | 526-541 |
| 3 | 881-890 | PRRARSVRYP | linker | 1-10 |
| 3 | 891-900 | RHSLSHFVNL | nsp3(NC_045512: + : 2720-8554) | 1697-1706 |
| 3 | 901-910 | PRRARSVRYP | linker | 1-10 |
| 3 | 911-918 | CLNRVCTN | nsp3(NC_045512: + : 2720-8554) | 1342-1349 |
| 3 | 919-928 | PRRARSVRYP | linker | 1-10 |
| 3 | 929-942 | FFSNVTWFHAIHVS | surface_glycoprotein@Bangladesh(NC_045512@Bangladesh: + : 21563-25384) | 58-71 |
| 3 | 943-952 | PRRARSVRYP | linker | 1-10 |
| 3 | 953-966 | FGTVYEKLKPVLDW | nsp2(NC_045512: + : 806-2719) | 437-450 |
| 3 | 967-976 | PRRARSVRYP | linker | 1-10 |
| 3 | 977-991 | TYGVGYQPYRVVVLS | surface_glycoprotein@Australia(NC_045512@Australia: + : 21563-25384) | 496-510 |
| 3 | 992-1001 | PRRARSVRYP | linker | 1-10 |
| 3 | 1002-1009 | FKLASHMY | nsp3(NC_045512: + : 2720-8554) | 96-103 |
| 3 | 1010-1019 | PRRARSVRYP | linker | 1-10 |
| 3 | 1020-1027 | ISNCVADY | surface_glycoprotein(NC_045512: + : 21563-25384) | 358-365 |
| 3 | 1028-1037 | PRRARSVRYP | linker | 1-10 |
| 3 | 1038-1045 | DSYFVVKR | RNA-dependent_RNA_polymerase(NC_045512: + : 13442-13468 + 13468-16236) | 67-74 |
| 3 | 1046-1055 | PRRARSVRYP | linker | 1-10 |
| 3 | 1056-1068 | AIKCVPQADVEWK | 3′-to-5′_exonuclease(NC_045512: + : 18040-19620) | 337-349 |
| 3 | 1069-1078 | PRRARSVRYP | linker | 1-10 |
| 3 | 1079-1088 | TQNVLYENQK | surface_glycoprotein(NC_045512: + : 21563-25384) | 912-921 |
| 3 | 1089-1098 | PRRARSVRYP | linker | 1-10 |
| 3 | 1099-1114 | FSNVTWFHAIHVSGTN | surface_glycoprotein@Peru(NC_045512@Peru: + : 21563-25384) | 59-74 |
| 3 | 1115-1124 | PRRARSVRYP | linker | 1-10 |
| 3 | 1125-1136 | ISVTTEILPVSM | surface_glycoprotein(NC_045512: + : 21563-25384) | 720-731 |
| 3 | 1137-1146 | PRRARSVRYP | linker | 1-10 |
| 3 | 1147-1156 | KQYGDCLGDI | surface_glycoprotein(NC_045512: + : 21563-25384) | 835-844 |
| 3 | 1157-1166 | PRRARSVRYP | linker | 1-10 |
| 3 | 1167-1174 | FTGCVIAW | surface_glycoprotein(NC_045512: + : 21563-25384) | 429-436 |
| 3 | 1175-1184 | PRRARSVRYP | linker | 1-10 |
| 3 | 1185-1194 | SSVLHSTQDF | surface_glycoprotein@Norway(NC_045512@Norway: + : 21563-25384) | 45-54 |
| 3 | 1195-1204 | PRRARSVRYP | linker | 1-10 |
| 3 | 1205-1212 | SLIDLQEL | surface_glycoprotein(NC_045512: + : 21563-25384) | 1196-1203 |
| 4 | 1-10 | ACIDCSARHI | nsp3(NC_045512: + : 2720-8554) | 1872-1881 |
| 4 | 11-20 | PRRARSVRYP | linker | 1-10 |
| 4 | 21-42 | INLVRDLPQGFSALEPLVDLPI | surface_glycoprotein@Aruba(NC_045512@Aruba: + : 21563-25384) | 206-227 |
| 4 | 43-52 | PRRARSVRYP | linker | 1-10 |
| 4 | 53-66 | RNLQHRLYECLYRN | RNA-dependent_RNA_polymerase(NC_045512: + : 13442-13468 + 13468-16236) | 721-734 |
| 4 | 67-76 | PRRARSVRYP | linker | 1-10 |
| 4 | 77-89 | GVNLVAVPTGYVD | 3′-to-5′_exonuclease(NC_045512: + : 18040-19620) | 114-126 |
| 4 | 90-99 | PRRARSVRYP | linker | 1-10 |
| 4 | 100-113 | TSGVGYQPYRVVVL | surface_glycoprotein@Aruba(NC_045512@Aruba: + : 21563-25384) | 496-509 |
| 4 | 114-123 | PRRARSVRYP | linker | 1-10 |
| 4 | 124-174 | HFAIGLALYYPSARIVYTACSHAAVD | helicase(NC_045512: + : 16237-18039) | 290-340 |
| | | ALCEKALKYLPIDKCSRIIPARARV | | |
| 4 | 175-184 | PRRARSVRYP | linker | 1-10 |
| 4 | 185-204 | YVYLPYPDPSRILGAGCFVD | RNA-dependent_RNA_polymerase(NC_045512: + : 13442-13468 + 13468-16236) | 826-845 |
| 4 | 205-214 | PRRARSVRYP | linker | 1-10 |
| 4 | 215-225 | KGVQIPCTCGK | nsp3(NC_045512: + : 2720-8554) | 963-973 |
| 4 | 226-235 | PRRARSVRYP | linker | 1-10 |
| 4 | 236-255 | DDRCILHCANFNVLFSTVFP | RNA-dependent_RNA_polymerase(NC_045512: + : 13442-13468 + 13468-16236) | 303-322 |
| 4 | 256-265 | PRRARSVRYP | linker | 1-10 |
| 4 | 266-278 | IVKFISTCACEIV | nsp2(NC_045512: + : 806-2719) | 468-480 |
| 4 | 279-288 | PRRARSVRYP | linker | 1-10 |
| 4 | 289-296 | NNLVVMAY | nsp2(NC_045512: + : 806-2719) | 413-420 |
| 4 | 297-306 | PRRARSVRYP | linker | 1-10 |
| 4 | 307-314 | RVEAFEYY | nsp3(NC_045512: + : 2720-8554) | 810-817 |
| 4 | 315-324 | PRRARSVRYP | linker | 1-10 |
| 4 | 325-339 | FSVAALTNNVAFQTV | RNA-dependent_RNA_polymerase(NC_045512: + : 13442-13468 + 13468-16236) | 396-410 |
| 4 | 340-349 | PRRARSVRYP | linker | 1-10 |
| 4 | 350-359 | FEYVSQPFLM | surface_glycoprotein@Aruba(NC_045512@Aruba: + : 21563-25384) | 164-173 |
| 4 | 360-369 | PRRARSVRYP | linker | 1-10 |
| 4 | 370-378 | EPLLKGVKL | surface_glycoprotein@Croatia(NC_045512@Croatia: + : 21563-25384) | 1258-1266 |
| 4 | 379-388 | PRRARSVRYP | linker | 1-10 |
| 4 | 389-398 | GVITHDVSSA | helicase(NC_045512: + : 16237-18039) | 478-487 |
| 4 | 399-408 | PRRARSVRYP | linker | 1-10 |
| 4 | 409-431 | TIATYCTGSIPCSVCLSGLDSLD | nsp3(NC_045512: + : 2720-8554) | 1459-1481 |
| 4 | 432-441 | PRRARSVRYP | linker | 1-10 |
| 4 | 442-455 | IKVCEFQFCNDPYY | surface_glycoprotein@CostaRica(NC_045512@CostaRica: + : 21563-25384) | 128-141 |
| 4 | 456-465 | PRRARSVRYP | linker | 1-10 |
| 4 | 466-482 | CSNLLLQYGSFCTQLNR | surface_glycoprotein(NC_045512: + : 21563-25384) | 749-765 |
| 4 | 483-492 | PRRARSVRYP | linker | 1-10 |
| 4 | 493-502 | DPIHSLRVCV | nsp3(NC_045512: + : 2720-8554) | 339-348 |
| 4 | 503-512 | PRRARSVRYP | linker | 1-10 |
| 4 | 513-527 | TISVTTEILPVSMAK | surface_glycoprotein@Mexico(NC_045512@Mexico: + : 21563-25384) | 719-733 |
| 4 | 528-537 | PRRARSVRYP | linker | 1-10 |
| 4 | 538-549 | TRAKVGILCIMS | helicase(NC_045512: + : 16237-18039) | 566-577 |
| 4 | 550-559 | PRRARSVRYP | linker | 1-10 |
| 4 | 560-572 | ISSVLNDILSRLD | surface_glycoprotein@Bangladesh(NC_045512@Bangladesh: + : 21563-25384) | 972-984 |
| 4 | 573-582 | PRRARSVRYP | linker | 1-10 |
| 4 | 583-602 | SEAVEAPLVGTPVCINGLML | nsp2(NC_045512: + : 806-2719) | 591-610 |
| 4 | 603-612 | PRRARSVRYP | linker | 1-10 |
| 4 | 613-621 | MPVCVETKA | nsp3(NC_045512: + : 2720-8554) | 571-579 |
| 4 | 622-631 | PRRARSVRYP | linker | 1-10 |
| 4 | 632-640 | VFSQCVNLT | surface_glycoprotein@Egypt(NC_045512@Egypt: + : 21563-25384) | 11-19 |
| 4 | 641-650 | PRRARSVRYP | linker | 1-10 |
| 4 | 651-672 | GYQPYRVVVLSFELLHAPATVC | surface_glycoprotein(NC_045512: + : 21563-25384) | 504-525 |
| 4 | 673-682 | PRRARSVRYP | linker | 1-10 |
| 4 | 683-693 | DNVLSTFISAA | nsp3(NC_045512: + : 2720-8554) | 1809-1819 |
| 4 | 694-703 | PRRARSVRYP | linker | 1-10 |
| 4 | 704-712 | VTANVNALL | RNA-dependent_RNA_polymerase(NC_045512: + : 13442-13468 + 13468-16236) | 700-708 |
| 4 | 713-722 | PRRARSVRYP | linker | 1-10 |
| 4 | 723-734 | VRTNVYLAVFDK | nsp3(NC_045512: + : 2720-8554) | 351-362 |
| 4 | 735-744 | PRRARSVRYP | linker | 1-10 |
| 4 | 745-763 | AIPINFTISVTTEILPVSM | surface_glycoprotein@Aruba(NC_045512@Aruba: + : 21563-25384) | 709-727 |
| 4 | 764-773 | PRRARSVRYP | linker | 1-10 |
| 4 | 774-783 | GTNLPLQLGF | 3′-to-5′_exonuclease(NC_045512: + : 18040-19620) | 102-111 |
| 4 | 784-793 | PRRARSVRYP | linker | 1-10 |
| 4 | 794-802 | YDLSVVNAR | helicase(NC_045512: + : 16237-18039) | 382-390 |
| 4 | 803-812 | PRRARSVRYP | linker | 1-10 |
| 4 | 813-831 | LEILDITPCSFGGVSVITP | surface_glycoprotein(NC_045512: + : 21563-25384) | 582-600 |
| 4 | 832-841 | PRRARSVRYP | linker | 1-10 |
| 4 | 842-861 | GYLPQNAVVKIYCPACHNSE | nsp2(NC_045512: + : 806-2719) | 178-197 |
| 4 | 862-871 | PRRARSVRYP | linker | 1-10 |
| 4 | 872-887 | FLVLLPLVSIQCVNLT | surface_glycoprotein@SmallNorthAmerica(NC_045512@ | 4-19 |
| | | | SmallNorthAmerica: + : 21563-25384) | |
| 4 | 888-897 | PRRARSVRYP | linker | 1-10 |
| 4 | 898-909 | FQTLLHRSYLTP | surface_glycoprotein@SmallAsia(NC_045512@SmallAsia: + : 21563-25384) | 230-241 |
| 4 | 910-919 | PRRARSVRYP | linker | 1-10 |
| 4 | 920-939 | FCKLHNWNCVNCDTFCAGST | nsp3(NC_045512: + : 2720-8554) | 1626-1645 |
| 4 | 940-949 | PRRARSVRYP | linker | 1-10 |
| 4 | 950-965 | LQSLQTYVTQQLIRAA | surface_glycoprotein(NC_045512: + : 21563-25384) | 100 1-1016 |
| 4 | 966-975 | PRRARSVRYP | linker | 1-10 |
| 4 | 976-989 | SYLFQHANLDSCKR | nsp3(NC_045512: + : 2720-8554) | 915-928 |
| 4 | 990-999 | PRRARSVRYP | linker | 1-10 |
| 4 | 1000-1011 | HEFCSQHTMLVK | RNA-dependent_RNA_polymerase(NC_045512: + : 13442-13468 + 13468-16236) | 810-821 |
| 4 | 1012-1021 | PRRARSVRYP | linker | 1-10 |
| 4 | 1022-1046 | SDIDITFLKKDAPYIVGDVVQEGVL | nsp3(NC_045512: + : 2720-8554) | 454-478 |
| 4 | 1047-1056 | PRRARSVRYP | linker | 1-10 |
| 4 | 1057-1072 | VREVLSDRELHLSWEV | helicase(NC_045512: + : 16237-18039) | 154-169 |
| 4 | 1073-1082 | PRRARSVRYP | linker | 1-10 |
| 4 | 1083-1106 | PINLVRDLPQGFSVLEPLVDLPIG | surface_glycoprotein@Belgium(NC_045512@Belgium: + : 21563-25384) | 205-228 |
| 4 | 1107-1116 | PRRARSVRYP | linker | 1-10 |
| 4 | 1117-1126 | VTGLFKDCSK | 3′-to-5′_exonuclease(NC_045512: + : 18040-19620) | 4-13 |
| 4 | 1127-1136 | PRRARSVRYP | linker | 1-10 |
| 4 | 1137-1144 | ARDLSLQF | nsp3(NC_045512: + : 2720-8554) | 1652-1659 |
| 4 | 1145-1154 | PRRARSVRYP | linker | 1-10 |
| 4 | 1155-1175 | AVLYQGVNCTEVPFAIHADQL | surface_glycoprotein@SmallAfrica(NC_045512@SmallAfrica: + : 21563-25384) | 605-625 |
| 4 | 1176-1185 | PRRARSVRYP | linker | 1-10 |
| 4 | 1186-1203 | IKVCEFQFCNDPFLGVYH | surface_glycoprotein@Aruba(NC_045512@Aruba: + : 21563-25384) | 125-142 |

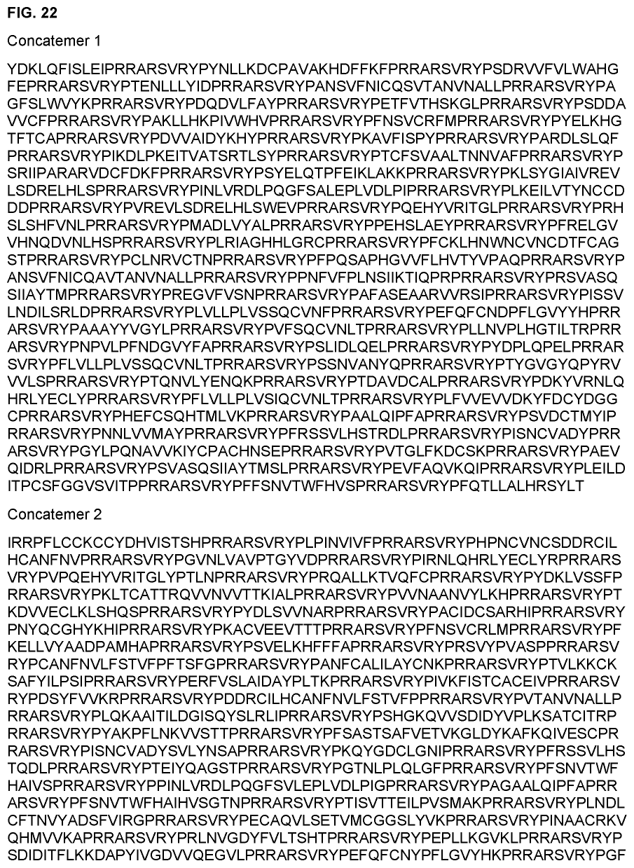

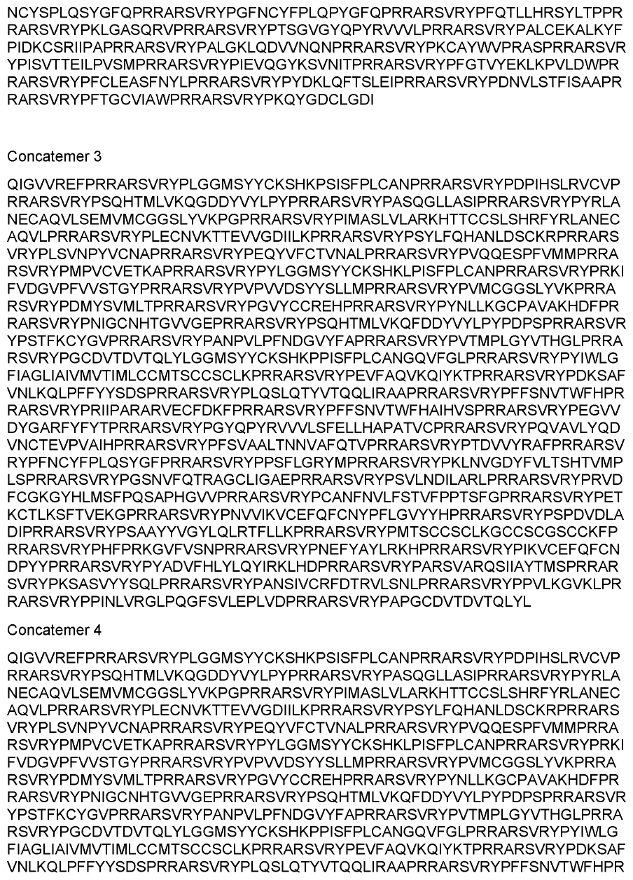

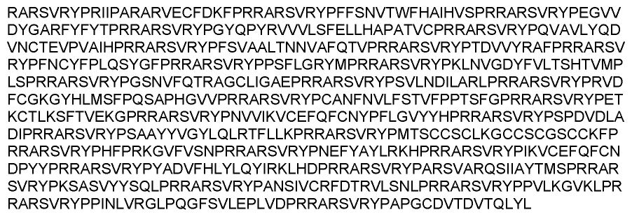

In certain embodiments, the vaccine comprises or expresses at least one of the concatemers of epitopes with intervening linker sequences (and as set forth in FIG. 22). In certain embodiments, the vaccine comprises or expresses at least two of the concatemers set forth below. In certain embodiments, the vaccine comprises or expresses three of the concatemers set forth below. In certain embodiments, the vaccine comprises or expresses all the concatemers set forth below.

Table 12

| | pos_in_ | | | pos_in_ |
|---|---|---|---|---|
| contig | contig | epitope | gene | gene |
| 1 | 1-11 | YDKLQFISLEI | helicase@SouthAfrica(NC_045512@SouthAfrica: + : 16237-18039) | 582-592 |
| 1 | 12-21 | PRRARSVRYP | linker | 1-10 |
| 1 | 22-39 | YNLLKDCPAVAKHDFFKF | RNA-dependent_RNA_polymerase@Aruba(NC_045512@Aruba: + : 13442-13468 + 13468-16236) | 87-104 |
| 1 | 40-49 | PRRARSVRYP | linker | 1-10 |
| 1 | 50-63 | SDRVVFVLWAHGFE | 3′-to-5′_exonuclease(NC_045512: + : 18040-19620) | 178-191 |
| 1 | 64-73 | PRRARSVRYP | linker | 1-10 |
| 1 | 74-82 | TENLLLYID | nsp3(NC_045512: + : 2720-8554) | 432-440 |
| 1 | 83-92 | PRRARSVRYP | linker | 1-10 |
| 1 | 93-111 | ANSVFNICQSVTANVNALL | RNA-dependent_RNA_polymerase@Croatia(NC_045512@ | 690-708 |
| | | | Croatia: + : 13442-13468 + 13468-16236) | |
| 1 | 112-121 | PRRARSVRYP | linker | 1-10 |
| 1 | 122-130 | AGFSLWVYK | 3′-to-5′_exonuclease(NC_045512: + : 18040-19620) | 504-512 |
| 1 | 131-140 | PRRARSVRYP | linker | 1-10 |
| 1 | 141-148 | DQDVLFAY | RNA-dependent_RNA_polymerase@FrenchGuiana(NC_045512@ | 523-530 |
| | | | FrenchGuiana: + : 13442-13468 + 13468-16236) | |
| 1 | 149-158 | PRRARSVRYP | linker | 1-10 |
| 1 | 159-168 | ETFVTHSKGL | nsp2(NC_045512: + : 806-2719) | 527-536 |
| 1 | 169-178 | PRRARSVRYP | linker | 1-10 |
| 1 | 179-186 | SDDAVVCF | RNA-dependent_RNA_polymerase@Aruba(NC_045512@Aruba: + : 13442-13468 + 13468-16236) | 759-766 |
| 1 | 187-196 | PRRARSVRYP | linker | 1-10 |
| 1 | 197-208 | AKLLHKPIVWHV | nsp3(NC_045512: + : 2720-8554) | 1165-1176 |
| 1 | 209-218 | PRRARSVRYP | linker | 1-10 |
| 1 | 219-226 | FNSVCRFM | helicase@Malaysia(NC_045512@Malaysia: + : 16237-18039) | 422-429 |
| 1 | 227-236 | PRRARSVRYP | linker | 1-10 |
| 1 | 237-247 | YELKHGTFTCA | nsp3(NC_045512: + : 2720-8554) | 996-1006 |
| 1 | 248-257 | PRRARSVRYP | linker | 1-10 |
| 1 | 258-267 | DVVAIDYKHY | nsp3(NC_045512: + : 2720-8554) | 1148-1157 |
| 1 | 268-277 | PRRARSVRYP | linker | 1-10 |
| 1 | 278-285 | KAVFISPY | helicase(NC_045512: + : 16237-18039) | 508-515 |
| 1 | 286-295 | PRRARSVRYP | linker | 1-10 |
| 1 | 296-303 | ARDLSLQF | nsp3(NC_045512: + : 2720-8554) | 1652-1659 |
| 1 | 304-313 | PRRARSVRYP | linker | 1-10 |
| 1 | 314-331 | IKDLPKEITVATSRTLSY | membrane_glycoprotein(NC_045512: + : 26523-27191) | 161-178 |
| 1 | 332-341 | PRRARSVRYP | linker | 1-10 |
| 1 | 342-355 | TCFSVAALTNNVAF | RNA-dependent_RNA_polymerase@Aruba(NC_045512@Aruba: + : 13442-13468 + 13468-16236) | 394-407 |
| 1 | 356-365 | PRRARSVRYP | linker | 1-10 |
| 1 | 366-381 | SRIIPARARVDCFDKF | helicase@Brazil(NC_045512@Brazil: + : 16237-18039) | 331-346 |
| 1 | 382-391 | PRRARSVRYP | linker | 1-10 |
| 1 | 392-406 | SYELQTPFEIKLAKK | nsp2(NC_045512: + : 806-2719) | 68-82 |
| 1 | 407-416 | PRRARSVRYP | linker | 1-10 |
| 1 | 417-437 | KLSYGIAIVREVLSDRELHLS | helicase@Colombia(NC_045512@Colombia: + : 16237-18039) | 146-166 |
| 1 | 438-447 | PRRARSVRYP | linker | 1-10 |
| 1 | 448-469 | INLVRDLPQGFSALEPLVDLPI | surface_glycoprotein@Aruba(NC_045512@Aruba: + : 21563-25384) | 206-227 |
| 1 | 470-479 | PRRARSVRYP | linker | 1-10 |
| 1 | 480-493 | LKEILVTYNCCDDD | RNA-dependent_RNA_polymerase(NC_045512: + : 13442-13468 + 13468-16236) | 142-155 |
| 1 | 494-503 | PRRARSVRYP | linker | 1-10 |
| 1 | 504-519 | VREVLSDRELHLSWEV | helicase(NC_045512: + : 16237-18039) | 154-169 |
| 1 | 520-529 | PRRARSVRYP | linker | 1-10 |
| 1 | 530-539 | QEHYVRITGL | helicase(NC_045512: + : 16237-18039) | 243-252 |
| 1 | 540-549 | PRRARSVRYP | linker | 1-10 |
| 1 | 550-559 | RHSLSHFVNL | nsp3(NC_045512: + : 2720-8554) | 1697-1706 |
| 1 | 560-569 | PRRARSVRYP | linker | 1-10 |
| 1 | 570-577 | MADLVYAL | RNA-dependent_RNA_polymerase(NC_045512: + : 13442-13468 + 13468-16236) | 124-131 |
| 1 | 578-587 | PRRARSVRYP | linker | 1-10 |
| 1 | 588-595 | PEHSLAEY | nsp2(NC_045512: + : 806-2719) | 200-207 |
| 1 | 596-605 | PRRARSVRYP | linker | 1-10 |
| 1 | 606-621 | FRELGVVHNQDVNLHS | RNA-dependent_RNA_polymerase(NC_045512: + : 13442-13468 + 13468-16236) | 348-363 |
| 1 | 622-631 | PRRARSVRYP | linker | 1-10 |
| 1 | 632-642 | LRIAGHHLGRC | membrane_glycoprotein(NC_045512: + : 26523-27191) | 149-159 |
| 1 | 643-652 | PRRARSVRYP | linker | 1-10 |
| 1 | 653-672 | FCKLHNWNCVNCDTFCAGST | nsp3(NC_045512: + : 2720-8554) | 1626-1645 |
| 1 | 673-682 | PRRARSVRYP | linker | 1-10 |
| 1 | 683-690 | CLNRVCTN | nsp3(NC_045512: + : 2720-8554) | 1342-1349 |
| 1 | 691-700 | PRRARSVRYP | linker | 1-10 |
| 1 | 701-720 | FPQSAPHGVVFLHVTYVPAQ | surface_glycoprotein@Aruba(NC_045512@Aruba: + : 21563-25384) | 1048-1067 |
| 1 | 721-730 | PRRARSVRYP | linker | 1-10 |
| 1 | 731-749 | ANSVFNICQAVTANVNALL | RNA-dependent_RNA_polymerase@Aruba(NC_045512@Aruba: + : 13442-13468 + 13468-16236) | 690-708 |
| 1 | 750-759 | PRRARSVRYP | linker | 1-10 |
| 1 | 760-776 | PNFVFPLNSIIKTIQPR | nsp2(NC_045512: + : 806-2719) | 91-107 |
| 1 | 777-786 | PRRARSVRYP | linker | 1-10 |
| 1 | 787-799 | RSVASQSIIAYTM | surface_glycoprotein@Aruba(NC_045512@Aruba: + : 21563-25384) | 681-693 |
| 1 | 800-809 | PRRARSVRYP | linker | 1-10 |
| 1 | 810-817 | REGVFVSN | surface_glycoprotein@Aruba(NC_045512@Aruba: + : 21563-25384) | 1087-1094 |
| 1 | 818-827 | PRRARSVRYP | linker | 1-10 |
| 1 | 828-840 | AFASEAARVVRSI | nsp2(NC_045512: + : 806-2719) | 355-367 |
| 1 | 841-850 | PRRARSVRYP | linker | 1-10 |
| 1 | 851-863 | ISSVLNDILSRLD | surface_glycoprotein@Bangladesh(NC_045512@Bangladesh: + : 21563-25384) | 972-984 |
| 1 | 864-873 | PRRARSVRYP | linker | 1-10 |
| 1 | 874-887 | LVLLPLVSSQCVNF | surface_glycoprotein@Brazil(NC_045512@Brazil: + : 21563-25384) | 5-18 |
| 1 | 888-897 | PRRARSVRYP | linker | 1-10 |
| 1 | 898-912 | EFQFCNDPFLGVYYH | surface_glycoprotein@Chile(NC_045512@Chile: + : 21563-25384) | 132-146 |
| 1 | 913-922 | PRRARSVRYP | linker | 1-10 |
| 1 | 923-931 | AAAYYVGYL | surface_glycoprotein(NC_045512: + : 21563-25384) | 262-270 |
| 1 | 932-941 | PRRARSVRYP | linker | 1-10 |
| 1 | 942-950 | VFSQCVNLT | surface_glycoprotein@Egypt(NC_045512@Egypt: + : 21563-25384) | 11-19 |
| 1 | 951-960 | PRRARSVRYP | linker | 1-10 |
| 1 | 961-973 | LLNVPLHGTILTR | membrane_glycoprotein(NC_045512: + : 26523-27191) | 119-131 |
| 1 | 974-983 | PRRARSVRYP | linker | 1-10 |
| 1 | 984-996 | NPVLPFNDGVYFA | surface_glycoprotein(NC_045512: + : 21563-25384) | 81-93 |
| 1 | 997-1006 | PRRARSVRYP | linker | 1-10 |
| 1 | 1007-1014 | SLIDLQEL | surface_glycoprotein(NC_045512: + : 21563-25384) | 1196-1203 |
| 1 | 1015-1024 | PRRARSVRYP | linker | 1-10 |
| 1 | 1025-1032 | YDPLQPEL | surface_glycoprotein(NC_045512: + : 21563-25384) | 1138-1145 |
| 1 | 1033-1042 | PRRARSVRYP | linker | 1-10 |
| 1 | 1043-1058 | FLVLLPLVSSQCVNLT | surface_glycoprotein@Aruba(NC_045512@Aruba: + : 21563-25384) | 4-19 |
| 1 | 1059-1068 | PRRARSVRYP | linker | 1-10 |
| 1 | 1069-1076 | SSNVANYQ | helicase@Austria(NC_045512@Austria: + : 16237-18039) | 263-270 |
| 1 | 1077-1086 | PRRARSVRYP | linker | 1-10 |
| 1 | 1087-1101 | TYGVGYQPYRVVVLS | surface_glycoprotein@Australia(NC_045512@Australia: + : 21563-25384) | 496-510 |
| 1 | 1102-1111 | PRRARSVRYP | linker | 1-10 |
| 1 | 1112-1121 | TQNVLYENQK | surface_glycoprotein(NC_045512: + : 21563-25384) | 912-921 |
| 1 | 1122-1131 | PRRARSVRYP | linker | 1-10 |
| 1 | 1132-1139 | TDAVDCAL | surface_glycoprotein(NC_045512: + : 21563-25384) | 286-293 |
| 1 | 1140-1149 | PRRARSVRYP | linker | 1-10 |
| 1 | 1150-1165 | DKYVRNLQHRLYECLY | RNA-dependent_RNA_polymerase@Aruba(NC_045512@Aruba: + : 13442-13468 + 13468-16236) | 717-732 |
| 1 | 1166-1175 | PRRARSVRYP | linker | 1-10 |
| 1 | 1176-1191 | FLVLLPLVSIQCVNLT | surface_glycoprotein@SmallNorthAmerica(NC_045512@SmallNorthAmerica: + : 21563-25384) | 4-19 |
| 1 | 1192-1201 | PRRARSVRYP | linker | 1-10 |
| 1 | 1202-1219 | LFVVEVVDKYFDCYDGGC | RNA-dependent_RNA_polymerase(NC_045512: + : 13442-13468 + 13468-16236) | 470-487 |
| 1 | 1220-1229 | PRRARSVRYP | linker | 1-10 |
| 1 | 1230-1241 | HEFCSQHTMLVK | RNA-dependent_RNA_polymerase(NC_045512: + : 13442-13468 + 13468-16236) | 810-821 |
| 1 | 1242-1251 | PRRARSVRYP | linker | 1-10 |
| 1 | 1252-1259 | AALQIPFA | surface_glycoprotein(NC_045512: + : 21563-25384) | 892-899 |
| 1 | 1260-1269 | PRRARSVRYP | linker | 1-10 |
| 1 | 1270-1277 | SVDCTMYI | surface_glycoprotein(NC_045512: + : 21563-25384) | 735-742 |
| 1 | 1278-1287 | PRRARSVRYP | linker | 1-10 |
| 1 | 1288-1295 | NNLVVMAY | nsp2(NC_045512: + : 806-2719) | 413-420 |
| 1 | 1296-1305 | PRRARSVRYP | linker | 1-10 |
| 1 | 1306-1317 | FRSSVLHSTRDL | surface_glycoprotein@Nigeria(NC_045512@Nigeria: + : 21563-25384) | 43-54 |
| 1 | 1318-1327 | PRRARSVRYP | linker | 1-10 |
| 1 | 1328-1335 | ISNCVADY | surface_glycoprotein(NC_045512: + : 21563-25384) | 358-365 |
| 1 | 1336-1345 | PRRARSVRYP | linker | 1-10 |
| 1 | 1346-1365 | GYLPQNAVVKIYCPACHNSE | nsp2(NC_045512: + : 806-2719) | 178-197 |
| 1 | 1366-1375 | PRRARSVRYP | linker | 1-10 |
| 1 | 1376-1385 | VTGLFKDCSK | 3′-to-5′_exonuclease(NC_045512: + : 18040-19620) | 4-13 |
| 1 | 1386-1395 | PRRARSVRYP | linker | 1-10 |
| 1 | 1396-1403 | AEVQIDRL | surface_glycoprotein(NC_045512: + : 21563-25384) | 989-996 |
| 1 | 1404-1413 | PRRARSVRYP | linker | 1-10 |
| 1 | 1414-1427 | SVASQSIIAYTMSL | surface_glycoprotein@Austria(NC_045512@Austria: + : 21563-25384) | 678-691 |
| 1 | 1428-1437 | PRRARSVRYP | linker | 1-10 |
| 1 | 1438-1446 | EVFAQVKQI | surface_glycoprotein(NC_045512: + : 21563-25384) | 780-788 |
| 1 | 1447-1456 | PRRARSVRYP | linker | 1-10 |
| 1 | 1457-1475 | LEILDITPCSFGGVSVITP | surface_glycoprotein(NC_045512: + : 21563-25384) | 582-600 |
| 1 | 1476-1485 | PRRARSVRYP | linker | 1-10 |
| 1 | 1486-1496 | FFSNVTWFHVS | surface_glycoprotein@Nigeria(NC_045512@Nigeria: + : 21563-25384) | 58-68 |
| 1 | 1497-1506 | PRRARSVRYP | linker | 1-10 |
| 1 | 1507-1519 | FQTLLALHRSYLT | surface_glycoprotein@Aruba(NC_045512@Aruba: + : 21563-25384) | 234-246 |
| 2 | 1-20 | IRRPFLCCKCCYDHVISTSH | helicase(NC_045512: + : 16237-18039) | 20-39 |
| 2 | 21-30 | PRRARSVRYP | linker | 1-10 |
| 2 | 31-38 | LPINVIVF | nsp3(NC_045512: + : 2720-8554) | 1718-1725 |
| 2 | 39-48 | PRRARSVRYP | linker | 1-10 |
| 2 | 49-69 | HPNCVNCSDDRCILHCANFNV | RNA- dependent_RNA_polymerase@Aruba(NC_045512@Aruba: + : 13442-13468 + 13468-16236) | 295-315 |
| 2 | 70-79 | PRRARSVRYP | linker | 1-10 |
| 2 | 80-92 | GVNLVAVPTGYVD | 3′-to-5′_exonuclease(NC_045512: + : 18040-19620) | 114-126 |
| 2 | 93-102 | PRRARSVRYP | linker | 1-10 |
| 2 | 103-116 | IRNLQHRLYECLYR | RNA-dependent_RNA_polymerase@Austria(NC_045512@Austria: + : 13442-13468 + 13468-16236) | 720-733 |
| 2 | 117-126 | PRRARSVRYP | linker | 1-10 |
| 2 | 127-143 | VPQEHYVRITGLYPTLN | helicase@Aruba(NC_045512@Aruba: + : 16237-18039) | 241-257 |
| 2 | 144-153 | PRRARSVRYP | linker | 1-10 |
| 2 | 154-164 | RQALLKTVQFC | RNA-dependent_RNA_polymerase@Aruba(NC_045512@Aruba: + : 13442-13468 + 13468-16236) | 183-193 |
| 2 | 165-174 | PRRARSVRYP | linker | 1-10 |
| 2 | 175-182 | YDKLVSSF | nsp3(NC_045512: + : 2720-8554) | 365-372 |
| 2 | 183-192 | PRRARSVRYP | linker | 1-10 |
| 2 | 193-212 | KLTCATTRQVVNVVTTKIAL | nsp3(NC_045512: + : 2720-8554) | 1923-1942 |
| 2 | 213-222 | PRRARSVRYP | linker | 1-10 |
| 2 | 223-233 | VVNAANVYLKH | nsp3(NC_045512: + : 2720-8554) | 239-249 |
| 2 | 234-243 | PRRARSVRYP | linker | 1-10 |
| 2 | 244-257 | TKDVVECLKLSHQS | nsp3(NC_045512: + : 2720-8554) | 1830-1843 |
| 2 | 258-267 | PRRARSVRYP | linker | 1-10 |
| 2 | 268-276 | YDLSVVNAR | helicase(NC_045512: + : 16237-18039) | 382-390 |
| 2 | 277-286 | PRRARSVRYP | linker | 1-10 |
| 2 | 287-296 | ACIDCSARHI | nsp3(NC_045512: + : 2720-8554) | 1872-1881 |
| 2 | 297-306 | PRRARSVRYP | linker | 1-10 |
| 2 | 307-316 | NYQCGHYKHI | nsp3(NC_045512: + : 2720-8554) | 1012-1021 |
| 2 | 317-326 | PRRARSVRYP | linker | 1-10 |
| 2 | 327-336 | KACVEEVTTT | nsp3(NC_045512: + : 2720-8554) | 415-424 |
| 2 | 337-346 | PRRARSVRYP | linker | 1-10 |
| 2 | 347-354 | FNSVCRLM | helicase@Aruba(NC_045512@Aruba: + : 16237-18039) | 422-429 |
| 2 | 355-364 | PRRARSVRYP | linker | 1-10 |
| 2 | 365-379 | FKELLVYAADPAMHA | RNA-dependent_RNA_polymerase(NC_045512: + : 13442-13468 + 13468-16236) | 368-382 |
| 2 | 380-389 | PRRARSVRYP | linker | 1-10 |
| 2 | 390-399 | SVELKHFFFA | RNA-dependent_RNA_polymerase(NC_045512: + : 13442-13468 + 13468-16236) | 434-443 |
| 2 | 400-409 | PRRARSVRYP | linker | 1-10 |
| 2 | 410-418 | RSVYPVASP | nsp2(NC_045512: + : 806-2719) | 121-129 |
| 2 | 419-428 | PRRARSVRYP | linker | 1-10 |
| 2 | 429-446 | CANFNVLFSTVFPFTSFG | RNA-dependent_RNA_polymerase@Nigeria(NC_045512@Nigeria: + : 13442-13468 + 13468-16236) | 310-327 |
| 2 | 447-456 | PRRARSVRYP | linker | 1-10 |
| 2 | 457-469 | ANFCALILAYCNK | nsp3(NC_045512: + : 2720-8554) | 890-902 |
| 2 | 470-479 | PRRARSVRYP | linker | 1-10 |
| 2 | 480-495 | TVLKKCKSAFYILPSI | nsp3(NC_045512: + : 2720-8554) | 526-541 |
| 2 | 496-505 | PRRARSVRYP | linker | 1-10 |
| 2 | 506-520 | ERFVSLAIDAYPLTK | RNA-dependent_RNA_polymerase(NC_045512: + : 13442-13468 + 13468-16236) | 857-871 |
| 2 | 521-530 | PRRARSVRYP | linker | 1-10 |
| 2 | 531-543 | IVKFISTCACEIV | nsp2(NC_045512: + : 806-2719) | 468-480 |
| 2 | 544-553 | PRRARSVRYP | linker | 1-10 |
| 2 | 554-561 | DSYFVVKR | RNA-dependent_RNA_polymerase(NC_045512: + : 13442-13468 + 13468-16236) | 67-74 |
| 2 | 562-571 | PRRARSVRYP | linker | 1-10 |
| 2 | 572-591 | DDRCILHCANFNVLFSTVFP | RNA-dependent_RNA_polymerase(NC_045512: + : 13442-13468 + 13468-16236) | 303-322 |
| 2 | 592-601 | PRRARSVRYP | linker | 1-10 |
| 2 | 602-610 | VTANVNALL | RNA-dependent_RNA_polymerase(NC_045512: + : 13442-13468 + 13468-16236) | 700-708 |
| 2 | 611-620 | PRRARSVRYP | linker | 1-10 |
| 2 | 621-640 | LQKAAITILDGISQYSLRLI | nsp2(NC_045512: + : 806-2719) | 382-401 |
| 2 | 641-650 | PRRARSVRYP | linker | 1-10 |
| 2 | 651-673 | SHGKQVVSDIDYVPLKSATCITR | 3′-to-5′_exonuclease(NC_045512: + : 18040-19620) | 454-476 |
| 2 | 674-683 | PRRARSVRYP | linker | 1-10 |
| 2 | 684-696 | YAKPFLNKVVSTT | nsp3(NC_045512: + : 2720-8554) | 1323-1335 |
| 2 | 697-706 | PRRARSVRYP | linker | 1-10 |
| 2 | 707-733 | FSASTSAFVETVKGLDYKA | nsp2(NC_045512: + : 806-2719) | 300-326 |
| | | FKQIVESC | | |
| 2 | 734-743 | PRRARSVRYP | linker | 1-10 |
| 2 | 744-758 | ISNCVADYSVLYNSA | surface_glycoprotein@Aruba(NC_045512@Aruba: + : 21563-25384) | 354-368 |
| 2 | 759-768 | PRRARSVRYP | linker | 1-10 |
| 2 | 769-778 | KQYGDCLGNI | surface_glycoprotein@Jordan(NC_045512@Jordan: + : 21563-25384) | 831-840 |
| 2 | 779-788 | PRRARSVRYP | linker | 1-10 |
| 2 | 789-800 | FRSSVLHSTQDL | surface_glycoprotein@Aruba(NC_045512@Aruba: + : 21563-25384) | 43-54 |
| 2 | 801-810 | PRRARSVRYP | linker | 1-10 |
| 2 | 811-819 | TEIYQAGST | surface_glycoprotein@Aruba(NC_045512@Aruba: + : 21563-25384) | 466-474 |
| 2 | 820-829 | PRRARSVRYP | linker | 1-10 |
| 2 | 830-839 | GTNLPLQLGF | 3′-to-5′_exonuclease(NC_045512: + : 18040-19620) | 102-111 |
| 2 | 840-849 | PRRARSVRYP | linker | 1-10 |
| 2 | 850-861 | FSNVTWFHAIVS | surface_glycoprotein@Ghana(NC_045512@Ghana: + : 21563-25384) | 59-70 |
| 2 | 862-871 | PRRARSVRYP | linker | 1-10 |
| 2 | 872-895 | PINLVRDLPQGFSVLEPLVDLPIG | surface_glycoprotein@Belgium(NC_045512@Belgium: + : 21563-25384) | 205-228 |
| 2 | 896-905 | PRRARSVRYP | surface_glycoprotein@Nigeria(NC_045512@Nigeria: + : 21563-25384) | 886-895 |
| 2 | 906-915 | AGAALQIPFA | linker | 1-10 |
| 2 | 916-925 | PRRARSVRYP | linker | 1-10 |
| 2 | 926-941 | FSNVTWFHAIHVSGTN | surface_glycoprotein@Peru(NC_045512@Peru: + : 21563-25384) | 59-74 |
| 2 | 942-951 | PRRARSVRYP | linker | 1-10 |
| 2 | 952-966 | TISVTTEILPVSMAK | surface_glycoprotein@Mexico(NC_045512@Mexico: + : 21563-25384) | 719-733 |
| 2 | 967-976 | PRRARSVRYP | linker | 1-10 |
| 2 | 977-994 | LNDLCFTNVYADSFVIRG | surface_glycoprotein(NC_045512: + : 21563-25384) | 387-404 |
| 2 | 995-1004 | PRRARSVRYP | linker | 1-10 |
| 2 | 1005-1023 | ECAQVLSETVMCGGSLYVK | RNA-dependent_RNA_polymerase@Aruba(NC_045512@Aruba: + : 13442-13468 + 13468-16236) | 658-676 |
| 2 | 1024-1033 | PRRARSVRYP | linker | 1-10 |
| 2 | 1034-1048 | INAACRKVQHMVVKA | 3′-to-5′_exonuclease(NC_045512: + : 18040-19620) | 305-319 |
| 2 | 1049-1058 | PRRARSVRYP | linker | 1-10 |
| 2 | 1059-1072 | RLNVGDYFVLTSHT | helicase@Austria(NC_045512@Austria: + : 16237-18039) | 218-231 |
| 2 | 1073-1082 | PRRARSVRYP | linker | 1-10 |
| 2 | 1083-1091 | EPLLKGVKL | surface_glycoprotein@Croatia(NC_045512@Croatia: + : 21563-25384) | 1258-1266 |
| 2 | 1092-1101 | PRRARSVRYP | linker | 1-10 |
| 2 | 1102-1126 | SDIDITFLKKDAPYIVGDVVQEGVL | nsp3(NC_045512: + : 2720-8554) | 454-478 |
| 2 | 1127-1136 | PRRARSVRYP | linker | 1-10 |
| 2 | 1137-1151 | EFQFCNYPFLGVYHK | surface_glycoprotein@Norway(NC_045512@Norway: + : 21563-25384) | 129-143 |
| 2 | 1152-1161 | PRRARSVRYP | linker | 1-10 |
| 2 | 1162-1175 | GFNCYSPLQSYGFQ | surface_glycoprotein@Peru(NC_045512@Peru: + : 21563-25384) | 485-498 |
| 2 | 1176-1185 | PRRARSVRYP | linker | 1-10 |
| 2 | 1186-1199 | GFNCYFPLQPYGFQ | surface_glycoprotein@SmallSouthAmerica(NC_045512@SmallSouthAmerica: + : 21563-25384) | 481-494 |
| 2 | 1200-1209 | PRRARSVRYP | linker | 1-10 |
| 2 | 1210-1221 | FQTLLHRSYLTP | surface_glycoprotein@SmallAsia(NC_045512@SmallAsia: + : 21563-25384) | 230-241 |
| 2 | 1222-1231 | PRRARSVRYP | linker | 1-10 |
| 2 | 1232-1239 | KLGASQRV | membrane_glycoprotein(NC_045512: + : 26523-27191) | 180-187 |
| 2 | 1240-1249 | PRRARSVRYP | linker | 1-10 |
| 2 | 1250-1263 | TSGVGYQPYRVVVL | surface_glycoprotein@Aruba(NC_045512@Aruba: + : 21563-25384) | 496-509 |
| 2 | 1264-1273 | PRRARSVRYP | linker | 1-10 |
| 2 | 1274-1294 | ALCEKALKYFPIDKCSRIIPA | helicase@Colombia(NC_045512@Colombia: + : 16237-18039) | 316-336 |
| 2 | 1295-1304 | PRRARSVRYP | linker | 1-10 |
| 2 | 1305-1316 | ALGKLQDVVNQN | surface_glycoprotein(NC_045512: + : 21563-25384) | 944-955 |
| 2 | 1317-1326 | PRRARSVRYP | linker | 1-10 |
| 2 | 1327-1336 | KCAYWVPRAS | nsp2(NC_045512: + : 806-2719) | 239-248 |
| 2 | 1337-1346 | PRRARSVRYP | linker | 1-10 |
| 2 | 1347-1358 | ISVTTEILPVSM | surface_glycoprotein(NC_045512: + : 21563-25384) | 720-731 |
| 2 | 1359-1368 | PRRARSVRYP | linker | 1-10 |
| 2 | 1369-1380 | IEVQGYKSVNIT | nsp3(NC_045512: + : 2720-8554) | 13-24 |
| 2 | 1381-1390 | PRRARSVRYP | linker | 1-10 |
| 2 | 1391-1404 | FGTVYEKLKPVLDW | nsp2(NC_045512: + : 806-2719) | 437-450 |
| 2 | 1405-1414 | PRRARSVRYP | linker | 1-10 |
| 2 | 1415-1424 | FCLEASFNYL | nsp3(NC_045512: + : 2720-8554) | 1391-1400 |
| 2 | 1425-1434 | PRRARSVRYP | linker | 1-10 |
| 2 | 1435-1445 | YDKLQFTSLEI | helicase@Aruba(NC_045512@Aruba: + : 16237-18039) | 582-592 |
| 2 | 1 446-1455 | PRRARSVRYP | linker | 1-10 |
| 2 | 1456-1466 | DNVLSTFISAA | nsp3(NC_045512: + : 2720-8554) | 1809-1819 |
| 2 | 1467-1476 | PRRARSVRYP | linker | 1-10 |
| 2 | 1477-1484 | FTGCVIAW | surface_glycoprotein(NC_045512: + : 21563-25384) | 429-436 |
| 2 | 1485-1494 | PRRARSVRYP | linker | 1-10 |
| 2 | 1495-1504 | KQYGDCLGDI | surface_glycoprotein(NC_045512: + : 21563-25384) | 835-844 |
| 3 | 1-8 | QIGVVREF | helicase(NC_045512: + : 16237-18039) | 492-499 |
| 3 | 9-18 | PRRARSVRYP | linker | 1-10 |
| 3 | 19-40 | LGGMSYYCKSHKPSISFPLCAN | helicase@NewZealand(NC_045512@NewZealand: + : 16237-18039) | 65-86 |
| 3 | 41-50 | PRRARSVRYP | linker | 1-10 |
| 3 | 51-60 | DPIHSLRVCV | nsp3(NC_045512: + : 2720-8554) | 339-348 |
| 3 | 61-70 | PRRARSVRYP | linker | 1-10 |
| 3 | 71-88 | SQHTMLVKQGDDYVYLPY | RNA-dependent_RNA_polymerase@Aruba(NC_045512@Aruba: + : 13442-13468 + 13468-16236) | 814-831 |
| 3 | 89-98 | PRRARSVRYP | linker | 1-10 |
| 3 | 99-107 | ASQGLLASI | RNA-dependent_RNA_polymerase@Austria(NC_045512@Austria: + : 13442-13468 + 13468-16236) | 108-117 |
| 3 | 771-779 | PRRARSVRYP | linker | 1-10 |
| 3 | 118-143 | YRLANECAQVLSEMVMCGGSLYVKPG | RNA-dependent_RNA_polymerase@Australia | 653-678 |
| | | | (NC_045512@Australia: + : 13442-13468 + 13468-16236) | |
| 3 | 144-153 | PRRARSVRYP | linker | 1-10 |
| 3 | 154-185 | IMASLVLARKHTTCCSLSHRF | RNA-dependent_RNA_polymerase(NC_045512: + : 13442-13468 + 13468-16236) | 632-663 |
| | | YRLANECAQVL | | |
| 3 | 186-195 | PRRARSVRYP | linker | 1-10 |
| 3 | 196-212 | LECNVKTTEVVGDIILK | nsp3(NC_045512: + : 2720-8554) | 1244-1260 |
| 3 | 213-222 | PRRARSVRYP | linker | 1-10 |
| 3 | 223-236 | SYLFQHANLDSCKR | nsp3(NC_045512: + : 2720-8554) | 915-928 |
| 3 | 237-246 | PRRARSVRYP | linker | 1-10 |
| 3 | 247-256 | LSVNPYVCNA | helicase@Cyprus(NC_045512@Cyprus: + : 16237-18039) | 43-52 |
| 3 | 257-266 | PRRARSVRYP | linker | 1-10 |
| 3 | 267-277 | EQYVFCTVNAL | helicase(NC_045512: + : 16237-18039) | 353-363 |
| 3 | 278-287 | PRRARSVRYP | linker | 1-10 |
| 3 | 288-297 | VQQESPFVMM | nsp3(NC_045512: + : 2720-8554) | 980-989 |
| 3 | 298-307 | PRRARSVRYP | linker | 1-10 |
| 3 | 308-316 | MPVCVETKA | nsp3(NC_045512: + : 2720-8554) | 571-579 |
| 3 | 317-326 | PRRARSVRYP | linker | 1-10 |
| 3 | 327-349 | YLGGMSYYCKSHKLPISFPLCAN | helicase@CostaRica(NC_045512@CostaRica: + : 16237-18039) | 64-86 |
| 3 | 350-359 | PRRARSVRYP | linker | 1-10 |
| 3 | 360-375 | RKIFVDGVPFVVSTGY | RNA-dependent_RNA_polymerase(NC_045512: + : 13442-13468 + 13468-16236) | 331-346 |
| 3 | 376-385 | PRRARSVRYP | linker | 1-10 |
| 3 | 386-397 | VPVVDSYYSLLM | RNA-dependent_RNA_polymerase(NC_045512: + : 13442-13468 + 13468-16236) | 231-242 |
| 3 | 398-407 | PRRARSVRYP | linker | 1-10 |
| 3 | 408-417 | VMCGGSLYVK | RNA-dependent_RNA_polymerase(NC_045512: + : 13442-13468 + 13468-16236) | 667-676 |
| 3 | 418-427 | PRRARSVRYP | linker | 1-10 |
| 3 | 428-435 | DMYSVMLT | RNA-dependent_RNA_polymerase@Malaysia(NC_045512@ | 901-908 |
| | | | Malaysia: + : 13442-13468 + 13468-16236) | |
| 3 | 436-445 | PRRARSVRYP | linker | 1-10 |
| 3 | 446-453 | GVYCCREH | nsp2(NC_045512: + : 806-2719) | 47-54 |
| 3 | 454-463 | PRRARSVRYP | linker | 1-10 |
| 3 | 464-478 | YNLLKGCPAVAKHDF | RNA-dependent_RNA_polymerase@Poland(NC_045512@Poland: + : 13442-13468 + 13468-16236) | 87-101 |
| 3 | 479-488 | PRRARSVRYP | linker | 1-10 |
| 3 | 489-500 | NIGCNHTGVVGE | nsp2(NC_045512: + : 806-2719) | 250-261 |
| 3 | 501-510 | PRRARSVRYP | linker | 1-10 |
| 3 | 511-532 | SQHTMLVKQFDDYVYLPYPDPS | RNA-dependent_RNA_polymerase@Estonia(NC_045512@ | 814-835 |
| | | | Estonia: + : 13442-13468 + 13468-16236) | |
| 3 | 533-542 | PRRARSVRYP | linker | 1-10 |
| 3 | 543-550 | STFKCYGV | surface_glycoprotein@Aruba(NC_045512@Aruba: + : 21563-25384) | 371-378 |
| 3 | 551-560 | PRRARSVRYP | linker | 1-10 |
| 3 | 561-574 | ANPVLPFNDGVYFA | surface_glycoprotein@Austria(NC_045512@Austria: + : 21563-25384) | 77-90 |
| 3 | 575-584 | PRRARSVRYP | linker | 1-10 |
| 3 | 585-596 | VTMPLGYVTHGL | nsp3(NC_045512: + : 2720-8554) | 628-639 |
| 3 | 597-606 | PRRARSVRYP | linker | 1-10 |
| 3 | 607-645 | GCDVTDVTQLYLGGMSYYCKS | helicase(NC_045512: + : 16237-18039) | 54-92 |
| | | HKPPISFPLCANGQVFGL | | |
| 3 | 646-655 | PRRARSVRYP | linker | 1-10 |
| 3 | 656-686 | YIWLGFIAGLIAIVMVTI | surface_glycoprotein@Aruba(NC_045512@Aruba: + : 21563-25384) | 1211-1241 |
| | | MLCCMTSCCSCLK | | |
| 3 | 687-696 | PRRARSVRYP | linker | 1-10 |
| 3 | 697-708 | EVFAQVKQIYKT | surface_glycoprotein@Aruba(NC_045512@Aruba: + : 21563-25384) | 776-787 |
| 3 | 709-718 | PRRARSVRYP | linker | 1-10 |
| 3 | 719-737 | DKSAFVNLKQLPFFYYSDS | 3′-to-5′_exonuclease(NC_045512: + : 18040-19620) | 432-450 |
| 3 | 738-747 | PRRARSVRYP | linker | 1-10 |
| 3 | 748-763 | LQSLQTYVTQQLIRAA | surface_glycoprotein(NC_045512: + : 21563-25384) | 1001-1016 |
| 3 | 764-773 | PRRARSVRYP | linker | 1-10 |
| 3 | 774-782 | FFSNVTWFH | surface_glycoprotein(NC_045512: + : 21563-25384) | 58-66 |
| 3 | 783-792 | PRRARSVRYP | linker | 1-10 |
| 3 | 793-807 | RIIPARARVECFDKF | helicase@Aruba(NC_045512@Aruba: + : 16237-18039) | 332-346 |
| 3 | 808-817 | PRRARSVRYP | linker | 1-10 |
| 3 | 818-831 | FFSNVTWFHAIHVS | surface_glycoprotein@Bangladesh(NC_045512@Bangladesh: + : 21563-25384) | 58-71 |
| 3 | 832-841 | PRRARSVRYP | linker | 1-10 |
| 3 | 842-855 | EGVVDYGARFYFYT | nsp3(NC_045512: + : 2720-8554) | 595-608 |
| 3 | 856-865 | PRRARSVRYP | linker | 1-10 |
| 3 | 866-887 | GYQPYRVVVLSFELLHAPATVC | surface_glycoprotein(NC_045512: + : 21563-25384) | 504-525 |
| 3 | 888-897 | PRRARSVRYP | linker | 1-10 |
| 3 | 898-916 | QVAVLYQDVNCTEVPVAIH | surface_glycoprotein@China(NC_045512@China: + : 21563-25384) | 607-625 |
| 3 | 917-926 | PRRARSVRYP | linker | 1-10 |
| 3 | 927-941 | FSVAALTNNVAFQTV | RNA-dependent_RNA_polymerase(NC_045512: + : 13442-13468 + 13468-16236) | 396-410 |
| 3 | 942-951 | PRRARSVRYP | linker | 1-10 |
| 3 | 952-959 | TDVVYRAF | RNA-dependent_RNA_polymerase@Aruba(NC_045512@Aruba: + : 13442-13468 + 13468-16236) | 28-35 |
| 3 | 960-969 | PRRARSVRYP | linker | 1-10 |
| 3 | 970-981 | FNCYFPLQSYGF | surface_glycoprotein(NC_045512: + : 21563-25384) | 486-497 |
| 3 | 982-991 | PRRARSVRYP | linker | 1-10 |
| 3 | 992-999 | PSFLGRYM | nsp3(NC_045512: + : 2720-8554) | 822-829 |
| 3 | 1000-1009 | PRRARSVRYP | linker | 1-10 |
| 3 | 1010-1028 | KLNVGDYFVLTSHTVMPLS | helicase(NC_045512: + : 16237-18039) | 218-236 |
| 3 | 1029-1038 | PRRARSVRYP | linker | 1-10 |
| 3 | 1039-1054 | GSNVFQTRAGCLIGAE | surface_glycoprotein(NC_045512: + : 21563-25384) | 639-654 |
| 3 | 1055-1064 | PRRARSVRYP | linker | 1-10 |
| 3 | 1065-1074 | SVLNDILARL | surface_glycoprotein@Aruba(NC_045512@Aruba: + : 21563-25384) | 971-980 |
| 3 | 1075-1084 | PRRARSVRYP | linker | 1-10 |
| 3 | 1085-1107 | RVDFCGKGYHLMSFPQSAPHGVV | surface_glycoprotein(NC_045512: + : 21563-25384) | 1039-1061 |
| 3 | 1108-1117 | PRRARSVRYP | linker | 1-10 |
| 3 | 1118-1135 | CANFNVLFSTVFPPTSFG | RNA dependent_RNA_polymerase@China(NC_045512@China: + : 13442-13468 + 13468-16236) | 310-327 |
| 3 | 1136-1145 | PRRARSVRYP | linker | 1-10 |
| 3 | 1146-1159 | ETKCTLKSFTVEKG | surface_glycoprotein@Aruba(NC_045512@Aruba: + : 21563-25384) | 294-307 |
| 3 | 1160-1169 | PRRARSVRYP | linker | 1-10 |
| 3 | 1170-1191 | NVVIKVCEFQFCNYPFLGVYYH | surface_glycoprotein@Brazil(NC_045512@Brazil: + : 21563-25384) | 125-146 |
| 3 | 1192-1201 | PRRARSVRYP | linker | 1-10 |
| 3 | 1202-1210 | SPDVDLADI | surface_glycoprotein@Chile(NC_045512@Chile: + : 21563-25384) | 1161-1169 |
| 3 | 1211-1220 | PRRARSVRYP | linker | 1-10 |
| 3 | 1221-1237 | SAAYYVGYLQLRTFLLK | surface_glycoprotein@Italy(NC_045512@Italy: + : 21563-25384) | 258-274 |
| 3 | 1238-1247 | PRRARSVRYP | linker | 1-10 |
| 3 | 1248-1267 | MTSCCSCLKGCCSCGSCCKF | surface_glycoprotein(NC_045512: + : 21563-25384) | 1237-1256 |
| 3 | 1268-1277 | PRRARSVRYP | linker | 1-10 |
| 3 | 1278-1288 | HFPRKGVFVSN | surface_glycoprotein@SmallAsia(NC_045512@SmallAsia: + : 21563-25384) | 1078-1088 |
| 3 | 1289-1298 | PRRARSVRYP | linker | 1-10 |
| 3 | 1299-1308 | NEFYAYLRKH | RNA-dependent_RNA_polymerase(NC_045512: + : 13442-13468 + 13468-16236) | 743-752 |
| 3 | 1309-1318 | PRRARSVRYP | linker | 1-10 |
| 3 | 1319-1332 | IKVCEFQFCNDPYY | surface_glycoprotein@CostaRica(NC_045512@CostaRica: + : 21563-25384) | 128-141 |
| 3 | 1333-1342 | PRRARSVRYP | linker | 1-10 |
| 3 | 1343-1359 | YADVFHLYLQYIRKLHD | RNA-dependent_RNA_polymerase(NC_045512: + : 13442-13468 + 13468-16236) | 877-893 |
| 3 | 1360-1369 | PRRARSVRYP | linker | 1-10 |
| 3 | 1370-1384 | ARSVARQSIIAYTMS | surface_glycoprotein@Mozambique(NC_045512@Mozambique: + : 21563-25384) | 680-694 |
| 3 | 1385-1394 | PRRARSVRYP | linker | 1-10 |
| 3 | 1395-1404 | KSASVYYSQL | nsp3(NC_045512: + : 2720-8554) | 1737-1746 |
| 3 | 1405-1414 | PRRARSVRYP | linker | 1-10 |
| 3 | 1415-1430 | ANSIVCRFDTRVLSNL | 3′-to-5′_exonuclease(NC_045512: + : 18040-19620) | 394-409 |
| 3 | 1431-1440 | PRRARSVRYP | linker | 1-10 |
| 3 | 1441-1448 | PVLKGVKL | surface_glycoprotein@Aruba(NC_045512@Aruba: + : 21563-25384) | 1259-1266 |
| 3 | 1449-1458 | PRRARSVRYP | linker | 1-10 |
| 3 | 1459-1478 | PINLVRGLPQGFSVLEPLVD | surface_glycoprotein@SmallAfrica(NC_045512@SmallAfrica: + : 21563-25384) | 209-228 |
| 3 | 1479-1488 | PRRARSVRYP | linker | 1-10 |
| 3 | 1489-1502 | APGCDVTDVTQLYL | helicase@Aruba(NC_045512@Aruba: + : 16237-18039) | 52-65 |
| 4 | 1-12 | VRTNVYLAVFDK | nsp3(NC_045512: + : 2720-8554) | 351-362 |
| 4 | 13-22 | PRRARSVRYP | linker | 1-10 |
| 4 | 23-33 | KGVQIPCTCGK | nsp3(NC_045512: + : 2720-8554) | 963-973 |
| 4 | 34-43 | PRRARSVRYP | linker | 1-10 |
| 4 | 44-66 | NHDLYCQVHGNAHVASCDAIMTR | 3′-to-5′_exonuclease(NC_045512: + : 18040-19620) | 256-278 |
| 4 | 67-76 | PRRARSVRYP | linker | 1-10 |
| 4 | 77-84 | YDFAVSKG | RNA-dependent_RNA_polymerase@Aruba(NC_045512@Aruba: + : 13442-13468 + 13468-16236) | 420-427 |
| 4 | 85-94 | PRRARSVRYP | linker | 1-10 |
| 4 | 95-104 | ISDYDYYRYN | RNA-dependent_RNA_polymerase@Aruba(NC_045512@Aruba: + : 13442-13468 + 13468-16236) | 450-459 |
| 4 | 105-114 | PRRARSVRYP | linker | 1-10 |
| 4 | 115-122 | FKLASHMY | nsp3(NC_045512: + : 2720-8554) | 96-103 |
| 4 | 123-132 | PRRARSVRYP | linker | 1-10 |
| 4 | 133-147 | SYFAIGLALYYPSAR | helicase@Austria(NC_045512@Austria: + : 16237-18039) | 289-303 |
| 4 | 148-157 | PRRARSVRYP | linker | 1-10 |
| 4 | 158-166 | ASQGLVASI | RNA-dependent_RNA_polymerase@Aruba(NC_045512@Aruba: + : 13442-13468 + 13468-16236) | 771-779 |
| 4 | 167-176 | PRRARSVRYP | linker | 1-10 |
| 4 | 177-186 | VVADAVIKTL | nsp3(NC_045512: + : 2720-8554) | 56-65 |
| 4 | 187-196 | PRRARSVRYP | linker | 1-10 |
| 4 | 197-208 | SSPDAVTAYNGY | nsp3(NC_045512: + : 2720-8554) | 660-671 |
| 4 | 209-218 | PRRARSVRYP | linker | 1-10 |
| 4 | 219-226 | SINVANYQ | helicase@Mozambique(NC_045512@Mozambique: + : 16237-18039) | 263-270 |
| 4 | 227-236 | PRRARSVRYP | linker | 1-10 |
| 4 | 237-245 | SRELKVTFF | nsp3(NC_045512: + : 2720-8554) | 1134-1142 |
| 4 | 246-255 | PRRARSVRYP | linker | 1-10 |
| 4 | 256-267 | TRAKVGILCIMS | helicase(NC_045512: + : 16237-18039) | 566-577 |
| 4 | 268-277 | PRRARSVRYP | linker | 1-10 |
| 4 | 278-287 | GVITHDVSSA | helicase(NC_045512: + : 16237-18039) | 478-487 |
| 4 | 288-297 | PRRARSVRYP | linker | 1-10 |
| 4 | 298-317 | SEAVEAPLVGTPVCINGLML | nsp2(NC_045512: + : 806-2719) | 591-610 |
| 4 | 318-327 | PRRARSVRYP | linker | 1-10 |
| 4 | 328-338 | AVHECFVKRVD | 3′-to-5′_exonuclease(NC_045512: + : 18040-19620) | 281-291 |
| 4 | 339-348 | PRRARSVRYP | linker | 1-10 |
| 4 | 349-362 | RNLQHRLYECLYRN | RNA-dependent_RNA_polymerase(NC_045512: + : 13442-13468 + 13468-16236) | 721-734 |
| 4 | 363-372 | PRRARSVRYP | linker | 1-10 |
| 4 | 373-388 | SLRCGACIRRPFLCCK | helicase@Aruba(NC_045512@Aruba: + : 16237-18039) | 13-28 |
| 4 | 389-398 | PRRARSVRYP | linker | 1-10 |
| 4 | 399-411 | KASCTLSEQLDFI | nsp2(NC_045512: + : 806-2719) | 30-42 |
| 4 | 412-421 | PRRARSVRYP | linker | 1-10 |
| 4 | 422-438 | AQSFLNRVCGVSAARLT | RNA-dependent_RNA_polymerase(NC_045512: + : 13442-13468 + 13468-16236) | 4-20 |
| 4 | 439-448 | PRRARSVRYP | linker | 1-10 |
| 4 | 449-463 | QIELKFNPPALQDAY | nsp3(NC_045512: + : 2720-8554) | 867-881 |
| 4 | 464-473 | PRRARSVRYP | linker | 1-10 |
| 4 | 474-491 | IKVCEFQFCNDPFLGVYH | surface_glycoprotein@Aruba(NC_045512@Aruba: + : 21563-25384) | 125-142 |
| 4 | 492-501 | PRRARSVRYP | linker | 1-10 |
| 4 | 502-516 | HYVRITGLYPTLNIL | helicase@Indonesia(NC_045512@Indonesia: + : 16237-18039) | 245-259 |
| 4 | 517-526 | PRRARSVRYP | linker | 1-10 |
| 4 | 527-534 | YDFVVSKG | RNA-dependent_RNA_polymerase@Japan(NC_045512@Japan: + : 13442-13468 + 13468-16236) | 420-427 |
| 4 | 535-544 | PRRARSVRYP | linker | 1-10 |
| 4 | 545-564 | YVYLPYPDPSRILGAGCFVD | RNA-dependent_RNA_polymerase(NC_045512: + : 13442-13468 + 13468-16236) | 826-845 |
| 4 | 565-574 | PRRARSVRYP | linker | 1-10 |
| 4 | 575-625 | HFAIGLALYYPSARIVYTACSHAAV | helicase(NC_045512: + : 16237-18039) | 290-340 |
| | | DALCEKALKYLPIDKCSRIIPARARV | | |
| 4 | 626-635 | PRRARSVRYP | linker | 1-10 |
| 4 | 636-655 | RAKHYVYIGDPAQLPAPRTL | helicase(NC_045512: + : 16237-18039) | 392-411 |
| 4 | 656-665 | PRRARSVRYP | linker | 1-10 |
| 4 | 666-678 | WNVVRIKIVQMLS | 3′-to-5′_exonuclease(NC_045512: + : 18040-19620) | 159-171 |
| 4 | 679-688 | PRRARSVRYP | linker | 1-10 |
| 4 | 689-696 | IETISLAG | nsp3(NC_045512: + : 2720-8554) | 684-691 |
| 4 | 697-706 | PRRARSVRYP | linker | 1-10 |
| 4 | 707-717 | SELVIGAVILR | membrane_glycoprotein(NC_045512: + : 26523-27191) | 136-146 |
| 4 | 718-727 | PRRARSVRYP | linker | 1-10 |
| 4 | 728-746 | AIPINFTISVTTEILPVSM | surface_glycoprotein@Aruba(NC_045512@Aruba: + : 21563-25384) | 709-727 |
| 4 | 747-756 | PRRARSVRYP | linker | 1-10 |
| 4 | 757-766 | FEYVSQPFLM | surface_glycoprotein@Aruba(NC_045512@Aruba: + : 21563-25384) | 164-173 |
| 4 | 767-776 | PRRARSVRYP | linker | 1-10 |
| 4 | 777-792 | TGLHPTQAPTHLSVDT | 3′-to-5′_exonuclease(NC_045512: + : 18040-19620) | 16-31 |
| 4 | 793-802 | PRRARSVRYP | linker | 1-10 |
| 4 | 803-823 | HPNCVNCLDDRCILHCANFNV | RNA- dependent_RNA_polymerase@Australia(NC_045512 | 295-315 |
| | | | @Australia: + : 13442-13468 + 13468-16236) | |
| 4 | 824-833 | PRRARSVRYP | linker | 1-10 |
| 4 | 834-844 | RFQLHRSYLTP | surface_glycoprotein@Austria(NC_045512@Austria: + : 21563-25384) | 233-243 |
| 4 | 845-854 | PRRARSVRYP | linker | 1-10 |
| 4 | 855-874 | PINLVRGLPQGFSALEPLVD | surface_glycoprotein@Austria(NC_045512@Austria: + : 21563-25384) | 205-224 |
| 4 | 875-884 | PRRARSVRYP | linker | 1-10 |
| 4 | 885-895 | KESVQTFFKLV | nsp2(NC_045512: + : 806-2719) | 492-502 |
| 4 | 896-905 | PRRARSVRYP | linker | 1-10 |
| 4 | 906-928 | LIAIVMVTIMLCCITSCCSCLKG | surface_glycoprotein@Mozambique(NC_045512@Mozambique: + : 21563-25384) | 1220-1242 |
| 4 | 929-938 | PRRARSVRYP | linker | 1-10 |
| 4 | 939-955 | CSNLLLQYGSFCTQLNR | surface_glycoprotein(NC_045512: + : 21563-25384) | 749-765 |
| 4 | 956-965 | PRRARSVRYP | linker | 1-10 |
| 4 | 966-977 | TRAKVGILCITS | helicase@SouthKorea(NC_045512@SouthKorea: + : 16237-18039) | 566-577 |
| 4 | 978-987 | PRRARSVRYP | linker | 1-10 |
| 4 | 988-998 | YDNLQFISLEI | helicase@Austria(NC_045512@Austria: + : 16237-18039) | 582-592 |
| 4 | 999-1008 | PRRARSVRYP | linker | 1-10 |
| 4 | 1009-1021 | AIKCVPQADVEWK | 3′-to-5′_exonuclease(NC_045512: + : 18040-19620) | 337-349 |
| 4 | 1022-1031 | PRRARSVRYP | linker | 1-10 |
| 4 | 1032-1044 | NFRVQPTESIVRF | surface_glycoprotein(NC_045512: + : 21563-25384) | 317-329 |
| 4 | 1045-1054 | PRRARSVRYP | linker | 1-10 |
| 4 | 1055-1067 | ATNVVIKVCEFQF | surface_glycoprotein(NC_045512: + : 21563-25384) | 123-135 |
| 4 | 1068-1077 | PRRARSVRYP | linker | 1-10 |
| 4 | 1078-1087 | SSVLHSTQDF | surface_glycoprotein@Norway(NC_045512@Norway: + : 21563-25384) | 45-54 |
| 4 | 1088-1097 | PRRARSVRYP | linker | 1-10 |
| 4 | 1098-1105 | RVEAFEYY | nsp3(NC_045512: + : 2720-8554) | 810-817 |
| 4 | 1106-1115 | PRRARSVRYP | linker | 1-10 |
| 4 | 1116-1141 | YRLANECAQVLSEIV | RNA-dependent_RNA_polymerase@Canada(NC_045512@Canada: + : 13442-13468 + 13468-16236) | 653-678 |
| | | MCGGSLYVKPG | | |
| 4 | 1142-1151 | PRRARSVRYP | linker | 1-10 |
| 4 | 1152-1174 | GKIQDSLFSTASALGKLQDVVNQ | surface_glycoprotein@NorthMacedonia(NC_045512@NorthMacedonia: + : 21563-25384) | 928-950 |
| 4 | 1175-1184 | PRRARSVRYP | linker | 1-10 |
| 4 | 1185-1205 | AVLYQGVNCTEVPFAIHADQL | surface_glycoprotein@SmallAfrica(NC_045512@SmallAfrica: + : 21563-25384) | 605-625 |
| 4 | 1206-1215 | PRRARSVRYP | linker | 1-10 |
| 4 | 1216-1227 | YPSLETIQITIS | nsp3(NC_045512: + : 2720-8554) | 1483-1494 |
| 4 | 1228-1237 | PRRARSVRYP | linker | 1-10 |
| 4 | 1238-1247 | GNCDVVIGIV | surface_glycoprotein(NC_045512: + : 21563-25384) | 1124-1133 |
| 4 | 1248-1257 | PRRARSVRYP | linker | 1-10 |
| 4 | 1258-1273 | EEIIYNLLKDCPAVAK | RNA-dependent_RNA_polymerase@Senegal(NC_045512 | 83-98 |
| | | | @Senegal: + : 13442-13468 + 13468-16236) | |
| 4 | 1274-1283 | PRRARSVRYP | linker | 1-10 |
| 4 | 1284-1298 | VNCTEVPVAIHADQL | surface_glycoprotein(NC_045512: + : 21563-25384) | 615-629 |
| 4 | 1299-1308 | PRRARSVRYP | linker | 1-10 |
| 4 | 1309-1331 | TIATYCTGSIPCSVCLSGLDSLD | nsp3(NC_045512: + : 2720-8554) | 1459-1481 |
| 4 | 1332-1341 | PRRARSVRYP | linker | 1-10 |
| 4 | 1342-1364 | TQLYLGGMSYYCKPHKPPISFPL | helicase@Iceland(NC_045512@Iceland: + : 16237-18039) | 61-83 |
| 4 | 1365-1374 | PRRARSVRYP | linker | 1-10 |
| 4 | 1375-1385 | VLYQGVNCTEV | surface_glycoprotein@Aruba(NC_045512@Aruba: + : 21563-25384) | 606-616 |
| 4 | 1386-1395 | PRRARSVRYP | linker | 1-10 |
| 4 | 1396-1406 | IEELFYSYATH | 3′-to-5′_exonuclease(NC_045512: + : 18040-19620) | 363-373 |
| 4 | 1407-1416 | PRRARSVRYP | linker | 1-10 |
| 4 | 1417-1431 | AYYVGYLQPRTFLLK | surface_glycoprotein@Aruba(NC_045512@Aruba: + : 21563-25384) | 260-274 |
| 4 | 1432-1441 | PRRARSVRYP | linker | 1-10 |
| 4 | 1442-1456 | RTCCLCDRRATCFST | 3′-to-5′_exonuclease(NC_045512: + : 18040-19620) | 205-219 |
| 4 | 1457-1466 | PRRARSVRYP | linker | 1-10 |
| 4 | 1467-1474 | EIYQAGSK | surface_glycoprotein@Mexico(NC_045512@Mexico: + : 21563-25384) | 471-478 |
| 4 | 1475-1484 | PRRARSVRYP | linker | 1-10 |
| 4 | 1485-1494 | ARDLICAQKF | surface_glycoprotein(NC_045512: + : 21563-25384) | 846-855 |
| 4 | 1495-1504 | PRRARSVRYP | linker | 1-10 |
| 4 | 1505-1519 | TYACWHHSIGFDYVY | 3′-to-5′_exonuclease(NC_045512: + : 18040-19620) | 223-237 |

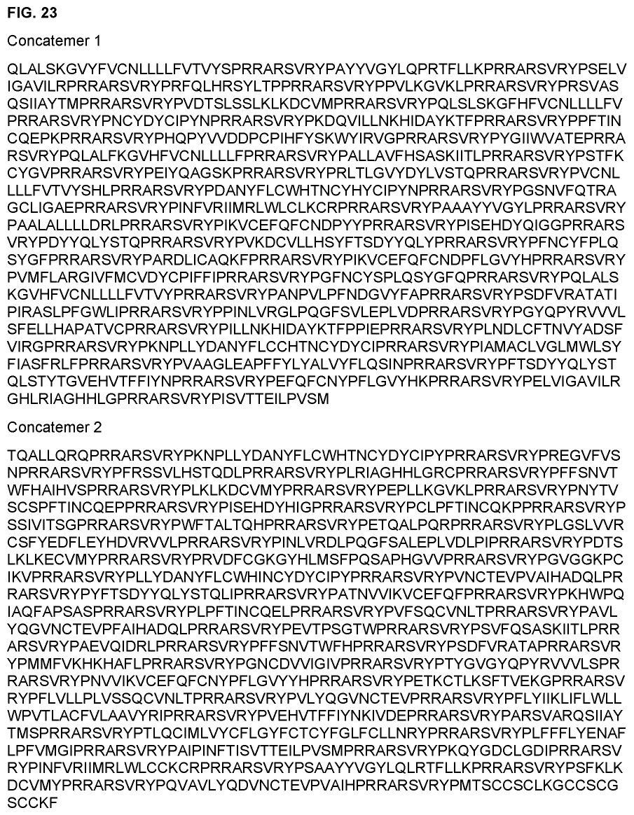

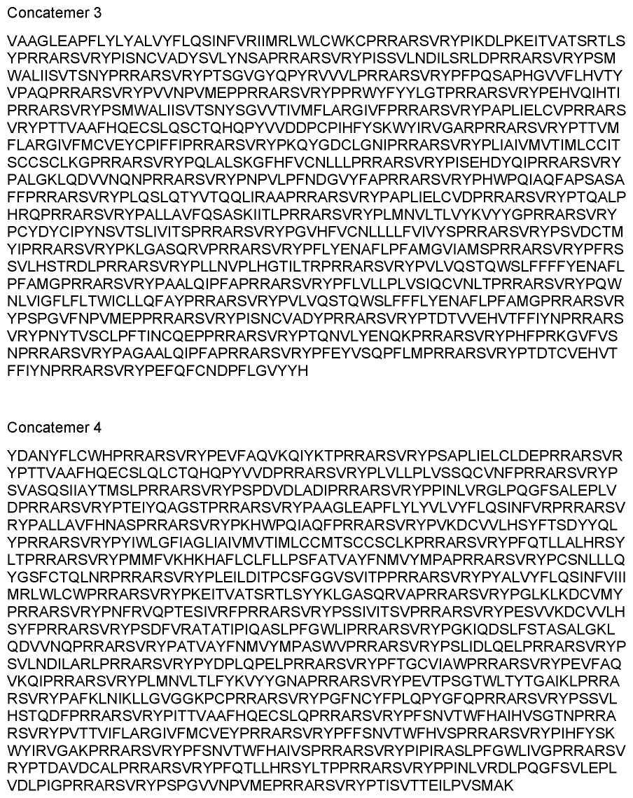

In certain embodiments, the vaccine comprises or expresses at least one of the concatemers of epitopes with intervening linker sequences (and as set forth in FIG. 23). In certain embodiments, the vaccine comprises or expresses at least two of the concatemers set forth below. In certain embodiments, the vaccine comprises or expresses three of the concatemers set forth below. In certain embodiments, the vaccine comprises or expresses all the concatemers set forth below.

TABLE 13

| | pos_in_ | | | pos_in_ |
|---|---|---|---|---|
| mmunoprec | concatemer | epitopes | gene | gene |
| 1 | 1-23 | QLALSKGVYFVCNLLLLFVTVYS | ORF3a_protein@Aruba(NC_045512@Aruba: + : 25393-26220) | 70-92 |
| 1 | 24-33 | PRRARSVRYP | linker | 1-10 |
| 1 | 34-48 | AYYVGYLQPRTFLLK | surface_glycoprotein@Aruba(NC_045512@Aruba: + : 21563-25384) | 260-274 |
| 1 | 49-58 | PRRARSVRYP | linker | 1-10 |
| 1 | 59-69 | SELVIGAVILR | membrane_glycoprotein(NC_045512: + : 26523-27191) | 136-146 |
| 1 | 70-79 | PRRARSVRYP | linker | 1-10 |
| 1 | 80-90 | RFQLHRSYLTP | surface_glycoprotein@Austria(NC_045512@Austria: + : 21563-25384) | 233-243 |
| 1 | 91-100 | PRRARSVRYP | linker | 1-10 |
| 1 | 101-108 | PVLKGVKL | surface_glycoprotein@Aruba(NC_045512@Aruba: + : 21563-25384) | 1259-1266 |
| 1 | 109-118 | PRRARSVRYP | linker | 1-10 |
| 1 | 119-131 | RSVASQSIIAYTM | surface_glycoprotein@Aruba(NC_045512@Aruba: + : 21563-25384) | 681-693 |
| 1 | 132-141 | PRRARSVRYP | linker | 1-10 |
| 1 | 142-156 | VDTSLSSLKLKDCVM | nsp6@Latvia(NC_045512@Latvia: + : 10973-11842) | 101-115 |
| 1 | 157-166 | PRRARSVRYP | linker | 1-10 |
| 1 | 167-185 | QLSLSKGFHFVCNLLLLFV | ORF3a_protein@Estonia(NC_045512@Estonia: + : 25393-26220) | 70-88 |
| 1 | 186-195 | PRRARSVRYP | linker | 1-10 |
| 1 | 196-205 | NCYDYCIPYN | ORF3a_protein(NC_045512: + : 25393-26220) | 152-161 |
| 1 | 206-215 | PRRARSVRYP | linker | 1-10 |
| 1 | 216-232 | KDQVILLNKHIDAYKTF | nucleocapsid_phosphoprotein@Aruba(NC_045512@Aruba: + : 28274-29533) | 347-363 |
| 1 | 233-242 | PRRARSVRYP | linker | 1-10 |
| 1 | 243-252 | PFTINCQEPK | ORF8_protein@India(NC_045512@India: + : 27894-28259) | 85-94 |
| 1 | 253-262 | PRRARSVRYP | linker | 1-10 |
| 1 | 263-285 | HQPYVVDDPCPIHFYSKWYIRVG | ORF8_protein(NC_045512: + : 27894-28259) | 28-50 |
| 1 | 286-295 | PRRARSVRYP | linker | 1-10 |
| 1 | 296-304 | YGIIWVATE | nucleocapsid_phosphoprotein@SmallSouthAmerica | 128-136 |
| | | | (NC_045512@SmallSouthAmerica: + : 28274-29533) | |
| 1 | 305-314 | PRRARSVRYP | linker | 1-10 |
| 1 | 315-332 | QLALFKGVHFVCNLLLLF | ORF3a_protein@CostaRica(NC_045512@CostaRica: + : 25393-26220) | 70-87 |
| 1 | 333-342 | PRRARSVRYP | linker | 1-10 |
| 1 | 343-357 | ALLAVFHSASKIITL | ORF3a_protein@Aruba(NC_045512@Aruba: + : 25393-26220) | 51-65 |
| 1 | 358-367 | PRRARSVRYP | linker | 1-10 |
| 1 | 368-375 | STFKCYGV | surface_glycoprotein@Aruba(NC_045512@Aruba: + : 21563-25384) | 371-378 |
| 1 | 376-385 | PRRARSVRYP | linker | 1-10 |
| 1 | 386-393 | EIYQAGSK | surface_glycoprotein@Mexico(NC_045512@Mexico: + : 21563-25384) | 471-478 |
| 1 | 394-403 | PRRARSVRYP | linker | 1-10 |
| 1 | 404-417 | RLTLGVYDYLVSTQ | nsp6(NC_045512: + : 10973-11842) | 236-249 |
| 1 | 418-427 | PRRARSVRYP | linker | 1-10 |
| 1 | 428-442 | VCNLLLLFVTVYSHL | ORF3a_protein@Croatia(NC_045512@Croatia: + : 25393-26220) | 80-94 |
| 1 | 443-452 | PRRARSVRYP | linker | 1-10 |
| 1 | 453-472 | DANYFLCWHTNCYHYCIPYN | ORF3a_protein@Indonesia(NC_045512@Indonesia: + : 25393-26220) | 142-161 |
| 1 | 473-482 | PRRARSVRYP | linker | 1-10 |
| 1 | 483-498 | GSNVFQTRAGCLIGAE | surface_glycoprotein(NC_045512: + : 21563-25384) | 639-654 |
| 1 | 499-508 | PRRARSVRYP | linker | 1-10 |
| 1 | 509-525 | INFVRIIMRLWLCLKCR | ORF3a_protein@Mozambique(NC_045512@Mozambique: + : 25393-26220) | 118-134 |
| 1 | 526-535 | PRRARSVRYP | linker | 1-10 |
| 1 | 536-544 | AAAYYVGYL | surface_glycoprotein(NC_045512: + : 21563-25384) | 262-270 |
| 1 | 545-554 | PRRARSVRYP | linker | 1-10 |
| 1 | 555-565 | AALALLLLDRL | nucleocapsid_phosphoprotein@Aruba(NC_045512@Aruba: + : 28274-29533) | 217-227 |
| 1 | 566-575 | PRRARSVRYP | linker | 1-10 |
| 1 | 576-589 | IKVCEFQFCNDPYY | surface_glycoprotein@CostaRica(NC_045512@CostaRica: + : 21563-25384) | 128-141 |
| 1 | 590-599 | PRRARSVRYP | linker | 1-10 |
| 1 | 600-609 | ISEHDYQIGG | ORF3a_protein@FrenchGuiana(NC_045512@FrenchGuiana: + : 25393-26220) | 179-188 |
| 1 | 610-619 | PRRARSVRYP | linker | 1-10 |
| 1 | 620-628 | DYYQLYSTQ | ORF3a_protein(NC_045512: + : 25393-26220) | 210-218 |
| 1 | 629-638 | PRRARSVRYP | linker | 1-10 |
| 1 | 639-657 | VKDCVLLHSYFTSDYYQLY | ORF3a_protein@Belgium(NC_045512@Belgium: + : 25393-26220) | 197-215 |
| 1 | 658-667 | PRRARSVRYP | linker | 1-10 |
| 1 | 668-679 | FNCYFPLQSYGF | surface_glycoprotein(NC_045512: + : 21563-25384) | 486-497 |
| 1 | 680-689 | PRRARSVRYP | linker | 1-10 |
| 1 | 690-699 | ARDLICAQKF | surface_glycoprotein(NC_045512: + : 21563-25384) | 846-855 |
| 1 | 700-709 | PRRARSVRYP | linker | 1-10 |
| 1 | 710-727 | IKVCEFQFCNDPFLGVYH | surface_glycoprotein@Aruba(NC_045512@Aruba: + : 21563-25384) | 125-142 |
| 1 | 728-737 | PRRARSVRYP | linker | 1-10 |
| 1 | 738-758 | VMFLARGIVFMCVDYCPIFFI | nsp6@Cyprus(NC_045512@Cyprus: + : 10973-11842) | 179-199 |
| 1 | 759-768 | PRRARSVRYP | linker | 1-10 |
| 1 | 769-782 | GFNCYSPLQSYGFQ | surface_glycoprotein@Peru(NC_045512@Peru: + : 21563-25384) | 485-498 |
| 1 | 783-792 | PRRARSVRYP | linker | 1-10 |
| 1 | 793-814 | QLALSKGVHFVCNLLLLFVTVY | ORF3a_protein@Australia(NC_045512@Australia: + : 25393-26220) | 70-91 |
| 1 | 815-824 | PRRARSVRYP | linker | 1-10 |
| 1 | 825-838 | ANPVLPFNDGVYFA | surface_glycoprotein@Austria(NC_045512@Austria: + : 21563-25384) | 77-90 |
| 1 | 839-848 | PRRARSVRYP | linker | 1-10 |
| 1 | 849-870 | SDFVRATATIPIRASLPFGWLI | ORF3a_protein@Belgium(NC_045512@Belgium: + : 25393-26220) | 26-47 |
| 1 | 871-880 | PRRARSVRYP | linker | 1-10 |
| 1 | 881-900 | PINLVRGLPQGFSVLEPLVD | surface_glycoprotein@SmallAfrica(NC_045512@SmallAfrica: + : 21563-25384) | 209-228 |
| 1 | 901-910 | PRRARSVRYP | linker | 1-10 |
| 1 | 911-932 | GYQPYRVVVLSFELLHAPATVC | surface_glycoprotein(NC_045512: + : 21563-25384) | 504-525 |
| 1 | 933-942 | PRRARSVRYP | linker | 1-10 |
| 1 | 943-959 | ILLNKHIDAYKTFPPIE | nucleocapsid_phosphoprotein@Chile(NC_045512@Chile: + : 28274-29533) | 351-367 |
| 1 | 960-969 | PRRARSVRYP | linker | 1-10 |
| 1 | 970-987 | LNDLCFTNVYADSFVIRG | surface_glycoprotein(NC_045512: + : 21563-25384) | 387-404 |
| 1 | 988-997 | PRRARSVRYP | linker | 1-10 |
| 1 | 998-1020 | KNPLLYDANYFLCCHTNCYDYCI | ORF3a_protein@Senegal(NC_045512@Senegal: + : 25393-26220) | 136-158 |
| 1 | 1021-1030 | PRRARSVRYP | linker | 1-10 |
| 1 | 1031-1052 | IAMACLVGLMWLSYFIASFRLF | membrane_glycoprotein@Aruba(NC_045512@Aruba: + : 26523-27191) | 82-103 |
| 1 | 1053-1062 | PRRARSVRYP | linker | 1-10 |
| 1 | 1063-1085 | VAAGLEAPFFYLYALVYFLQSIN | ORF3a_protein@Ecuador NC_045512@Ecuador + : 25393-26220) | 97-119 |
| 1 | 1086-1095 | PRRARSVRYP | linker | 1-10 |
| 1 | 1096-1123 | FTSDYYQLYSTQLSTYTGVEHVTFFIYN | ORF3a_protein@Latvia(NC_045512@Latvia: + : 25393-26220) | 207-234 |
| 1 | 1124-1133 | PRRARSVRYP | linker | 1-10 |
| 1 | 1134-1148 | EFQFCNYPFLGVYHK | surface_glycoprotein@Norway(NC_045512@Norway: + : 21563-25384) | 129-143 |
| 1 | 1149-1158 | PRRARSVRYP | linker | 1-10 |
| 1 | 1159-1179 | ELVIGAVILRGHLRIAGHHLG | membr ne_glycoprotein@Aruba(NC_045512@Aruba: + : 26523-27191) | 137-157 |
| 1 | 1180-1189 | PRRARSVRYP | linker | 1-10 |
| 1 | 1190-1201 | ISVTTEILPVSM | surface_glycoprotein(NC_045512: + : 21563-25384) | 720-731 |
| 2 | 1-8 | TQALLQRQ | nucleocapsid_phosphoprotein@Costa Rica(NC_045512@CostaRica: + : 28274-29533) | 379-386 |
| 2 | 9-18 | PRRARSVRYP | linker | 1-10 |
| 2 | 19-43 | KNPLLYDANYFLCWHTNCYDYCIPY | ORF3a_protein@Aruba(NC_045512@Aruba: + : 25393-26220) | 136-160 |
| 2 | 44-53 | PRRARSVRYP | linker | 1-10 |
| 2 | 54-61 | REGVFVSN | surface_glycoprotein@Aruba(NC_045512@Aruba: + : 21563-25384) | 1087-1094 |
| 2 | 62-71 | PRRARSVRYP | linker | 1-10 |
| 2 | 72-83 | FRSSVLHSTQDL | surface_glycoprotein@Aruba(NC_045512@Aruba: + : 21563-25384) | 43-54 |
| 2 | 84-93 | PRRARSVRYP | linker | 1-10 |
| 2 | 94-104 | LRIAGHHLGRC | membr ne_glycoprotein(NC_045512: + : 26523-27191) | 149-159 |
| 2 | 105-114 | PRRARSVRYP | linker | 1-10 |
| 2 | 115-128 | FFSNVTWFHAIHVS | surface_glycoprotein@Bangladesh(NC_045512@Bangladesh: + : 21563-25384) | 58-71 |
| 2 | 129-138 | PRRARSVRYP | linker | 1-10 |
| 2 | 139-147 | LKLKDCVMY | nsp6@Aruba(NC_045512@Aruba: + : 10973-11842) | 105-113 |
| 2 | 148-157 | PRRARSVRYP | linker | 1-10 |
| 2 | 158-166 | EPLLKGVKL | surface_glycoprotein@Croatia(NC_045512@Croatia: + : 21563-25384) | 1258-1266 |
| 2 | 167-176 | PRRARSVRYP | linker | 1-10 |
| 2 | 177-192 | NYTVSCSPFTINCQEP | ORF8_protein@CostaRica(NC_045512@CostaRica: + : 27894-28259) | 78-93 |
| 2 | 193-202 | PRRARSVRYP | linker | 1-10 |
| 2 | 203-211 | ISEHDYHIG | ORF3a_protein@NorthMacedonia(NC_045512@NorthMacedonia: + : 25393-26220) | 179-187 |
| 2 | 212-221 | PRRARSVRYP | linker | 1-10 |
| 2 | 222-232 | CLPFTINCQKP | ORF8_protein@Brazil(NC_045512@Brazil: + : 27894-28259) | 83-93 |
| 2 | 233-242 | PRRARSVRYP | linker | 1-10 |
| 2 | 243-250 | SSIVITSG | ORF3a_protein@SouthKorea(NC_045512@SouthKorea: + : 25393-26220) | 165-172 |
| 2 | 251-260 | PRRARSVRYP | linker | 1-10 |
| 2 | 261-268 | WFTALTQH | nucleocapsid_phosphoprotein(NC_045512: + : 28274-29533) | 52-59 |
| 2 | 269-278 | PRRARSVRYP | linker | 1-10 |
| 2 | 279-286 | ETQALPQR | nucleocapsid_phosphoprotein@SmallNorthAmerica | 378-385 |
| | | | (NC_045512@SmallNorthAmerica: + : 28274-29533) | |
| 2 | 287-296 | PRRARSVRYP | linker | 1-10 |
| 2 | 297-320 | LGSLVVRCSFYEDFLEYHDVRVVL | ORF8_protein(NC_045512: + : 27894-28259) | 95-118 |
| 2 | 321-330 | PRRARSVRYP | linker | 1-10 |
| 2 | 331-352 | INLVRDLPQGFSALEPLVDLPI | surface_glycoprotein@Aruba(NC_045512@Aruba: + : 21563-25384) | 206-227 |
| 2 | 353-362 | PRRARSVRYP | linker | 1-10 |
| 2 | 363-374 | DTSLKLKECVMY | nsp6@SmallAsia(NC_045512@SmallAsia: + : 10973-11842) | 102-113 |
| 2 | 375-384 | PRRARSVRYP | linker | 1-10 |
| 2 | 385-407 | RVDFCGKGYHLMSFPQSAPHGVV | surface_glycoprotein(NC_045512: + : 21563-25384) | 1039-1061 |
| 2 | 408-417 | PRRARSVRYP | linker | 1-10 |
| 2 | 418-427 | GVGGKPCIKV | nsp6@Aruba(NC_045512@Aruba: + : 10973-11842) | 274-283 |
| 2 | 428-437 | PRRARSVRYP | linker | 1-10 |
| 2 | 438-459 | LLYDANYFLCWHINCYDYCIPY | ORF3a_protein@Estonia(NC_045512@Estonia: + : 25393-26220) | 139-160 |
| 2 | 460-469 | PRRARSVRYP | linker | 1-10 |
| 2 | 470-484 | VNCTEVPVAIHADQL | surface_glycoprotein(NC_045512: + : 21563-25384) | 615-629 |
| 2 | 485-494 | PRRARSVRYP | linker | 1-10 |
| 2 | 495-509 | YFTSDYYQLYSTQLI | ORF3a_protein@Indonesia(NC_045512@Indonesia: + : 25393-26220) | 206-220 |
| 2 | 510-519 | PRRARSVRYP | linker | 1-10 |
| 2 | 520-532 | ATNVVIKVCEFQF | surface_glycoprotein(NC_045512: + : 21563-25384) | 123-135 |
| 2 | 533-542 | PRRARSVRYP | linker | 1-10 |
| 2 | 543-556 | KHWPQIAQFAPSAS | nucleocapsid_phosphoprotein@Australia(NC_045512@Australia: + : 28274-29533) | 299-312 |
| 2 | 557-566 | PRRARSVRYP | linker | 1-10 |
| 2 | 567-576 | LPFTINCQEL | ORF8_protein@Mozambique(NC_045512@Mozambique: + : 27894-28259) | 84-93 |
| 2 | 577-586 | PRRARSVRYP | linker | 1-10 |
| 2 | 587-595 | VFSQCVNLT | surface_glycoprotein@Egypt(NC_045512@Egypt: + : 21563-25384) | 11-19 |
| 2 | 596-605 | PRRARSVRYP | linker | 1-10 |
| 2 | 606-626 | AVLYQGVNCTEVPFAIHADQL | surface_glycoprotein@SmallAfrica(NC_045512@SmallAfrica: + : 21563-25384) | 605-625 |
| 2 | 627-636 | PRRARSVRYP | linker | 1-10 |
| 2 | 637-644 | EVTPSGTW | nucleocapsid_phosphoprotein(NC_045512: + : 28274-29533) | 323-330 |
| 2 | 645-654 | PRRARSVRYP | linker | 1-10 |
| 2 | 655-666 | SVFQSASKIITL | ORF3a_protein@Iceland(NC_045512@Iceland: + : 25393-26220) | 54-65 |
| 2 | 667-676 | PRRARSVRYP | linker | 1-10 |
| 2 | 677-684 | AEVQIDRL | surface_glycoprotein(NC_045512: + : 21563-25384) | 989-996 |
| 2 | 685-694 | PRRARSVRYP | linker | 1-10 |
| 2 | 695-703 | FFSNVTWFH | surface_glycoprotein(NC_045512: + : 21563-25384) | 58-66 |
| 2 | 704-713 | PRRARSVRYP | linker | 1-10 |
| 2 | 714-721 | SDFVRATA | ORF3a_protein(NC_045512: + : 25393-26220) | 26-33 |
| 2 | 722-731 | PRRARSVRYP | linker | 1-10 |
| 2 | 732-742 | MMFVKHKHAFL | nsp6@Aruba(NC_045512@Aruba: + : 10973-11842) | 57-67 |
| 2 | 743-752 | PRRARSVRYP | linker | 1-10 |
| 2 | 753-762 | GNCDVVIGIV | surface_glycoprotein(NC_045512: + : 21563-25384) | 1124-1133 |
| 2 | 763-772 | PRRARSVRYP | linker | 1-10 |
| 2 | 773-787 | TYGVGYQPYRVVVLS | surface_glycoprotein@Australia(NC_045512@Australia: + : 21563-25384) | 496-510 |
| 2 | 788-797 | PRRARSVRYP | linker | 1-10 |
| 2 | 798-819 | NVVIKVCEFQFCNYPFLGVYYH | surface_glycoprotein@Brazil(NC_045512@Brazil: + : 21563-25384) | 125-146 |
| 2 | 820-829 | PRRARSVRYP | linker | 1-10 |
| 2 | 830-843 | ETKCTLKSFTVEKG | surface_glycoprotein@Aruba(NC_045512@Aruba: + : 21563-25384) | 294-307 |
| 2 | 844-853 | PRRARSVRYP | linker | 1-10 |
| 2 | 854-869 | FLVLLPLVSSQCVNLT | surface_glycoprotein@Aruba(NC_045512@Aruba: + : 21563-25384) | 4-19 |
| 2 | 870-879 | PRRARSVRYP | linker | 1-10 |
| 2 | 880-890 | VLYQGVNCTEV | surface_glycoprotein@Aruba(NC_045512@Aruba: + : 21563-25384) | 606-616 |
| 2 | 891-900 | PRRARSVRYP | linker | 1-10 |
| 2 | 901-929 | FLYIIKLIFLWLLWPVT | membrane_glycoprotein@Aruba(NC_045512@Aruba: + : 26523-27191) | 45-73 |
| | | LACFVLAAVYRI | | |
| 2 | 930-939 | PRRARSVRYP | linker | 1-10 |
| 2 | 940-954 | VEHVTFFIYNKIVDE | ORF3a_protein(NC_045512: + : 25393-26220) | 225-239 |
| 2 | 955-964 | PRRARSVRYP | linker | 1-10 |
| 2 | 965-979 | ARSVARQSIIAYTMS | surface_glycoprotein@Mozambique(NC_045512@Mozambique: + : 21563-25384) | 680-694 |
| 2 | 980-989 | PRRARSVRYP | linker | 1-10 |
| 2 | 990-1018 | TLQCIMLVYCFLGYFC | nsp6@Aruba(NC_045512@Aruba: + : 10973-11842) | 203-231 |
| | | TCYFGLFCLLNRY | | |
| 2 | 1019-1028 | PRRARSVRYP | linker | 1-10 |
| 2 | 1029-1045 | LFFFLYENAFLPFVMGI | nsp6@SmallSouthAmerica(NC_045512@SmallSouthAmerica: + : 10973-11842) | 33-49 |
| 2 | 1046-1055 | PRRARSVRYP | linker | 1-10 |
| 2 | 1056-1074 | AIPINFTISVTTEILPVSM | surface_glycoprotein@Aruba(NC_045512@Aruba: + : 21563-25384) | 709-727 |
| 2 | 1075-1084 | PRRARSVRYP | linker | 1-10 |
| 2 | 1085-1094 | KQYGDCLGDI | surface_glycoprotein(NC_045512: + : 21563-25384) 835-844 | |
| 2 | 1095-1104 | PRRARSVRYP | linker | 1-10 |
| 2 | 1105-1121 | INFVRIIMRLWLCCKCR | ORF3a_protein@Australia(NC_045512@Australia: + : 25393-26220) | 118-134 |
| 2 | 1122-1131 | PRRARSVRYP | linker | 1-10 |
| 2 | 1132-1148 | SAAYYVGYLQLRTFLLK | surface_glycoprotein@Italy(NC_045512@Italy: + : 21563-25384) | 258-274 |
| 2 | 1149-1158 | PRRARSVRYP | linker | 1-10 |
| 2 | 1159-1168 | SFKLKDCVMY | nsp6@Malaysia(NC_045512@Malaysia: + : 10973-11842) | 107-116 |
| 2 | 1169-1178 | PRRARSVRYP | linker | 1-10 |
| 2 | 1179-1197 | QVAVLYQDVNCTEVPVAIH | surface_glycoprotein@China(NC_045512@China: + : 21563-25384) | 607-625 |
| 2 | 1198-1207 | PRRARSVRYP | linker | 1-10 |
| 2 | 1208-1227 | MTSCCSCLKGCCSCGSCCKF | surface_glycoprotein(NC_045512: + : 21563-25384) 1237- | 1256 |
| 3 | 1-37 | VAAGLEAPFLYLYALVYFLQ | ORF3a_protein@Aruba(NC_045512@Aruba: + : 25393-26220) | 97-133 |
| | | SINFVRIIMRLWLCWKC | | |
| 3 | 38-47 | PRRARSVRYP | linker | 1-10 |
| 3 | 48-65 | IKDLPKEITVATSRTLSY | membrane_glycoprotein(NC_045512: + : 26523-27191) | 161-178 |
| 3 | 66-75 | PRRARSVRYP | linker | 1-10 |
| 3 | 76-90 | ISNCVADYSVLYNSA | surface_glycoprotein@Aruba(NC_045512@Aruba: + : 21563-25384) | 354-368 |
| 3 | 91-100 | PRRARSVRYP | linker | 1-10 |
| 3 | 101-113 | ISSVLNDILSRLD | surface_glycoprotein@Bangladesh(NC_045512@Bangladesh: + : 21563-25384) | 972-984 |
| 3 | 114-123 | PRRARSVRYP | linker | 1-10 |
| 3 | 124-136 | SMWALIISVTSNY | nsp6@Aruba(NC_045512@Aruba: + : 10973-11842) | 160-172 |
| 3 | 137-146 | PRRARSVRYP | linker | 1-10 |
| 3 | 147-160 | TSGVGYQPYRVVVL | surface_glycoprotein@Aruba(NC_045512@Aruba: + : 21563-25384) | 496-509 |
| 3 | 161-170 | PRRARSVRYP | linker | 1-10 |
| 3 | 171-190 | FPQSAPHGVVFLHVTYVPAQ | surface_glycoprotein@Aruba(NC_045512@Aruba: + : 21563-25384) | 1048-1067 |
| 3 | 191-200 | PRRARSVRYP | linker | 1-10 |
| 3 | 201-208 | VVNPVMEP | ORF3a_protein(NC_045512: + : 25393-26220) | 255-262 |
| 3 | 209-218 | PRRARSVRYP | linker | 1-10 |
| 3 | 219-228 | PRWYFYYLGT | nucleocapsid_phosphoprotein@Aruba(NC_045512@Aruba: + : 28274-29533) | 106-115 |
| 3 | 229-238 | PRRARSVRYP | linker | 1-10 |
| 3 | 239-246 | EHVQIHTI | ORF3a_protein(NC_045512: + : 25393-26220) | 242-249 |
| 3 | 247-256 | PRRARSVRYP | linker | 1-10 |
| 3 | 257-285 | SMWALIISVTSNYSGVVTIV | nsp6@SmallSouthAmerica(NC_045512@SmallSouthAmerica: + : 10973-11842) | 160-188 |
| | | MFLARGIVF | | |
| 3 | 286-295 | PRRARSVRYP | linker | 1-10 |
| 3 | 296-303 | APLIELCV | ORF8_protein(NC_045512: + : 27894-28259) | 55-62 |
| 3 | 304-313 | PRRARSVRYP | linker | 1-10 |
| 3 | 314-355 | TTVAAFHQECSLQSCTQHQPYVV | ORF8_protein@Bangladesh(NC_045512@Bangladesh: + : 27894-28259) | 11-52 |
| | | DDPCPIHFYSKWYIRVGAR | | |
| 3 | 356-365 | PRRARSVRYP | linker | 1-10 |
| 3 | 366-388 | TTVMFLARGIVFMCVEYCPIFFI | nsp6@Aruba(NC_045512@Aruba: + : 10973-11842) | 177-199 |
| 3 | 389-398 | PRRARSVRYP | linker | 1-10 |
| 3 | 399-408 | KQYGDCLGNI | surface_glycoprotein@Jordan(NC_045512@Jordan: + : 21563-25384) | 831-840 |
| 3 | 409-418 | PRRARSVRYP | linker | 1-10 |
| 3 | 419-441 | LIAIVMVTIMLCCITSCCSCLKG | surface_glycoprotein@Mozambique(NC_045512@Mozambique: + : 21563-25384) | 1220-1242 |
| 3 | 442-451 | PRRARSVRYP | linker | 1-10 |
| 3 | 452-467 | QLALSKGFHFVCNLLL | ORF3a_protein@Iceland(NC_045512@Iceland: + : 25393-26220) | 70-85 |
| 3 | 468-477 | PRRARSVRYP | linker | 1-10 |
| 3 | 478-485 | ISEHDYQI | ORF3a_protein@Aruba(NC_045512@Aruba: + : 25393-26220) | 179-186 |
| 3 | 486-495 | PRRARSVRYP | linker | 1-10 |
| 3 | 496-507 | ALGKLQDVVNQN | surface_glycoprotein(NC_045512: + : 21563-25384) | 944-955 |
| 3 | 508-517 | PRRARSVRYP | linker | 1-10 |
| 3 | 518-530 | NPVLPFNDGVYFA | surface_glycoprotein(NC_045512: + : 21563-25384) | 81-93 |
| 3 | 531-540 | PRRARSVRYP | linker | 1-10 |
| 3 | 541-556 | HWPQIAQFAPSASAFF | nucleocapsid_phosphoprotein@Aruba(NC_045512@Aruba: + : 28274-29533) | 300-315 |
| 3 | 557-566 | PRRARSVRYP | linker | 1-10 |
| 3 | 567-582 | LQSLQTYVTQQLIRAA | surface_glycoprotein(NC_045512: + : 21563-25384) | 1001-1016 |
| 3 | 583-592 | PRRARSVRYP | linker | 1-10 |
| 3 | 593-601 | APLIELCVD | ORF8_protein@Bangladesh(NC_045512@Bangladesh: + : 27894-28259) | 55-63 |
| 3 | 602-611 | PRRARSVRYP | linker | 1-10 |
| 3 | 612-619 | TQALPHRQ | nucleocapsid_phosphoprotein@Mozambique(NC_045512@Mozambique: + : 28274-29533) | 379-386 |
| 3 | 620-629 | PRRARSVRYP | linker | 1-10 |
| 3 | 630-644 | ALLAVFQSASKIITL | ORF3a_protein@Bangladesh(NC_045512@Bangladesh: + : 25393-26220) | 51-65 |
| 3 | 645-654 | PRRARSVRYP | linker | 1-10 |
| 3 | 655-668 | LMNVLTLVYKVYYG | nsp6@Aruba(NC_045512@Aruba: + : 10973-11842) | 139-152 |
| 3 | 669-678 | PRRARSVRYP | linker | 1-10 |
| 3 | 679-697 | CYDYCIPYNSVTSLIVITS | ORF3a_protein@Netherlands(NC_045512@Netherlands: + : 25393-26220) | 153-171 |
| 3 | 698-707 | PRRARSVRYP | linker | 1-10 |
| 3 | 708-724 | GVHFVCNLLLLFVIVYS | ORF3a_protein@Malaysia(NC_045512@Malaysia: + : 25393-26220) | 76-92 |
| 3 | 725-734 | PRRARSVRYP | linker | 1-10 |
| 3 | 735-742 | SVDCTMYI | surface_glycoprotein(NC_045512: + : 21563-25384) | 735-742 |
| 3 | 743-752 | PRRARSVRYP | linker | 1-10 |
| 3 | 753-760 | KLGASQRV | membrane_glycoprotein(NC_045512: + : 26523-27191) | 180-187 |
| 3 | 761-770 | PRRARSVRYP | linker | 1-10 |
| 3 | 771-788 | FLYENAFLPFAMGVIAMS | nsp6@Mexico(NC_045512@Mexico: + : 10973-11842) | 36-53 |
| 3 | 789-798 | PRRARSVRYP | linker | 1-10 |
| 3 | 799-810 | FRSSVLHSTRDL | surface_glycoprotein@Nigeria(NC_045512@Nigeria: + : 21563-25384) | 43-54 |
| 3 | 811-820 | PRRARSVRYP | linker | 1-10 |
| 3 | 821-833 | LLNVPLHGTILTR | membrane_glycoprotein(NC_045512: + : 26523-27191) | 119-131 |
| 3 | 834-843 | PRRARSVRYP | linker | 1-10 |
| 3 | 844-868 | VLVQSTQWSLFFFFYENAFLPFAMG | nsp6@Iceland(NC_045512@Iceland: + : 10973-11842) | 24-48 |
| 3 | 869-878 | PRRARSVRYP | linker | 1-10 |
| 3 | 879-886 | AALQIPFA | surface_glycoprotein(NC_045512: + : 21563-25384) | 892-899 |
| 3 | 887-896 | PRRARSVRYP | linker | 1-10 |
| 3 | 897-912 | FLVLLPLVSIQCVNLT | surface_glycoprotein@SmallNorthAmerica | 4-19 |
| | | | (NC_045512@SmallNorthAmerica: + : 21563-25384) | |
| 3 | 913-922 | PRRARSVRYP | linker | 1-10 |
| 3 | 923-943 | QWNLVIGFLFLTWICLLQFAY | membrane_glycoprotein@Aruba(NC_045512@Aruba: + : 26523-27191) | 19-39 |
| 3 | 944-953 | PRRARSVRYP | linker | 1-10 |
| 3 | 954-978 | VLVQSTQWSLFFFLYENAFLPFAMG | nsp6@Aruba(NC_045512@Aruba: + : 10973-11842) | 24-48 |
| 3 | 979-988 | PRRARSVRYP | linker | 1-10 |
| 3 | 989-999 | SPGVFNPVMEP | ORF3a_protein@Colombia(NC_045512@Colombia: + : 25393-26220) | 252-262 |
| 3 | 1000-1009 | PRRARSVRYP | linker | 1-10 |
| 3 | 1010-1017 | ISNCVADY | surface_glycoprotein(NC_045512: + : 21563-25384) | 358-365 |
| 3 | 1018-1027 | PRRARSVRYP | linker | 1-10 |
| 3 | 1028-1041 | TDTVVEHVTFFIYN | ORF3a_protein@Costa Rica(NC_045512@CostaRica: + : 25393-26220) | 221-234 |
| 3 | 1042-1051 | PRRARSVRYP | linker | 1-10 |
| 3 | 1052-1067 | NYTVSCLPFTINCQEP | ORF8_protein@Bangladesh(NC_045512@Bangladesh: + : 27894-28259) | 78-93 |
| 3 | 1068-1077 | PRRARSVRYP | linker | 1-10 |
| 3 | 1078-1087 | TQNVLYENQK | surface_glycoprotein(NC_045512: + : 21563-25384) | 912-921 |
| 3 | 1088-1097 | PRRARSVRYP | linker | 1-10 |
| 3 | 1098-1108 | HFPRKGVFVSN | surface_glycoprotein@SmallAsia(NC_045512@SmallAsia: + : 21563-25384) | 1078-1088 |
| 3 | 1109-1118 | PRRARSVRYP | linker | 1-10 |
| 3 | 1119-1128 | AGAALQIPFA | surface_glycoprotein@Nigeria(NC_045512@Nigeria: + : 21563-25384) | 886-895 |
| 3 | 1129-1138 | PRRARSVRYP | linker | 1-10 |
| 3 | 1139-1148 | FEYVSQPFLM | surface_glycoprotein@Aruba(NC_045512@Aruba: + : 21563-25384) | 164-173 |
| 3 | 1149-1158 | PRRARSVRYP | linker | 1-10 |
| 3 | 1159-1172 | TDTCVEHVTFFIYN | ORF3a_protein@Luxembourg(NC_045512@Luxembourg: + : 25393-26220) | 221-234 |
| 3 | 1173-1182 | PRRARSVRYP | surface_glycoprotein@Chile(NC_045512@Chile: + : 21563-25384) | 132-146 |
| 3 | 1183-1197 | EFQFCNDPFLGVYYH | linker | 1-10 |
| 4 | 1-10 | YDANYFLCWH | ORF3a_protein(NC_045512: + : 25393-26220) | 141-150 |
| 4 | 11-20 | PRRARSVRYP | linker | 1-10 |
| 4 | 21-32 | EVFAQVKQIYKT | surface_glycoprotein@Aruba(NC_045512@Aruba: + : 21563-25384) | 776-787 |
| 4 | 33-42 | PRRARSVRYP | linker | 1-10 |
| 4 | 43-53 | SAPLIELCLDE | ORF8_protein@Russia(NC_045512@Russia: + : 27894-28259) | 54-64 |
| 4 | 54-63 | PRRARSVRYP | linker | 1-10 |
| 4 | 64-87 | TTVAAFHQECSLQLCTQHQPYVVD | ORF8_protein@USA(NC_045512@USA: + : 27894-28259) | 11-34 |
| 4 | 88-97 | PRRARSVRYP | linker | 1-10 |
| 4 | 98-111 | LVLLPLVSSQCVNF | surface_glycoprotein@Brazil(NC_045512@Brazil: + : 21563-25384) | 5-18 |
| 4 | 112-121 | PRRARSVRYP | linker | 1-10 |
| 4 | 122-135 | SVASQSIIAYTMSL | surface_glycoprotein@Austria(NC_045512@Austria: + : 21563-25384) | 678-691 |
| 4 | 136-145 | PRRARSVRYP | linker | 1-10 |
| 4 | 146-154 | SPDVDLADI | surface_glycoprotein@Chile(NC_045512@Chile: + : 21563-25384) | 1161-1169 |
| 4 | 155-164 | PRRARSVRYP | linker | 1-10 |
| 4 | 165-184 | PINLVRGLPQGFSALEPLVD | surface_glycoprotein@Austria(NC_045512@Austria: + : 21563-25384) | 205-224 |
| 4 | 185-194 | PRRARSVRYP | linker | 1-10 |
| 4 | 195-203 | TEIYQAGST | surface_glycoprotein@Aruba(NC_045512@Aruba: + : 21563-25384) | 466-474 |
| 4 | 204-213 | PRRARSVRYP | linker | 1-10 |
| 4 | 214-238 | AAGLEAPFLYLYVLVYFLQSINFVR | ORF3a_protein@Norway(NC_045512@Norway: + : 25393-26220) | 98-122 |
| 4 | 239-248 | PRRARSVRYP | linker | 1-10 |
| 4 | 249-258 | ALLAVFHNAS | ORF3a_protein@SmallSouthAmerica(NC_045512@SmallSouthAmerica: + : 25393-26220) | 51-60 |
| 4 | 259-268 | PRRARSVRYP | linker | 1-10 |
| 4 | 269-277 | KHWPQIAQF | nucleocapsid_phosphoprotein(NC_045512: + : 28274-29533) | 299-307 |
| 4 | 278-287 | PRRARSVRYP | linker | 1-10 |
| 4 | 288-306 | VKDCVVLHSYFTSDYYQLY | ORF3a_protein@Aruba(NC_045512@Aruba: + : 25393-26220) | 197-215 |
| 4 | 307-316 | PRRARSVRYP | linker | 1-10 |
| 4 | 317-347 | YIWLGFIAGLIAIVMVTIM | surface_glycoprotein@Aruba(NC_045512@Aruba: + : 21563-25384) | 1211-1241 |
| | | LCCMTSCCSCLK | | |
| 4 | 348-357 | PRRARSVRYP | linker | 1-10 |
| 4 | 358-370 | FQTLLALHRSYLT | surface_glycoprotein@Aruba(NC_045512@Aruba: + : 21563-25384) | 234-246 |
| 4 | 371-380 | PRRARSVRYP | linker | 1-10 |
| 4 | 57-88 | MMFVKHKHAFLCLFLLPS | nsp6@Indonesia(NC_045512@Indonesia: + : 10973-11842) | 381-412 |
| | | FATVAYFNMVYMPA | | |
| 4 | 413-422 | PRRARSVRYP | linker | 1-10 |
| 4 | 423-439 | CSNLLLQYGSFCTQLNR | surface_glycoprotein(NC_045512: + : 21563-25384) | 749-765 |
| 4 | 440-449 | PRRARSVRYP | linker | 1-10 |
| 4 | 450-468 | LEILDITPCSFGGVSVITP | surface_glycoprotein(NC_045512: + : 21563-25384) | 582-600 |
| 4 | 469-478 | PRRARSVRYP | linker | 1-10 |
| 4 | 479-501 | YALVYFLQSINFVIIIMRLWLCW | ORF3a_protein@Denmark(NC_045512@Denmark: + : 25393-26220) | 109-131 |
| 4 | 502-511 | PRRARSVRYP | linker | 1-10 |
| 4 | 512-534 | KEITVATSRTLSYYKLGASQRVA | membrane_glycoprotein@Aruba(NC_045512@Aruba: + : 26523-27191) | 166-188 |
| 4 | 535-544 | PRRARSVRYP | linker | 1-10 |
| 4 | 545-554 | GLKLKDCVMY | nsp6@Peru(NC_045512@Peru: + : 10973-11842) | 107-116 |
| 4 | 555-564 | PRRARSVRYP | linker | 1-10 |
| 4 | 565-577 | NFRVQPTESIVRF | surface_glycoprotein(NC_045512: + : 21563-25384) | 317-329 |
| 4 | 578-587 | PRRARSVRYP | linker | 1-10 |
| 4 | 588-595 | SSIVITSV | ORF3a_protein@Aruba(NC_045512@Aruba: + : 25393-26220) | 165-172 |
| 4 | 596-605 | PRRARSVRYP | linker | 1-10 |
| 4 | 606-619 | ESVVKDCVVLHSYF | ORF3a_protein@CostaRica(NC_045512@CostaRica: + : 25393-26220) | 194-207 |
| 4 | 620-629 | PRRARSVRYP | linker | 1-10 |
| 4 | 630-651 | SDFVRATATIPIQASLPFGWLI | ORF3a_protein@Aruba(NC_045512@Aruba: + : 25393-26220) | 26-47 |
| 4 | 652-661 | PRRARSVRYP | linker | 1-10 |
| 4 | 662-684 | GKIQDSLFSTASALGKLQDVVNQ | surface_glycoprotein@NorthMacedonia | 928-950 |
| | | | (NC_045512@NorthMacedonia: + : 21563-25384) | |
| 4 | 685-694 | PRRARSVRYP | linker | 1-10 |
| 4 | 695-710 | ATVAYFNMVYMPASWV | nsp6@Aruba(NC_045512@Aruba: + : 10973-11842) | 76-91 |
| 4 | 711-720 | PRRARSVRYP | linker | 1-10 |
| 4 | 721-728 | SLIDLQEL | surface_glycoprotein(NC_045512: + : 21563-25384) | 1196-1203 |
| 4 | 729-738 | PRRARSVRYP | linker | 1-10 |
| 4 | 739-748 | SVLNDILARL | surface_glycoprotein@Aruba(NC_045512@Aruba: + : 21563-25384) | 971-980 |
| 4 | 749-758 | PRRARSVRYP | linker | 1-10 |
| 4 | 759-766 | YDPLQPEL | surface_glycoprotein(NC_045512: + : 21563-25384) | 1138-1145 |
| 4 | 767-776 | PRRARSVRYP | linker | 1-10 |
| 4 | 777-784 | FTGCVIAW | surface_glycoprotein(NC_045512: + : 21563-25384) | 429-436 |
| 4 | 785-794 | PRRARSVRYP | linker | 1-10 |
| 4 | 795-803 | EVFAQVKQI | surface_glycoprotein(NC_045512: + : 21563-25384) | 780-788 |
| 4 | 804-813 | PRRARSVRYP | linker | 1-10 |
| 4 | 814-829 | LMNVLTLFYKVYYGNA | nsp6@Chile(NC_045512@Chile: + : 10973-11842) | 142-157 |
| 4 | 830-839 | PRRARSVRYP | linker | 1-10 |
| 4 | 840-856 | EVTPSGTWLTYTGAIKL | nucleocapsid_phosphoprotein@Bulgaria(NC_045512@Bulgaria: + : 28274-29533) | 323-339 |
| 4 | 857-866 | PRRARSVRYP | linker | 1-10 |
| 4 | 867-882 | AFKLNIKLLGVGGKPC | nsp6(NC_045512: + : 10973-11842) | 268-283 |
| 4 | 883-892 | PRRARSVRYP | linker | 1-10 |
| 4 | 893-906 | GFNCYFPLQPYGFQ | surface_glycoprotein@SmallSouthAmerica | 481-494 |
| | | | (NC_045512@SmallSouthAmerica: + : 21563-25384) | |
| 4 | 907-916 | PRRARSVRYP | linker | 1-10 |
| 4 | 917-926 | SSVLHSTQDF | surface_glycoprotein@Norway(NC_045512@Norway: + : 21563-25384) | 45-54 |
| 4 | 927-936 | PRRARSVRYP | linker | 1-10 |
| 4 | 937-950 | ITTVAAFHQECSLQ | ORF8_protein@Aruba(NC_045512@Aruba: + : 27894-28259) | 10-23 |
| 4 | 951-960 | PRRARSVRYP | linker | 1-10 |
| 4 | 961-976 | FSNVTWFHAIHVSGTN | surface_glycoprotein@Peru(NC_045512@Peru: + : 21563-25384) | 59-74 |
| 4 | 977-986 | PRRARSVRYP | linker | 1-10 |
| 4 | 987-1004 | VTTVIFLARGIVFMCVEY | nsp6@Malaysia(NC_045512@Malaysia: + : 10973-11842) | 179-196 |
| 4 | 1005-1014 | PRRARSVRYP | linker | 1-10 |
| 4 | 1015-1025 | FFSNVTWFHVS | surface_glycoprotein@Nigeria(NC_045512@Nigeria: + : 21563-25384) | 58-68 |
| 4 | 1026-1035 | PRRARSVRYP | linker | 1-10 |
| 4 | 1036-1049 | IHFYSKWYIRVGAK | ORF8_protein@Mozambique(NC_045512@Mozambique: + : 27894-28259) | 39-52 |
| 4 | 1050-1059 | PRRARSVRYP | linker | 1-10 |
| 4 | 1060-1071 | FSNVTWFHAIVS | surface_glycoprotein@Ghana(NC_045512@Ghana: + : 21563-25384) | 59-70 |
| 4 | 1072-1081 | PRRARSVRYP | linker | 1-10 |
| 4 | 1082-1096 | IPIRASLPFGWLIVG | ORF3a_protein@Iceland(NC_045512@Iceland: + : 25393-26220) | 35-49 |
| 4 | 1097-1106 | PRRARSVRYP | linker | 1-10 |
| 4 | 1107-1114 | TDAVDCAL | surface_glycoprotein(NC_045512: + : 21563-25384) | 286-293 |
| 4 | 1115-1124 | PRRARSVRYP | linker | 1-10 |
| 4 | 1125-1136 | FQTLLHRSYLTP | surface_glycoprotein@SmallAsia(NC_045512@SmallAsia: + : 21563-25384) | 230-241 |
| 4 | 1137-1146 | PRRARSVRYP | linker | 1-10 |
| 4 | 1147-1170 | PINLVRDLPQGFSVLEPLVDLPIG | surface_glycoprotein@Belgium(NC_045512@Belgium: + : 21563-25384) | 205-228 |
| 4 | 1171-1180 | PRRARSVRYP | linker | 1-10 |
| 4 | 1181-1190 | SPGVVNPVME | ORF3a_protein@Brazil(NC_045512@Brazil: + : 25393-26220) | 252-261 |
| 4 | 1191-1200 | PRRARSVRYP | linker | 1-10 |
| 4 | 1201-1215 | TISVTTEILPVSMAK | surface_glycoprotein@Mexico(NC_045512@Mexico: + : 21563-25384) | 719-733 |

### Immune Response Enhancing Components:

In certain embodiments, the vaccines comprise or are capable of expressing one or more additional components to enhance the immune response to the one or more viral immunogens. These components may include, for example, targeting molecules, elements which enhance antigen processing, immunostimulatory molecules such as cytokines and other adjuvants. In certain embodiments, the vaccine comprises one or more fusion polypeptides comprising the one or more additional components and one or more concatemers of epitopes. The additional components include but are not limited to one or more targeting molecules/motifs. In certain embodiments where the vaccine comprises more than more concatemer, each concatemer is fused to one or more targeting molecules. The targeting molecules may be the same for all immunogens in the vaccine or different.

It is known in the art that targeting the immunogen to antigen presenting cells (APCs), including but not limited to dendritic cells, or enhancing antigen processing by APCs enhances immunogenicity.

In specific embodiments, the fusion protein comprises one or more CD74 or fragment thereof and one or more concatemer(s) of epitopes. In certain embodiments, the fusion protein comprises the CD74 transmembrane and cytoplasmic domain and one or more concatemer(s) of epitopes. In certain embodiments, the CD74 transmembrane and cytoplasmic domain has the sequence set forth below:

HRRRSRSCREDQKPVMDDQRDLISNNEQLPMLGRRP
GAPESKCSRGALYTGFSILVTLLLAGQATTAYFLY

In specific embodiments, the fusion protein comprises one or more human leukocyte antigen (HLA) or fragment thereof and the one or more immunogen or fragment thereof. In specific embodiments, the fusion protein comprises the HLA transmembrane and cytoplasmic domain and immunogen or fragment thereof.

In specific embodiment, the HLA fragment comprises the following sequence:

MVKCATLSVDSGQASDSSAAQTYSGGKRDSSKRRW
MVAAVVAGTIVAGLLVLGAIIGV

In certain embodiments, the targeting moiety is a chimeric targeting moiety comprising portions of different molecules. For example, the targeting moiety may comprise the cytoplasmic domain from one molecule fused to the transmembrane domain of another molecule. In certain embodiments, the targeting moiety comprises the CD74 cytoplasmic domain fused to an HLA transmembrane sequence. In specific embodiments, the targeting moiety comprises the following sequence:

HRRRSRSCREDQKPVMDDQRDLISNNEQLPMLGRR
PGAPESKCSRWMVAAVVAGTIVAGLLVLGAIIGV

### Other Elements:

The proteins of the present invention may also include tags. Appropriate tags are known in the art and include but are not limited to HA-, FLAG®- or myc- or alpha tags.

### Exemplary Vaccines

Non-limiting exemplary vaccines or concatemers expressed by exemplary vaccines are set forth in the figures.

### Vaccine Formulations

The vaccines formulations may also comprise pharmaceutically acceptable carriers, excipients and/or adjuvants. Adjuvants and carriers suitable for administering genetic vaccines and immunogens are known in the art. Conventional carriers and adjuvants are for example reviewed in Kiyono et al. 1996.

A vaccine adjuvant is a component that potentiates the immune responses to an antigen and/or modulates it towards the desired immune responses. A vaccine may include one or more adjuvants. Exemplary adjuvants include mineral salts including but not limited to aluminium salts (such as amorphous aluminum hydroxyphosphate sulfate (AAHS), aluminum hydroxide, aluminum phosphate, potassium aluminum sulfate (Alum)) and calcium phosphate gels; Oil emulsions and surfactant based formulations, including but not limited to MF59 (microfluidised detergent mmunoprec oil-in-water emulsion), QS21 (purified saponin), AS02 [SBAS2] (oil-in-water emulsion+MPL+QS-21), Montanide ISA-51 and ISA-720 (mmunoprec water-in-oil emulsion); Particulate adjuvants, including but not limited to virosomes (unilamellar liposomal vehicles incorporating influenza haemagglutinin), AS04 ([SBAS4] Al salt with MPL), ISCOMS (structured complex of saponins and lipids), polylactide co-glycolide (PLG). And; microbial derivatives (natural and synthetic), including but not limited to monophosphoryl lipid A (MPL), Detox (MPL+M. Phlei cell wall skeleton), AGP [RC-529] (synthetic acylated monosaccharide), DC_Chol (lipoidal immunostimulators able to self mmunopr into liposomes), OM-174 (lipid A derivative), CpG motifs (synthetic oligonucleotides containing immunostimulatory CpG motifs), modified LT and CT (genetically modified bacterial toxins to provide non-toxic adjuvant effects); endogenous human immunomodulators, including but not limited to hGM-CSF or hIL-12 (cytokines that can be administered either as protein or plasmid encoded), Immudaptin (C3d tandem array) and inert vehicles, such as gold particles.

The vaccine formulations may also comprise a stabilizer. Suitable stabilizer are known in the art and include but are not limited to amino acids, antioxidants, cyclodextrins, proteins, sugars/sugar alcohols, and surfactants. See for example Morefield, AAPS J. 2011 June; 13(2): 191-200; https://www.ncbi.nlm.nih.gov/pmc/articles/PMC3085699/).

The vaccine can be incorporated into liposomes, microspheres or other polymer matrices. Liposomes can consist of phospholipids or other lipids, and can be nontoxic, physiologically acceptable and metabolizable carriers that are relatively simple to make and administer.

Previously, it has been found that a SARS-COV-2 SAM lipid nanoparticle (LNP) vaccine induced high neutralizing antibody titers in mice (Mckay et al., Nat Commun 11, 3523 (2020). https://doi.org/10.1038/s41467-020-17409-9). Briefly, the LNP (described in US patent U.S. Pat. No. 10,221,127) contains an ionizable cationic lipid phosphatidylcholine/cholesterol/PEG-lipid. The SAM RNA were encapsulated in LNP using a self-assembly process in which an aqueous solution of SAM RNA at pH=4.0 is rapidly mixed with an ethanolic lipid mixture. LNP.

Accordingly, in certain embodiments, the vaccines formulations comprise lipid nanoparticle delivery formulations of nucleic acid-based vaccines. Optionally, the lipid is cationic. Appropriate cationic lipids are known in the art. Non-limiting examples include phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA). Also see for example, U.S. Pat. No. 10,221,127 (incorporated by reference) and Reichmuth A M et al. (Therapeutic Delivery. 2016; 7(5):319-334. DOI: 10.4155/tde-2016-0006). In specific embodiments, the vaccines formulations comprise lipid nanoparticle delivery formulations of SAM RNA vaccines. In specific embodiments, the LNPs comprise an ionizable cationic lipid (phosphatidylcholine:cholesterol/PEG-lipid (50:10:38.5:1.5 mol/mol). In certain embodiments, the RNA to total lipid ratio in the LNP is approximately 0.05 (wt/wt). In certain embodiments, the LNPs have a diameter of ˜80 nm.

In other embodiments, Charge-Altering Releasable Transporters (CARTs) as a mRNA delivery platform.

Accordingly, in certain embodiments, the vaccines viral vector-based vaccines or nucleic acid-based vaccines. In specific embodiments, the vaccines are SAM RNA-based vaccines. Optionally, the SAM vaccines are in lipid nanoparticle formulations.

### Method of Vaccination

Also provided herein is a method of treating, protecting against, and/or preventing disease associated with the infectious agent in a subject in need thereof by administering the vaccine to the subject. For example, a worker skilled in the art would readily appreciate that a SARS-COV-2 vaccine may be used treating, protecting against, and/or preventing disease associated with SARS-COV-2 (i.e. COVID 19). Administration of the vaccine to the subject can induce or elicit a specific immune response against the vaccine target in the subject.

The subject may be a human or other animals, including but not limited to other mammals, such as non-human primates, cats, dogs, equines (including but not limited to horses, donkeys and zebras), camels, sheep, goats, and bovines (including but not limited to cows).

The induced immune response can be used to treat, prevent, and/or protect against disease related to the vaccine target. For example, a SARS-COV-2 vaccine to the subject can induce or elicit a specific immune response against the SARS-COV-2 in the subject. The induced immune response provides the subject administered the vaccine with protection against the vaccine target, such as a SARS-COV-2 vaccine provides resistance to SARS-COV-2.

The induced immune response can include an induced humoral immune response and/or an induced cellular immune response. The induced humoral immune response can include IgG antibodies and/or neutralizing antibodies that are reactive to the antigen. The induced cellular immune response can include a CD8+ T cell response.

The number of vaccine doses for effective treatment can be 1, 2, 3, 4, 5, 6, 7, 8, 9, or 10.

In certain embodiments, a single type of vaccine is used. In other embodiments, multiple types of vaccines are used. For example, in certain embodiments, a prime and boost strategy of vaccination is used. In such embodiments, one vaccine expresses epitopes are linked to CD74/HLA targeting sequences is used (this may promote DC cross priming of T cells). A second vaccine expresses epitopes that lack all signal sequences and transmembrane domains is used (this may promote endogenous antigen presentation). These may be administered together or in 2 separate immunization of priming and boosting to promote optimum T cell response.

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, an equine, a bovine, a pig, a sheep, a camel, a cat, a dog, a rat, or a mouse.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997). The nucleic acid of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. The vaccine can be delivered to the interstitial spaces of tissues of an individual (Felgner et al., U.S. Pat. Nos. 5,580,859 and 5,703,055. The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be administered via electroporation, such as by a method described in U.S. Pat. No. 7,664,545. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359. The electroporation may be carried out via a minimally invasive device.

A method of inducing an antigen-specific immune response in a subject, the method comprising administering to the subject the vaccine comprising at least one nucleic acid sequence of SEQ ID NO. 1-51 or a mutated variant thereof capable of expressing a polypeptide in an amount effective to produce an antigen-specific immune response in the subject.

### EXAMPLES

### Example 1: Identification and Quantification of Peptides Eluted from Hla Molecules from Cultured Cells and from Patient Samples

Peptide analysis from cultured cells: Peptide epitopes in the SARS-COV-2 proteins will be mapped. Using human THP-1 (monocytic) and Calu-3 (lung epithelial) cells, we will treat them with a recombinant version of each SARS-COV-2 protein. The duration of antigen exposure will be optimized for maximum presentation. After antigen challenge, we will first collect host-processed, HLA-bound peptides from the cells by immunoprecipitation (IP) of the HLA, followed by peptide elution using acid stripping (as performed previously). We will use anti-HLA-A (IgG EP1395Y, Abcam 52922), anti HLA-B (IgG1 JOAN-1, Thermo MA1-35410), and anti-HLA-C (IgG EPR6749, Abcam 126722) antibodies to mmunoprecipitated human HLA I proteins and their ligands. For MHC II, we will use the L243 antibody against HLA-DRB1 (IgG2a, Abcam 136320), or other antibodies against several variants, such as the HL-40 antibody against HLA-DR and -DP (IgG2a, Abcam 8085). The IgG will be conjugated to magnetic Protein G beads and cross-linked with disuccinimidyl suberate so that the IgG is not eluted and the beads can be re-used. Post-challenge, cells will be solubilized in a detergent-containing buffer, and HLA molecules will be immunoprecipitated with the IgG conjugated magnetic beads. HLA-bound peptides will then be eluted in 10% acetic acid, separated from larger proteins by a 5000 molecular weight cutoff filter and then detected by mass spectrometry, as performed previously.

Peptide analysis from COVID-19 Patient Cohort: Vancouver General Hospital (VGH) and Surrey Memorial Hospital (SMH) receive >70% of SARS-COV-2 patients in British Columbia who require hospitalization. As of Mar. 30, 2020, 56 patients have been enrolled in the two centers (VGH & SMH). Clinical care will be guided by the discretion of attending intensive care physicians in accordance with the international guidelines for COVID-19 management from the Surviving Sepsis Campaign and the British Columbia COVID-19 Therapeutics Committee recommendations. Clinical data include: date of ICU admission, sex, age, symptoms of viral illness, comorbidities, length of ICU and hospital stay, length of mechanical ventilation, development of acute respiratory distress syndrome, medications administered specific to treatment of SARS-COV-2, length of other treatment modalities. PBMCs will be collected on day 14 and day 21. We will experimentally mapping peptide epitopes in the SARS-COV-2 proteins, in a manner similar to the peptide analysis from cultured cells. We will obtain COVID-19 patient peripheral blood mononuclear cells (PBMCs) from the COVID-19 patient cohort in Vancouver. We will elute the HLA-bound SARS-COV-2 peptides from the PBMCs, and map the peptides using Mass Spectrometry.

HLA haplotype sequencing: In order to begin to understand if some HLA genes confer resistance or susceptibility to SARS-COV-2, as has been noted for other human coronaviruses, we will sequence HLA genes from COVID-19 patients. From the COVID-19 patient PBMC samples, all HLA Class I and Class II HLA genes will be sequenced at single base-pair resolution. 0.6-1.4 μg of high-quality genomic DNA (gDNA) is required for the amplification of HLA-A, HLA-B, HLA-C, HLA-DRB1/3, HLA-DRB4, HLA-DRB5, HLADPA1, HLA-DPB1, HLA-DQA1 and HLA-DQB1 regions. Each patient will be genotyped once. The next generation sequencing tool for HLA typing, Holotype HLA v3.0 (Omixon), is a set of predetermined PCR primers and reagents that provides deep and even coverage of the whole gene region for all 11 HLA class 1 and class 2 genes, with balance between alleles at the same locus. Targeted whole gene libraries will be multiplexed and sequenced on the II lumina MiSeq platform using the 300-cycle Standard flow cell. Data Analysis of fastq files will be done using the Omixon Twin v4.0.1 Software. Polymorphic sites will be identified within all the regions.

### Example 2: Creation of Hla Tetramers for Use in Vaccine Testing and Evaluation of Patient Immune Status

HLA tetramers are essentially four HLA molecules together in a tetramer, such that their peptide-binding pockets are exposed. Specific kits for making tetramers with specific HLA haplotypes will be purchased from Mbl International Corp, i.e. QuickSwitch Quant HLA-A*02:01 Tetramer Kit-APC (#TB-7300-K2). These HLA tetramer molecules can then be loaded with a relevant peptide, identified by the studies above. In a tetramer assay, these HLA-tetramers are used to detect T cell with receptors that recognize the peptide:HLA complex. The HLA tetramers are further labeled with a fluorophore (Tetramer Staining Guide, MBL Int. Corp.), allowing tetramer-bound T-cells to be analyzed with flow cytometry. This enables us to quantitate and to investigate the specificity and functionality of the cellular immune response to a SARS-COV-2 viral infection and to an eventual vaccine administration, as HLA tetramer binding to a T cell indicates that a person has encountered the pathogen previously and built an immune response to that pathogen. Mapping HLA-peptide epitopes of SARS-COV-2 proteins will allow generation of HLA Tetramers, which can then be used to quantify T cell specificity in human populations. A tetramer reagent such as this would be invaluable for monitoring patients' immune responses in real time and may inform clinical treatment and vaccine performance.

### Example 3: Creation of “String-of-Beads” Vaccines for Covid-19

Self-Amplifying mRNA (SAM) Vaccines production: | “String-of-Beads” SAM candidates vaccines will be produced asdescribed above.

Lipid nanoparticle (LNP) formulation of the RNA-based vaccines: SAM vaccine candidates will be encapsulated state-of-the-art lipid nanoparticles based on clinically approved formulations. Encapsulation uses a self-assembly process, in which an aqueous solution of mRNA at pH=4.0 is rapidly mixed with a solution of lipids dissolved in ethanol. LNPs to be used in this study are similar in composition to those described previously, which contain an ionizable cationic lipid (phosphatidylcholine:cholesterol/PEG-lipid (50:10:38.5:1.5 mol/mol)) and will be encapsulated at an RNA to total lipid ratio of ˜0.05 (wt/wt). This should result in LNPs with a diameter of ˜80 nm as measured by dynamic light scattering using a Zetasizer Nano ZS (Malvern Instruments Ltd., Malvern, UK) instrument. All SAM-LNP formulations will be stored at −80° ° C. at a concentration of mRNA of ˜1 μg/μl.

Validation of recombinant gene and protein expression: Human HEK293 cell and hamster BHK-21 cells will be transfected with the SAM-lipid nanoparticle formulations. RT-PCR and Western blots will be used to test for expression of the constructs from the vaccine candidates. Cell lysates will be run on SDS-PAGE gels and Western blots will be performed with FLAG-specific antibodies.

Testing vaccines in a hamster COVID-19 disease model. The SAM-lipid nanoparticle (SAM-LNP) vaccines will be tested in a hamster model of COVID-19. Vaccine constructs will be tested for their performance in evoking immune responses against the SARS-COV-2 spike protein and to elicit protection in a SARS-COV-2 lethal viral challenge model in Syrian hamsters.

Hamster Vaccination: SAM-LNPs vaccine candidates will be diluted in phosphate-buffered saline (PBS) and injected into animals intramuscularly (i.m.) with a 3/10 cc 29½G insulin syringe. Four sites of injection (30 μl each) over the lower back will be used. For a dose response curve, hamsters will be vaccinated (Primed) on day 0 and receive a booster injection on day 14. Groups will consist of a minimum of 5 animals per group for each vaccine tested, and a minimum of 3 dose ranges (e.g. 0.005 mg/kg-0.250 mg/kg) will be used for each vaccine23. A control group of unimmunized hamsters will be included. Body weights will be determined every day.

Measuring Cellular Immune Responses: Peripheral blood monocytic cells (PBMCs) will be obtained on day 0, 7, 14 and 28 post-infection. We will harvest PBMCs to compare lymphocyte populations (T cell: CD4+, CD8+; B cell) using hamster-specific antibodies available from the Monoclonal Antibody Center at Washington State University. Inflammatory cytokines profiling will be undertaken using ELISA kits (MBL Intl) in samples collected at day 0, 4, 7, 14, 28 after challenge. Quantitative reverse transcriptase polymerase chain reaction (qRT-PCR) will also be used to verify cytokine production from isolated cells and tissues, as previously described. Using blood obtained from the vaccinated hamsters above, we will screen for the production of antibodies which bind to recombinant SARS-COV-2 proteins, using screening assays, e.g. Western blot; ELISA assay; EliSpot.

Viral Challenge: The optimal dose of vaccine to use will be determined from the dose response trial. Hamsters will be vaccinated (Primed) on day 0 and receive a booster injection on day 14. Groups will consist of a minimum of 15 animals per group for each vaccine tested. At 28 days post vaccination, hamsters will be challenged with SARS-COV-2 at a dose of 10 e5 pfu in roughly 30 μl PBS via intranasal administration. Hamsters will be monitored every day for temperature, weight, and survival. Losing over 20% of their body weight will be considered a humane endpoint, and animals will be sacrificed. Tissues and cells will be harvested and examined, as described above, and bronchoalveolar lavage cell suspension will also be obtained at time of mortality.

### Example 4: Combinatorially Identified Epitopes and Experimental Data

There appears to be a good concordance between the epitopes identified by the method described in PCT/CA2021/051666 (incorporated by reference) and those found experimentally in infection survivors. With typical parameters, the method described in PCT/CA2021/051666 would identify 251 highly immunogenic loci that are needed to confer a broad population-wide immunity against SARS-CoV-2, and 113 of those (or the 63% of the 180 detected ones) correspond with peptides that have been identified in survivors. It should also be noted that the dataset of detected peptides is by no means exhaustive as it is derived from a sample of survivors that is not representative of the general population, so more of the peptides predicted by PCT/CA2021/0516 might in fact be present in the general population. A pre-gene breakdown of the results is included in the following Table.

| | Predicted by | Not predicted by | Predicted by | |
|---|---|---|---|---|
| | PCT/ | PCT/ | PCT/ | |
| | CA2021/ | CA2021/ | CA2021/ | |
| | 051666 | 051666 | 051666 | |
| | and | and | and not | |
| | experim. | experim. | experim. | |
| Gene | detected | detected | detected | TOTAL |
| surface_glycoprotein | 26 | 7 | 13 | 46 |
| | (57% of total, | (15% of total, | (28% of total) | |
| | 79% of detected) | 21% of detected) | | |
| nucleocapsid_phosphoprotein | 7 | 7 | 2 | 16 |
| | (44% of total. | (44% of total, | (13% of total) | |
| | 50% of detected) | 50% of detected) | | |
| nsp6 | 5 | 1 | 0 | 6 |
| | (83% of total, | (17% of total, | (0% of total) | |
| | 83% of detected) | 17% of detected) | | |
| membrane_glycoprotein | 4 | 2 | 1 | 7 |
| | (57% of total, | (29% of total, | (14% of total) | |
| | 67% of detected) | 33% of detected) | | |
| ORF3a_protein | 4 | 1 | 2 | 7 |
| | (57% of total, | (14% of total, | | |
| | 80% of detected) | 20% of detected) | (29% of total) | |
| 3′-to-5′_exonuclease | 9 | 4 | 5 | 18 |
| | (50% of total, | (22% of total, | (28% of total) | |
| | 69% of detected) | 31% of detected) | | |
| RNA-dependent_RNA_polymerase | 18 | 7 | 10 | 35 |
| | (51% of total, | (20% of total, | (29% of total) | |
| | 72% of detected) | 28% of detected) | | |
| ORF8_protein | 3 | 0 | 1 | 4 |
| | (75% of total, | (0% of total, | (25% of total) | |
| | 100% of detected) | 0% of detected) | | |
| nsp2 | 11 | 7 | 7 | 25 |
| | (44% of total, | (28% of total, | (28% of total) | |
| | 61% of detected) | 39% of detected) | | |
| helicase | 8 | 2 | 8 | 18 |
| | (44% of total, | (11% of total, | (44% of total) | |
| | 80% of detected) | 20% of detected) | | |
| nsp3 | 18 | 29 | 22 | 69 |
| | (26% of total, | (42% of total, | (32% of total) | |
| | 38% of detected) | 62% of detected) | | |
| TOTAL | 113 | 67 | 71 | 251 |
| | (45% of total, | (27% of total, | (28% of total) | |
| | 63% of detected) | 37% of detected) | | |

In more detail, this is the full list of viral loci, with whether they have been predicted/detected:

| | | | Identification |
|---|---|---|---|
| | | | (J = PCT/ |
| | | | CA2021/ |
| | | | 051666; |
| | | | E = |
| | | | experimentally |
| | Low | High | detected |
| | AA | AA | peptides; |
| Viral gene | position | position | J, E = both) |
| 3′-to-5′_exonuclease | 1 | 14 | J, E |
| 3′-to-5′_exonuclease | 16 | 30 | J |
| 3′-to-5′_exonuclease | 64 | 72 | E |
| 3′-to-5′_exonuclease | 102 | 125 | J, E |
| 3′-to-5′_exonuclease | 156 | 199 | J, E |
| 3′-to-5′_exonuclease | 205 | 219 | J |
| 3′-to-5′_exonuclease | 223 | 237 | J |
| 3′-to-5′_exonuclease | 240 | 249 | E |
| 3′-to-5′_exonuclease | 252 | 291 | J, E |
| 3′-to-5′_exonuclease | 305 | 319 | J |
| 3′-to-5′_exonuclease | 320 | 328 | E |
| 3′-to-5′_exonuclease | 337 | 351 | J, E |
| 3′-to-5′_exonuclease | 363 | 381 | J, E |
| 3′-to-5′_exonuclease | 394 | 409 | J |
| 3′-to-5′_exonuclease | 411 | 419 | E |
| 3′-to-5′_exonuclease | 428 | 450 | J, E |
| 3′-to-5′_exonuclease | 454 | 476 | J, E |
| 3′-to-5′_exonuclease | 492 | 512 | J, E |
| helicase | 1 | 39 | J, E |
| helicase | 41 | 92 | J, E |
| helicase | 128 | 135 | J |
| helicase | 146 | 169 | J, E |
| helicase | 173 | 181 | E |
| helicase | 205 | 213 | J |
| helicase | 218 | 270 | J, E |
| helicase | 289 | 375 | J, E |
| helicase | 382 | 390 | J |
| helicase | 392 | 411 | J, E |
| helicase | 422 | 430 | J |
| helicase | 438 | 447 | J |
| helicase | 448 | 456 | E |
| helicase | 478 | 487 | J |
| helicase | 492 | 499 | J |
| helicase | 507 | 533 | J, E |
| helicase | 566 | 579 | J |
| helicase | 582 | 600 | J, E |
| membrane_glycoprotein | 19 | 73 | J, E |
| membrane_glycoprotein | 82 | 116 | J, E |
| membrane_glycoprotein | 119 | 132 | J |
| membrane_glycoprotein | 136 | 159 | J, E |
| membrane_glycoprotein | 161 | 196 | J, E |
| membrane_glycoprotein | 198 | 206 | E |
| membrane_glycoprotein | 213 | 22 | E |
| nsp2 | 14 | 24 | J, E |
| nsp2 | 30 | 49 | J, E |
| nsp2 | 61 | 81 | J, E |
| nsp2 | 86 | 108 | J, E |
| nsp2 | 112 | 120 | E |
| nsp2 | 121 | 129 | J |
| nsp2 | 156 | 163 | J |
| nsp2 | 178 | 198 | J, E |
| nsp2 | 199 | 207 | J, E |
| nsp2 | 216 | 224 | E |
| nsp2 | 239 | 248 | J |
| nsp2 | 265 | 273 | E |
| nsp2 | 288 | 326 | J, E |
| nsp2 | 347 | 367 | J |
| nsp2 | 382 | 401 | J |
| nsp2 | 403 | 412 | E |
| nsp2 | 413 | 420 | J |
| nsp2 | 429 | 450 | J, E |
| nsp2 | 461 | 487 | J, E |
| nsp2 | 492 | 513 | J, E |
| nsp2 | 522 | 536 | J, E |
| nsp2 | 551 | 559 | E |
| nsp2 | 561 | 569 | E |
| nsp2 | 598 | 610 | J |
| nsp2 | 625 | 636 | E |
| nsp3 | 1 | 27 | J, E |
| nsp3 | 42 | 50 | E |
| nsp3 | 53 | 68 | J, E |
| nsp3 | 81 | 105 | J, E |
| nsp3 | 182 | 201 | E |
| nsp3 | 239 | 249 | J |
| nsp3 | 265 | 273 | E |
| nsp3 | 325 | 338 | E |
| nsp3 | 339 | 348 | J |
| nsp3 | 351 | 364 | J, E |
| nsp3 | 367 | 375 | E |
| nsp3 | 396 | 404 | E |
| nsp3 | 415 | 424 | J |
| nsp3 | 430 | 452 | J, E |
| nsp3 | 454 | 476 | J, E |
| nsp3 | 494 | 502 | E |
| nsp3 | 503 | 511 | E |
| nsp3 | 525 | 542 | J |
| nsp3 | 569 | 579 | J, E |
| nsp3 | 595 | 608 | J |
| nsp3 | 615 | 627 | E |
| nsp3 | 628 | 639 | J |
| nsp3 | 647 | 655 | E |
| nsp3 | 660 | 671 | J |
| nsp3 | 684 | 691 | J |
| nsp3 | 718 | 728 | E |
| nsp3 | 733 | 741 | E |
| nsp3 | 748 | 756 | E |
| nsp3 | 810 | 817 | J |
| nsp3 | 819 | 832 | J, E |
| nsp3 | 851 | 859 | E |
| nsp3 | 867 | 882 | J |
| nsp3 | 890 | 904 | J, E |
| nsp3 | 950 | 958 | E |
| nsp3 | 963 | 973 | J |
| nsp3 | 980 | 994 | J, E |
| nsp3 | 995 | 1006 | J |
| nsp3 | 1012 | 1021 | J |
| nsp3 | 1073 | 1089 | E |
| nsp3 | 1112 | 1120 | E |
| nsp3 | 1131 | 1161 | J, E |
| nsp3 | 1165 | 1176 | J |
| nsp3 | 1199 | 1207 | E |
| nsp3 | 1236 | 1268 | J, E |
| nsp3 | 1274 | 1289 | E |
| nsp3 | 1291 | 1299 | E |
| nsp3 | 1303 | 1312 | E |
| nsp3 | 1327 | 1335 | E |
| nsp3 | 1342 | 1359 | J, E |
| nsp3 | 1383 | 1400 | J, E |
| nsp3 | 1404 | 1432 | E |
| nsp3 | 1452 | 1481 | J, E |
| nsp3 | 1483 | 1494 | J |
| nsp3 | 1508 | 1523 | E |
| nsp3 | 1529 | 1565 | E |
| nsp3 | 1574 | 1582 | E |
| nsp3 | 1587 | 1595 | E |
| nsp3 | 1626 | 1645 | J |
| nsp3 | 1652 | 1659 | J |
| nsp3 | 1665 | 1673 | E |
| nsp3 | 1697 | 1706 | J |
| nsp3 | 1718 | 1725 | J |
| nsp3 | 1737 | 1759 | J, E |
| nsp3 | 1771 | 1794 | E |
| nsp3 | 1809 | 1819 | J, E |
| nsp3 | 1830 | 1843 | J |
| nsp3 | 1866 | 1881 | J, E |
| nsp3 | 1885 | 1897 | J |
| nsp3 | 1923 | 1942 | J |
| nsp6 | 5 | 13 | E |
| nsp6 | 24 | 124 | J, E |
| nsp6 | 139 | 155 | J, E |
| nsp6 | 160 | 234 | J, E |
| nsp6 | 236 | 249 | J, E |
| nsp6 | 268 | 286 | J, E |
| nucleocapsid_phosphoprotein | 11 | 19 | J, E |
| nucleocapsid_phosphoprotein | 41 | 59 | J, E |
| nucleocapsid_phosphoprotein | 75 | 87 | E |
| nucleocapsid_phosphoprotein | 93 | 101 | E |
| nucleocapsid_phosphoprotein | 104 | 115 | J, E |
| nucleocapsid_phosphoprotein | 128 | 136 | J |
| nucleocapsid_phosphoprotein | 138 | 146 | E |
| nucleocapsid_phosphoprotein | 150 | 158 | E |
| nucleocapsid_phosphoprotein | 189 | 196 | E |
| nucleocapsid_phosphoprotein | 217 | 234 | J, E |
| nucleocapsid_phosphoprotein | 257 | 265 | E |
| nucleocapsid_phosphoprotein | 266 | 274 | J, E |
| nucleocapsid_phosphoprotein | 289 | 298 | E |
| nucleocapsid_phosphoprotein | 299 | 316 | J, E |
| nucleocapsid_phosphoprotein | 323 | 369 | J, E |
| nucleocapsid_phosphoprotein | 375 | 386 | J |
| ORF3a_protein | 2 | 10 | E |
| ORF3a_protein | 25 | 65 | J, E |
| ORF3a_protein | 70 | 134 | J, E |
| ORF3a_protein | 136 | 175 | J, E |
| ORF3a_protein | 176 | 188 | J |
| ORF3a_protein | 194 | 249 | J, E |
| ORF3a_protein | 252 | 262 | J |
| ORF8_protein | 6 | 52 | J, E |
| ORF8_protein | 54 | 64 | J |
| ORF8_protein | 73 | 93 | J, E |
| ORF8_protein | 95 | 118 | J, E |
| RNA-dependent_RNA_polymerase | 4 | 20 | J |
| RNA-dependent_RNA_polymerase | 28 | 35 | J |
| RNA-dependent_RNA_polymerase | 64 | 74 | J, E |
| RNA-dependent_RNA_polymerase | 83 | 104 | J, E |
| RNA-dependent_RNA_polymerase | 122 | 133 | J, E |
| RNA-dependent_RNA_polymerase | 142 | 155 | J |
| RNA-dependent_RNA_polymerase | 183 | 196 | J |
| RNA-dependent_RNA_polymerase | 229 | 256 | J, E |
| RNA-dependent_RNA_polymerase | 263 | 271 | E |
| RNA-dependent_RNA_polymerase | 295 | 329 | J, E |
| RNA-dependent_RNA_polymerase | 331 | 346 | J, E |
| RNA-dependent_RNA_polymerase | 348 | 363 | J |
| RNA-dependent_RNA_polymerase | 365 | 382 | J, E |
| RNA-dependent_RNA_polymerase | 394 | 410 | J, E |
| RNA-dependent_RNA_polymerase | 420 | 427 | J |
| RNA-dependent_RNA_polymerase | 434 | 443 | J |
| RNA-dependent_RNA_polymerase | 450 | 487 | J, E |
| RNA-dependent_RNA_polymerase | 523 | 530 | J |
| RNA-dependent_RNA_polymerase | 536 | 548 | E |
| RNA-dependent_RNA_polymerase | 557 | 564 | J |
| RNA-dependent_RNA_polymerase | 574 | 589 | E |
| RNA-dependent_RNA_polymerase | 603 | 618 | J, E |
| RNA-dependent_RNA_polymerase | 624 | 678 | J, E |
| RNA-dependent_RNA_polymerase | 681 | 689 | E |
| RNA-dependent_RNA_polymerase | 691 | 708 | J, E |
| RNA-dependent_RNA_polymerase | 717 | 734 | J |
| RNA-dependent_RNA_polymerase | 738 | 752 | J, E |
| RNA-dependent_RNA_polymerase | 754 | 766 | J, E |
| RNA-dependent_RNA_polymerase | 771 | 792 | J, E |
| RNA-dependent_RNA_polymerase | 793 | 801 | E |
| RNA-dependent_RNA_polymerase | 810 | 845 | J, E |
| RNA-dependent_RNA_polymerase | 854 | 871 | J, E |
| RNA-dependent_RNA_polymerase | 877 | 893 | J, E |
| RNA-dependent_RNA_polymerase | 895 | 915 | E |
| RNA-dependent_RNA_polymerase | 926 | 932 | E |
| surface_glycoprotein | 2 | 19 | J, E |
| surface_glycoprotein | 24 | 36 | E |
| surface_glycoprotein | 38 | 54 | J, E |
| surface_glycoprotein | 55 | 74 | J, E |
| surface_glycoprotein | 78 | 93 | J |
| surface_glycoprotein | 109 | 117 | E |
| surface_glycoprotein | 123 | 146 | J |
| surface_glycoprotein | 151 | 160 | E |
| surface_glycoprotein | 162 | 179 | J, E |
| surface_glycoprotein | 202 | 250 | J, E |
| surface_glycoprotein | 256 | 278 | J, E |
| surface_glycoprotein | 286 | 293 | J |
| surface_glycoprotein | 298 | 311 | J |
| surface_glycoprotein | 318 | 337 | J, E |
| surface_glycoprotein | 343 | 382 | J, E |
| surface_glycoprotein | 387 | 404 | J |
| surface_glycoprotein | 411 | 419 | E |
| surface_glycoprotein | 424 | 436 | J, E |
| surface_glycoprotein | 445 | 459 | E |
| surface_glycoprotein | 463 | 528 | J, E |
| surface_glycoprotein | 582 | 600 | J |
| surface_glycoprotein | 601 | 629 | J, E |
| surface_glycoprotein | 635 | 654 | J, E |
| surface_glycoprotein | 657 | 671 | E |
| surface_glycoprotein | 680 | 706 | J, E |
| surface_glycoprotein | 710 | 733 | J, E |
| surface_glycoprotein | 735 | 742 | J |
| surface_glycoprotein | 746 | 767 | J, E |
| surface_glycoprotein | 780 | 794 | J, E |
| surface_glycoprotein | 801 | 815 | J, E |
| surface_glycoprotein | 821 | 829 | E |
| surface_glycoprotein | 835 | 844 | J |
| surface_glycoprotein | 846 | 855 | J |
| surface_glycoprotein | 857 | 881 | J, E |
| surface_glycoprotein | 882 | 906 | J, E |
| surface_glycoprotein | 912 | 927 | J, E |
| surface_glycoprotein | 928 | 955 | J, E |
| surface_glycoprotein | 958 | 1016 | J, E |
| surface_glycoprotein | 1039 | 1071 | J, E |
| surface_glycoprotein | 1082 | 1092 | J |
| surface_glycoprotein | 1096 | 1133 | J, E |
| surface_glycoprotein | 1138 | 1145 | J |
| surface_glycoprotein | 1152 | 1160 | J |
| surface_glycoprotein | 1183 | 1203 | J, E |
| surface_glycoprotein | 1208 | 1262 | J, E |
| surface_glycoprotein | 1263 | 1270 | J |

It should also be noted that, according to the contents of the current invention and depending on the viral gene, the method described in PCT/CA2021/0516 was used to predict peptides coming from both the wild-type virus and from variants. A table detailing the viral loci, the corresponding number of wild-type and variant peptides, and the corresponding number of HLA I & II alleles that are expected to be protected by these specific sets of peptides, is enclosed below.

| | # | # alleles | # peptides | # alleles |
|---|---|---|---|---|
| | wild-type | protected | from | protected |
| Gene | peptides | by w.t.p. | variants | by p.f.v. |
| surface_glycoprotein | 30 | 122 | 80 | 122 |
| nucleocapsid_ | 3 | 99 | 16 | 103 |
| phosphoprotein | | | | |
| nsp6 | 2 | 69 | 23 | 122 |
| endoRNAse | 10 | 85 | — | — |
| nsp7 | 2 | 54 | — | — |
| 3C-like_proteinase | 8 | 96 | — | — |
| nsp8 | 7 | 110 | — | — |
| membrane_glycoprotein | 5 | 108 | 11 | 122 |
| nsp9 | 4 | 77 | — | — |
| ORF3a_protein | 6 | 90 | 44 | 122 |
| nsp10 | 4 | 84 | — | — |
| leader_protein | 3 | 100 | — | — |
| 3′-to-5′_exonuclease | 17 | 122 | — | — |
| RNA-dependent_ | 22 | 122 | 34 | 122 |
| RNA_polymerase | | | | |
| ORF8_protein | 3 | 86 | 11 | 115 |
| ORF6_protein | 3 | 103 | — | — |
| 2′-O-ribose_ | 8 | 110 | — | — |
| methyltransferase | | | | |
| ORF7a_protein | 3 | 91 | — | — |
| nsp2 | 18 | 122 | — | — |
| envelope_protein | 1 | 81 | — | — |
| nsp3 | 41 | 119 | — | — |
| helicase | 14 | 122 | 38 | 122 |
| nsp4 | 10 | 106 | — | — |

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Titles, headings, or the like are provided to enhance the reader's comprehension of this document, and should not be read as limiting the scope of the present invention.

We claim:

1. A vaccine comprising or capable of expressing one or more concatemers of epitopes from a viral pathogen; such as a sarbecovirus pathogen, said vaccine optionally further comprising an adjuvant.

2. The vaccine of claim 1, wherein said vaccine comprises or expresses epitopes for all MHC I and MHC II alleles with a frequency >1% in the target population; optionally the population is a geographically restricted population and optionally epitopes that bind MHC I or MHC II alleles associated with autoimmune disease are excluded.

3. (canceled)

4. (canceled)

5. The vaccine of claim 1, wherein at least a portion of said epitopes are from conserved proteins from said viral pathogen, are universal (common) epitopes of said viral pathogen or variant specific epitopes.

6. (canceled)

7. (canceled)

8. The vaccine of claim 1, wherein a linker separates each of said epitopes.

9. (canceled)

10. The vaccine of claim 1, wherein at least one of the one or more epitopes is as set forth in SEQ ID NOs 1-789 or set forth in SEQ ID NOs 798-851.

11. A vaccine comprising or capable of expressing one or more concatemers of epitopes set forth in SEQ ID NOs 1-789 or SEQ ID NOs 798-851.

12. (canceled)

13. The vaccine of claim 1, wherein said vaccine is a viral vector-based vaccine.

14. The vaccine of claim 13, wherein the viral vector is an adenoviral vector, a vesicular stomatitis virus vector or a vaccinia vector.

15. The vaccine of claim 1, wherein said vaccine is a nucleic acid-based vaccine.

16. The vaccine of claim 15, wherein said vaccine is a SAM RNA-based vaccine.

17. The vaccine of claim 16, wherein said SAM RNA-based vaccine is encapsulated in a lipid nanoparticle (LNP).

18. The vaccine of claim 17, wherein the LNP comprises a cationic lipid.

19. The vaccine of claim 17, wherein the LNP comprises phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA).

20. (canceled)

21. A method of treating, protecting against, and/or preventing infection by a target viral pathogen or generating an immune response against said target viral pathogen in a subject in need thereof, said method comprising administering one or more of the vaccines of claim 1 to the subject, wherein optionally said viral pathogen is a sarbecovirus such as SARS-COV-2.

22. (canceled)

23. The method of claim 1, wherein said vaccine is administered more than once, optionally wherein a prime and boost strategy of vaccination is utilized.

24. (canceled)

25. The method of claim 21, wherein the subject is a mammal, optionally a human, a cat, dog, horse, sheep, goat, camel or cow.

26. (canceled)

27. (canceled)

28. (canceled)

29. (canceled)
